SEQUENCE LISTING (221 sequences)

SEQ ID NO: 1 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 2 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 3 - protein, 391 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGSCCVADWQMPPPYVVLDLPQETLEEETPGANLWPTTITFLTLFLLSLFYSTALTVTSVRGPSGKREGPQY

SEQ ID NO: 4 - protein, 338 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVQVSVVMAEVDGT

SEQ ID NO: 5 - protein, 322 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEANLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 6 - protein, 322 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEAGLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 7 - protein, 322 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEAQLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 8 - protein, 322 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEATLTCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 9 - protein, 322 aa, Homo sapiens
ASPTSPKVFPLSLDSTPQDGNVVVACLVQGFFPQEPLSVTWSESGQGVTARNFPPSQDASGDLYTTSSQLTLPATQCPDGKSVTCHVKHYTNPSQDVTVPCRVPPPPPCCHPRLSLHRPALEDLLLGSEATISCTLTGLRDASGATFTWTPSSGKSAVQGPPERDLCGCYSVSSVLPGSAQPWNHGETFTCTAAHPELKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 10 - DNA, 1020 bp, Homo sapiens
  source 1..1020: mol_type = genomic DNA
gccagccccaccagccccaaggtgttccccctgagcctggacagcaccccccaggacggcaacgtggtggtggcctgcctggtgcagggcttcttcccccaggagcccctgagcgtgacctggagcgagagcggccagaacgtgaccgccaggaacttcccccccagccaggacgccagcggcgacctgtacaccaccagcagccagctgaccctgcccgccacccagtgccccgacggcaagagcgtgacctgccacgtgaagcactacaccaaccccagccaggacgtgaccgtgccctgccccgtgccccccccccccccctgctgccaccccaggctgagcctgcacaggcccgccctggaggacctgctgctgggcagcgaggccaacctgacctgcaccctgaccggcctgagggacgccagcggcgccaccttcacctggacccccagcagcggcaagagcgccgtgcagggcccccccgagagggacctgtgcggctgctacagcgtgagcagcgtgctgcccggctgcgcccagccctggaaccacggcgagaccttcacctgcaccgccgcccaccccgagctgaagacccccctgaccgccaacatcaccaagagcggcaacaccttcaggcccgaggtgcacctgctgcccccccccagcgaggagctggccctgaacgagctggtgaccctgacctgcctggccaggggcttcagccccaaggacgtgctggtgaggtggctgcagggcagccaggagctgcccagggagaagtacctgacctgggccagcaggcaggagcccagccagggcaccaccaccttcgccgtgaccagcatcctgagggtggccgccgaggactggaagaagggcgacaccttcagctgcatggtgggccacgaggccctgcccctggccttcacccagaagaccatcgacaggctggccggcaagcccacccacgtgaacgtgagcgtggtgatggccgaggtggacggcacctgctac

SEQ ID NO: 11 - DNA, 1020 bp, Homo sapiens
  source 1..1020: mol_type = genomic DNA
gccagccccaccagccccaaggtgttccccctgagcctggacagcaccccccaggacggcaacgtggtggtggcctgcctggtgcagggcttcttcccccaggagcccctgagcgtgacctggagcgagagcggccagaacgtgaccgccaggaacttcccccccagccaggacgccagcggcgacctgtacaccaccagcagccagctgaccctgcccgccacccagtgccccgacggcaagagcgtgacctgccacgtgaagcactacaccaacagcagccaggacgtgaccgtgccctgcagggtgccccccccccccccctgctgccaccccaggctgagcctgcacaggcccgccctggaggacctgctgctgggcagcgaggccaacctgacctgcaccctgaccggcctgagggacgccagcggcgccaccttcacctggacccccagcagcggcaagagcgccgtgcagggcccccccgagagggacctgtgcggctgctacagcgtgagcagcgtgctgcccggctgcgcccagccctggaaccacggcgagaccttcacctgcaccgccgcccaccccgagctgaagacccccctgaccgccaacatcaccaagagcggcaacaccttcaggcccgaggtgcacctgctgcccccccccagcgaggagctggccctgaacgagctggtgaccctgacctgcctggccaggggcttcagccccaaggacgtgctggtgaggtggctgcagggcagccaggagctgcccagggagaagtacctgacctgggccagcaggcaggagcccagccagggcaccaccacctacgccgtgaccagcatcctgagggtggccgccgaggactggaagaagggcgagaccttcagctgcatggtgggccacgaggccctgcccctggccttcacccagaagaccatcgacaggatggccggcaagcccacccacatcaacgtgagcgtggtgatggccgaggccgacggcacctgctac

SEQ ID NO: 12 - DNA, 1173 bp, Homo sapiens
  source 1..1173: mol_type = genomic DNA
gccagccccaccagccccaaggtgttccccctgagcctggacagcaccccccaggacggcaacgtggtggtggcctgcctggtgcagggcttcttcccccaggagcccctgagcgtgacctggagcgagagcggccagaacgtgaccgccaggaacttcccccccagccaggacgccagcggcgacctgtacaccaccagcagccagctgaccctgcccgccacccagtgccccgacggcaagagcgtgacctgccacgtgaagcactacaccaacagcagccaggacgtgaccgtgccctgcagggtgccccccccccccccctgctgccaccccaggctgagcctgcacaggcccgccctggaggacctgctgctgggcagcgaggccaacctgacctgcaccctgaccggcctgagggacgccagcggcgccaccttcacctggacccccagcagcggcaagagcgccgtgcagggcccccccgagagggacctgtgcggctgctacagcgtgagcagcgtgctgcccggctgcgcccagccctggaaccacggcgagaccttcacctgcaccgccgcccaccccgagctgaagacccccctgaccgccaacatcaccaagagcggcaacaccttcaggcccgaggtgcacctgctgcccccccccagcgaggagctggccctgaacgagctggtgaccctgacctgcctggccaggggcttcagccccaaggacgtgctggtgaggtggctgcagggcagccaggagctgcccagggagaagtacctgacctgggccagcaggcaggagcccagccagggcaccaccacctacgccgtgaccagcatcctgagggtggccgccgaggactggaagaagggcgagaccttcagctgcatggtgggccacgaggccctgcccctggccttcacccagaagaccatcgacaggatggccggcagctgctgcgtggccgactggcagatgccccccccctacgtggtgctggacctgccccaggagaccctggaggaggagacccccggcgccaacctgtggcccaccaccatcaccttcctgaccctgttcctgctgagcctgttctacagcaccgccctgaccgtgaccagcgtgaggggccccagcggcaagagggagggcccccagtac

SEQ ID NO: 13 - DNA, 1014 bp, Homo sapiens
  source 1..1014: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaagcccacccatgtccaagtgtctgttgtcatggcggaggtggacggcacc

SEQ ID NO: 14 - DNA, 966 bp, Homo sapiens
  source 1..966: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccaacctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 15 - DNA, 966 bp, Homo sapiens
  source 1..966: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccggcctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 16 - DNA, 966 bp, Homo sapiens
  source 1..966: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggcccagctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 17 - DNA, 966 bp, Homo sapiens
  source 1..966: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccaccctgacatgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 18 - DNA, 966 bp, Homo sapiens
  source 1..966: mol_type = genomic DNA
gctagcccaacctctcctaaggtgttccctctgagcctggacagcacccctcaggatggaaatgtggtggtggcctgtctggtgcagggattcttcccacaagagcccctgtccgtgacttggagcgaatctggacagggcgtgaccgccagaaacttcccaccttctcaggacgcctctggcgacctgtacaccacctcttctcagctgaccctgcctgccacacagtgccctgatggcaagtctgtgacctgccacgtgaagcactacaccaatcctagccaggacgtgaccgtgccttgcagagttcctcctcctccaccttgctgtcaccctcggctgtctctgcacagacccgctctggaagatctgctgctgggctctgaggccaccatcagctgtaccctgaccggcctgagagatgcttctggcgccacctttacctggacaccttccagcggaaagtccgctgttcagggacctcctgagagggacctgtgcggctgttactctgtgtctagtgtgctgcctggcagcgcccagccttggaatcatggcgagacattcacctgtaccgctgctcaccccgagctgaaaacccctctgaccgccacactgtccaagtccggcaacaccttccggcctgaagtgcatctgctgcctccacctagcgaggaactggccctgaatgagctggtcaccctgacctgtctggccaggggctttagccctaaggacgtgctcgttagatggctgcagggctcccaagagctgcccagagagaagtatctgacctgggcctctcggcaagagccatctcagggcaccacaacctttgccgtgaccagcatcctgagagtggccgccgaagattggaagaagggcgacaccttcagctgcatggtcggacatgaagccctgcctctggctttcacccagaaaaccatcgacagactggccggcaag

SEQ ID NO: 19 - skipped

SEQ ID NO: 20 - protein, 102 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCP

SEQ ID NO: 21 - protein, 101 aa, Homo sapiens
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 22 - protein, 131 aa, Homo sapiens
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 23 - protein, 107 aa, synthetic construct [sequence omitted]

SEQ ID NO: 24 - protein, 322 aa, synthetic construct; identical to SEQ ID NO: 9

SEQ ID NO: 25-32 - skipped (8 entries)

SEQ ID NO: 33 - protein, 9 aa, synthetic construct
CYGGRGDTP

SEQ ID NO: 34 - protein, 8 aa, synthetic construct
GFTFSTYG

SEQ ID NO: 35 - protein, 8 aa, synthetic construct
GYTFTSYG

SEQ ID NO: 36 - protein, 8 aa, synthetic construct
GFSLSNYD

SEQ ID NO: 37 - protein, 8 aa, synthetic construct
GFSLTNYG

SEQ ID NO: 38 - protein, 10 aa, synthetic construct
GGSVSSGDYY

SEQ ID NO: 39 - protein, 9 aa, synthetic construct
GYSISSDFA

SEQ ID NO: 40 - protein, 8 aa, synthetic construct
GFTFRNYG

SEQ ID NO: 41 - protein, 8 aa, synthetic construct
GFTFNKYA

SEQ ID NO: 42 - protein, 8 aa, synthetic construct
GYTFTSYW

SEQ ID NO: 43 - protein, 8 aa, synthetic construct
GFTFTDYK

SEQ ID NO: 44 - protein, 9 aa, synthetic construct
GYSISRDFA

SEQ ID NO: 45 - protein, 8 aa, synthetic construct
GYTFTSHW

SEQ ID NO: 46 - protein, 10 aa, synthetic construct
GGSISSGDYY

SEQ ID NO: 47 - protein, 8 aa, synthetic construct
GYTFTNYY

SEQ ID NO: 48 - protein, 8 aa, synthetic construct
GYDFTNYA

SEQ ID NO: 49 - protein, 10 aa, synthetic construct
GGSVSSGSYY

SEQ ID NO: 50 - protein, 5 aa, synthetic construct
HYDMS

SEQ ID NO: 51 - protein, 7 aa, synthetic construct
GFAFSHY

SEQ ID NO: 52 - protein, 5 aa, synthetic construct
EYPIH

SEQ ID NO: 53 - protein, 7 aa, synthetic construct
GYTFTEY

SEQ ID NO: 54 - protein, 6 aa, synthetic construct
TRYWLH

SEQ ID NO: 55-56 - skipped (2 entries)

SEQ ID NO: 57 - protein, 8 aa, synthetic construct
IWDDGSYK

SEQ ID NO: 58 - protein, 8 aa, synthetic construct
ISASNGNT

SEQ ID NO: 59 - protein, 7 aa, synthetic construct
IWSGGNT

SEQ ID NO: 60 - protein, 7 aa, synthetic construct
IYYSGNT

SEQ ID NO: 61 - protein, 7 aa, synthetic construct
ISYSGNT

SEQ ID NO: 62 - protein, 8 aa, synthetic construct
IWYDGSDK

SEQ ID NO: 63 - protein, 10 aa, synthetic construct
IRSKYNNYAT

SEQ ID NO: 64 - protein, 8 aa, synthetic construct
IYPGSRST

SEQ ID NO: 65 - protein, 8 aa, synthetic construct
FNPNSGYS

SEQ ID NO: 66 - protein, 8 aa, synthetic construct
IYPGDGDT

SEQ ID NO: 67 - protein, 7 aa, synthetic construct
ISYNGNT

SEQ ID NO: 68 - protein, 8 aa, synthetic construct
FNPSNGRT

SEQ ID NO: 69 - protein, 8 aa, synthetic construct
INPSSGRN

SEQ ID NO: 70 - protein, 7 aa, synthetic construct
IYYSGST

SEQ ID NO: 71 - protein, 8 aa, synthetic construct
INPTSGGS

SEQ ID NO: 72 - protein, 16 aa, synthetic construct
HIYYSGNTNYNPSLKS

SEQ ID NO: 73 - protein, 8 aa, synthetic construct
INANTGDP

SEQ ID NO: 74 - protein, 6 aa, synthetic construct
ASGGDI

SEQ ID NO: 75 - protein, 17 aa, synthetic construct
YIASGGDITYYADTVKG

SEQ ID NO: 76 - protein, 6 aa, synthetic construct
YTDIGK

SEQ ID NO: 77 - protein, 15 aa, synthetic construct
YTDIGKPTYAEEFKG

SEQ ID NO: 78 - protein, 17 aa, synthetic construct
EINPSSGRTNYNEKFKS

SEQ ID NO: 79-80 - skipped (2 entries)

SEQ ID NO: 81 - protein, 18 aa, synthetic construct
ARDGITMVRGVMKDYFDY

SEQ ID NO: 82 - protein, 10 aa, synthetic construct
ARVYADYADY

SEQ ID NO: 83 - protein, 13 aa, synthetic construct
ARALDYYDYEFAY

SEQ ID NO: 84 - protein, 13 aa, synthetic construct
ARALTYYDYEFAY

SEQ ID NO: 85 - protein, 11 aa, synthetic construct
VRDRVTGAFDI

SEQ ID NO: 86 - protein, 9 aa, synthetic construct
VTAGRGFPY

SEQ ID NO: 87 - protein, 17 aa, synthetic construct
ARDGYDILTGNPRDFDY

SEQ ID NO: 88 - protein, 16 aa, synthetic construct
VRHGNFGNSYISYWAY

SEQ ID NO: 89 - protein, 16 aa, synthetic construct
TRNGDYYVSSGDAMDY

SEQ ID NO: 90 - protein, 13 aa, synthetic construct
ARLSPGGYYVMDA

SEQ ID NO: 91 - protein, 12 aa, synthetic construct
ARYDAPGYAMDY

SEQ ID NO: 92 - protein, 9 aa, synthetic construct
VTASRGFPY

SEQ ID NO: 93 - protein, 14 aa, synthetic construct
ASRDYDYDGRYFDY

SEQ ID NO: 94 - protein, 12 aa, synthetic construct
VRYYGYDEAMDY

SEQ ID NO: 95 - protein, 13 aa, synthetic construct
ARVSIFGVGTFDY

SEQ ID NO: 96 - protein, 16 aa, synthetic construct
ARQGLWFDSDGRGFDF

SEQ ID NO: 97 - protein, 9 aa, synthetic construct
DRVTGAFDI

SEQ ID NO: 98 - protein, 13 aa, synthetic construct
TRERFLEWLHFDY

SEQ ID NO: 99 - protein, 12 aa, synthetic construct
ARNPISIPAFDI

SEQ ID NO: 100 - protein, 12 aa, synthetic construct
SSYGNNGDALDF

SEQ ID NO: 101 - protein, 9 aa, synthetic construct
DRYDSLFDY

SEQ ID NO: 102 - protein, 11 aa, synthetic construct
WDYDEGTWFAY

SEQ ID NO: 103-104 - skipped (2 entries)

SEQ ID NO: 105 - protein, 6 aa, synthetic construct
QDISSA

SEQ ID NO: 106 - protein, 6 aa, synthetic construct
QGINTW

SEQ ID NO: 107 - protein, 6 aa, synthetic construct
QSIGTN

SEQ ID NO: 108 - protein, 6 aa, synthetic construct
QDISNY

SEQ ID NO: 109 - protein, 6 aa, synthetic construct
QDINSN

SEQ ID NO: 110 - protein, 11 aa, synthetic construct
QSLVHSDGNTY

SEQ ID NO: 111 - protein, 9 aa, synthetic construct
TGAVTSGNY

SEQ ID NO: 112 - protein, 6 aa, synthetic construct
QDIGNY

SEQ ID NO: 113 - protein, 6 aa, synthetic construct
QGINNY

SEQ ID NO: 114 - protein, 6 aa, synthetic construct
QDINNY

SEQ ID NO: 115 - protein, 5 aa, synthetic construct
SSVTY

SEQ ID NO: 116 - protein, 11 aa, synthetic construct
KSLLHSNGITY

SEQ ID NO: 117 - protein, 6 aa, synthetic construct
QSVSSY

SEQ ID NO: 118 - protein, 11 aa, synthetic construct
QNIVHSNGNTY

SEQ ID NO: 119 - protein, 11 aa, synthetic construct
QASQDISNYLN

SEQ ID NO: 120 - protein, 6 aa, synthetic construct
QSISSY

SEQ ID NO: 121 - protein, 6 aa, synthetic construct
NIGSKS

SEQ ID NO: 122 - protein, 11 aa, synthetic construct
QSLVHSNGNTY

SEQ ID NO: 123 - protein, 16 aa, synthetic construct
RSSQSLVHSNGNTYLH

SEQ ID NO: 124 - protein, 16 aa, synthetic construct
RSSQSLVHSNGNTYLH

SEQ ID NO: 125 - protein, 11 aa, synthetic construct
QSLVHSNGNTY

SEQ ID NO: 126 - protein, 11 aa, synthetic construct
RASENIYSYLA

SEQ ID NO: 127-139 - skipped (13 entries)

SEQ ID NO: 140 - protein, 7 aa, synthetic construct
DASNLET

SEQ ID NO: 141 - protein, 4 aa, synthetic construct
YDSD

SEQ ID NO: 142 - protein, 7 aa, synthetic construct
KVSNRFS

SEQ ID NO: 143 - protein, 7 aa, synthetic construct
NARTLVE

SEQ ID NO: 144-145 - skipped (2 entries)

SEQ ID NO: 146 - protein, 9 aa, synthetic construct
QQFNSYPLT

SEQ ID NO: 147 - protein, 9 aa, synthetic construct
QQANSFPLT

SEQ ID NO: 148 - protein, 9 aa, synthetic construct
QQNNEWPTS

SEQ ID NO: 149 - protein, 9 aa, synthetic construct
QQNNNWPTT

SEQ ID NO: 150 - protein, 9 aa, synthetic construct
QHFDHLPLA

SEQ ID NO: 151 - protein, 9 aa, synthetic construct
VQYAQFPWT

SEQ ID NO: 152 - protein, 9 aa, synthetic construct
MQSTHVPRT

SEQ ID NO: 153 - protein, 9 aa, synthetic construct
VLWYSNRWV

SEQ ID NO: 154 - protein, 9 aa, synthetic construct
QHYNTVPPT

SEQ ID NO: 155 - protein, 8 aa, synthetic construct
LQHNSFPT

SEQ ID NO: 156 - protein, 9 aa, synthetic construct
LQYDNLLYT

SEQ ID NO: 157 - protein, 9 aa, synthetic construct
QQWSSHIFT

SEQ ID NO: 158 - protein, 9 aa, synthetic construct
AQNLELPYT

SEQ ID NO: 159 - protein, 9 aa, synthetic construct
HQYGSTPLT

SEQ ID NO: 160 - protein, 9 aa, synthetic construct
FQYSHVPWT

SEQ ID NO: 161 - protein, 9 aa, synthetic construct
QQSYSTPPT

SEQ ID NO: 162 - protein, 9 aa, synthetic construct
QQNNNWPTS

SEQ ID NO: 163 - protein, 11 aa, synthetic construct
QVWDTSSDHVL

SEQ ID NO: 164 - protein, 8 aa, synthetic construct
SQSTHVLT

SEQ ID NO: 165 - protein, 9 aa, synthetic construct
SQSTHVPWT

SEQ ID NO: 166 - protein, 9 aa, synthetic construct
QHHYGPPYT

SEQ ID NO: 167 - protein, 20 aa, synthetic construct
METDTLLLWVLLLWVPGSTG

SEQ ID NO: 168 - skipped

SEQ ID NO: 169 - protein, 125 aa, synthetic construct [sequence omitted]

SEQ ID NO: 170 - protein, 220 aa, synthetic construct
QVQLVQSGAEVKKPGASVKVSCKASGYTFTSYGFSWVRQAPGQGLEWMGWISASNGNTYYAQKLQGRVTMTTDTSTSTAYMELRSLRSDDTAVYYCARVYADYADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 171 - protein, 119 aa, synthetic construct
QVQLQESGPGLVKPSETLSLTCTVSGFSLSNYDVHWVRQAPGKGLEWLGVIWSGGNTDYNTPFTSRLTISVDTSKNQFSLKLSSVTAADTAVYYCARALDYYDYEFAYWGQGTLVTVSS

SEQ ID NO: 172 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 173 - protein, 119 aa, synthetic construct [sequence omitted]

SEQ ID NO: 174 - protein, 116 aa, synthetic construct
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 175 - protein, 124 aa, synthetic construct
QVQLVESGGGVVQSGRSLRLSCAASGFTFRNYGMHWVRQAPGKCLEWVAVIWYDGSDKYYADSVRGRFTISRDNSKNTLYLQMNSLRAEDTAVYYCARDGYDILTGNPRDFDYWGQGTLVTVSS

SEQ ID NO: 176 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 177 - protein, 123 aa, synthetic construct
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSS

SEQ ID NO: 178 - protein, 120 aa, synthetic construct [sequence omitted]

SEQ ID NO: 179 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSS

SEQ ID NO: 180 - protein, 119 aa, synthetic construct
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 181 - protein, 116 aa, synthetic construct
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 182 - protein, 121 aa, synthetic construct [sequence omitted]

SEQ ID NO: 183 - protein, 119 aa, synthetic construct
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSS

SEQ ID NO: 184 - protein, 121 aa, synthetic construct [sequence omitted]

SEQ ID NO: 185 - protein, 123 aa, synthetic construct [sequence omitted]

SEQ ID NO: 186 - protein, 120 aa, synthetic construct
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 187 - protein, 119 aa, synthetic construct
EVQLVESGGGLVQPGGSLRLSCAASGFSLTNYGVHWVRQAPGKGLEWLGVIWSGGNTDYGNEFTSRFTISRDNAKNSLYLQMNSLRAEDTAVYYCARALDYYDYEFAYWGQGTMVTVSS

SEQ ID NO: 188 - protein, 119 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVST

SEQ ID NO: 189 - protein, 120 aa, synthetic construct
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCARNPISIPAFDIWGQGTMVTVSS

SEQ ID NO: 190 - protein, 121 aa, synthetic construct
EMQLVESGGGFVKPGGSLKLSCAASGFAFSHYDMSWVRQTPKQRLEWVAYIASGGDITYYADTVKGRFTISRDNAQNTLYLQMSSLKSEDTAMFYCSRSSYGNNGDALDFWGQGTSVTVSS

SEQ ID NO: 191 - protein, 118 aa, synthetic construct
QIQLVQSGPELKKPGETVKISCKASGYTFTEYPIHWVKQAPGKGFKWMGMIYTDIGKPTYAEEFKGRFAFSLETSASTAYLQINNLKNEDTATYFCVRDRYDSLFDYWGQGTTLTVSS

SEQ ID NO: 192 - protein, 120 aa, synthetic construct
QVQLVQSGAEVKKPGASVKVSCKASGYTFTRYWLHWVRQAPGQGLEWIGEINPSSGRTNYNEKFKSRATLTVDKSISTAYMELSRLRSDDTAVYYCARWDYDEGTWFAYWGQGTLVTVSS

SEQ ID NO: 193 - protein, 120 aa, synthetic construct
QVQLVQSGAEVKKPGASVKVSCKASGYAFTGYTIHWVRQAPGQRLEWMGWFYPGSGTLKYSEKFQGRVTITRDKSLSTAYMELSSLRSEDTAVYYCARHGTGTLMAMDYWGQGTLVTVSS

SEQ ID NO: 194 - protein, 120 aa, synthetic construct
EVQLVESGGGLVQPGGSLRLSCAASGFTISTNAMSWVRQAPGKGLEWIGVITGRDITYYASWAKGRFTISRDNSKNTLYLQMNSLRAEDTAVYYCARDGGSSAITSNNIWGQGTLVTVSS

SEQ ID NO: 195 - protein, 107 aa, synthetic construct [sequence omitted]

SEQ ID NO: 196 - protein, 107 aa, synthetic construct
DIQMTQSPSSVSASVGDRVTITCRASQGINTWLAWYQQKPGKAPKLLIYAASSLKSGVPSRFSGSGSGTDFTLTISSLQPEDFATYYCQQANSFPLTFGGGTKVEIK

SEQ ID NO: 197 - protein, 107 aa, synthetic construct
EIVLTQSPDFQSVTPKEKVTITCRASQSIGTNIHWYQQKPDQSPKLLIKYASESISGIPSRFSGSGSGTDFTLTINSLEAEDAATYYCQQNNEWPTSFGQGTKLEIK

SEQ ID NO: 198 - protein, 107 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 199 - protein, 107 aa, synthetic construct
DIQMTQSPSTLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 200 - protein, 107 aa, synthetic construct
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 201 - protein, 112 aa, synthetic construct
DTVMTQTPLSSHVTLGQPASISCRSSQSLVHSDGNTYLSWLQQRPGQPPRLLIYRISRRFSGVPDRFSGSGAGTDFTLEISRVEAEDVGVYYCMQSTHVPRTFGCGTKVEIK

SEQ ID NO: 202 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 203 - protein, 106 aa, synthetic construct
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEI

SEQ ID NO: 204 - protein, 106 aa, synthetic construct [sequence omitted]

SEQ ID NO: 205 - protein, 107 aa, synthetic construct
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 206 - protein, 107 aa, synthetic construct; identical to SEQ ID NO: 200

SEQ ID NO: 207 - protein, 106 aa, synthetic construct [sequence omitted]

SEQ ID NO: 208 - protein, 112 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 209 - protein, 107 aa, synthetic construct [sequence omitted]

SEQ ID NO: 210 - protein, 112 aa, synthetic construct [sequence omitted]

SEQ ID NO: 211 - protein, 107 aa, synthetic construct [sequence omitted]

SEQ ID NO: 212 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 213 - protein, 107 aa, synthetic construct
EIVLTQSPATLSLSPGERATLSCRASQSIGTNIHWYQQKPGQAPRLLIKYASESISGIPARFSGSGSGTDFTLTISSLEPEDFAVYYCQQNNNWPTSFGGGTKVEIK

SEQ ID NO: 214 - protein, 108 aa, synthetic construct
QPVLTQPPSVSVAPGKTARITCGGNNIGSKSVHWYQQKPGQAPVLVIYYDSDRPSGIPERFSGSNSGNTATLTISRVEAGDEADYYCQVWDTSSDHVLFGGGTKLTVL

SEQ ID NO: 215 - protein, 111 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVLTFGSGTKLEIK

SEQ ID NO: 216 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIK

SEQ ID NO: 217 - protein, 107 aa, synthetic construct
DIQMTQSPSFLSASVGDRVTITCRASENIYSYLAWYQQKPGKAPKLLVYNARTLVEGVPSRFSGSGSGTEFTLTISSLQPEDFATYYCQHHYGPPYTFGQGTKVEIK

SEQ ID NO: 218 - protein, 112 aa, synthetic construct
DVVMTQTPLSLSVTPGQPASISCKSSQSLANSYGNTYLSWYLHKPGQSPQLLIYGISNRFSGVPDRFSGSGSGTDFTLKISRVEAEDVGVYYCLQGTHQPPTFGQGTKLEIK

SEQ ID NO: 219 - protein, 112 aa, synthetic construct
DVVMTQSPSTLSASVGDRVTINCQASESISSWLAWYQQKPGKAPKLLIYEASKLASGVPSRFSGSGSGTEFTLTISSLQPDDFATYYCQGYFYFISRTYVNSFGGGTKVEIK

SEQ ID NO: 220 - protein, 15 aa, synthetic construct
GGGGSGGGGSGGGGS

SEQ ID NO: 221 - protein, 19 aa, synthetic construct
MGWSCIILFLVATATGVHS

(19) United States
(12) Patent Application Publication
(10) Pub. No.: US 2026/0144867 A1
(43) Pub. Date: May 28, 2026

(54) ENGINEERED IGA ANTIBODIES AND METHODS OF USE

(71) Applicant: TigaTx, Inc., Boston, MA (US)

(72) Inventors: Geert VAN TETERING, Utrecht (NL); Elina Makino, Boston, MA (US); Howard Stern, Boston, MA (US)

(21) Appl. No.: 19/461,172

(22) Filed: Jan. 27, 2026

Related U.S. Application Data
(63) Continuation of application No. PCT/US24/39908, filed on Jul. 26, 2024.
(60) Provisional application No. 63/516,428, filed on Jul. 28, 2023.

(51) Int. Cl. A61K 39/395; A61K 39/00; A61K 45/06; A61P 35/00; C07K 16/28

(52) U.S. Cl. CPC A61K 39/3955; A61K 45/06; A61P 35/00; C07K 16/2863; A61K 2039/505; A61K 2039/545; C07K 2317/732; C07K 2317/92; C07K 2317/94

(57) ABSTRACT

Provided herein are engineered IgA antibodies that comprises: (a) an EGFR binding domain and (b) an IgA heavy chain constant region. Also, provided herein are methods of treating cancer in a subject in need thereof, the method comprising administering to the subject an effective amount of the engineered antibody.

### CROSS-REFERENCED APPLICATIONS

This application is a continuation of International Application No. PCT/US2024/039908, filed Jul. 26, 2024, which claims the benefit of priority to U.S. Provisional Application No. 63/516,428, filed on Jul. 28, 2023 the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 199828-714601_PCT_SL.xml, which was created on Jul. 26, 2024, and is 189,604 bytes in size, is hereby incorporated by reference in its entirety.

### FIELD

The disclosure generally relates to antibodies that bind epidermal growth factor receptor (EGFR).

### BACKGROUND OF THE DISCLOSURE

Monoclonal antibodies that target specific antigens implicated in diseases or conditions are emerging as an attractive treatment for ameliorating the disease or condition in human subjects. Over the years an increasing number of monoclonal antibodies (mainly IgG based antibodies) targeting different tumor antigens have been approved for use in cancer therapies. However, their clinical efficacy and side effects, especially side effects related to IgG-based antibody monotherapy, remain problematic. Therefore, it is of interest to develop new antibody therapies with increased clinical efficacy and/or decreased incidence/severity of side effects.

### SUMMARY OF THE DISCLOSURE

Provided herein are engineered antibodies comprising: (a) an epidermal growth factor receptor (EGFR) binding domain that comprises a heavy chain variable region (VH) domain and a light chain variable region (VL) domain; and (b) an immunoglobulin A (IgA) heavy chain constant region that comprises at least one mutation, relative to a wild-type IgA heavy chain constant region having an amino acid sequence of SEQ ID NO: 1, wherein the mutation results in one or more of: reduced glycosylation, reduced aggregation, improved thermal stability, increased mechanical stability, or increased circulatory half-life, each relative to a corresponding antibody that comprises the wild-type IgA heavy chain constant region. In some embodiments, the VH domain comprises three heavy chain complementarity determining regions (CDR-Hs), which are: (1) a CDR-H1 comprising any one of amino acid sequences at least one of the heavy chain complementarity-determining regions (CDRs) of SEQ ID NO: 34-54, or a variant thereof comprising 1 to 3 substitutions, deletions or insertions, (2) a CDR-H2 comprising any one of amino acid sequences of SEQ ID NO: 57-78, or a variant thereof comprising 1 to 3 substitutions, deletions or insertions, (3) a CDR-H3 comprising any one of amino acid sequences of SEQ ID NO: 81-102, or a variant thereof comprising 1 to 3 substitutions, deletions or insertions. In some embodiments, the VL domain comprises three light chain complementarity determining regions (CDR-Ls), which are (1) a CDR-L1 comprising any one of amino acid sequences of SEQ ID NO: 105-126, or a variant thereof comprising 1 to 3 substitutions, deletions or insertions, (2) a CDR-L2 comprising any one of amino acid sequences of SEQ ID NO: 129-143, or a variant thereof comprising 1 to 3 substitutions, deletions or insertions, and (3) a CDR-L3 comprising any one of amino acid sequences of SEQ ID NO: 146-166, or a variant thereof comprising 1 to 3 substitutions, deletions or insertions. In some embodiments, the CDR-Hs and the CDR-Ls are selected according to any one of combinations provided in TABLE 9. In some embodiments, the engineered antibody comprises: the CDR-H1 comprising an amino acid sequence that is at least 80% identical to any one of the amino acid sequences of SEQ ID NO: 34-54; the CDR-H2 comprising an amino acid sequence that is at least 80% identical to any one of the amino acid sequences of SEQ ID NO: 57-78; the CDR-H3 comprising an amino acid sequence that is at least 80% identical to any one of the amino acid sequences of SEQ ID NO: 81-102; the CDR-L1 comprising an amino acid sequence that is at least 80% identical to any one of the amino acid sequences of SEQ ID NO: 105-126; the CDR-L2 comprising an amino acid sequence that is identical to any one of the amino acid sequences of SEQ ID NO: 129-143; and the CDR-L3 comprising an amino acid sequence that is at least 80% identical to any one of the amino acid sequences of SEQ ID NO: 146-166. In some embodiments, the engineered antibody comprises: the CDR-H1 comprising an amino acid sequence that is identical to any one of the amino acid sequences of SEQ ID NO: 34-54; the CDR-H2 comprising an amino acid sequence that is identical to any one of the amino acid sequences of SEQ ID NO: 57-78; the CDR-H3 comprising an amino acid sequence that is identical to any one of the amino acid sequences of SEQ ID NO: 81-102; the CDR-L1 comprising an amino acid sequence that is identical to any one of the amino acid sequences of SEQ ID NO: 105-126; the CDR-L2 comprising an amino acid sequence that is identical to any one of the amino acid sequences of SEQ ID NO: 129-143; and the CDR-L3 comprising an amino acid sequence that is identical to any one of the amino acid sequences of SEQ ID NO: 146-166. In some embodiments, the VH domain comprises an amino acid sequence that is at least 80% identical to any one of the amino acid sequences recited in TABLE 5. In some embodiments, the VL domain comprises an amino acid sequence that is at least 80% identical to any one of the amino acid sequences recited in TABLE 7. In some embodiments, the VH domain and the VL domain are selected according to any one of combinations provided in TABLE 8. In some embodiments, the engineered antibody comprises an IgA light chain constant region that comprises an amino acid sequence that is at least 80% identical to SEQ ID NO: 23. In some embodiments, the IgA heavy chain constant region comprises an IgA CH1 region, an IgA CH2 region, and an IgA CH3 region. In some embodiments, the at least one mutation is present in the IgA CH1 region and is an N45.2 substitution, a P124 substitution, or a combination thereof, numbering according to IMGT scheme, each relative to a corresponding antibody that comprises the wild-type IgA heavy chain constant region of SEQ ID NO: 1. In some embodiments, the at least one mutation is: (a) an N45.2 substitution selected from the group consisting of: N45.2G and N45.2A; (b) a P124R substitution; or (c) any combination thereof, numbering according to IMGT scheme, each relative to a corresponding antibody that comprises the wild-type IgA heavy chain constant region of SEQ ID NO: 1. In some embodiments, the at least one mutation is present in the IgA CH2 region and is: (a) an N20 substitution; (b) an L21 substitution; (c) a T22 substitution; (d) a C92 substitution; (e) an N120 substitution; (f) an I121 substitution; or (g) a T122 substitution, numbering according to IMGT scheme, each relative to a corresponding antibody that comprises the wild-type IgA heavy chain constant region of SEQ ID NO: 1. In some embodiments, the at least one mutation is: (a) an N20 substitution selected from the group consisting of: N20G, N20Q, and N20T; (b) an L21I substitution; (c) a T22S substitution; (d) a C92S substitution; (e) an N120T substitution; (f) an I121L substitution; (g) a T122S substitution; or (h) any combination thereof, numbering according to IMGT scheme, each relative to a corresponding antibody that comprises the wild-type IgA heavy chain constant region of SEQ ID NO: 1. In some embodiments, the at least one mutation is present in the IgA CH3 region and is: (a) an H5 substitution; (b) an L7 substitution; (c) a P10 substitution; (d) a T22 substitution; (e) an L79 substitution; (f) a W81 substitution; (g) an A85.1 substitution; (h) a T86 substitution; (i) a 188 substitution; (j) an N135 substitution; (k) a C147 deletion; (l) a Y148 deletion; (m) a deletion of P131-Y148; or (n) a combination thereof, numbering according to IMGT scheme, relative to the corresponding residue in the wild-type IgA heavy chain constant region of SEQ ID NO: 1. In some embodiments, the at least one mutation is: (a) an H5 substitution selected from the group consisting of: HSC, H5Y, HSF, H5M and H5W; (b) an L7 substitution selected from the group consisting of: L7F, L7Y, L7M, L7W, L7H and L7I; (c) a P10C substitution; (d) a T22 substitution selected from the group consisting of: T22V, T22I, T22L and T22A; (e) an L79 substitution selected from the group consisting of: L79V, L79T, L79A and L79I; (f) a W81 substitution selected from the group consisting of: W81T, W81L, W81A, W81V and W81I; (g) an A85.1 substitution selected from the group consisting of: A85.1F, A85.1Y, A85.1M, A85.1W and A85.1H; (h) a T86 substitution selected from the group consisting of: T86Y, T86F, T86M, T86W and T86H; (i) a 188 substitution selected from the group consisting of: I88L, 188A, 188V and I88T; or (j) any combination thereof, numbering according to IMGT scheme, relative to the corresponding residue in the wild-type IgA heavy chain constant region of SEQ ID NO: 1. In some embodiments, the at least one mutation is: (a) an N135Q substitution; (b) a C147 deletion; (c) a Y148 deletion; or (d) any combination thereof, numbering according to IMGT scheme, relative to the corresponding residue in the wild-type IgA heavy chain constant region of SEQ ID NO: 1. In some embodiments, the at least one mutation is a deletion of P131-Y148, numbering according to IMGT scheme, relative to the corresponding residue in the wild-type IgA heavy chain constant region of SEQ ID NO: 1. In some embodiments, the IgA heavy chain constant region comprises an amino acid sequence that is at least 80% identical to the IgA heavy chain constant region of SEQ ID NO: 3. In some embodiments, the engineered antibody is monomeric. In some embodiments, the EGFR binding domain binds to an EGFR polypeptide variant, wherein the EGFR variant comprises EGFRvIII, exon 19 deletions, L858R substitutions in exon 21, a C797S substitution, or a T790M substitution. In some embodiments, the VH region comprises an amino acid sequence of SEQ ID NO: 173, and the VL region comprises an amino acid sequence of SEQ ID NO: 211. In some embodiments, the VH region comprises an amino acid sequence of SEQ ID NO: 172, and the VL region comprises an amino acid sequence of SEQ ID NO: 198. In some embodiments, the VH region comprises an amino acid sequence of SEQ ID NO: 182, and the VL region comprises an amino acid sequence of SEQ ID NO: 207. In some embodiments, the VH region comprises an amino acid sequence of SEQ ID NO: 184, and the VL region comprises an amino acid sequence of SEQ ID NO: 209. In some embodiments, the engineered antibody is capable of inducing antibody dependent cell cytotoxicity (ADCC) via an immune effector cell. In some embodiments, the immune effector cell is a neutrophil, a T cell, an eosinophil, or a macrophage. In some embodiments, the engineered antibody is a chimeric antibody, a single chain antibody, a humanized antibody, a human antibody, a monoclonal antibody, a deimmunized antibody, a bispecific antibody, a multispecific antibody, a multivalent antibody, or a combination thereof. In some embodiments, the engineered antibody is a bispecific antibody. In some embodiments, the engineered antibody further comprises a binding domain that binds to a polypeptide antigen selected from the group consisting of: MET, cMet, CD28, HER2, HER3, IGF-IR, CD3, PD1, PD-L1, VEGFR2, FcGR3, and 4-1BB.

Also provided herein are engineered antibodies comprising: (a) an epidermal growth factor receptor (EGFR) binding domain that binds to domain III of an EGFR polypeptide or a variant thereof, and (b) an immunoglobulin A (IgA) constant domain that comprises an IgA heavy chain constant region having at least one mutation, relative to a wild-type IgA heavy chain constant region having an amino acid sequence of SEQ ID NO: 1, wherein the mutation results in one or more of: reduced glycosylation, reduced aggregation, improved thermal stability, increased mechanical stability, or increased circulatory half-life, each relative to a corresponding antibody that comprises the wild-type IgA heavy chain constant region. In some embodiments, the IgA constant domain comprises: (a) an IgA heavy chain constant region having an amino acid sequence of SEQ ID NO: 3; and (b) an IgA light chain constant region having an amino acid sequence of SEQ ID NO: 2. In some embodiments, the IgA constant domain comprises an IgA heavy chain constant region that comprises an IgA CH1, CH2, and CH3 domain, wherein the IgA heavy chain constant region comprises the following mutations: (a) an N45.2G substitution in the CH1 domain; (b) a P124R substitution in the CH1 domain; (c) a C92S substitution in the CH2 domain; (d) an N120T substitution in the CH2 domain; (e) an I121L substitution in the CH2 domain, and (f) a T122S substitution in the CH2 domain, numbering according to IMGT scheme, each relative to a corresponding antibody that comprises the wild-type IgA heavy chain constant region of SEQ ID NO: 1. In some embodiments, the EGFR binding domain binds to epitope of the EGFR polypeptide or a variant thereof that comprises any one of the following EGFR amino acid residues: P349, F352, D355, P362, D355, Q384, P387, Q408, H409, F412, I438, K443, K465, I467, or S468.

Also provided herein are engineered antibodies comprising: (a) an epidermal growth factor receptor (EGFR) binding domain that binds to domain II of an EGFR polypeptide or a variant thereof, and (b) an immunoglobulin A (IgA) constant domain that comprises an IgA heavy chain constant region having at least one mutation, relative to a wild-type IgA heavy chain constant region having an amino acid sequence of SEQ ID NO: 1, wherein the mutation results in one or more of: reduced glycosylation, reduced aggregation, improved thermal stability, increased mechanical stability, or increased circulatory half-life, each relative to the wild-type IgA heavy chain constant region. In some embodiments, the IgA constant domain comprises: (a) an IgA heavy chain constant region having an amino acid sequence of SEQ ID NO: 3; and (b) an IgA light chain constant region having an amino acid sequence of SEQ ID NO: 2. In some embodiments, the IgA constant domain comprises an IgA heavy chain constant region that comprises an IgA CH1, CH2, and CH3 domain, wherein the IgA heavy chain constant region comprises the following mutations: (a) an N45.2G substitution in the CH1 domain; (b) a P124R substitution in the CH1 domain; (c) a C92S substitution in the CH2 domain; (d) an N120T substitution in the CH2 domain; (e) an I121L substitution in the CH2 domain, and (f) a T122S substitution in the CH2 domain, numbering according to IMGT scheme, each relative to a corresponding antibody that comprises the wild-type IgA heavy chain constant region of SEQ ID NO: 1.

Also provided herein are engineered antibodies comprising: (a) an epidermal growth factor receptor (EGFR) binding domain that comprises a heavy chain variable region (VH) domain and a light chain variable region (VL) domain; and (b) an immunoglobulin A (IgA) constant domain that comprises: (i) a heavy chain constant region having an amino acid sequence of SEQ ID NO: 5, and (ii) a light chain constant domain having an amino acid sequence of SEQ ID NO: 23. In some embodiments, the VH region comprises an amino acid sequence of SEQ ID NO: 173, and the VL region comprises an amino acid sequence of SEQ ID NO: 211. In some embodiments, the VH region comprises an amino acid sequence of SEQ ID NO: 172, and the VL region comprises an amino acid sequence of SEQ ID NO: 198. In some embodiments, the VH region comprises an amino acid sequence of SEQ ID NO: 182, and the VL region comprises an amino acid sequence of SEQ ID NO: 207. In some embodiments, the VH region comprises an amino acid sequence of SEQ ID NO: 184, and the VL region comprises an amino acid sequence of SEQ ID NO: 209. In some embodiments, the VH domain comprises three heavy chain complementarity determining regions (CDR-Hs), which are: (1) a CDR-H1 comprising any one of amino acid sequences at least one of the heavy chain complementarity-determining regions (CDRs) of SEQ ID NO: 34-54, or a variant thereof comprising 1 to 3 substitutions, deletions or insertions, (2) a CDR-H2 comprising any one of amino acid sequences of SEQ ID NO: 57-78, or a variant thereof comprising 1 to 3 substitutions, deletions or insertions, (3) a CDR-H3 comprising any one of amino acid sequences of SEQ ID NO: 81-102, or a variant thereof comprising 1 to 3 substitutions, deletions or insertions. In some embodiments, the VL domain comprises three light chain complementarity determining regions (CDR-Ls), which are (1) a CDR-L1 comprising any one of amino acid sequences of SEQ ID NO: 105-126, or a variant thereof comprising 1 to 3 substitutions, deletions or insertions, (2) a CDR-L2 comprising any one of amino acid sequences of SEQ ID NO: 129-143, or a variant thereof comprising 1 to 3 substitutions, deletions or insertions, and (3) a CDR-L3 comprising any one of amino acid sequences of SEQ ID NO: 146-166, or a variant thereof comprising 1 to 3 substitutions, deletions or insertions. In some embodiments, the CDR-Hs and the CDR-Ls are selected according to any one of combinations provided in TABLE 9.

Also provided herein are engineered antibodies comprising: (a) an epidermal growth factor receptor (EGFR) binding domain that comprises a heavy chain variable region (VH) domain and a light chain variable region (VL) domain; and (b) an immunoglobulin A (IgA) heavy chain constant region that comprises an IgA CH1, CH2, and CH3 domain, wherein the IgA heavy chain constant region comprises the following mutations: (i) an N45.2G substitution in the CH1 domain, (ii) a P124R substitution in the CH1 domain, (iii) a C92S substitution in the CH2 domain, (iv) an N120T substitution in the CH2 domain, (v) an I121L substitution in the CH2 domain, and (vi) a T122S substitution in the CH2 domain, numbering according to IMGT scheme, each relative to a corresponding antibody that comprises the wild-type IgA heavy chain constant region of SEQ ID NO: 1. In some embodiments, the VH domain comprises three heavy chain complementarity determining regions (CDR-Hs), which are: (1) a CDR-H1 comprising any one of amino acid sequences at least one of the heavy chain complementarity-determining regions (CDRs) of SEQ ID NO: 34-54, or a variant thereof comprising 1 to 3 substitutions, deletions or insertions, (2) a CDR-H2 comprising any one of amino acid sequences of SEQ ID NO: 57-78, or a variant thereof comprising 1 to 3 substitutions, deletions or insertions, (3) a CDR-H3 comprising any one of amino acid sequences of SEQ ID NO: 81-102, or a variant thereof comprising 1 to 3 substitutions, deletions or insertions. In some embodiments, the VL domain comprises three light chain complementarity determining regions (CDR-Ls), which are (1) a CDR-L1 comprising any one of amino acid sequences of SEQ ID NO: 105-126, or a variant thereof comprising 1 to 3 substitutions, deletions or insertions, (2) a CDR-L2 comprising any one of amino acid sequences of SEQ ID NO: 129-143, or a variant thereof comprising 1 to 3 substitutions, deletions or insertions, and (3) a CDR-L3 comprising any one of amino acid sequences of SEQ ID NO: 146-166, or a variant thereof comprising 1 to 3 substitutions, deletions or insertions. In some embodiments, the CDR-Hs and the CDR-Ls are selected according to any one of combinations provided in TABLE 9. In some embodiments, the IgA heavy chain constant region further comprises the following mutations in the CH3 domain: (a) an N135Q substitution; (b) a C147 deletion; and (c) a Y148 deletion, numbering according to IMGT scheme, each relative to a corresponding antibody that comprises the wild-type IgA heavy chain constant region of SEQ ID NO: 1. In some embodiments, the IgA heavy chain constant region further comprises a deletion of P131-Y148 in the CH3 domain, numbering according to IMGT scheme, relative to a corresponding antibody that comprises the wild-type IgA heavy chain constant region of SEQ ID NO: 1. In some embodiments, the IgA heavy chain constant region further comprises the following mutations in the CH2 domain: (a) an N20 substitution selected from the group consisting of: N20G, N20Q, and N20T; (b) an L21I substitution, and (c) a T22S substitution, numbering according to IMGT scheme, each relative to a corresponding antibody that comprises the wild-type IgA heavy chain constant region of SEQ ID NO: 1. In some embodiments, the VH region comprises an amino acid sequence of SEQ ID NO: 173, and the VL region comprises an amino acid sequence of SEQ ID NO: 211. In some embodiments, the VH region comprises an amino acid sequence of SEQ ID NO: 172, and the VL region comprises an amino acid sequence of SEQ ID NO: 198. In some embodiments, the VH region comprises an amino acid sequence of SEQ ID NO: 182, and the VL region comprises an amino acid sequence of SEQ ID NO: 207. In some embodiments, the VH region comprises an amino acid sequence of SEQ ID NO: 184, and the VL region comprises an amino acid sequence of SEQ ID NO: 209.

Also provided herein are engineered epidermal growth factor receptor (EGFR) binding antibodies or functional EGFR binding fragments thereof that comprise (a) an EGFR binding domain that comprises a heavy chain variable (VH) region and a light chain variable (VL) region, wherein the VH region comprises an amino acid sequence of SEQ ID NO: 173 and the VL region comprises an amino acid sequence of SEQ ID NO: 211; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are engineered epidermal growth factor receptor (EGFR) binding antibodies or functional EGFR binding fragments thereof that comprise (a) n EGFR binding domain that comprises a heavy chain variable (VH) region and a light chain variable (VL) region, wherein the VH region comprises an amino acid sequence of SEQ ID NO: 172 and the VL region comprises an amino acid sequence of SEQ ID NO: 198; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are engineered epidermal growth factor receptor (EGFR) binding antibodies or functional EGFR binding fragments thereof that comprise (a) an EGFR binding domain that comprises a heavy chain variable (VH) region and a light chain variable (VL) region, wherein the VH region comprises an amino acid sequence of SEQ ID NO: 182 and the VL region comprises an amino acid sequence of SEQ ID NO: 207; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are engineered epidermal growth factor receptor (EGFR) binding antibodies or functional EGFR binding fragments thereof that comprise (a) an EGFR binding domain that comprises a heavy chain variable (VH) region and a light chain variable (VL) region, wherein the VH region comprises an amino acid sequence of SEQ ID NO: 184 and the VL region comprises an amino acid sequence of SEQ ID NO: 209; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are pharmaceutical compositions comprising engineered antibodies described herein, and a pharmaceutically acceptable carrier.

Also provided herein are methods of treating a cancer in a subject in need thereof. In some embodiments, the method comprises administering to the subject an effective amount of an engineered antibody that comprises: (a) an epidermal growth factor receptor (EGFR) binding domain that comprises a heavy chain variable region (VH) domain and a light chain variable region (VL) domain; and (b) an immunoglobulin A (IgA) heavy chain constant region that comprises at least one mutation, relative to a wild-type IgA heavy chain constant region having an amino acid sequence of SEQ ID NO: 1, wherein the mutation results in one or more of: reduced glycosylation, reduced aggregation, improved thermal stability, increased mechanical stability, or increased circulatory half-life, each relative to the wild-type IgA heavy chain constant region. In some embodiments, the VH domain comprises three heavy chain complementarity determining regions (CDR-Hs), which are: (1) a CDR-H1 comprising any one of amino acid sequences at least one of the heavy chain complementarity-determining regions (CDRs) of SEQ ID NO: 34-54, or a variant thereof comprising 1 to 3 substitutions, deletions or insertions, (2) a CDR-H2 comprising any one of amino acid sequences of SEQ ID NO: 57-78, or a variant thereof comprising 1 to 3 substitutions, deletions or insertions, (3) a CDR-H3 comprising any one of amino acid sequences of SEQ ID NO: 81-102, or a variant thereof comprising 1 to 3 substitutions, deletions or insertions. In some embodiments, the VL domain comprises three light chain complementarity determining regions (CDR-Ls), which are (1) a CDR-L1 comprising any one of amino acid sequences of SEQ ID NO: 105-126, or a variant thereof comprising 1 to 3 substitutions, deletions or insertions, (2) a CDR-L2 comprising any one of amino acid sequences of SEQ ID NO: 129-143, or a variant thereof comprising 1 to 3 substitutions, deletions or insertions, and (3) a CDR-L3 comprising any one of amino acid sequences of SEQ ID NO: 146-166, or a variant thereof comprising 1 to 3 substitutions, deletions or insertions. In some embodiments, the CDR-Hs and the CDR-Ls are selected according to any one of combinations provided in TABLE 9. In some embodiments, the VH domain comprises three heavy chain complementarity determining regions (CDR-Hs), which are: (1) a CDR-H1 comprising any one of amino acid sequences at least one of the heavy chain complementarity-determining regions (CDRs) of SEQ ID NO: 34-54, or a variant thereof comprising 1 to 3 substitutions, deletions or insertions, (2) a CDR-H2 comprising any one of amino acid sequences of SEQ ID NO: 57-78, or a variant thereof comprising 1 to 3 substitutions, deletions or insertions, (3) a CDR-H3 comprising any one of amino acid sequences of SEQ ID NO: 81-102, or a variant thereof comprising 1 to 3 substitutions, deletions or insertions. In some embodiments, the VL domain comprises three light chain complementarity determining regions (CDR-Ls), which are (1) a CDR-L1 comprising any one of amino acid sequences of SEQ ID NO: 105-126, or a variant thereof comprising 1 to 3 substitutions, deletions or insertions, (2) a CDR-L2 comprising any one of amino acid sequences of SEQ ID NO: 129-143, or a variant thereof comprising 1 to 3 substitutions, deletions or insertions, and (3) a CDR-L3 comprising any one of amino acid sequences of SEQ ID NO: 146-166, or a variant thereof comprising 1 to 3 substitutions, deletions or insertions. In some embodiments, the CDR-Hs and the CDR-Ls are selected according to any one of combinations provided in TABLE 9. In some embodiments, the mutation results in one or more of: reduced glycosylation, reduced aggregation, improved thermal stability, increased mechanical stability, or increased circulatory half-life, each relative to the wild-type IgA heavy chain constant region. In some embodiments, the IgA heavy chain constant region comprises an IgA CH1, CH2, and CH3 domain, wherein the IgA heavy chain constant region comprises the following mutations: (a) an N45.2G substitution in the CH1 domain; (b) a P124R substitution in the CH1 domain; (c) a C92S substitution in the CH2 domain; (d) an N120T substitution in the CH2 domain; (e) an I121L substitution in the CH2 domain; and (f) a T122S substitution in the CH2 domain, numbering according to IMGT scheme, each relative to a corresponding antibody that comprises the wild-type IgA heavy chain constant region of SEQ ID NO: 1. In some embodiments, the IgA heavy chain constant region further comprises the following mutations in the CH3 domain: (a) an N135Q substitution; (b) a C147 deletion; and (c) a Y148 deletion, numbering according to IMGT scheme, each relative to a corresponding antibody that comprises the wild-type IgA heavy chain constant region of SEQ ID NO: 1. In some embodiments, the IgA heavy chain constant region further comprises a deletion of P131-Y148 in the CH3 domain, numbering according to IMGT scheme, relative to a corresponding antibody that comprises the wild-type IgA heavy chain constant region of SEQ ID NO: 1. In some embodiments, the IgA heavy chain constant region further comprises the following mutations in the CH2 domain: (a) an N20 substitution selected from the group consisting of: N20G, N20Q, and N20T; (b) an L211 substitution; and (c) a T22S substitution, numbering according to IMGT scheme, each relative to a corresponding antibody that comprises the wild-type IgA heavy chain constant region of SEQ ID NO: 1. In some embodiments, the cancer is a solid tumor cancer. In some embodiments, the cancer is selected from the group consisting of: lung cancer, head and neck cancer, colon cancer, rectal cancer, pancreatic cancer, breast cancer, ovarian cancer, bladder cancer, kidney cancer, mesothelioma and a glioblastoma. In some embodiments, the cancer is an adenocarcinoma, a squamous cell carcinoma, or a large-cell carcinoma. In some embodiments, the cancer is colorectal cancer. In some embodiments, the cancer is head and neck squamous cell carcinoma. In some embodiments, the cancer is non-small cell lung cancer. In some embodiments, the engineered antibody inhibits tumor growth associated with the cancer. In some embodiments, the administering is subcutaneous, intravenous, intradermal, intraperitoneal, oral, intramuscular, or intracranial. In some embodiments, the engineered antibody is administered to the subject in combination with a second therapeutic agent. In some embodiments, the second therapeutic agent comprises an anti-cancer agent, a chemotherapeutic agent, radiation therapy, a cytotoxic agent, a corticosteroid, an immunotherapy agent, a dietary supplement, or an antioxidant. In some embodiments, the second therapeutic agent is administered prior to, concurrently, or after administering the engineered antibody. In some embodiments, the subject is a rodent, a non-human primate or a human. In some embodiments, the subject is a rodent, and wherein the effective amount is administered at a dosage of from 1 mg/kg to 25 mg/kg administered subcutaneously, intravenously or intraperitoneally twice weekly for 35-40 days. In some embodiments, the effective amount is administered intravenously every seven days for 5 weeks at a dosage of 25 mg/kg except for the third dose, which is delivered at 12.5 mg/kg. In some embodiments, the effective amount is administered intravenously at a dosage of from 1 mg/kg to 25 mg/kg twice weekly.

Also provided herein are methods of treating cancer in a subject in need thereof, the methods comprise administering to the subject an effective amount of an engineered epidermal growth factor receptor (EGFR) binding antibody or a functional EGFR binding fragment thereof that comprises (a) an EGFR binding domain that comprises a heavy chain variable (VH) region and a light chain variable (VL) region, wherein the VH region comprises an amino acid sequence of SEQ ID NO: 173 and the VL region comprises an amino acid sequence of SEQ ID NO: 211; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are methods of treating cancer in a subject in need thereof, the methods comprise administering to the subject an effective amount of an engineered epidermal growth factor receptor (EGFR) binding antibody or a functional EGFR binding fragment thereof that comprises (a) an EGFR binding domain that comprises a heavy chain variable (VH) region and a light chain variable (VL) region, wherein the VH region comprises an amino acid sequence of SEQ ID NO: 172 and the VL region comprises an amino acid sequence of SEQ ID NO: 198; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are methods of treating cancer in a subject in need thereof, the methods comprise administering to the subject an effective amount of an engineered epidermal growth factor receptor (EGFR) binding antibody or a functional EGFR binding fragment thereof that comprises (a) an EGFR binding domain that comprises a heavy chain variable (VH) region and a light chain variable (VL) region, wherein the VH region comprises an amino acid sequence of SEQ ID NO: 182 and the VL region comprises an amino acid sequence of SEQ ID NO: 207; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are methods of treating cancer in a subject in need thereof, the methods comprise administering to the subject an effective amount of an engineered epidermal growth factor receptor (EGFR) binding antibody or a functional EGFR binding fragment thereof that comprises (a) an EGFR binding domain that comprises a heavy chain variable (VH) region and a light chain variable (VL) region, wherein the VH region comprises an amino acid sequence of SEQ ID NO: 184 and the VL region comprises an amino acid sequence of SEQ ID NO: 209; and (b) an immunoglobulin A (IgA) constant domain that comprises a heavy chain constant region having an amino acid sequence of SEQ ID NO: 5 and a light chain constant domain having an amino acid sequence of SEQ ID NO: 23.

Also provided herein are isolated nucleic acids encoding engineered antibodies described herein.

Also provided herein are host cells expressing engineered antibodies described herein.

Also provided herein are compositions comprising a first therapeutic agent and a second therapeutic agent, wherein the first therapeutic agent comprises any one of engineered antibodies described herein and the second therapeutic agent binds to MET, cMet, CD28, HER2, HER3, IGF-IR, CD3, PD1, PD-L1, VEGFR2, FcGR3, 4-1BB, or a combination thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 depicts the amino acid sequence of the human IgA2 heavy chain having the accession number UniProt Reference No.: A0A0G2JMB2 (SEQ ID NO: 1). The highlighted amino acids depict residues that are modified in some embodiments described herein. The underlined sequence indicates the CH3 tail piece, which in some embodiments is partially or fully deleted.

FIG. 2 depicts the amino acid sequence of the human IgA2 heavy chain having the accession number UniProt Reference No.: P01877-1 (SEQ ID NO: 2). The highlighted amino acids depict residues that are modified in some embodiments described herein. The underlined sequence indicates the CH3 tail piece, which in some embodiments is partially or fully deleted.

FIG. 3 depicts the amino acid sequence of the human IgA2 heavy chain having the accession number UniProt Reference No.: A0A286YEY5 (SEQ ID NO: 3). The highlighted amino acids depict residues that are modified in some embodiments described herein.

FIGS. 4A-4D show schematic representations of engineered anti-EGFR IgA variants and a wild type IgA (IgA2(m1)). FIG. 4A is a representation of wild type (WT) IgA2m1. IgA2m1 contains four N-glycosylation sites in its CH domains, including 1 glycosylation site in its tailpiece. FIG. 4B is a representation of an engineered anti-EGFR IgA3.0+ (plus) variant. The engineered anti-EGFR IgA3.0+ variant is generated by engineering an anti-EGFR IgA2m1 antibody to contain a stabilized heavy and light chain linkage via: (i) a CH1-P124R mutation, and (ii) removal of two free cysteines of which one is mutated to serine (CH2-C92S) and the second (CH3-CHS-C147del) is removed by deletion of two final amino acids of the tailpiece. In addition, three N-linked glycosylation sites were removed by substituting critical amino acids in three N-glycosylation motifs. Mutation in the three N-glycosylation motifs include CH1-N45.2G; CH2-N120T-I121L-T122S; CH3-CHS-N135Q. FIG. 4C is a representation of an engineered anti-EGFR IgA3.0− (IgA3.0min) variant that contains a deletion of the entire tailpiece (CH3-CHS-P131-Y148del). The anti-EGFR IgA3.0min variant contains a stabilized heavy and light chain linkage (CH1-P124R mutation), deletion of entire tailpiece (CH3-CHS-P131-Y148del), lacks two free cysteines of which 1 is mutated to serine (CH2-C92S) and the second (CH3-CHS-C147del) is deletion of the tailpiece. In addition, three N-linked glycosylation sites have been removed by substituting critical amino acids in 2 N-glycosylation motifs i.e., CH1-N45.2G and CH2-N120T-I121L-T122S and deletion of CH3-CHS-N135Qdel by deletion of tailpiece. FIG. 4D shows a representation of an engineered anti-EGFR IgA4.0 variant that contains all the features of the anti-EGFR IgA3.0min and further contains a mutation in the final N-linked glycosylation motif CH2-N20. Therefore, the anti-EGFR IgA4.0 variant contains a stabilized heavy and light chain linkage (CH1-P124R mutation), deletion of entire tailpiece (CH3-CHS-P131-Y148del), lacks two free cysteines of which 1 is mutated to serine (CH2-C92S) and the second (CH3-CHS-C147del) is deletion of the tailpiece. In addition, four N-linked glycosylation sites have been removed by substituting critical amino acids in four N-glycosylation motifs (CH1-N45.2G; CH2-N120T-I121L-T122S; CH3-CHS-N135Q; and one of CH2-N20G; CH2-N20Q; CH2-N20T; or CH2-N20T-L21I-T22S). The anti-EGFR IgA4.0 variant is an aglycosylated IgA.

FIG. 5 shows a process flow of anti-EGFR IgA3.0min DS manufacturing.

FIGS. 6A-6B show genetic maps of vector components included in expression plasmids that were used during the manufacturing of the nonclinical product lots. FIG. 6A shows a genetic map of vector components included in the expression plasmids of heavy chain of anti-EGFR IgA3.0min. FIG. 6B shows a genetic map of vector components included in the expression plasmids of light chain of anti-EGFR IgA3.0min.

FIGS. 7A-7F shows images of SDS-PAGE gel image of various anti-EGFR IgA3.0min drug substances (DS) to visualize the intermediate products on size and purity after Capto L antibody capture and Size exclusion by SEC, and results of HPLC-SEC profiles of the anti-EGFR IgA3.0min antibodies obtained from production runs. Three anti-EGFR IgA3.0min drug substance (DS) were produced: (1) DS1 comprised a VH sequence of SEQ ID NO: 173 and a VL sequence of SEQ ID NO: 211; (2) DS2 comprised a VH sequence of SEQ ID NO: 172 and a VL sequence of SEQ ID NO: 198; and (3) DS3 comprised a VH sequence of SEQ ID NO: 182 and a VL sequence of SEQ ID NO: 207. Specifically, FIGS. 7A, 7C and 7E depict reducing (left) and non-reducing (right) SDS-PAGE gel images of anti-EGFR IgA3.0min DS1, anti-EGFR IgA3.0min DS2, and anti-EGFR IgA3.0min DS3 to visualize the intermediate products on size and purity after Capto L antibody capture and Size exclusion by SEC, respectively. Expected band sizes are indicated: Full anti-EGFR IgA3.0min antibody, 150 kDa; anti-EGFR IgA3.0min antibody HC and LC, 75 kDa and 25 kDa respectively. FIGS. 7B, 7D and 7F show results of HPLC-SEC profiles of the anti-EGFR IgA3.0min DS1, anti-EGFR IgA3.0min DS2, and anti-EGFR IgA3.0min DS3 antibodies obtained from production runs, respectively. Antibody purity is indicated in percentages.

FIG. 8 shows a process flow chart of release testing for anti-EGFR IgA3.0min DP.

FIGS. 9A-9E show results of binding measurements of four anti-EGFR IgA3.0min DP to EGFR. Briefly, four variants included: (1) anti-EGFR IgA3.0min DP1 comprising a VH sequence of SEQ ID NO: 173 and a VL sequence of SEQ ID NO: 211; (2) anti-EGFR IgA3.0min DP2 comprising a VH sequence of SEQ ID NO: 172 and a VL sequence of SEQ ID NO: 198; (3) anti-EGFR IgA3.0min DP3 comprising a VH sequence of SEQ ID NO: 182 and a VL sequence of SEQ ID NO: 207; and (4) anti-EGFR IgA3.0min DP4 comprising a VH sequence of SEQ ID NO: 184 and a VL sequence of SEQ ID NO: 209. FIGS. 9A-9D show results of Surface Plasmon resonance (SPR) binding measurements of anti-EGFR IgA3.0min DP1, anti-EGFR IgA3.0min DP2, anti-EGFR IgA3.0min DP3 and anti-EGFR IgA3.0min DP4 to Immobilized His-tagged EGFR on an Ni²⁺-nitrilotriacetic acid sensor chip, respectively. All measurements were carried out on System, BiaCore T200. FIG. 9E shows results of binding of anti-EGFR IgA3.0min DP1 to EGFR-expressing A431 and A1207 cell lines, and also the D562 cell lines which lowly express EGFR, measured by FACS. Various concentrations of antibodies were incubated with cells followed by addition of fluorescently labeled anti IgA secondary antibody and detected by flow cytometry. A no-primary antibody control was included, with just the addition of the fluorescently labeled anti IgA secondary antibody to the A431 cell line.

FIGS. 10A-10B show results of dose dependent inhibition of EGF Binding by each anti-EGFR IgA3.0min DP. Specifically, FIG. 10A shows results of dose dependent inhibition of EGF binding by four anti-EGFR IgA3.0min DP, wherein (1) anti-EGFR IgA3.0min DP1 comprised a VH sequence of SEQ ID NO: 173 and a VL sequence of SEQ ID NO: 211; (2) anti-EGFR IgA3.0min DP2 comprised a VH sequence of SEQ ID NO: 172 and a VL sequence of SEQ ID NO: 198; (3) anti-EGFR IgA3.0min DP3 comprised a VH sequence of SEQ ID NO: 182 and a VL sequence of SEQ ID NO: 207; and (4) anti-EGFR IgA3.0min DP4 comprised a VH sequence of SEQ ID NO: 184 and a VL sequence of SEQ ID NO: 209. FIG. 10B is an alternate representation of the same data as FIG. 10A for anti-EGFR IgA3.0min DP1. As shown, mean fluorescent intensity (MFI), a measure of the amount of fluorescently labelled EGF bound to EGFR, was measured using a competitive FACS assay across increasing concentrations of anti-EGFR IgA3.0min. MFI is reduced in a dose-dependent manner. Anti-EGFR IgA3.0min had no effect on the isotype control, demonstrating specific binding of the EGFR.

FIGS. 11A-11E show results of cell viability following treatment with each of four anti-EGFR IgA3.0min DP. Briefly, four variants include: (1) anti-EGFR IgA3.0min DP1 comprising a VH sequence of SEQ ID NO: 173 and a VL sequence of SEQ ID NO: 211; (2) anti-EGFR IgA3.0min DP2 comprising a VH sequence of SEQ ID NO: 172 and a VL sequence of SEQ ID NO: 198; (3) anti-EGFR IgA3.0min DP3 comprising a VH sequence of SEQ ID NO: 182 and a VL sequence of SEQ ID NO: 207; and (4) anti-EGFR IgA3.0min DP4 comprising a VH sequence of SEQ ID NO: 184 and a VL sequence of SEQ ID NO: 209. FIGS. 11A-11B show results of all four anti-EGFR IgA3.0min DP induced CDC (e.g., cell apoptosis). As shown, cell viability of A431 or Human Fibroblast cell lines was evaluated via the Sulforhodamine B assay kit in the presence of increasing concentrations of each anti-EGFR IgA3.0min DP. The survival of EGFR-highly expressing A431 cells was majorly reduced by each anti-EGFR IgA3.0min DP in a dose dependent manner. No change in Human Fibroblast survival is observed. FIGS. 11C-11D show alternate representations cell viability of A431 or Human Fibroblast cell lines following treatment with anti-EGFR IgA3.0min DP1 as shown in FIGS. 11A-11B, respectively. FIG. 11E shows alternate representation of cell viability assay for anti-EGFR IgA3.0min DPL.

FIGS. 12A-12E illustrate anti-EGFR IgA3.0min DP-induced ADCC for each of four anti-EGFR IgA3.0min DP, which included: (1) anti-EGFR IgA3.0min DP1 comprising a VH sequence of SEQ ID NO: 173 and a VL sequence of SEQ ID NO: 211; (2) anti-EGFR IgA3.0min DP2 comprising a VH sequence of SEQ ID NO: 172 and a VL sequence of SEQ ID NO: 198; (3) anti-EGFR IgA3.0min DP3 comprising a VH sequence of SEQ ID NO: 182 and a VL sequence of SEQ ID NO: 207; and (4) anti-EGFR IgA3.0min DP4 comprising a VH sequence of SEQ ID NO: 184 and a VL sequence of SEQ ID NO: 209. The specific cell lysis of an A431 cancer cell line was analyzed as a function of the concentration of each anti-EGFR IgA3.0min DP via either purified neutrophils from three donors (FIG. 12A), or via whole blood lysate cleared of erythrocytes (FIG. 12B). FIG. 12C is an alternate representations of specific cell lysis of an A431 cancer cell line as a function of the concentration of anti-EGFR IgA3.0min DP1 via purified neutrophils from three donors as shown in FIG. 12A. Similarly, FIG. 12D is an alternate representations of specific cell lysis of an A431 cancer cell line as a function of the concentration of anti-EGFR IgA3.0min DP1 via whole blood lysate cleared of erythrocytes from three donors as shown in FIG. 12B. Furthermore, the E:T ratio was calculated for three donors based on the specific lysis of the A431 cancer cell line via the neutrophils from the three donors (FIG. 12E).

FIGS. 13A-13C show results of an in vivo study of tumor growth in A431- and A549-Xenograft tumor model mice. Briefly, activity of four anti-EGFR IgA3.0min DP was tested, wherein the four anti-EGFR IgA3.0min DP included: (1) anti-EGFR IgA3.0min DP1 comprising a VH sequence of SEQ ID NO: 173 and a VL sequence of SEQ ID NO: 211; (2) anti-EGFR IgA3.0min DP2 comprising a VH sequence of SEQ ID NO: 172 and a VL sequence of SEQ ID NO: 198; (3) anti-EGFR IgA3.0min DP3 comprising a VH sequence of SEQ ID NO: 182 and a VL sequence of SEQ ID NO: 207; and (4) anti-EGFR IgA3.0min DP4 comprising a VH sequence of SEQ ID NO: 184 and a VL sequence of SEQ ID NO: 209. The activity of each anti-EGFR IgA3.0min DP was tested in vivo after a twice weekly dosing in xenograft tumor bearing CD89 Tg NXG mice injected with lowly expressing EGFR cancer cell lines (A549—FIG. 13A). FIG. 13B is an alternate representation of effect of the treatment of anti-EGFR IgA3.0min DP1 on lowly expressing EGFR (A549—FIG. 13B). FIG. 13C shows effects of the treatment of anti-EGFR IgA3.0min DP1 on highly expressing EGFR (A431—FIG. 13A). Each cancer cell line was transduced with the luciferase gene. PBS vehicle control was performed. Longitudinal monitoring of tumor growth by bioluminescent imaging (BLI), and the percentage increase in bioluminescent signal reported over time.

FIG. 14 depicts the pharmacokinetic profile of an anti-EGFR IgA3.0min DP following a single 3 mg/kg intravenous (IV) administration or intraperitoneal (IP) administration in immunocompromised NSG mice, wherein the anti-EGFR IgA3.0min DP comprised a VH sequence of SEQ ID NO: 173 and a VL sequence of SEQ ID NO: 211. The plasma concentration of the anti-EGFR IgA3.0min DP1 was measured over a 28-day period, and the mean plasma concentration (f geometric standard deviation shown by bars) was plotted as a function of time. N=4 per dosage group.

FIGS. 15A-15B depict pharmacokinetic profiles of anti-EGFR IgA3.0min DP administered in Non-human primates (NHP): (i) as a single 25 mg/kg intravenous (IV) administration (FIG. 15A); or (ii) in four 25 mg/kg IV administrations followed by a 12.5 mg/kg IV administration, each spaces 7 days apart (FIG. 15B). The anti-EGFR IgA3.0min DP comprised a VH sequence of SEQ ID NO: 173 and a VL sequence of SEQ ID NO: 211.

FIG. 16 summarizes overall clinical study design.

### DETAILED DESCRIPTION OF THE DISCLOSURE

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features can also be provided separately or in any suitable combination. Conversely, although the present disclosure may be described herein in the context of separate embodiments for clarity, the present disclosure can also be implemented in a single embodiment.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

Natural amino acids may be referred to herein by their conventional one or three letter abbreviations as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid. In some aspects, X can be asparagine (N), glutamine (Q), histidine (H), lysine (K), or arginine (R).

As used herein, the terms, “comprising” (and any form of comprising, such as “comprise” and “comprises”), “having” (and any form of having, such as “have” and “has”), “including” (and any form of including, such as “includes” and “include”) and “containing” (and any form of containing, such as “contains” and “contain”) are inclusive or open-ended and do not exclude additional, un-recited elements or method steps.

As used herein, the term, “about” or “approximately,” means within an acceptable error range for the particular value and includes a range of up to 10% of a given value or within an order of magnitude of the given value. Where particular values are described in the application and claims, unless otherwise stated the term “about” meaning within an acceptable error range for the particular value should be assumed.

As used herein, the term, “antibody,” refers to an immunoglobulin (Ig), whether naturally occurring or whether partially or fully synthetically produced and includes whole antibodies and fragments of antibodies unless explicitly stated.

A “whole antibody,” as used herein, refers to antibody that consists of four polypeptides: two heavy chain regions and two light chain regions, each containing: (i) a variable region that has: three complementarity determining regions (CDRs) that form the “hypervariable region” of an antibody and are responsible for binding to an antigen, where the three CDRs are separated by framework residues; and (ii) a constant region.

As used herein, the term, “Complementarity Determining Regions” (CDRs), refers to the amino acid residues of an antibody heavy chain variable region and light chain carriable region that are necessary for antigen binding.

As used herein, the term, “framework residues” or “FR,” refers to residues in the variable region other than the CDRs.

A “heavy chain region” or “heavy chain polypeptide,” as used herein, refers to an antibody portion that contains an N-terminal heavy chain variable (VH) region a C-terminal heavy chain constant (CH) region having one or more of: a CH1 domain, a hinge, a CH2 domain, a CH3 domain, and a CH3 tail piece (CS).

As used herein, the term, “hinge,” refers to a flexible domain of a heavy chain region that joins the CH1 domain to the CH2 domain, which allows the two N-terminal antigen binding regions to move independently.

A “light chain region” or “light chain polypeptide,” as used herein, refers to an antibody portion that contains an N-terminal light chain variable (VL) region and a C-terminal light chain constant (CL) region. Kappa (“κ”) and lambda (“λ”) light chains refer to the two major light chain isotypes.

As used herein, the term, “Fc domain” or “Fc comprising domain,” refers to portion of an antibody or from a non-antibody that can bind to an Fc receptor and includes a portion of a heavy chain constant (CH) region.

As used herein, the terms, “fragment of an antibody,” “antibody fragment,” “functional fragment of an antibody,” “antigen-binding portion” and grammatical equivalent thereof, are used interchangeably and refer to one or more fragments or portions of an antibody that retain the ability to specifically bind to an antigen and contains one or more complementary determining regions (CDRs).

A “Fab fragment” refers to a monovalent fragment consisting of the VL, VH, CL, and CH1 domains.

A “F(ab′)2” fragment refers to a bivalent fragment comprising two Fab fragments linked by a disulfide bridge at the stalk region.

An “Fv fragment” refers to a fragment consisting of the VL and VH domains of a single arm of an antibody.

A “single chain Fv (scFv)” refers to a monovalent fragment consisting of the two domains of the Fv fragment (i.e., VL and VH) joined by a synthetic linker which enables the two domains to be synthesized as a single polypeptide chain.

A “diabody” refers to a dimer of polypeptide chains, where each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites.

As used herein, the term, “monoclonal antibodies,” refers to antibodies that are produced by a single clone of B-cells and bind to the same epitope.

As used herein, the term, “polyclonal antibodies,” refers to antibodies that are produced by different B-cells and bind to different epitopes of the same antigen.

As used herein, the term, “chimeric antibody,” refers to an antibody that comprises an amino acid sequence derived from two different species or, or two different sources, and includes synthetic molecules.

The term, “recombinant antibody,” refers to an antibody that is expressed from a cell or cell line transfected with an expression vector (or possibly more than one expression vector, typically two expression vectors) comprising the coding sequence of the antibody, where said coding sequence is not naturally associated with the cell.

As used herein, the term, “recombinant human antibody,” refers to human antibodies that are prepared, expressed, created or isolated by recombinant means, including (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences.

As used herein, the term, “humanized antibody,” refers to non-human antibodies that contain amino acid sequences from a corresponding human antibody and are not naturally present in the non-human antibody.

As used herein, the term, “antigen,” refers to a molecule or portion of a molecule that can interact with a binding site of an antibody or fragment thereof and includes molecules or portions of molecules (epitopes) that can bind to antibodies.

As used herein, the term, “epitope,” refers to a region of an antigen that interacts with a specific antigen binding site in the variable region of an antibody molecule known as a paratope. Epitopes may be either conformational or linear. A “conformational epitope” is produced by spatially juxtaposed amino acids from different segments of the linear polypeptide chain. A “linear epitope” is produced by adjacent amino acid residues in a polypeptide chain.

As used herein, the term, “antigen binding domain,” refers to the portion of an antibody or a non-antibody that binds to or recognizes an antigen or a fragment thereof and includes the heavy chain variable (VH) region and light chain variable (VL) region.

As used herein, the term, “recognize,” refers to the association or binding between an antigen binding domain and an antigen.

As used herein, the terms “specifically binds” or “preferentially binds” refer binding to a target with greater affinity and/or avidity than binding to other polypeptides.

The term, “affinity,” as used herein, is represented by the equilibrium constant for the dissociation (K_{D}) of an antigen with an antigen-binding protein, and is a measure of the binding strength between an antigen and an antigen-binding domain: the lower the value of the K_{D}, the stronger the binding strength between an antigen and the antigen-binding domain. An antibody or antigen-binding fragment thereof is said to be “specific for” a first target or antigen compared to a second target or antigen when it binds to the first antigen with an affinity a K_{D} value that is at least 50 times lower than the K_{D} value for binding to another target or polypeptide.

The term, “avidity,” refers to the affinity of an antigen binding domain for an antigen based on the number of pertinent antigen binding sites present on the antigen-binding domain.

The term, “neutralizing activity,” as used herein, refers to the ability of the antibodies or a functional fragment thereof to block binding of a cognate ligand to a target antigen.

The term, “target cell,” as used herein, refers to a cell that can be targeted by the antibodies or a functional fragment thereof of the present disclosure.

As used herein, the terms, “polynucleotide” and “nucleic acid molecule,” are used interchangeably, and refer to polymers of deoxyribonucleotides, ribonucleotides, modified nucleotides, and/or their analogs, of any length, and includes DNA and RNA.

The term, “operably linked” or “transcriptional control,” refers to functional linkage between a regulatory sequence and a nucleic acid sequence resulting in expression of the latter.

As used herein, the term, “modification” or “mutation,” refers to an amino acid substitution, insertion, deletion in an antibody or fragment thereof, compared to a corresponding amino acid in a WT antibody.

As used herein, the term, “conservative substitution,” “conservative modification,” or “conservative mutation,” refers to the replacement of one amino acid by another amino acid that is chemically similar to the first amino acid and can be exchanged with the first amino acid in a polypeptide structure without significant disturbance or alteration of the polypeptide structure or function.

As used herein, the term, “non-conservative mutation,” “non-conservative modification,” or “non-conservative substitution,” refers to replacement of one amino acid by another amino acid that is chemically dissimilar to the first amino acid. When the second amino acid replaces the first amino acid in a nonconservative mutation or substitution, it results in disturbance or alteration of the polypeptide structure or function, which includes enhancement of the polypeptide structure or function.

As used herein, the term, “improved property,” when referring to one or more modifications of an antibody or fragment thereof, refers to a characteristic associated with the one or more modifications that is improved compared to a corresponding unmodified antibody.

As used herein, the terms, “corresponding antibody,” “corresponding unmodified antibody,” “corresponding wild type antibody,” and “corresponding antibody lacking one or more modifications,” refer to a wildtype antibody or fragment thereof having an amino acid sequence that corresponds to an amino acid sequence of an antibody or fragment thereof having one or more modifications at each amino acid residue other than the one or more modifications.

The term, “increased stability” or “improved stability,” as used herein when referring to an improved property, includes increased thermostability and/or decreased aggregation, as measured by a higher retention of a biological activity (e.g., ADCC, binding to an antigen, or binding to a FcαR) of an antibody or a functional fragment thereof after a period of incubation at a temperature, as compared to a corresponding antibody.

The term, “reduced aggregation” or “decreased aggregation,” as used herein when referring to an improved property, includes a reduction of aggregation of an antibody or a functional fragment thereof of the present disclosure with other antibody molecules and/or with other macromolecule including serum proteins such as albumin, as compared to aggregation exhibited by a corresponding antibody.

The term, “biodistribution” as used herein when referring to an improved property, refers to cellular and/or tissue and/or organ distribution of an antibody or a functional fragment thereof disclosed herein after administration or delivery to a subject. As used herein, the term “increased biodistribution” generally refers to an increase in the distribution of an IgA antibody or a functional fragment thereof disclosed herein at a target site such as a tumor or a tumor cell, as compared to administration of either a vehicle or a corresponding WT IgA antibody.

As used herein, the term, “in vivo half-life” or “circulating half-life,” refers to the time required for half the quantity of an antibody or fragment thereof to be cleared from circulation when administered in the animal. The term “increased circulating half-life” as used herein when referring to an improved property refers to a greater persistence in the serum or plasma and/or a longer period of time required to reduce to half the maximal measured serum or plasma concentration of an antibody or fragment thereof, as compared to administration of either a vehicle or a corresponding WT IgA antibody.

The term, “Fc-receptor mediated effector cell function,” refers to effector functions, such as phagocytosis, antibody dependent cellular cytotoxicity (ADCC), inflammatory mediator release, lysozyme production, and superoxide anion production which are triggered by binding of immunoglobulin, e.g., IgA, to an Fc receptor on an immune effector cell.

The term, “ADCC activity,” refers to the ability of an antibody to elicit lysis of a target cell (e.g. cancer cell) via an immune effector cell.

The term, “complement dependent cytotoxicity” or “CDC,” refers to the ability of an antibody to lyse a target cell (e.g. cancer cell) in the presence of complement.

The term, “glycosylation,” as used herein, refers to the covalent linking of one or more carbohydrates to a polypeptide.

The term, “isolated,” as used herein, refers separation or alteration of a molecule from its natural state. A nucleic acid or a polypeptide (e.g., antibodies or antigen binding fragment thereof disclosed herein) that is present in a cell and present with coexisting materials in its natural state is not “isolated,” but the same nucleic acid or polypeptide partially or completely separated from the cell and coexisting materials is “isolated.”

The term, “substantially purified,” as used herein, refers to a state in which an antibody or functional fragment thereof is substantially free of cellular material, naturally coexisting material, or other contaminating material from the cell or tissue source from which it is derived, or is substantially free from chemical precursors or other chemicals when chemically synthesized. As used herein, “substantially free” refers to a preparation having less than about 30% by dry weight of cellular material, naturally coexisting material, or other contaminating material.

The term, “leader sequence,” refers to a sequence of amino acid residues located at the N-terminus of a polypeptide that facilitates secretion of a polypeptide from a mammalian cell.

The term, “label,” refers to a compound or composition that is directly or indirectly detectable and is conjugated directly or indirectly to the antibody. A label is “directly detectable” when the label is detectable by itself (e.g., radioisotope labels or fluorescent labels) or when the label catalyzes a chemical alteration of a substrate that produces a detectable change (e.g. an enzymatic label). A label is “indirectly detectable” when the label is not detectable on its own, but binds to another agent that is directly detectable.

As used herein, the term, “fusion protein,” refers to a polypeptide that comprises an amino acid sequence of an antibody or fragment thereof and an amino acid sequence of a heterologous polypeptide (i.e., an unrelated polypeptide).

As used herein, the term, “cancer,” “tumor,” “proliferative disease,” “malignancy,” or “malignant disease,” relates to the physiological condition in mammals characterized by a cell having malignant properties. A “malignant property” includes uncontrolled growth, cellular intrusion, and metastasis formation. These malignant properties differentiate cancers from benign tumors, which typically do not invade or metastasize.

As used herein, the terms, “disease”, “disorder”, and “condition,” are used interchangeably to refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

As used herein, the terms, “in need thereof,” patient in need thereof,” and “subject in need thereof,” when used in the context of a therapeutic or prophylactic treatment, refer to a person having a disease, being diagnosed with a disease, being in need of preventing a disease, or being at risk of developing the disease.

As used herein, the terms, “treat,” “treatment,” “treating” and “amelioration,” refer to therapeutic and prophylactic treatments, wherein the object is to reverse, alleviate, ameliorate, inhibit, prevent, slow down, or stop the progression or severity of a condition associated with a disease or disorder. Treating includes reducing or alleviating at least one adverse effect or symptom of a condition, disease or disorder associated with a disease or disorder. Treatment is “effective” if one or more symptoms or clinical markers are reduced or if the progression of a disease is reduced or halted.

As used herein, the term, “administering,” refers to the placement of an antibody or fragment thereof into a subject by a method or route that results in at least partial delivery of the antibody or fragment thereof at a desired site. Administering can be by any appropriate route which results in an effective treatment in the subject.

The term, “combination therapy,” refers to agents that are given substantially contemporaneously, either simultaneously or sequentially.

The term, “therapeutically effective amount,” refers to an amount of an agent that is effective to achieve the desired therapeutic result in a given period of time.

The term, “prophylactically effective amount,” refers to an amount of an agent that is effective to achieve the desired prophylactic result in a given time period.

The terms, “parenteral administration” and “administered parenterally,” as used herein, refer to modes of administration other than enteral and topical administration, usually by injection.

The terms, “systemic administration,” “administered systemically”, “peripheral administration” and “administered peripherally,” as used herein, refer to administration other than directly into a target site, tissue, or organ, such as a tumor site, such that it enters the subject's circulatory system and, thus, is subject to metabolism and other like processes.

The term, “pharmaceutically acceptable,” as used herein, refers to those compounds, materials, compositions, and/or dosage forms that are within the scope of sound medical judgment and are suitable for contact with or administration to tissues of human beings and animals without excessive toxicity, irritation, allergic response, or other problem or complication, commensurate with a reasonable benefit/risk ratio.

As used herein, the terms, “subject”, “patient”, “individual” and like terms, are used interchangeably to refer to a vertebrate, a mammal, a primate, or a human.

As used herein, the term, “host cell,” refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

### INTRODUCTION

Described herein are engineered anti-EGFR IgA antibodies having an IgA backbone and an anti-EGFR binding domain. In some embodiments, the engineered antibody has one or more of: reduced N-glycosylation profile, a removal of C-terminal residues in the heavy chain constant region, or amino acid substitutions that favor a stabilized monomeric form. In some embodiments, the engineered IgA antibody is directed against human EGFR, with a Fc domain that recognizes FcαRI receptors (CD89) on immune cells such as neutrophils, macrophages, and eosinophils. Accordingly, in some embodiments, the engineered antibody is involved the selective recruitment of FcαRI expressing immune effector cells to the EGFR tumor antigen on the cell surface. In some embodiments, the engineered antibody selectively recruits neutrophils. Neutrophils are capable of killing tumor cells directly by releasing reactive oxygen species (ROS) and reactive nitrogen species (RNS). Accordingly, in some embodiments, the engineered antibody induces ADCC in EGFR tumor antigen expressing cells. Alternatively, in some embodiments, the engineered antibody induces direct cytotoxicity to EGFR-positive tumor cells, in the absence of immune effector cells, via antagonistic binding to EGFR and abrogation of EGF signaling. Additionally, in some embodiments, the engineered antibody-induced cytotoxicity enhances T-cell activation and attract proinflammatory (M1) macrophages.

### Anti-EGFR IgA Antibodies

Disclosed herein are engineered anti-EGFR IgA antibodies. IgA antibodies make up 15-20% of serum immunoglobulins. At the mucosal surface, IgA is the most abundant and it plays a significant role in the body's immune response. IgA can be highly versatile, existing as monomeric, dimeric, polymeric, and secretory IgA at mucosal tissues. IgA has two subclasses (IgA1 and IgA2) and can be produced as a monomeric as well as a dimeric form and secretory form. In some embodiments, an anti-EGFR IgA antibody can be monomeric. In some embodiments, an anti-EGFR IgA antibody can comprise one or more IgA1 amino acid sequences. In some embodiments, an anti-EGFR IgA antibody can comprise one or more IgA2 amino acid sequences. In some embodiments, an anti-EGFR IgA antibody can comprise one or more IgA1 amino acid sequences and one or more IgA2 amino acid sequences. IgA1 and IgA2 differ in their hinge region and carbohydrate content. Monomeric IgA1 is the predominant subclass in serum and is susceptible to cleavage by bacterial enzymes, while IgA2 is more resistant to enzymatic degradation and is found mainly in mucosal secretions.

In some embodiments, an anti-EGFR IgA antibody is an IgA2 antibody of allotype: IgA2m(1), IgA2m(2), or IgA2n. In some embodiments, an IgA2m(1) antibody is a Caucasian IgA2m(1) antibody. In some embodiments, an IgA2m(2) antibody is an African IgA2m(2) antibody or an Asian IgA2m(2) antibody. In some embodiments, IgA2m(1) also comprises a superior potential efficacy and safety profile, owing to the low glycosylation levels and lack of association with IgA nephropathy compared to IgG1 and IgA1/IgA2m(2).

In some embodiments, an anti-EGFR IgA antibody is a therapeutic antibody. In some embodiments, an anti-EGFR IgA antibody can be a humanized antibody. In some embodiments, an anti-EGFR IgA antibody can be a chimeric antibody. In some embodiments, an anti-EGFR IgA antibody can be a human antibody. In some embodiments, an anti-EGFR IgA antibody is a recombinant antibody. In some embodiments, an anti-EGFR IgA antibody is a recombinant human antibody. In some embodiments, an anti-EGFR IgA antibody can be a monospecific antibody. In some embodiments, an anti-EGFR IgA antibody can be a bispecific antibody. In some embodiments, an anti-EGFR IgA antibody can be a tri-specific antibody. In some embodiments, an anti-EGFR IgA antibody can be a multi-specific antibody.

In some embodiments, an anti-EGFR IgA antibody can be a chimeric antibody, a single chain antibody, a humanized antibody, a human antibody, a monoclonal antibody, a deimmunized antibody, a bispecific antibody, a multispecific antibody, a multivalent antibody, or a combination thereof. In some embodiments, an anti-EGFR IgA antibody can be a bispecific antibody that binds EGFR and a second antigen. In some embodiments, an anti-EGFR IgA antibody co-engages two antigens at the cell surface. In some examples, a binding of the anti-EGFR IgA antibody to two different antigens is sequential. For example, a binding of the anti-EGFR IgA antibody to a first antigen occurs first and thereby restricts a space explored by a second antibody arm. Consequentially, there can be a significant increase in local concentration of the second antigen, which can facilitate a binding of the second antibody arm.

### Constant Region

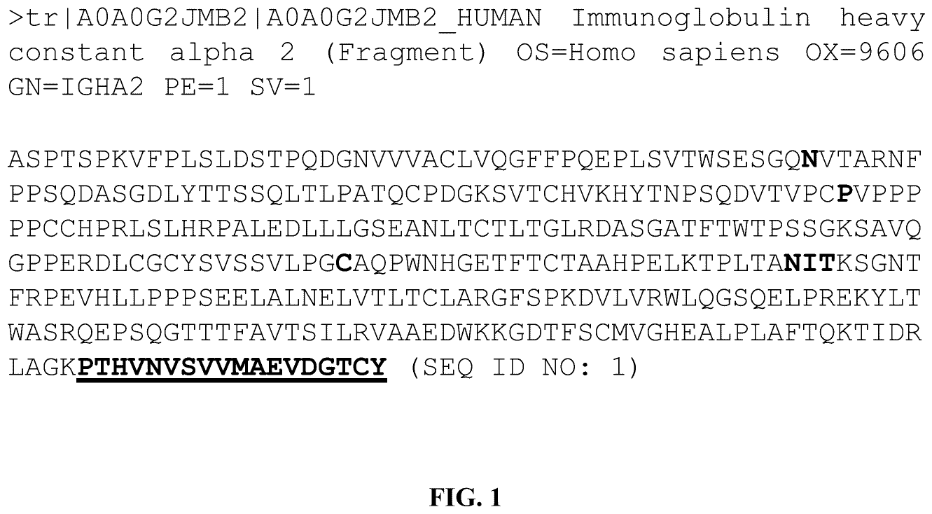

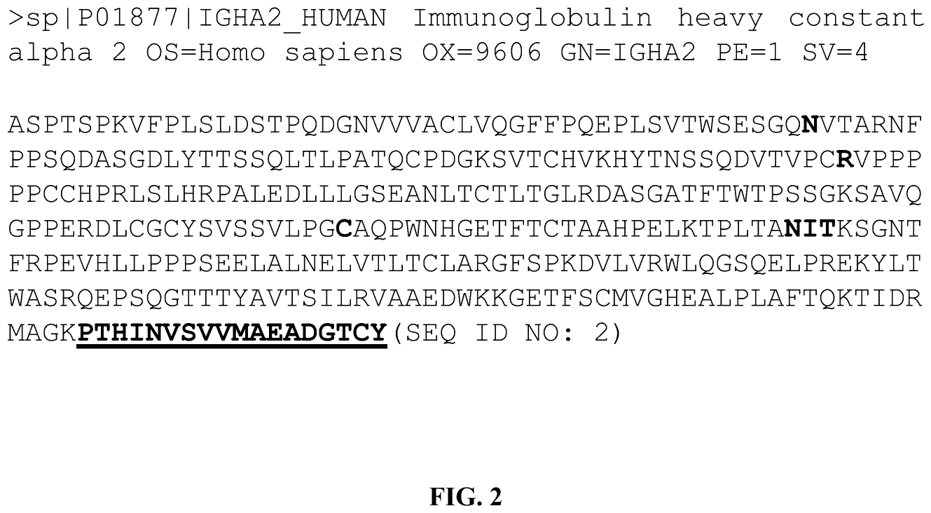

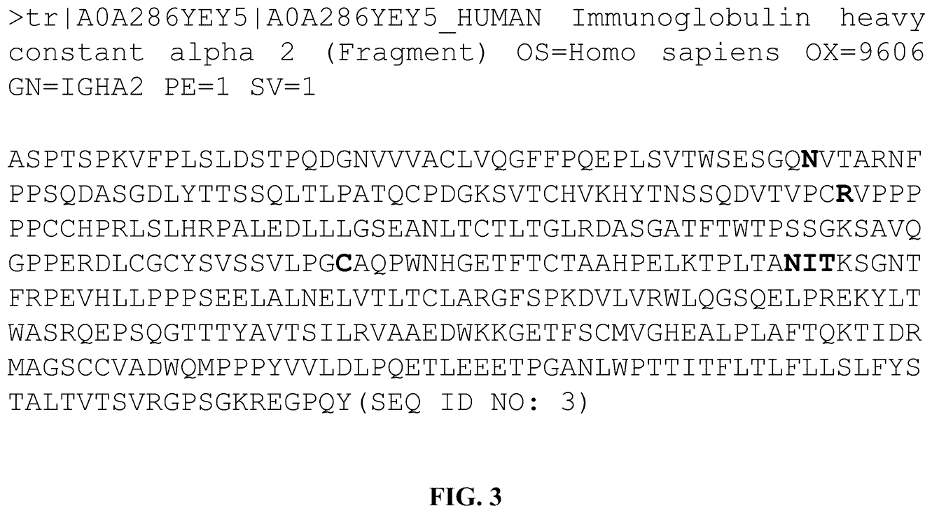

In some embodiments, an anti-EGFR IgA antibody comprises an IgA heavy chain constant region or afunctional variant thereof. TABLE 1 lists exemplary amino acid sequences of IgA heavy chain constant region and nucleotide sequences encoding the same. FIGS. 1-3 provides three representative amino acid sequences of the human IgA2 heavy chain from TABLE 1. In some embodiments, an IgA heavy chain constant region comprises an amino acid sequence that is at least 60%, at least 70%, at least 75%, at least 80%, at least 85%, at least 90%, at least 95%, or at least 99% identical to any one of the amino acid sequences of TABLE 1 (SEQ ID NOs: 1-9). In some embodiments, an IgA heavy chain constant region is encoded by a nucleotide sequence that is at least 60%, at least 70%, at least 75%, at least 80%, at least 85%, at least 90%, at least 95%, or at least 99% identical to any one of the nucleotide sequences of TABLE 1.

TABLE 1
Exemplary Amino Acid Sequences of IgA Heavy Chain Constant Regions

| Name | Amino Acid Sequence | Nucleotide Sequence |
|---|---|---|
| IgA2m1 | ASPTSPKVFPLSLDSTPQDGNVVVA | Gccagccccaccagccccaaggtgttccccctgagcctggacagcaccc |
| | CLVQGFFPQEPLSVTWSESGQNVTA | cccaggacggcaacgtggtggtggcctgcctggtgcagggcttcttcccc |
| | RNFPPSQDASGDLYTTSSQLTLPATQ | caggagcccctgagcgtgacctggagcgagagcggccagaacgtgacc |
| | CPDGKSVTCHVKHYTNPSQDVTVP | gccaggaacttcccccccagccaggacgccagcggcgacctgtacacca |
| | CPVPPPPPCCHPRLSLHRPALEDLLL | ccagcagccagctgaccctgcccgccacccagtgccccgacggcaaga |
| | GSEANLTCTLTGLRDASGATFTWTP | gcgtgacctgccacgtgaagcactacaccaaccccagccaggacgtgac |
| | SSGKSAVQGPPERDLCGCYSVSSVL | cgtgccctgccccgtgccccccccccccccctgctgccaccccaggctga |
| | PGCAQPWNHGETFTCTAAHPELKTP | gcctgcacaggcccgccctggaggacctgctgctgggcagcgaggcca |
| | LTANITKSGNTFRPEVHLLPPPSEEL | acctgacctgcaccctgaccggcctgagggacgccagcggcgccacctt |
| | ALNELVTLTCLARGFSPKDVLVRWL | cacctggacccccagcagcggcaagagcgccgtgcagggcccccccga |
| | QGSQELPREKYLTWASRQEPSQGTT | gagggacctgtgcggctgctacagcgtgagcagcgtgctgcccggctgc |
| | TFAVTSILRVAAEDWKKGDTFSCM | gcccagccctggaaccacggcgagaccttcacctgcaccgccgcccacc |
| | VGHEALPLAFTQKTIDRLAGKPTHV | ccgagctgaagacccccctgaccgccaacatcaccaagagggcaacac |
| | NVSVVMAEVDGTCY (SEQ ID NO: 1) | cttcaggcccgaggtgcacctgctgcccccccccagcgaggagctggcc |
| | | ctgaacgagctggtgaccctgacctgcctggccaggggcttcagccccaa |
| | | ggacgtgctggtgaggtggctgcagggcagccaggagctgcccaggga |
| | | gaagtacctgacctgggccagcaggcaggagcccagccagggcaccac |
| | | caccttcgccgtgaccagcatcctgagggtggccgccgaggactggaag |
| | | aagggcgacaccttcagctgcatggtgggccacgaggccctgcccctgg |
| | | ccttcacccagaagaccatcgacaggctggccggcaagcccacccacgt |
| | | gaacgtgagcgtggtgatggccgaggtggacggcacctgctac (SEQ |
| | | ID NO: 10) |
| IgA2m2 | ASPTSPKVFPLSLDSTPQDGNVVVA | Gccagccccaccagccccaaggtgttccccctgagcctggacagcaccc |
| | CLVQGFFPQEPLSVTWSESGQNVTA | cccaggacggcaacgtggtggtggcctgcctggtgcagggcttcttcccc |
| | RNFPPSQDASGDLYTTSSQLTLPATQ | caggagcccctgagcgtgacctggagcgagagcggccagaacgtgacc |
| | CPDGKSVTCHVKHYTNSSQDVTVP | gccaggaacttcccccccagccaggacgccagcggcgacctgtacacca |
| | CRVPPPPPCCHPRLSLHRPALEDLLL | ccagcagccagctgaccctgcccgccacccagtgccccgacggcaaga |
| | GSEANLTCTLTGLRDASGATFTWTP | gcgtgacctgccacgtgaagcactacaccaacagcagccaggacgtgac |
| | SSGKSAVQGPPERDLCGCYSVSSVL | cgtgccctgcagggtgccccccccccccccctgctgccaccccaggctga |
| | PGCAQPWNHGETFTCTAAHPELKTP | gcctgcacaggcccgccctggaggacctgctgctgggcagcgaggcca |
| | LTANITKSGNTFRPEVHLLPPPSEEL | acctgacctgcaccctgaccggcctgagggacgccagcggcgccacctt |
| | ALNELVTLTCLARGFSPKDVLVRWL | cacctggacccccagcagcggcaagagcgccgtgcagggcccccccga |
| | QGSQELPREKYLTWASRQEPSQGTT | gagggacctgtgcggctgctacagcgtgagcagcgtgctgcccggctgc |
| | TYAVTSILRVAAEDWKKGETFSCM | gcccagccctggaaccacggcgagaccttcacctgcaccgccgcccacc |
| | VGHEALPLAFTQKTIDRMAGKPTHI | ccgagctgaagacccccctgaccgccaacatcaccaagagcggcaacac |
| | NVSVVMAEADGTCY (SEQ ID NO: 2) | cttcaggcccgaggtgcacctgctgcccccccccagcgaggagctggcc |
| | | ctgaacgagctggtgaccctgacctgcctggccaggggcttcagccccaa |
| | | ggacgtgctggtgaggtggctgcagggcagccaggagctgcccaggga |
| | | gaagtacctgacctgggccagcaggcaggagcccagccagggcaccac |
| | | cacctacgccgtgaccagcatcctgagggtggccgccgaggactggaag |
| | | aagggcgagaccttcagctgcatggtgggccacgaggccctgcccctgg |
| | | ccttcacccagaagaccatcgacaggatggccggcaagcccacccacatc |
| | | aacgtgagcgtggtgatggccgaggccgacggcacctgctac (SEQ |
| | | ID NO: 11) |
| IgA2_ | ASPTSPKVFPLSLDSTPQDGNVVVA | gccagccccaccagccccaaggtgttccccctgagcctggacagcaccc |
| A0A286YEY5 | CLVQGFFPQEPLSVTWSESGQNVTA | cccaggacggcaacgtggtggtggcctgcctggtgcagggcttcttcccc |
| | RNFPPSQDASGDLYTTSSQLTLPATQ | caggagcccctgagcgtgacctggagcgagagcggccagaacgtgacc |
| | CPDGKSVTCHVKHYTNSSQDVTVP | gccaggaacttcccccccagccaggacgccagcggcgacctgtacacca |
| | CRVPPPPPCCHPRLSLHRPALEDLLL | ccagcagccagctgaccctgcccgccacccagtgccccgacggcaaga |
| | GSEANLTCTLTGLRDASGATFTWTP | gcgtgacctgccacgtgaagcactacaccaacagcagccaggacgtgac |
| | SSGKSAVQGPPERDLCGCYSVSSVL | cgtgccctgcagggtgccccccccccccccctgctgccaccccaggctga |
| | PGCAQPWNHGETFTCTAAHPELKTP | gcctgcacaggcccgccctggaggacctgctgctgggcagcgaggcca |
| | LTANITKSGNTFRPEVHLLPPPSEEL | acctgacctgcaccctgaccggcctgagggacgccagcggcgccacctt |
| | ALNELVTLTCLARGFSPKDVLVRWL | cacctggacccccagcagcggcaagagcgccgtgcagggcccccccga |
| | QGSQELPREKYLTWASRQEPSQGTT | gagggacctgtgcggctgctacagcgtgagcagcgtgctgcccggctgc |
| | TYAVTSILRVAAEDWKKGETFSCM | gcccagccctggaaccacggcgagaccttcacctgcaccgccgcccacc |
| | VGHEALPLAFTQKTIDRMAGSCCVA | ccgagctgaagacccccctgaccgccaacatcaccaagagcggcaacac |
| | DWQMPPPYVVLDLPQETLEEETPGA | cttcaggcccgaggtgcacctgctgcccccccccagcgaggagctggcc |
| | NLWPTTITFLTLFLLSLFYSTALTVTS | ctgaacgagctggtgaccctgacctgcctggccaggggcttcagccccaa |
| | VRGPSGKREGPQY (SEQ ID NO: 3) | ggacgtgctggtgaggtggctgcagggcagccaggagctgcccaggga |
| | | gaagtacctgacctgggccagcaggcaggagcccagccagggcaccac |
| | | cacctacgccgtgaccagcatcctgagggtggccgccgaggactggaag |
| | | aagggcgagaccttcagctgcatggtgggccacgaggccctgcccctgg |
| | | ccttcacccagaagaccatcgacaggatggccggcagctgctgcgtggcc |
| | | gactggcagatgccccccccctacgtggtgctggacctgccccaggagac |
| | | cctggaggaggagacccccggcgccaacctgtggcccaccaccatcacc |
| | | ttcctgaccctgttcctgctgagcctgttctacagcaccgccctgaccgtgac |
| | | cagcgtgaggggccccagcggcaagagggagggcccccagtac |
| | | (SEQ ID NO: 12) |
| IgA3.0+ | ASPTSPKVFPLSLDSTPQDGNVVVA | Gctagcccaacctctcctaaggtgttccctctgagcctggacagcacccct |
| | CLVQGFFPQEPLSVTWSESGQGVTA | caggatggaaatgtggtggtggcctgtctggtgcagggattcttcccacaa |
| | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEANLTCTLTGLRDASGATFTWTP | acgtgaagcactacaccaatcctagccaggacgtgaccgtgccttgcaga |
| | SSGKSAVQGPPERDLCGCYSVSSVL | gttcctcctcctccaccttgctgtcaccctcggctgtctctgcacagacccgc |
| | PGSAQPWNHGETFTCTAAHPELKTP | tctggaagatctgctgctgggctctgaggccaacctgacatgtaccctgacc |
| | LTATLSKSGNTFRPEVHLLPPPSEEL | ggcctgagagatgcttctggcgccacctttacctggacaccttccagcgga |
| | ALNELVTLTCLARGFSPKDVLVRWL | aagtccgctgttcagggacctcctgagagggacctgtgcggctgttactctg |
| | QGSQELPREKYLTWASRQEPSQGTT | tgtctagtgtgctgcctggcagcgcccagccttggaatcatggcgagacatt |
| | TFAVTSILRVAAEDWKKGDTFSCM | cacctgtaccgctgctcaccccgagctgaaaacccctctgaccgccacact |
| | VGHEALPLAFTQKTIDRLAGKPTHV | gtccaagtccggcaacaccttccggcctgaagtgcatctgctgcctccacct |
| | QVSVVMAEVDGT (SEQ ID NO: 4) | agcgaggaactggccctgaatgagctggtcaccctgacctgtctggccag |
| | | gggctttagccctaaggacgtgctcgttagatggctgcagggctcccaaga |
| | | gctgcccagagagaagtatctgacctgggcctctcggcaagagccatctc |
| | | agggcaccacaacctttgccgtgaccagcatcctgagagtggccgccgaa |
| | | gattggaagaagggcgacaccttcagctgcatggtcggacatgaagccct |
| | | gcctctggctttcacccagaaaaccatcgacagactggccggcaagccca |
| | | cccatgtccaagtgtctgttgtcatggcggaggtggacggcacc (SEQ |
| | | ID NO: 13) |
| IgA3.0 | ASPTSPKVFPLSLDSTPQDGNVVVA | Gctagcccaacctctcctaaggtgttccctctgagcctggacagcacccct |
| min | CLVQGFFPQEPLSVTWSESGQGVTA | caggatggaaatgtggtggtggcctgtctggtgcagggattcttcccacaa |
| | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEANLTCTLTGLRDASGATFTWTP | acgtgaagcactacaccaatcctagccaggacgtgaccgtgccttgcaga |
| | SSGKSAVQGPPERDLCGCYSVSSVL | gttcctcctcctccaccttgctgtcaccctcggctgtctctgcacagacccgc |
| | PGSAQPWNHGETFTCTAAHPELKTP | tctggaagatctgctgctgggctctgaggccaacctgacatgtaccctgacc |
| | LTATLSKSGNTFRPEVHLLPPPSEEL | ggcctgagagatgcttctggcgccacctttacctggacaccttccagcgga |
| | ALNELVTLTCLARGFSPKDVLVRWL | aagtccgctgttcagggacctcctgagagggacctgtgcggctgttactctg |
| | QGSQELPREKYLTWASRQEPSQGTT | tgtctagtgtgctgcctggcagcgcccagccttggaatcatggcgagacatt |
| | TFAVTSILRVAAEDWKKGDTFSCM | cacctgtaccgctgctcaccccgagctgaaaacccctctgaccgccacact |
| | VGHEALPLAFTQKTIDRLAGK (SEQ | gtccaagtccggcaacaccttccggcctgaagtgcatctgctgcctccacct |
| | ID NO: 5) | agcgaggaactggccctgaatgagctggtcaccctgacctgtctggccag |
| | | gggctttagccctaaggacgtgctcgttagatggctgcagggctcccaaga |
| | | gctgcccagagagaagtatctgacctgggcctctcggcaagagccatctc |
| | | agggcaccacaacctttgccgtgaccagcatcctgagagtggccgccgaa |
| | | gattggaagaagggcgacaccttcagctgcatggtcggacatgaagccct |
| | | gcctctggctttcacccagaaaaccatcgacagactggccggcaag |
| | | (SEQ ID NO: 14) |
| IgA4.0_ | ASPTSPKVFPLSLDSTPQDGNVVVA | Gctagcccaacctctcctaaggtgttccctctgagcctggacagcacccct |
| NG | CLVQGFFPQEPLSVTWSESGQGVTA | caggatggaaatgtggtggtggcctgtctggtgcagggattcttcccacaa |
| | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEAGLTCTLTGLRDASGATFTWTP | acgtgaagcactacaccaatcctagccaggacgtgaccgtgccttgcaga |
| | SSGKSAVQGPPERDLCGCYSVSSVL | gttcctcctcctccaccttgctgtcaccctcggctgtctctgcacagacccgc |
| | PGSAQPWNHGETFTCTAAHPELKTP | tctggaagatctgctgctgggctctgaggccggcctgacatgtaccctgac |
| | LTATLSKSGNTFRPEVHLLPPPSEEL | cggcctgagagatgcttctggcgccacctttacctggacaccttccagcgg |
| | ALNELVTLTCLARGFSPKDVLVRWL | aaagtccgctgttcagggacctcctgagagggacctgtgcggctgttactct |
| | QGSQELPREKYLTWASRQEPSQGTT | gtgtctagtgtgctgcctggcagcgcccagccttggaatcatggcgagaca |
| | TFAVTSILRVAAEDWKKGDTFSCM | ttcacctgtaccgctgctcaccccgagctgaaaacccctctgaccgccaca |
| | VGHEALPLAFTQKTIDRLAGK (SEQ | ctgtccaagtccggcaacaccttccggcctgaagtgcatctgctgcctccac |
| | ID NO: 6) | ctagcgaggaactggccctgaatgagctggtcaccctgacctgtctggcca |
| | | ggggctttagccctaaggacgtgctcgttagatggctgcagggctcccaag |
| | | agctgcccagagagaagtatctgacctgggcctctcggcaagagccatct |
| | | cagggcaccacaacctttgccgtgaccagcatcctgagagtggccgccga |
| | | agattggaagaagggcgacaccttcagctgcatggtcggacatgaagccc |
| | | tgcctctggctttcacccagaaaaccatcgacagactggccggcaag |
| | | (SEQ ID NO: 15) |
| IgA4.0_ | ASPTSPKVFPLSLDSTPQDGNVVVA | Gctagcccaacctctcctaaggtgttccctctgagcctggacagcacccct |
| NQ | CLVQGFFPQEPLSVTWSESGQGVTA | caggatggaaatgtggtggtggcctgtctggtgcagggattcttcccacaa |
| | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEAQLTCTLTGLRDASGATFTWTP | acgtgaagcactacaccaatcctagccaggacgtgaccgtgccttgcaga |
| | SSGKSAVQGPPERDLCGCYSVSSVL | gttcctcctcctccaccttgctgtcaccctcggctgtctctgcacagacccgc |
| | PGSAQPWNHGETFTCTAAHPELKTP | tctggaagatctgctgctgggctctgaggcccagctgacatgtaccctgac |
| | LTATLSKSGNTFRPEVHLLPPPSEEL | cggcctgagagatgcttctggcgccacctttacctggacaccttccagcgg |
| | ALNELVTLTCLARGFSPKDVLVRWL | aaagtccgctgttcagggacctcctgagagggacctgtgcggctgttactct |
| | QGSQELPREKYLTWASRQEPSQGTT | gtgtctagtgtgctgcctggcagcgcccagccttggaatcatggcgagaca |
| | TFAVTSILRVAAEDWKKGDTFSCM | ttcacctgtaccgctgctcaccccgagctgaaaacccctctgaccgccaca |
| | VGHEALPLAFTQKTIDRLAGK (SEQ | ctgtccaagtccggcaacaccttccggcctgaagtgcatctgctgcctccac |
| | ID NO: 7) | ctagcgaggaactggccctgaatgagctggtcaccctgacctgtctggcca |
| | | ggggctttagccctaaggacgtgctcgttagatggctgcagggctcccaag |
| | | agctgcccagagagaagtatctgacctgggcctctcggcaagagccatct |
| | | cagggcaccacaacctttgccgtgaccagcatcctgagagtggccgccga |
| | | agattggaagaagggcgacaccttcagctgcatggtcggacatgaagccc |
| | | tgcctctggctttcacccagaaaaccatcgacagactggccggcaag |
| | | (SEQ ID NO: 16) |
| IgA4.0_ | ASPTSPKVFPLSLDSTPQDGNVVVA | Gctagcccaacctctcctaaggtgttccctctgagcctggacagcacccct |
| NT | CLVQGFFPQEPLSVTWSESGQGVTA | caggatggaaatgtggtggtggcctgtctggtgcagggattcttcccacaa |
| | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEATLTCTLTGLRDASGATFTWTP | acgtgaagcactacaccaatcctagccaggacgtgaccgtgccttgcaga |
| | SSGKSAVQGPPERDLCGCYSVSSVL | gttcctcctcctccaccttgctgtcaccctcggctgtctctgcacagacccgc |
| | PGSAQPWNHGETFTCTAAHPELKTP | tctggaagatctgctgctgggctctgaggccaccctgacatgtaccctgacc |
| | LTATLSKSGNTFRPEVHLLPPPSEEL | ggcctgagagatgcttctggcgccacctttacctggacaccttccagcgga |
| | ALNELVTLTCLARGFSPKDVLVRWL | aagtccgctgttcagggacctcctgagagggacctgtgcggctgttactctg |
| | QGSQELPREKYLTWASRQEPSQGTT | tgtctagtgtgctgcctggcagcgcccagccttggaatcatggcgagacatt |
| | TFAVTSILRVAAEDWKKGDTFSCM | cacctgtaccgctgctcaccccgagctgaaaacccctctgaccgccacact |
| | VGHEALPLAFTQKTIDRLAGK (SEQ | gtccaagtccggcaacaccttccggcctgaagtgcatctgctgcctccacct |
| | ID NO: 8) | agcgaggaactggccctgaatgagctggtcaccctgacctgtctggccag |
| | | gggctttagccctaaggacgtgctcgttagatggctgcagggctcccaaga |
| | | gctgcccagagagaagtatctgacctgggcctctcggcaagagccatctc |
| | | agggcaccacaacctttgccgtgaccagcatcctgagagtggccgccgaa |
| | | gattggaagaagggcgacaccttcagctgcatggtcggacatgaagccct |
| | | gcctctggctttcacccagaaaaccatcgacagactggccggcaag |
| | | (SEQ ID NO: 17) |
| IgA4.0_ | ASPTSPKVFPLSLDSTPQDGNVVVA | Gctagcccaacctctcctaaggtgttccctctgagcctggacagcacccct |
| NLT- | CLVQGFFPQEPLSVTWSESGQGVTA | caggatggaaatgtggtggtggcctgtctggtgcagggattcttcccacaa |
| TIS | RNFPPSQDASGDLYTTSSQLTLPATQ | gagcccctgtccgtgacttggagcgaatctggacagggcgtgaccgccag |
| | CPDGKSVTCHVKHYTNPSQDVTVP | aaacttcccaccttctcaggacgcctctggcgacctgtacaccacctcttctc |
| | CRVPPPPPCCHPRLSLHRPALEDLLL | agctgaccctgcctgccacacagtgccctgatggcaagtctgtgacctgcc |
| | GSEATISCTLTGLRDASGATFTWTPS | acgtgaagcactacaccaatcctagccaggacgtgaccgtgccttgcaga |
| | SGKSAVQGPPERDLCGCYSVSSVLP | gttcctcctcctccaccttgctgtcaccctcggctgtctctgcacagacccgc |
| | GSAQPWNHGETFTCTAAHPELKTPL | tctggaagatctgctgctgggctctgaggccaccatcagctgtaccctgacc |
| | TATLSKSGNTFRPEVHLLPPPSEELA | ggcctgagagatgcttctggcgccacctttacctggacaccttccagcgga |
| | LNELVTLTCLARGFSPKDVLVRWLQ | aagtccgctgttcagggacctcctgagagggacctgtgcggctgttactctg |
| | GSQELPREKYLTWASRQEPSQGTTT | tgtctagtgtgctgcctggcagcgcccagccttggaatcatggcgagacatt |
| | FAVTSILRVAAEDWKKGDTFSCMV | cacctgtaccgctgctcaccccgagctgaaaacccctctgaccgccacact |
| | GHEALPLAFTQKTIDRLAGK (SEQ ID | gtccaagtccggcaacaccttccggcctgaagtgcatctgctgcctccacct |
| | NO: 9) | agcgaggaactggccctgaatgagctggtcaccctgacctgtctggccag |
| | | gggctttagccctaaggacgtgctcgttagatggctgcagggctcccaaga |
| | | gctgcccagagagaagtatctgacctgggcctctcggcaagagccatctc |
| | | agggcaccacaacctttgccgtgaccagcatcctgagagtggccgccgaa |
| | | gattggaagaagggcgacaccttcagctgcatggtcggacatgaagccct |
| | | gcctctggctttcacccagaaaaccatcgacagactggccggcaag |
| | | (SEQ ID NO: 18) |

In some embodiments, an IgA heavy chain constant region comprises one or more heavy chain constant domains (e.g., a CH1 domain, a CH2 domain, a CH3 domain, or any combination thereof). TABLE 2 lists exemplary amino acid sequences of IgA2 CH1 domain, IgA CH2 domain and IgA CH3 domain. In some embodiments, an IgA heavy chain constant region (e.g., IgA CH1 domain, IgA CH2 domain and IgA CH3 domain) comprises an amino acid sequence that is at least 60%, at least 70%, at least 75%, at least 80%, at least 85% b, at least 90% b, at least 95%, at least 99%, or 100% identical to any of the amino acid sequences of TABLE 2. In some embodiments, a heavy chain constant region comprises one or more of an IgA CH3 domain, an IgA CH2 domain, or an IgA CH1 domain, or any combination thereof. In some embodiments, an ani-EGFR IgA antibody comprises a heavy chain constant region comprising one or more amino acid of an IgG CH3 domain, an IgG CH2 domain, or an IgG CH1 domain. In some embodiments, an anti-EGFR IgA antibody comprises a heavy chain constant region comprising an IgA CH3 domain, an IgA CH2 domain, and an IgA CH1 domain. In some embodiments, an anti-EGFR IgA antibody comprises a heavy chain constant region comprising an IgA CH3 domain, an IgA CH2 domain, and an IgG CH1 domain. In some embodiments, an anti-EGFR IgA antibody is an IgA2 antibody that comprises a heavy chain constant region comprising one or more of IgA2 CH3 domain, an IgA2 CH2 domain, an IgA2 CH1 domain, or any combination thereof.

TABLE 2
Exemplary Sequences of IgA Heavy Chain Constant
Domains

| | SEQ | |
|---|---|---|
| | ID | |
| Name | NO: | Amino Acid Sequence |
| CH1 domain of WT | 20 | ASPTSPKVFPLSLDSTPQDGNVVVACL |
| IgA2 heavy chain | | VQGFFPQEPLSVTWSESGQNVTARNFP |
| constant domain | | PSQDASGDLYTTSSQLTLPATQCPDGK |
| | | SVTCHVKHYTNPSQDVTVPCP |
| CH2 domain of WT | 21 | CCHPRLSLHRPALEDLLLGSEANLTCT |
| IgA2 heavy chain | | LTGLRDASGATFTWTPSSGKSAVQGPP |
| constant domain | | ERDLCGCYSVSSVLPGCAQPWNHGETF |
| | | TCTAAHPELKTPLTANITKS |
| CH3 domain of WT | 22 | GNTFRPEVHLLPPPSEELALNELVTLT |
| IgA2 heavy chain | | CLARGFSPKDVLVRWLQGSQELPREKY |
| constant domain | | LTWASRQEPSQGTTTFAVTSILRVAAE |
| including C- | | DWKKGDTFSCMVGHEALPLAFTQKTID |
| terminal | | RLAGKPTHVNVSVVMAEVDGTCY |
| Tail-piece | | |

In some embodiments, an anti-EGFR IgA antibody comprises a light chain constant region (CL). TABLE 3 lists exemplary amino acid sequences of IgA light chain constant regions. In some embodiments, a light chain constant region comprises an amino acid sequence that is at least 50%, at least 60%, at least 70%, at least 75%, at least 80%, at least 85% b, at least 90% b, at least 95%, at least 99% b, or 100% identical to any one of the amino acid sequences of TABLE 3 (SEQ ID NO: 23 or SEQ ID NO: 24). In some embodiments, a light chain constant region comprises a kappa light chain constant region.

TABLE 3
Exemplary Sequences of IgA Light Chain Constant
Regions

| | SEQ | |
|---|---|---|
| | ID | |
| Name | NO: | Amino Acid Sequence |
| kappa | 23 | RTVAAPSVFIFPPSDEQLKSGTASVVCLLNNF |
| light chain | | YPREAKVQWKVDNALQSGNSQESVTEQDSKDS |
| constant | | TYSLSSTLTLSKADYEKHKVYACEVTHQGLSS |
| region 1 | | PVTKSFNRGEC |
| kappa | 24 | ASPTSPKVFPLSLDSTPQDGNVVVACLVQGFF |
| light chain | | PQEPLSVTWSESGQGVTARNFPPSQDASGDLY |
| constant | | TTSSQLTLPATQCPDGKSVTCHVKHYTNPSQD |
| region 2 | | VTVPCRVPPPPPCCHPRLSLHRPALEDLLLGS |
| | | EATISCTLTGLRDASGATFTWTPSSGKSAVQG |
| | | PPERDLCGCYSVSSVLPGSAQPWNHGETFTCT |
| | | AAHPELKTPLTATLSKSGNTFRPEVHLLPPPS |
| | | EELALNELVTLTCLARGFSPKDVLVRWLQGSQ |
| | | ELPREKYLTWASRQEPSQGTTTFAVTSILRVA |
| | | AEDWKKGDTFSCMVGHEALPLAFTQKTIDRLA |
| | | GK |

In some embodiments, an anti-EGFR IgA antibody comprises an IgG light chain variable region. In some embodiments, an anti-EGFR IgA antibody comprises an IgG heavy chain variable region. In some embodiments, an anti-EGFR IgA antibody comprises an IgG light chain variable region and an IgG heavy chain variable region.

In some embodiments, an anti-EGFR IgA antibody can comprise at least a portion of the Fc domain. In some embodiments, an anti-EGFR IgA antibody comprises a heavy chain constant region comprising a CH3 domain, CH2 domain, and CH1 domain. In some embodiments, an anti-EGFR IgA antibody comprises a light chain constant region comprising a CL domain.

In some embodiments, an antibody or a functional fragment thereof provided herein comprises an IgA heavy chain constant region comprises at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 4-9. In some embodiments, an antibody or a functional fragment thereof provided herein comprises at least one IgA heavy chain constant domain comprises at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 21-22. In some embodiments, an antibody or a functional fragment thereof provided herein comprises an IgA light chain constant domain comprises at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of SEQ ID NO: 23 or 24. In some embodiments, an antibody or a functional fragment thereof provided herein comprises a variable heavy chain domain from an IgG antibody. In some embodiments, an antibody or a functional fragment thereof provided herein comprises a variable light chain domain from an IgG antibody.

### Variable Region

In one aspect, an antibody or a functional antigen-binding fragment thereof comprises a heavy chain variable region (VH) sequence. In some embodiments, the VH comprises three CDRs, CDR-H1, CDR-H2 and CDR-H3. TABLE 4 lists exemplary amino acid sequences for CDRs of VH. TABLE 5 lists exemplary amino acid sequences of VH CDRs.

TABLE 4
Exemplary Amino Acid Sequences of VH CDRs

| SEQ | | SEQ | | SEQ | |
|---|---|---|---|---|---|
| ID | | ID | | ID | |
| NO: | CDR-H1 | NO: | CDR-H2 | NO: | CDR-H3 |
| 34 | GFTFSTYG | 57 | IWDDGSYK | 81 | ARDGITMVRGVMKDYFDY |
| 35 | GYTFTSYG | 58 | ISASNGNT | 82 | ARVYADYADY |
| 36 | GFSLSNYD | 59 | IWSGGNT | 83 | ARALDYYDYEFAY |
| 37 | GFSLTNYG | 60 | IYYSGNT | 84 | ARALTYYDYEFAY |
| 38 | GGSVSSGDYY | 61 | ISYSGNT | 85 | VRDRVTGAFDI |
| 39 | GYSISSDFA | 62 | IWYDGSDK | 86 | VTAGRGFPY |
| 40 | GFTFRNYG | 63 | IRSKYNNYAT | 87 | ARDGYDILTGNPRDFDY |
| 41 | GFTFNKYA | 64 | IYPGSRST | 88 | VRHGNFGNSYISYWAY |
| 42 | GYTFTSYW | 65 | FNPNSGYS | 89 | TRNGDYYVSSGDAMDY |
| 43 | GFTFTDYK | 66 | IYPGDGDT | 90 | ARLSPGGYYVMDA |
| 44 | GYSISRDFA | 67 | ISYNGNT | 91 | ARYDAPGYAMDY |
| 45 | GYTFTSHW | 68 | FNPSNGRT | 92 | VTASRGFPY |
| 46 | GGSISSGDYY | 69 | INPSSGRN | 93 | ASRDYDYDGRYFDY |
| 47 | GYTFTNYY | 70 | IYYSGST | 94 | VRYYGYDEAMDY |
| 48 | GYDFTNYA | 71 | INPTSGGS | 95 | ARVSIFGVGTFDY |
| 49 | GGSVSSGSYY | 72 | HIYYSGNTNYNPSLKS | 96 | ARQGLWFDSDGRGFDF |
| 50 | HYDMS | 73 | INANTGDP | 97 | DRVTGAFDI |
| 51 | GFAFSHY | 74 | ASGGDI | 98 | TRERFLEWLHFDY |
| 52 | EYPIH | 75 | YIASGGDITYYADTVKG | 99 | ARNPISIPAFDI |
| 53 | GYTFTEY | 76 | YTDIGK | 100 | SSYGNNGDALDF |
| 54 | TRYWLH | 77 | YTDIGKPTYAEEFKG | 101 | DRYDSLFDY |
| | | 78 | EINPSSGRTNYNEKFKS | 102 | WDYDEGTWFAY |

TABLE 5
Exemplary VH Amino Acid Sequences

| SEQ | |
|---|---|
| ID | |
| NO: | Exemplary VH Amino Acid Sequences |
| 169 | QVQLVESGGGVVQPGRSLRLSCAASGFTFSTYGMHWVRQAPGKGLEWVAVIWDDGSYK |
| | YYGDSVKGRFTISRDNSKNTLYLQMNSLRAEDTAVYYCARDGITMVRGVMKDYFDYW |
| | GQGTLVTVSS |
| 170 | QVQLVQSGAEVKKPGASVKVSCKASGYTFTSYGFSWVRQAPGQGLEWMGWISASNGNT |
| | YYAQKLQGRVTMTTDTSTSTAYMELRSLRSDDTAVYYCARVYADYADYWGQGTLVTV |
| | SSASTKGPSVFPLAPSSKSTSGGTAALGCLVKDYFPEPVTVSWNSGALTSGVHTFPAVLQS |
| | SGLYSLSSVVTVPSSSLGTQTYICNVNHKPSNTKVDKKVEPKSC |
| 171 | QVQLQESGPGLVKPSETLSLTCTVSGFSLSNYDVHWVRQAPGKGLEWLGVIWSGGNTDY |
| | NTPFTSRLTISVDTSKNQFSLKLSSVTAADTAVYYCARALDYYDYEFAYWGQGTLVTVSS |
| 172 | QVQLKQSGPGLVQPSQSLSITCTVSGFSLTNYGVHWVRQSPGKGLEWLGVIWSGGNTDY |
| | NTPFTSRLSINKDNSKSQVFFKMNSLQSNDTAIYYCARALTYYDYEFAYWGQGTLVTVSA |
| 173 | QVQLQESGPGLVKPSETLSLTCTVSGGSVSSGDYYWTWIRQSPGKGLEWIGHIYYSGNTN |
| | YNPSLKSRLTISIDTSKTQFSLKLSSVTAADTAIYYCVRDRVTGAFDIWGQGTMVTVSS |
| 174 | QVQLQESGPGLVKPSQTLSLTCTVSGYSISSDFAWNWIRQPPGKGLEWMGYISYSGNTRY |
| | QPSLKSRITISRDTSKNQFFLKLNSVTAADTATYYCVTAGRGFPYWGQGTLVTVSS |
| 175 | QVQLVESGGGVVQSGRSLRLSCAASGFTFRNYGMHWVRQAPGKCLEWVAVIWYDGSD |
| | KYYADSVRGRFTISRDNSKNTLYLQMNSLRAEDTAVYYCARDGYDILTGNPRDFDYWG |
| | QGTLVTVSS |
| 176 | EVQLVESGGGLVQPGGSLKLSCAASGFTFNKYAMNWVRQAPGKGLEWVARIRSKYNNY |
| | ATYYADSVKDRFTISRDDSKNTAYLQMNNLKTEDTAVYYCVRHGNFGNSYISYWAYWG |
| | QGTLVTVSS |
| 177 | EVQLQQPGSELVRPGASVKLSCKASGYTFTSYWMHWVKQRPGQGLEWIGNIYPGSRSTN |
| | YDEKFKSKATLTVDTSSSTAYMQLSSLTSEDSAVYYCTRNGDYYVSSGDAMDYWGQGT |
| | SVTVSS |
| 178 | QVQLVQSGAEVKKPGSSVKVSCKASGFTFTDYKIHWVRQAPGQGLEWMGYFNPNSGYS |
| | TYAQKFQGRVTITADKSTSTAYMELSSLRSEDTAVYYCARLSPGGYYVMDAWGQGTTV |
| | TVSS |
| 179 | QVQLKQSGPGLVQPSQSLSITCTVSGFSLTNYGVHWVRQSPGKGLEWLGVIWSGGNTDY |
| | NTPFTSRLSINKDNSKSQVFFKMNSLQSNDTAIYYCARALTYYDYEFAYWGQGTLVTVSS |
| 180 | QVQLVQSGAEVAKPGASVKLSCKASGYTFTSYWMQWVKQRPGQGLECIGTIYPGDGDT |
| | TYTQKFQGKATLTADKSSSTAYMQLSSLRSEDSAVYYCARYDAPGYAMDYWGQGTLVT |
| | VSS |
| 181 | EVQLQESGPGLVKPSQTLSLTCTVSGYSISRDFAWNWIRQPPGKGLEWMGYISYNGNTRY |
| | QPSLKSRITISRDTSKNQFFLKLNSVTAADTATYYCVTASRGFPYWGQGTLVTVSS |
| 182 | QVQLVQSGAEVKKPGASVKVSCKASGYTFTSHWMHWVRQAPGQGLEWIGEFNPSNGRT |
| | NYNEKFKSKATMTVDTSTNTAYMELSSLRSEDTAVYYCASRDYDYDGRYFDYWGQGTL |
| | VTVSS |
| 183 | QVQLQQPGAELVEPGGSVKLSCKASGYTFTSHWMHWVKQRPGQGLEWIGEINPSSGRNN |
| | YNEKFKSKATLTVDKSSSTAYMQFSSLTSEDSAVYYCVRYYGYDEAMDYWGQGTSVTV |
| | SS |
| 184 | QVQLQESGPGLVKPSQTLSLTCTVSGGSISSGDYYWSWIRQPPGKGLEWIGYIYYSGSTDY |
| | NPSLKSRVTMSVDTSKNQFSLKVNSVTAADTAVYYCARVSIFGVGTFDYWGQGTLVTVS |
| | S |
| 185 | QVQLQQSGAEVKKPGSSVKVSCKASGYTFTNYYIYWVRQAPGQGLEWIGGINPTSGGSN |
| | FNEKFKTRVTITADESSTTAYMELSSLRSEDTAFYFCTRQGLWFDSDGRGFDFWGQGTTV |
| | TVSS |
| 186 | QVQLVQSGSELKKPGASVKISCKASGYDFTNYAMNWVRQAPGHGLEWMGWINANTGD |
| | PTYAQGFTGRFVFSLDTSVSTAYLQISSLKAEDSAVYYCTRERFLEWLHFDYWGQGTLVT |
| | VSS |
| 187 | EVQLVESGGGLVQPGGSLRLSCAASGFSLTNYGVHWVRQAPGKGLEWLGVIWSGGNTD |
| | YGNEFTSRFTISRDNAKNSLYLQMNSLRAEDTAVYYCARALDYYDYEFAYWGQGTMVT |
| | VSS |
| 188 | QVQLKQSGPGLVQPSQSLSITCTVSGFSLTNYGVHWVRQSPGKGLEWLGVIWSGGNTDY |
| | NTPFTSRLSINKDNSKSQVFFKMNSLQSNDTAIYYCARALTYYDYEFAYWGQGTLVTVST |
| 189 | QVQLQESGPGLVKPSETLSLTCTVSGGSVSSGSYYWSWIRQPPGKGLEWIGYIYYSGSTN |
| | YNPSLKSRVTISVDTSKNQFSLKLSSVTAADTAVYYCARNPISIPAFDIWGQGTMVTVSS |
| 190 | EMQLVESGGGFVKPGGSLKLSCAASGFAFSHYDMSWVRQTPKQRLEWVAYIASGGDITY |
| | YADTVKGRFTISRDNAQNTLYLQMSSLKSEDTAMFYCSRSSYGNNGDALDFWGQGTSVT |
| | VSS |
| 191 | QIQLVQSGPELKKPGETVKISCKASGYTFTEYPIHWVKQAPGKGFKWMGMIYTDIGKPTY |
| | AEEFKGRFAFSLETSASTAYLQINNLKNEDTATYFCVRDRYDSLFDYWGQGTTLTVSS |
| 192 | QVQLVQSGAEVKKPGASVKVSCKASGYTFTRYWLHWVRQAPGQGLEWIGEINPSSGRT |
| | NYNEKFKSRATLTVDKSISTAYMELSRLRSDDTAVYYCARWDYDEGTWFAYWGQGTLV |
| | TVSS |
| 193 | QVQLVQSGAEVKKPGASVKVSCKASGYAFTGYTIHWVRQAPGQRLEWMGWFYPGSGT |
| | LKYSEKFQGRVTITRDKSLSTAYMELSSLRSEDTAVYYCARHGTGTLMAMDYWGQGTL |
| | VTVSS |
| 194 | EVQLVESGGGLVQPGGSLRLSCAASGFTISTNAMSWVRQAPGKGLEWIGVITGRDITYYA |
| | SWAKGRFTISRDNSKNTLYLQMNSLRAEDTAVYYCARDGGSSAITSNNIWGQGTLVTVS |
| | S |

In some embodiments, a VH sequence comprises at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 169-194. In some embodiments, a VH sequence having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity contains substitutions (e.g., conservative substitutions), insertions, or deletions relative to a corresponding wildtype antibody amino acid sequence, while retaining the ability to bind to the same antigen as the corresponding wildtype antibody. In some embodiments, a total of 1 to 10 amino acids have been substituted, inserted and/or deleted in the amino acid sequence of any one of SEQ ID NOs: 169-194. In some embodiments, substitutions, insertions, or deletions occur in regions outside the CDRs (e.g., in the FRs). Optionally, an antibody comprises a VH sequence of anyone of SEQ ID NOs: 169-194, including one or more post-translational modifications of that sequence.

In a some embodiment, a VH comprises one, two or three CDRs selected from: (a) CDR-H1 comprising the amino acid sequence of any one of SEQ ID NOs: 34-54, or a variant thereof comprising 1 to 3 substitutions, deletions or insertions; (b) CDR-H2 comprising the amino acid sequence of any one of SEQ ID NO: 57-78, or a variant thereof comprising 1 to 3 substitutions, deletions or insertions; and (c) CDR-H3 comprising the amino acid sequence of any one of SEQ ID NO: 81-102, or a variant thereof comprising 1 to 3 substitutions, deletions or insertions.

In one aspect, an antibody or a functional antigen-binding fragment thereof is provided, wherein the antibody or functional antigen-binding fragment thereof comprises a light chain variable region (VL) sequence. In some embodiments, a VL sequence comprises three CDRs, CDR-L1, CDR-L2 and CDR-L3. TABLE 6 lists exemplary amino acid sequences for VL CDRs. TABLE 7 lists exemplary VL amino acid sequences.

TABLE 6
Exemplary Amino Acid Sequences of CDR-Ls

| SEQ | | SEQ | | SEQ | |
|---|---|---|---|---|---|
| ID | | ID | | ID | |
| NO: | CDR-L1 | NO: | CDR-L2 | NO: | CDR-L3 |
| 105 | QDISSA | 129 | DAS | 146 | QQFNSYPLT |
| 106 | QGINTW | 130 | AAS | 147 | QQANSFPLT |
| 107 | QSIGTN | 131 | YAS | 148 | QQNNEWPTS |
| 108 | QDISNY | 132 | HGT | 149 | QQNNNWPTT |
| 109 | QDINSN | 133 | RIS | 150 | QHFDHLPLA |
| 110 | QSLVHSDGNTY | 134 | GTK | 151 | VQYAQFPWT |
| 111 | TGAVTSGNY | 135 | YTS | 152 | MQSTHVPRT |
| 112 | QDIGNY | 136 | NTN | 153 | VLWYSNRWV |
| 113 | QGINNY | 137 | DTS | 154 | QHYNTVPPT |
| 114 | QDINNY | 138 | QMS | 155 | LQHNSFPT |
| 115 | SSVTY | 139 | KVS | 156 | LQYDNLLYT |
| 116 | KSLLHSNGITY | 140 | DASNLET | 157 | QQWSSHIFT |
| 117 | QSVSSY | 141 | YDSD | 158 | AQNLELPYT |
| 118 | QNIVHSNGNTY | 142 | KVSNRFS | 159 | HQYGSTPLT |
| 119 | QASQDISNYLN | 143 | NARTLVE | 160 | FQYSHVPWT |
| 120 | QSISSY | | | 161 | QQSYSTPPT |
| 121 | NIGSKS | | | 162 | QQNNNWPTS |
| 122 | QSLVHSNGNTY | | | 163 | QVWDTSSDHVL |
| 123 | RSSQSLVHSNGNTYLH | | | 164 | SQSTHVLT |
| 124 | RSSQSLVHSNGNTYLH | | | 165 | SQSTHVPWT |
| 125 | QSLVHSNGNTY | | | 166 | QHHYGPPYT |
| 126 | RASENIYSYLA | | | | |

TABLE 7
Exemplary VL Amino Acid Sequences

| SEQ ID | |
|---|---|
| NO: | Exemplary VL Amino Acid Sequences |
| 195 | AIQLTQSPSSLSASVGDRVTITCRASQDISSALVWYQQKPGKAPKLLIYDASSLESGVPS |
| | RFSGSESGTDFTLTISSLOPEDFATYYCQQFNSYPLTFGGGTKVEIK |
| 196 | DIQMTQSPSSVSASVGDRVTITCRASQGINTWLAWYQQKPGKAPKLLIYAASSLKSGV |
| | PSRFSGSGSGTDFTLTISSLQPEDFATYYCQQANSFPLTFGGGTKVEIK |
| 197 | EIVLTQSPDFQSVTPKEKVTITCRASQSIGTNIHWYQQKPDQSPKLLIKYASESISGIPSRF |
| | SGSGSGTDFTLTINSLEAEDAATYYCQQNNEWPTSFGQGTKLEIK |
| 198 | DILLTQSPVILSVSPGERVSFSCRASQSIGTNIHWYQQRINGSPRLLIKYASESISGIPSRF |
| | SGSGSGTDFTLSINSVESEDIADYYCQQNNNWPTTFGAGTKLELK |
| 199 | DIQMTQSPSTLSASVGDRVTITCQASQDISNYLNWYQQKPGKAPKLLIYDASNLETGVP |
| | SRFSGSGSGTDFTFTISSLQPEDIATYFCQHFDHLPLAFGGGTKVEIK |
| 200 | DIQMTQSPSSMSVSVGDRVTITCHSSQDINSNIGWLQQKPGKSFKGLIYHGTNLDDGVP |
| | SRFSGSGSGTDYTLTISSLQPEDFATYYCVQYAQFPWTFGGGTKLEIK |
| 201 | DTVMTQTPLSSHVTLGQPASISCRSSQSLVHSDGNTYLSWLQQRPGQPPRLLIYRISRRF |
| | SGVPDRFSGSGAGTDFTLEISRVEAEDVGVYYCMQSTHVPRTFGCGTKVEIK |
| 202 | QTVVTQEPSLTVSPGGTVTLTCGSSTGAVTSGNYPNWVQQKPGQAPRGLIGGTKFLAP |
| | GTPARFSGSLLGGKAALTLSGVQPEDEAEYYCVLWYSNRWVFGGGTKLTVL |
| 203 | DIQMTQTTSSLSASLGDRVTISCRTSQDIGNYLNWYQQKPDGTVKLLIYYTSRLHSGVP |
| | SRFSGSGSGTDFSLTINNVEQEDVATYFCQHYNTVPPTFGGGTKLEI |
| 204 | DIQMTQSPSSLSASVGDRVTITCRASQGINNYLNWYQQKPGKAPKRLIYNTNNLQTGV |
| | PSRFSGSGSGTEFTLTISSLQPEDFATYYCLQHNSFPTFGQGTKLEIK |
| 205 | DIQMTQSPSSLSASVGDRVTITCRASQDINNYLAWYQHKPGKGPKLLIHYTSTLHPGIP |
| | SRFSGSGSGRDYSFSISSLEPEDIATYYCLQYDNLLYTFGQGTKLEIK |
| 206 | DIQMTQSPSSMSVSVGDRVTITCHSSQDINSNIGWLQQKPGKSFKGLIYHGTNLDDGVP |
| | SRFSGSGSGTDYTLTISSLQPEDFATYYCVQYAQFPWTFGGGTKLEIK |
| 207 | DIQMTQSPSSLSASVGDRVTITCSASSSVTYMYWYQQKPGKAPKLLIYDTSNLASGVPS |
| | RFSGSGSGTDYTFTISSLQPEDIATYYCQQWSSHIFTFGQGTKVEIK |
| 208 | DIVMTQAAFSNPVTLGTSASISCRSSKSLLHSNGITYLYWYLQKPGQSPQLLIYQMSNL |
| | ASGVPDRFSSSGSGTDFTLRISRVEAEDVGVYYCAQNLELPYTFGGGTKLEIK |
| 209 | EIVMTQSPATLSLSPGERATLSCRASQSVSSYLAWYQQKPGQAPRLLIYDASNRATGIP |
| | ARFSGSGSGTDFTLTISSLEPEDFAVYYCHQYGSTPLTFGGGTKAEIK |
| 210 | DIQMTQSPSSLSASVGDRVTITCRSSQNIVHSNGNTYLDWYQQTPGKAPKLLIYKVSNR |
| | FSGVPSRFSGSGSGTDFTFTISSLQPEDIATYYCFQYSHVPWTFGQGTKLQIT |
| 211 | DIQMTQSPSSLSASVGDRVTITCQASQDISNYLNWYQQKPGKAPKLLIYDASNLETGVP |
| | SRFSGSGSGTDFTFTISSLQPEDIATYFCQHFDHLPLAFGGGTKVEIK |
| 212 | DIQMTQSPSSLSASVGDRVTITCRASQSISSYLNWYQQKPGKAPKLLIYAASSLQSGVPS |
| | RFSGSGSGTDFTLTISSLQPEDFATYYCQQSYSTPPTFGQGTKVEIK |
| 213 | EIVLTQSPATLSLSPGERATLSCRASQSIGTNIHWYQQKPGQAPRLLIKYASESISGIPAR |
| | FSGSGSGTDFTLTISSLEPEDFAVYYCQQNNNWPTSFGGGTKVEIK |
| 214 | QPVLTQPPSVSVAPGKTARITCGGNNIGSKSVHWYQQKPGQAPVLVIYYDSDRPSGIPE |
| | RFSGSNSGNTATLTISRVEAGDEADYYCQVWDTSSDHVLFGGGTKLTVL |
| 215 | DVVMTQTPLSLPVSLGDQASISCRSSQSLVHSNGNTYLHWYLQKPGQSPKLLIYKVSN |
| | RFSGVPDRFSGSGSGTDFTLKISRVEAEDLGVYFCSQSTHVLTFGSGTKLEI K |
| 216 | DVVMTQTPLSLPVSLGDQASISCRSSQSLVHSNGNTYLHWYLQKPGQSPKLLIYKVSN |
| | RFSGVPDRFSGSGSGTDFTLKISRVEAEDLGVYFCSQSTHVPWTFGGGTKLE IK |
| 217 | DIQMTQSPSFLSASVGDRVTITCRASENIYSYLAWYQQKPGKAPKLLVYNARTLVEGV |
| | PSRFSGSGSGTEFTLTISSLQPEDFATYYCQHHYGPPYTFGQGTKVEIK |
| 218 | DVVMTQTPLSLSVTPGQPASISCKSSQSLANSYGNTYLSWYLHKPGQSPQLLIYGISNR |
| | FSGVPDRFSGSGSGTDFTLKISRVEAEDVGVYYCLQGTHQPPTFGQGTKLEIK |
| 219 | DVVMTQSPSTLSASVGDRVTINCQASESISSWLAWYQQKPGKAPKLLIYEASKLASGV |
| | PSRFSGSGSGTEFTLTISSLQPDDFATYYCQGYFYFISRTYVNSFGGGTKVEIK |

In some embodiments, a VL sequence comprises at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 195-219. In some embodiments, a VL sequence having at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% identity contains substitutions (e.g., conservative substitutions), insertions, or deletions relative to a corresponding wildtype antibody amino acid sequence, while retaining the ability to bind to the same antigen as the corresponding wildtype antibody. In some embodiments, a total of 1 to 10 amino acids have been substituted, inserted and/or deleted in any one of the amino acid sequences of any one of SEQ ID NOs: 195-219. In some embodiments, substitutions, insertions, or deletions occur in regions outside the CDRs (e.g., in the FRs). Optionally, an antibody or a functional antigen-binding fragment thereof comprises a VL sequence of any one of SEQ ID NO: 195-219, including post-translational modifications of that sequence.

In some embodiments, a VL sequence comprises one, two or three CDRs selected from (a) CDR-L1 comprising an amino acid sequence of any one of SEQ ID NOS: 105-126, or a variant thereof comprising 1 to 3 substitutions, deletions or insertions; (b) CDR-L2 comprising an amino acid sequence of any one of SEQ ID NOS: 129-143, or a variant thereof comprising 1 to 3 substitutions, deletions or insertions; and (c) CDR-L3 comprising an amino acid sequence of any one of SEQ ID NOS: 146-166, or a variant thereof comprising 1 to 3 substitutions, deletions or insertions.

In one aspect, an anti-EGFR IgA antibody or a functional antigen-binding fragment thereof is provided, wherein the antibody or functional antigen-binding fragment thereof comprises a VH as in any of the embodiments provided above, and a VL as in any of the embodiments provided above. In some embodiments, an antibody or a functional antigen-binding fragment thereof comprises a VH and a VL, wherein the VH comprises the amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 169-194, and wherein the VL comprises the amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence of any one of SEQ ID NOs: 195-219, and optionally including post-translational modifications of those sequences. In some embodiments, a VH sequence comprises an amino acid sequence of any one of SEQ ID NOS: 169-194, and wherein a VL sequence comprises an amino acid sequence of any one of SEQ ID NOS: 195-219, and optionally including post-translational modifications of those sequences.

In one aspect, an anti-EGFR IgA antibody or a functional antigen-binding fragment thereof is provided, wherein the antibody or functional antigen-binding fragment thereof comprises a combination of a VH sequence and a VL sequence recited in Table 8, wherein the VH comprises the amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 169-194, and wherein the VL comprises the amino acid sequence having at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 195-219. In some embodiments, an antibody or functional antigen-binding fragment thereof comprises a combination of a VH sequence and a VL sequence to TABLE 8.

TABLE 8
Exemplary Combinations of VH Sequence and VL Sequence

| Comb. No: | VH of SEQ ID NO: | VL of SEQ ID NO: |
|---|---|---|
| 1 | 169 | 195 |
| 2 | 170 | 196 |
| 3 | 171 | 197 |
| 4 | 172 | 198 |
| 5 | 173 | 199 |
| 6 | 174 | 200 |
| 7 | 175 | 201 |
| 8 | 176 | 202 |
| 9 | 177 | 203 |
| 10 | 178 | 204 |
| 11 | 179 | 198 |
| 12 | 180 | 205 |
| 13 | 181 | 206 |
| 14 | 182 | 207 |
| 15 | 183 | 208 |
| 16 | 184 | 209 |
| 17 | 185 | 210 |
| 18 | 173 | 211 |
| 19 | 186 | 212 |
| 20 | 187 | 213 |
| 21 | 188 | 198 |
| 22 | 189 | 214 |
| 23 | 190 | 215 |
| 24 | 191 | 216 |
| 25 | 192 | 217 |
| 26 | 193 | 218 |
| 27 | 194 | 219 |

In one aspect, an anti-EGFR IgA antibody or a functional antigen-binding fragment thereof is provided, wherein the antibody or functional antigen-binding fragment thereof comprises a CDR-H3 selected from any CDR-H3 in TABLE 4 or a variant thereof comprising 1 to 3 substitutions, deletions or insertions, and a CDR-L3 selected from any CDR-L3 in TABLE 6 or a variant thereof comprising 1 to 3 substitutions, deletions or insertions, wherein the selected CDR-H3 and CDR-L3 are paired according to TABLE 9. In one aspect, an antibody or functional antigen-binding fragment thereof is provided, wherein the antibody or functional antigen-binding fragment thereof comprises a CDR-H2 selected from any CDR-H2 in TABLE 4 or a variant thereof comprising 1 to 3 substitutions, deletions or insertions, and a CDR-L2 selected from any CDR-L2 in TABLE 6 or a variant thereof comprising 1 to 3 substitutions, deletions or insertions, wherein the selected CDR-H2 and CDR-L2 are paired according to TABLE 9. In one aspect, an antibody or functional antigen-binding fragment thereof is provided, wherein the antibody or functional antigen-binding fragment thereof comprises a CDR-H1 selected from any CDR-H1 in TABLE 4 or a variant thereof comprising 1 to 3 substitutions, deletions or insertions, and a CDR-L1 selected from any CDR-L1 in TABLE 6 or a variant thereof comprising 1 to 3 substitutions, deletions or insertions, wherein the selected CDR-H1 and CDR-L1 are paired according to TABLE 9. In one aspect, the present disclosure provides an antibody or functional antigen-binding fragment thereof comprising a VH comprising an amino acid sequence of SEQ ID NO: 173, including post-translational modifications of those sequences, and a VL comprising an amino acid sequence of SEQ ID NO: 211, including post-translational modifications of those sequences. In one aspect, the present disclosure provides an antibody or functional antigen-binding fragment thereof comprising a VH comprising an amino acid sequence of SEQ ID NO: 172, including post-translational modifications of those sequences, and a VL comprising an amino acid sequence of SEQ ID NO: 198, including post-translational modifications of those sequences. In one aspect, the present disclosure provides an antibody or functional antigen-binding fragment thereof comprising a VH comprising an amino acid sequence of SEQ ID NO: 182, including post-translational modifications of those sequences, and a VL comprising the amino acid sequence of SEQ ID NO: 207, including post-translational modifications of those sequences. In one aspect, the present disclosure provides an antibody or a functional antigen-binding fragment thereof comprising a VH comprising an amino acid sequence of SEQ ID NO: 184, including post-translational modifications of those sequences, and a VL comprising an amino acid sequence of SEQ ID NO: 209, including post-translational modifications of those sequences.

In one aspect, the present disclosure provides an antibody or a functional antigen-binding fragment thereof comprising at least one, two, three, four, five, or six CDRs selected from (a) CDR-H1 comprising the amino acid sequence of SEQ ID NO: 38; (b) CDR-H2 comprising the amino acid sequence of SEQ ID NO: 72; (c) CDR-H3 comprising the amino acid sequence of SEQ ID NO: 97; (d) CDR-L1 comprising the amino acid sequence of SEQ ID NO: 119; (e) CDR-L2 comprising the amino acid sequence of SEQ ID NO: 140; and (f) CDR-L3 comprising the amino acid sequence of SEQ ID NO: 150. In one aspect, the present disclosure provides an antibody or a functional antigen-binding fragment thereof comprising at least one, two, three, four, five, or six CDRs selected from (a) CDR-H1 comprising the amino acid sequence of SEQ ID NO: 37; (b) CDR-H2 comprising the amino acid sequence of SEQ ID NO: 59; (c) CDR-H3 comprising the amino acid sequence of SEQ ID NO: 84; (d) CDR-L1 comprising the amino acid sequence of SEQ ID NO: 107; (e) CDR-L2 comprising the amino acid sequence of SEQ ID NO: 131; and (f) CDR-L3 comprising the amino acid sequence of SEQ ID NO: 149. In one aspect, the present disclosure provides an antibody or a functional antigen-binding fragment thereof comprising at least one, two, three, four, five, or six CDRs selected from (a) CDR-H1 comprising the amino acid sequence of SEQ ID NO: 45; (b) CDR-H2 comprising the amino acid sequence of SEQ ID NO: 68; (c) CDR-H3 comprising the amino acid sequence of SEQ ID NO: 93; (d) CDR-L1 comprising the amino acid sequence of SEQ ID NO: 115; (e) CDR-L2 comprising the amino acid sequence of SEQ ID NO: 137; and (f) CDR-L3 comprising the amino acid sequence of SEQ ID NO: 157. In one aspect, the present disclosure provides an antibody or a functional antigen-binding fragment thereof comprising at least one, two, three, four, five, or six CDRs selected from (a) CDR-H1 comprising the amino acid sequence of SEQ ID NO: 46; (b) CDR-H2 comprising the amino acid sequence of SEQ ID NO: 70; (c) CDR-3 comprising the amino acid sequence of SEQ ID NO: 95; (d) CDR-L1 comprising the amino acid sequence of SEQ ID NO: 117; (e) CDR-L2 comprising the amino acid sequence of SEQ ID NO: 129; and (N) CDR-L3 comprising the amino acid sequence of SEQ ID NO: 159.

In one aspect, an anti-EGFR IgA antibody or a functional antigen-binding fragment thereof is provided, wherein the antibody or a functional antigen-binding fragment thereof comprises: (a) a CDR-H1 comprising any one of amino acid sequences of SEQ ID NO: 34-54, or a variant thereof comprising 1 to 3 substitutions, deletions or insertions; (b) a CDR-H2 comprising any one of amino acid sequences of SEQ ID NO: 57-78, or a variant thereof comprising 1 to 3 substitutions, deletions or insertions; (c) a CDR-H3 comprising any one of amino acid sequences of SEQ ID NO: 81-102, or a variant thereof comprising 1 to 3 substitutions, deletions or insertions; (d) a CDR-L1 comprising any one of amino acid sequences of SEQ ID NO: 105-126, or a variant thereof comprising 1 to 3 substitutions, deletions or insertions; (e) a CDR-L2 comprising any one of amino acid sequences of SEQ ID NO: 129-143, or a variant thereof comprising 1 to 3 substitutions, deletions or insertions; and (f) a CDR-L3 comprising any one of amino acid sequences of SEQ ID NO: 146-166, or a variant thereof comprising 1 to 3 substitutions, deletions or insertions. In some embodiments, the CDR-H1, the CDR-H2, the CDR-H3, the CDR-L1, the CDR-L2, and the CDR-L3 are selected according to any one of the combinations provided in TABLE 9.

TABLE 9
Exemplary Combinations of CDRs

| | CDR-H1 of | CDR-H2 of | CDR-H3 of | CDR-L1 of | CDR-L2 of | CDR-L3 of |
|---|---|---|---|---|---|---|
| Comb. | SEQ ID | SEQ ID | SEQ ID | SEQ ID | SEQ ID | SEQ ID |
| No. | NO: | NO: | NO: | NO: | NO: | NO: |
| 28 | 34 | 57 | 81 | 105 | 129 | 146 |
| 29 | 35 | 58 | 82 | 106 | 130 | 147 |
| 30 | 36 | 59 | 83 | 107 | 131 | 148 |
| 31 | 37 | 59 | 84 | 107 | 131 | 149 |
| 32 | 38 | 60 | 85 | 108 | 129 | 150 |
| 33 | 39 | 61 | 86 | 109 | 132 | 151 |
| 34 | 40 | 62 | 87 | 110 | 133 | 152 |
| 35 | 41 | 63 | 88 | 111 | 134 | 153 |
| 36 | 42 | 64 | 89 | 112 | 135 | 154 |
| 37 | 43 | 65 | 90 | 113 | 136 | 155 |
| 38 | 42 | 66 | 91 | 114 | 135 | 156 |
| 39 | 44 | 67 | 92 | 109 | 132 | 151 |
| 40 | 45 | 68 | 93 | 115 | 137 | 157 |
| 41 | 45 | 69 | 94 | 116 | 138 | 158 |
| 42 | 46 | 70 | 95 | 117 | 129 | 159 |
| 43 | 47 | 71 | 96 | 118 | 139 | 160 |
| 44 | 38 | 72 | 97 | 119 | 140 | 150 |
| 45 | 48 | 73 | 98 | 120 | 130 | 161 |
| 46 | 37 | 59 | 83 | 107 | 131 | 162 |
| 47 | 49 | 70 | 99 | 121 | 141 | 163 |
| 48 | 50 | 74 | 100 | 122 | 139 | 164 |
| 49 | 50 | 75 | 100 | 122 | 142 | 164 |
| 50 | 50 | 74 | 100 | 122 | 142 | 164 |
| 51 | 50 | 75 | 100 | 122 | 139 | 164 |
| 52 | 51 | 74 | 100 | 123 | 139 | 164 |
| 53 | 51 | 75 | 100 | 123 | 142 | 164 |
| 54 | 51 | 74 | 100 | 123 | 142 | 164 |
| 55 | 51 | 75 | 100 | 123 | 139 | 164 |
| 56 | 52 | 76 | 101 | 124 | 139 | 165 |
| 57 | 52 | 77 | 101 | 124 | 142 | 165 |
| 58 | 52 | 76 | 101 | 124 | 142 | 165 |
| 59 | 52 | 77 | 101 | 124 | 139 | 165 |
| 60 | 53 | 76 | 101 | 125 | 139 | 165 |
| 61 | 53 | 77 | 101 | 125 | 142 | 165 |
| 62 | 53 | 76 | 101 | 125 | 142 | 165 |
| 63 | 53 | 77 | 101 | 125 | 139 | 165 |
| 64 | 54 | 78 | 102 | 126 | 143 | 166 |

### IgA Antibody Modifications

Described herein are anti-EGFR IgA antibodies comprising one or more amino acid insertions, substitutions, and/or deletions. In some embodiments, anti-EGFR IgA antibodies disclosed herein are conjugated to one or more therapeutic agents.

In some embodiments, the amino acid numbering of an anti-EGFR IgA antibody described herein is indicated according to IMGT unique numbering for C-DOMAIN and C-LIKE-DOMAIN (as disclosed in “IMGT unique numbering for immunoglobulin and T cell receptor constant domains and Ig superfamily C-like domains.” Dev Comp Immunol. 2005; 29(3):185-203, the entire contents of which is incorporated by reference herein). In some embodiments, the amino acid modifications disclosed herein are relative to amino acid residues at select positions in a corresponding WT IgA heavy chain constant region (e.g., WT IgA2 heavy chain constant region) comprising an amino acid sequence set forth in SEQ ID NO: 1.

In some embodiments, an anti-EGFR IgA antibody disclosed herein comprises a deletion of at least four glycosylation sites within the constant region. In some embodiments, an anti-EGFR IgA antibody disclosed herein comprises a deletion of at least three N-linked glycosylation sites in the constant region of the antibody. In some embodiments, an anti-EGFR IgA antibody comprises a deletion of at least three N-linked glycosylation sites in the constant region of the antibody and at least one O-linked glycosylation site in the constant region of the antibody. In some embodiments, the anti-EGFR IgA antibodies or functional fragments disclosed herein comprise one or more modifications corresponding to the modifications disclosed in Table 10 in an IgA heavy chain constant region. In some embodiments, the anti-EGFR IgA antibodies or functional fragments disclosed herein comprise one or more modifications corresponding to the modifications in Table 10 in an IgA1 heavy chain constant region. In some embodiments, the anti-EGFR IgA antibodies or functional fragments disclosed herein comprise one or more modifications corresponding to the modifications in Table 10 in an IgA2 heavy chain constant region. In some embodiments, one or more modifications are in an amino acid residue within a CH1 domain, CH2 domain and/or CH3 domain of a IgA1 heavy chain constant region.

In some embodiments, the anti-EGFR IgA antibodies comprise a deleted tail piece. In some embodiments, the anti-EGFR IgA antibodies comprise one or more modifications, wherein the one or more modifications are in one or more amino acid residues within a CH1 domain, a CH2 domain and/or a CH3 domain of an IgA2 heavy chain constant region. In some embodiments, an IgA CH1 domain comprises an amino acid sequence that at least about 80%, 85%, 90%, 95%, 99% or 100% identical to an amino acid sequence set forth in SEQ ID NO: 20. In some embodiments, an IgA CH2 domain comprises an amino acid sequence that at least about 80%, 85%, 90%, 95%, 99% or 100% identical to an amino acid sequence set forth in SEQ ID NO: 21. In some embodiments, an IgA CH3 domain comprises an amino acid sequence that at least about 80%, 85%, 90%, 95%, 99% or 100% identical to an amino acid sequence set forth in SEQ ID NO: 22. In some embodiments, an anti-EGFR IgA antibody (e.g. IgA2 antibody) or a functional fragment thereof as disclosed herein comprises a deletion of the 3-20, 3-19, 3-18, 3-17, 3-16, 3-15, 3-14, 3-13, 3-12, 3-11, 3-10, 3-9, 3-8, 3-7, 3-6, 3-5, 3-4 C-terminal amino acids. In some embodiments, the C-terminal amino acids include amino acids 131-148 of the IgA2 antibody, numbering according to IMGT scheme.

In some embodiments, an anti-EGFR IgA antibody is an anti-EGFR IgA2 antibody. In some embodiments, an IgA2 antibody comprises a deletion of amino acids 131-148, numbering according to IMGT scheme. In some embodiments, an IgA2 antibody comprises a deletion of amino acids 147-148, numbering according to IMGT scheme. In some embodiments, an IgA2 antibody comprises a deletion of amino acids 146-148, numbering according to IMGT scheme. In some embodiments, an IgA2 antibody comprises a deletion of amino acids 145-148, numbering according to IMGT scheme. In some embodiments, an IgA2 antibody comprises a deletion of amino acids 144-148, numbering according to IMGT scheme. In some embodiments, an IgA2 antibody comprises a deletion of amino acids 143-148, numbering according to IMGT scheme. In some embodiments, an IgA2 antibody comprises a deletion of amino acids 142-148, numbering according to IMGT scheme. In some embodiments, an IgA2 antibody comprises a deletion of amino acids 141-148, numbering according to IMGT scheme. In some embodiments, an IgA2 antibody comprises a deletion of amino acids 140-148, numbering according to IMGT scheme. In some embodiments, an IgA2 antibody comprises a deletion of amino acids 139-148, numbering according to IMGT scheme. In some embodiments, an IgA2 antibody comprises a deletion of amino acids 138-148, numbering according to IMGT scheme. In some embodiments, an IgA2 antibody comprises a deletion of amino acids 137-148, numbering according to IMGT scheme. In some embodiments, an IgA2 antibody comprises a deletion of amino acids 136-148, numbering according to IMGT scheme. In some embodiments, an IgA2 antibody comprises a deletion of amino acids 135-148, numbering according to IMGT scheme. In some embodiments, an IgA2 antibody comprises a deletion of amino acids 134-148, numbering according to IMGT scheme. In some embodiments, an IgA2 antibody comprises a deletion of amino acids 133-148, numbering according to IMGT scheme. In some embodiments, an IgA2 antibody comprises a deletion of amino acids 132-148, numbering according to IMGT scheme. In some embodiments, an IgA2 antibody comprises a deletion of amino acids P131-Y148, numbering according to IMGT scheme.

In some embodiments, an anti-EGFR IgA antibody comprises a mutation of the C-terminal asparagine (N) amino acid. In some embodiments, the mutation is a non-conservative amino acid substitution. In some embodiments, the mutation deletes the glycosylation site of the C-terminal asparagine (N) amino acid of the IgA. In some embodiments, an IgA2 antibody comprises a mutation of N135, numbering according to IMGT scheme. In some embodiments, an IgA2 antibody comprises a non-conservative mutation of N135, numbering according to IMGT scheme. In some embodiments, an IgA2 antibody comprises a N135Q mutation, numbering according to IMGT scheme.

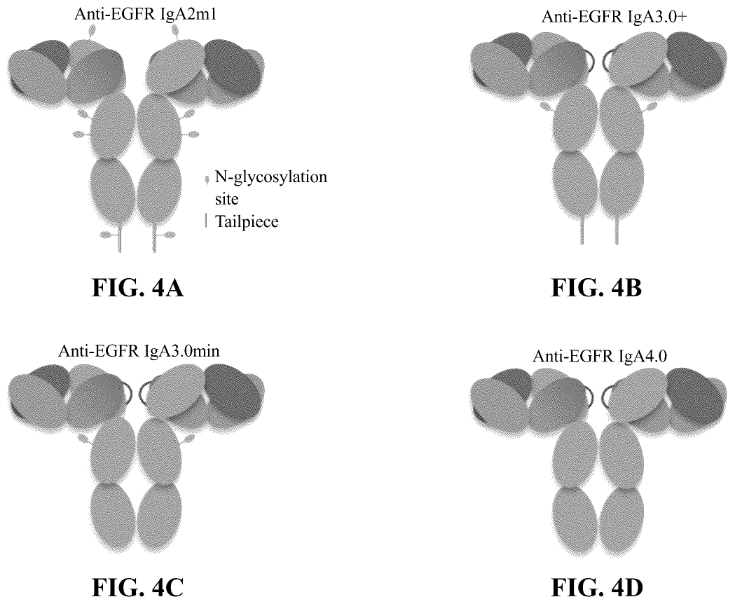

In some embodiments, the anti-EGFR IgA antibodies or functional fragments thereof of the present disclosure exhibit reduced glycosylation compared to a corresponding WT IgA. From a pharmaceutical perspective, more glycosylation sites may lead to batch-to-batch variations in the product, which could have safety and efficacy implications. Accordingly, in some embodiments, an engineered anti-EGFR IgA antibody as described herein comprises reduced heterogeneity associated with glycosylation. Alternatively, in some embodiments, an engineered anti-EGFR IgA antibody comprises a reduced glycosylation profile (FIGS. 4A-4D). In some embodiments, reduced glycosylation is achieved by modifying amino acid residues that are near or within a naturally occurring glycosylation motif or a naturally occurring glycosylation site, for example, an N-linked glycosylation site that contains the amino acid sequence NXT or NXS. In some embodiments, an anti-EGFR IgA antibody disclosed herein exhibits reduced glycosylation by at least about 2% b, at least 5%, at least 10%, at least 12%, at least 15%, at least 20%, at least 25%, at least 50%, at least 65%, at least 70%, at least 75%, at least 85%, at least 90%, at least 95%, or more relative to a corresponding WT IgA. In some embodiments, the antibodies or a functional fragment disclosed herein are partially glycosylated relative to a corresponding WT IgA antibody, for example, less than 100%, 90%, 80%, 70%, 60%, 50%, 40%, 30%, 20%, 10%, 5% or less. In some embodiments, the anti-EGFR IgA antibodies or functional fragments thereof disclosed herein are completely glycosylated relative to a corresponding WT IgA antibody.

In some embodiments, an anti-EGFR IgA antibody or a functional fragment thereof disclosed herein comprises a modification in at least two naturally occurring glycosylation sites in the IgA heavy chain constant region. In some embodiments, an anti-EGFR IgA antibody or a functional fragment thereof disclosed herein comprises a modification in at least three naturally occurring glycosylation sites in the IgA heavy chain constant region. In some embodiments, an anti-EGFR IgA antibody or a functional fragment thereof disclosed herein comprises a modification in at least four naturally occurring glycosylation site. In some embodiments, a glycosylation site comprises a N-linked glycosylation site. In some embodiments, a glycosylation site comprises a naturally occurring asparagine residue. In some embodiments, a glycosylation site is in a CH2 region, in a CH3 region, and/or a CH1 domain. In some embodiments, an IgA heavy chain constant region comprises a modification at CH1—N45.2 or P124; CH2—N20, L21, T22, C92, N120, I121, or T122; CH3—N135, C147, or Y148, or a combination thereof, numbering according to IMGT scheme. In some embodiments, an IgA heavy chain constant region comprises an amino acid substitution that is CH1—N45.2G, N45.2A, or P124R; CH2—N20G, N20Q, N20T, L21I, T22S, C92S, N120T, I121L, or T122S; CH3—N135Q, a C147 deletion, or a Y148 deletion, or a combination thereof, numbering according to IMGT scheme. In some embodiments, provided herein is an aglycosylated antibody or a functional fragment thereof. In some embodiments, an aglycosylated antibody comprises a modification at all four naturally occurring glycosylation site in IgA2 heavy chain constant region. In some embodiments, an aglycosylated antibody provided herein comprises a modification at residues; CH1—N45.2 and P124; CH2—N20, L21, T22, C92, N120, I121, and T122; and CH3—N135, C147, and Y148, numbering according to IMGT scheme. In some embodiments, an aglycosylated antibody provided herein comprises a modification at residues; CH1—N45.2 and P124; CH2—N20, C92, N120, I121, or T122; and CH3 N135, C147, and Y148, numbering according to IMGT scheme.

In some embodiments, an aglycosylated antibody provided herein comprises a modification at residues; CH1—N45.2 or P124; CH2—N20, L21, T22, C92, N120, I121, or T122; or a deletion of the C-terminal CH3 tailpiece residues P131-Y148, numbering according to IMGT scheme. In some embodiments, an aglycosylated antibody provided herein comprises a modification at residues; CH1—N45.2 and P124; CH2—N20, L21, T22, C92, N120, I121, and T122; and a deletion of the C-terminal CH3 tailpiece residues P131-Y148. numbering according to IMGT scheme.

In some embodiments, an antibody or a functional fragment thereof comprises a modification at a CH1 domain that comprises: a N45.2 substitution or a P124 substitution; a modification at a CH2 domain that comprises: an N20 substitution, a L21 substitution, a T22 substitution, a C92 substitution, a N120 substitution, an I121 substitution, or a T122 substitution; or a modification at a CH3 domain that comprises: an H5 substitution, an L7 substitution, a P10 substitution, a T22 substitution, an L79 substitution, a W81 substitution, an A85.1 substitution, a T86 substitution, an 188 substitution, an N135 substitution, a C147 deletion, a Y148 deletion, or a deletion of P131-Y148; numbering according to IMGT scheme. In some embodiments, an antibody or a functional fragment thereof comprises a modification at a CH1 domain that comprises: an N45.2 substitution selected from the group consisting of: N45.2G and N45.2A, a P124R substitution, or any combination thereof; a modification at a CH2 domain that comprises: an N20 substitution selected from the group consisting of: N20G, N20Q, and N20T, an L211 substitution, a T22S substitution, a C92S substitution, a N120T substitution, an I121L substitution, a T122S substitution, or any combination thereof; or a modification at a CH3 domain that comprises: an H5 substitution selected from the group consisting of: H5C, H5Y, H5F, H5M and H5W, an L7 substitution selected from the group consisting of: L7F, L7Y, L7M, L7W, L7H and L7I, a P10C substitution, a T22 substitution selected from the group consisting of: T22V, T22I, T22L and T22A, an L79 substitution selected from the group consisting of: L79V, L79T, L79A and L79I, a W81 substitution selected from the group consisting of: W81T, W81L, W81A, W81V and W81I, an A85.1 substitution selected from the group consisting of: A85.1F, A85.1Y, A85.1M. A85.1W and A85.1H, a T86 substitution selected from the group consisting of: T86Y, T86F, T86M, T86W and T86H, a 188 substitution selected from the group consisting of: I88L, 188A, 188V and I88T, an N135Q substitution, a C147 deletion, a Y148 deletion, or a deletion of P131-Y148; numbering according to IMGT scheme.

In some embodiments, an IgA heavy chain constant region comprises an IgA CH1, CH2, and CH3 domain, wherein the IgA heavy chain constant region comprises the following mutations: an N45.2G substitution in the CH1 domain, a P124R substitution in the CH1 domain, a C92S substitution in the CH2 domain, an N120T substitution in the CH2 domain, an I121L substitution in the CH2 domain, and a T122S substitution in the CH2 domain, numbering according to IMGT scheme, each relative to the corresponding residue in the wild-type IgA heavy chain constant region of SEQ ID NO: 1. In some embodiments, the IgA heavy chain constant region further comprises the following mutations in the CH3 domain: an N135Q substitution, a C147 deletion, and a Y148 deletion; or a deletion of P131-Y148 in the CH3 domain, numbering according to IMGT scheme, relative to the corresponding residue in the wild-type IgA heavy chain constant region of SEQ ID NO: 1. In some embodiments, the IgA heavy chain constant region further comprises the following mutations in the CH2 domain: an N20 substitution selected from the group consisting of: N20G, N20Q, and N20T; an L211 substitution; and a T22S substitution, numbering according to IMGT scheme, each relative to the corresponding residue in the wild-type IgA heavy chain constant region of SEQ ID NO: 1

In some embodiments, an antibody comprises a mutation of N45.2, numbering according to IMGT scheme. In some embodiments, an IgA2 antibody comprises a non-conservative mutation of N45.2, numbering according to IMGT scheme. In some embodiments, an IgA2 antibody comprises a N45.2G, numbering according to IMGT scheme. In some embodiments, an IgA2 antibody has an increased circulating half-life compared to an IgA2 antibody that does not have a mutation in the N45.2 amino acid.

In some embodiments, an antibody comprises a mutation of P124, numbering according to IMGT scheme. In some embodiments, an IgA2 antibody comprises a non-conservative mutation of P124, numbering according to IMGT scheme. In some embodiments, an IgA2 antibody comprises a P124R, numbering according to IMGT scheme. In some embodiments, an IgA2 antibody has an increased circulating half-life compared to an IgA2 antibody that does not have a mutation in the P124 amino acid.

In some embodiments, an IgA2 antibody has an increased stability compared to an IgA2 antibody that does not have a mutation in the P124 amino acid. In some embodiments, an increased stability refers to one or more of: reduced glycosylation, reduced aggregation, improved thermal stability, increased mechanical stability, or increased circulatory half-life.

In some embodiments, an antibody as described herein comprises a mutation of C92, numbering according to IMGT scheme. In some embodiments, an antibody comprises a non-conservative mutation of C92, numbering according to IMGT scheme. In some embodiments, an antibody comprises a C92S, numbering according to IMGT scheme. In some embodiments, an antibody has a decreased aggregation compared to an antibody that does not have a mutation in the C92 amino acid. In some embodiments, an antibody has a decreased aggregation with serum proteins compared to an antibody that does not have a mutation in the C92 amino acid. In some embodiments, an antibody has a decreased aggregation in vitro or in vivo compared to an antibody that does not have a mutation in the C92 amino acid.

In some embodiments, an IgA2 antibody comprises a mutation of C92 according to IMGT scheme. In some embodiments, an IgA2 antibody comprises a non-conservative mutation of C92, numbering according to IMGT scheme. In some embodiments, an IgA2 antibody comprises a C92S, numbering according to IMGT scheme. In some embodiments, an IgA2 antibody has a decreased aggregation compared to an IgA2 antibody that does not have a mutation in the C92 amino acid. In some embodiments, an IgA2 antibody has a decreased aggregation with serum proteins compared to an IgA2 antibody that does not have a mutation in the C92 amino acid. In some embodiments, an IgA2 antibody has a decreased aggregation in vitro or in vivo compared to an IgA2 antibody that does not have a mutation in the C92 amino acid.

In some embodiments, an antibody comprises a mutation of N120, numbering according to IMGT scheme. In some embodiments, an antibody comprises a non-conservative mutation of N120, numbering according to IMGT scheme. In some embodiments, an antibody comprises a N120T, numbering according to IMGT scheme. In some embodiments, an antibody has an increased circulating half-life compared to an IgA2 antibody that does not have a mutation in the N120 amino acid.

In some embodiments, an antibody comprises a mutation of I121, numbering according to IMGT scheme. In some embodiments, an antibody comprises a non-conservative mutation of I121, numbering according to IMGT scheme. In some embodiments, an antibody comprises an I121L, numbering according to IMGT scheme. In some embodiments, an antibody has an increased circulating half-life compared to an IgA2 antibody that does not have a mutation in the I121 amino acid.

In some embodiments, an antibody comprises a mutation of T122, numbering according to IMGT scheme. In some embodiments, an antibody comprises a non-conservative mutation of T122, numbering according to IMGT scheme. In some embodiments, an antibody comprises a T122S, numbering according to IMGT scheme. In some embodiments, an IgA2 antibody has an increased circulating half-life compared to an IgA2 antibody that does not have a mutation in the T122 amino acid.

In some embodiments, an IgA2 antibody comprises a mutation of N120, numbering according to IMGT scheme. In some embodiments, an IgA2 antibody comprises a non-conservative mutation of N120, numbering according to IMGT scheme. In some embodiments, an IgA2 antibody comprises a N120T, numbering according to IMGT scheme. In some embodiments, an IgA2 antibody has an increased circulating half-life compared to an IgA2 antibody that does not have a mutation in the N120 amino acid.

In some embodiments, an IgA2 antibody comprises a mutation of I121, according to IMGT scheme. In some embodiments, an IgA2 antibody comprises a non-conservative mutation of I121, numbering according to IMGT scheme. In some embodiments, an IgA2 antibody comprises an I121L, numbering according to IMGT scheme. In some embodiments, an IgA2 antibody has an increased circulating half-life compared to an IgA2 antibody that does not have a mutation in the I121 amino acid.

In some embodiments, an IgA2 antibody comprises a mutation of T122, numbering according to IMGT scheme. In some embodiments, an IgA2 antibody comprises a non-conservative mutation of T122, numbering according to IMGT scheme. In some embodiments, an IgA2 antibody comprises a T122S, numbering according to IMGT scheme. In some embodiments, an IgA2 antibody has an increased circulating half-life compared to an IgA2 antibody that does not have a mutation in the T122 amino acid.

In some embodiments, an IgA2 antibody comprises a mutation of N20, according to IMGT scheme. In some embodiments, an IgA2 antibody comprises a non-conservative mutation of N20, numbering according to IMGT scheme. In some embodiments, an IgA2 antibody comprises an N20G, N20Q, or N20T, numbering according to IMGT scheme. In some embodiments, an IgA2 antibody has an increased circulating half-life compared to an IgA2 antibody that does not have a mutation in the N20 amino acid.

In some embodiments, an IgA2 antibody comprises a mutation of L21, according to IMGT scheme. In some embodiments, an IgA2 antibody comprises a non-conservative mutation of L21, numbering according to IMGT scheme. In some embodiments, an IgA2 antibody comprises an L21I, numbering according to IMGT scheme. In some embodiments, an IgA2 antibody has an increased circulating half-life compared to an IgA2 antibody that does not have a mutation in the L21 amino acid.

In some embodiments, an IgA2 antibody comprises a mutation of T22, according to IMGT scheme. In some embodiments, an IgA2 antibody comprises a non-conservative mutation of T22, numbering according to IMGT scheme. In some embodiments, an IgA2 antibody comprises an T22S, numbering according to IMGT scheme. In some embodiments, an IgA2 antibody has an increased circulating half-life compared to an IgA2 antibody that does not have a mutation in the T22S amino acid.

In some embodiments, an IgA2 antibody comprises a mutation of C147, numbering according to IMGT scheme. In some embodiments, an IgA2 antibody comprises a non-conservative mutation of C147, numbering according to IMGT scheme. In some embodiments, an IgA2 antibody comprises a deletion of amino acid C147, numbering according to IMGT scheme. In some embodiments, an IgA2 antibody has a decreased aggregation compared to an IgA2 antibody that does not have a mutation in the C147 amino acid.

In some embodiments, an IgA2 antibody comprises a mutation of Y148, numbering according to IMGT scheme. In some embodiments, an IgA2 antibody comprises a non-conservative mutation of Y148, numbering according to IMGT scheme. In some embodiments, an IgA2 antibody comprises a deletion of amino acid Y148, numbering according to IMGT scheme.

In some embodiments, an anti-EGFR IgA antibody comprises one or more albumin binding domains. In some embodiments, one or more albumin binding domains are fused to a light chain or heavy chain of a IgA constant region. In some embodiments, one or more albumin binding domains are fused to a heavy chain of a IgA constant region. In some embodiments, one or more albumin binding domains are fused to a C-terminal region of a CH3 region of a heavy chain of an IgA constant region. In some embodiments, an IgA2 antibody has an increased circulating half-life compared to an IgA2 antibody that does not comprise one or more albumin binding domains. In some embodiments, an IgA2 antibody comprises one or more albumin binding domain and has circulating half-life within that of 1%, 54, or 10% of a corresponding IgG antibody. In some embodiments, an IgA2 antibody comprises one or more albumin binding domain and has circulating half-life greater than that of a corresponding IgG antibody.

In some embodiments, one or more mutation or deletion results in increased or decreased circulating half-life of the anti-EGFR IgA antibody. In some embodiments, one or more mutation or deletion results in increased circulating half-life of the anti-EGFR IgA antibody. For example, one or more mutations can increase the serum half-life of the anti-EGFR IgA antibody to up to 21 days or more in humans. Furthermore, one or more mutations can increase the serum half-life of the anti-EGFR IgA antibody to up to 9 days or more in mice. In some embodiments, one or more mutations can increase the serum half-life of the anti-EGFR IgA antibody to a level comparable to that of an immunoglobulin G (IgG) molecule. In some embodiments, one or more mutation or deletion results in decreased circulating half-life of the anti-EGFR IgA antibody.

In some embodiments, one or more mutations can increase serum half-life of an anti-EGFR IgA antibody for at least about 7 days to about 30 days or more. In some embodiments, one or more mutations can increase serum half-life of an anti-EGFR IgA antibody for at least about 7 days. In some embodiments, one or more mutations can increase serum half-life of an anti-EGFR IgA antibody for at most about 30 days. In some embodiments, one or more mutations can increase serum half-life of an anti-EGFR IgA antibody for about 7 days to about 8 days, about 7 days to about 9 days, about 7 days to about 10 days, about 7 days to about 15 days, about 7 days to about 20 days, about 7 days to about 25 days, about 7 days to about 30 days, about 8 days to about 9 days, about 8 days to about 10 days, about 8 days to about 15 days, about 8 days to about 20 days, about 8 days to about 25 days, about 8 days to about 30 days, about 9 days to about 10 days, about 9 days to about 15 days, about 9 days to about 20 days, about 9 days to about 25 days, about 9 days to about 30 days, about 10 days to about 15 days, about 10 days to about 20 days, about 10 days to about 25 days, about 10 days to about 30 days, about 15 days to about 20 days, about 15 days to about 25 days, about 15 days to about 30 days, about 20 days to about 25 days, about 20 days to about 30 days, or about 25 days to about 30 days. In some embodiments, one or more mutations can increase serum half-life of an anti-EGFR IgA antibody for about 7 days, about 8 days, about 9 days, about 10 days, about 15 days, about 20 days, about 25 days, or about 30 days. Accordingly, in some embodiments, an antibodies or a functional fragment thereof disclosed herein exhibit a greater circulating half-life compared to a corresponding WT IgA antibody. In some embodiments, the antibodies exhibit a circulating half-life that is greater by at least about 2%, 5%, 10%, 12%, 15%, 20%, at 25%, 50%, 65%, 70%, 75%, 85%, 90%, 95%, 99%, 100%, 150%, and 200%, relative to a corresponding WT IgA antibody.

In some embodiments, an anti-EGFR IgA antibody exhibits increased stability. In some embodiments, one or more mutations (e.g., insertion, substitution, and/or deletion) result in increased stability of the anti-EGFR IgA antibody compared to a corresponding IgA antibody which does not comprise the one or mutation. Accordingly, in some embodiments, one or more mutations (e.g., insertion, substitution, and/or deletion) of an anti-EGFR IgA antibody compared to a corresponding IgA antibody result in one or more of: reduced glycosylation, reduced aggregation, improved thermal stability, increased mechanical stability, or increased circulatory half-life.

In some embodiments, an anti-EGFR IgA antibody exhibits decreased aggregation. Antibody aggregation is a more common manifestation of physical instability. Protein aggregates generally have reduced activity and more importantly, greater immunogenicity potential because of the multiplicity of epitopes and/or conformational changes. For example, in some embodiments, an anti-EGFR IgA antibody binds to an epitope of the EGFR polypeptide or a variant thereof that comprises any one of the following EGFR amino acid residues: P349, F352, D355, P362, D355, Q384, P387, Q408, H409, F412, I438, K443, K465, I467, or S468. Immunoglobulin aggregates are known to cause serious renal failure and anaphylactoid reactions such as headache, fever, and chills. It is therefore advantageous to decrease aggregation in antibody therapeutics. Additionally, the aggregate level in commercial intravenous immunoglobulin products is limited to less than 5% based on the World Health Organization (WHO) standards. In some embodiments, one or more mutations results in decreased aggregation. In some embodiments, one or more mutation and/or one or more deletion results in decreased aggregation of an anti-EGFR IgA antibody compared to a corresponding IgA antibody which does not comprise the one or mutation and/or one or more deletion. In some embodiments, the antibodies or functional fragments thereof disclosed herein exhibit a decreased aggregation compared to a corresponding WT IgA antibody. In some embodiments, the antibodies exhibit aggregation that is decreased by at least about 2%, at least 5%, at least 10%, at least 12%, at least 15%, at least 20%, at least 25%, at least 50%, at least 65%, at least 70%, at least 75%, at least 85%, at least 90%, at least 95%, at least 99%, at least 100%, at least 150%, and at least 200%, relative to a corresponding WT IgA antibody. In some embodiments, the antibodies or functional fragments thereof disclosed herein exhibit a decreased aggregation with serum protein compared to a corresponding WT IgA antibody. In some embodiments, the antibodies exhibit aggregation that is decreased by at least 2%, at least 5%, at least 10%, at least 12%, at least 15%, at least 20%, at least 25%, at least 50%, at least 65%, at least 70%, at least 75%, at least 85%, at least 90%, at least 95%, at least 99%, at least 100%, at least 150%, and at least 200%, relative to a corresponding WT IgA antibody.

In some embodiments, the anti-EGFR IgA antibodies provided herein have an aggregate level ranging from at least about 0.1% to about 5% at most. In some embodiments, the IgA antibodies provided herein have an aggregate level ranging from at least about 0.1%. In some embodiments, the IgA antibodies provided herein have an aggregate level ranging from at most about 5%. In some embodiments, the anti-EGFR IgA antibodies provided herein have an aggregate level ranging from about 0.1% to about 0.5%, about 0.1% to about 1%, about 0.1% to about 2%, about 0.1% to about 3%, about 0.1% to about 4%, about 0.1% to about 5%, about 0.5% to about 1%, about 0.5% to about 2%, about 0.5% to about 3%, about 0.5% to about 4%, about 0.5% to about 5%, about 1% to about 2%, about 1% to about 3%, about 1% to about 4%, about 1% to about 5%, about 2% to about 3%, about 2% to about 4%, about 2% to about 5%, about 3% to about 4%, about 3% to about 5%, or about 4% to about 5%. In some embodiments, the IgA antibodies provided herein have an aggregate level ranging from about 0.1%, about 0.5%, about 1%, about 2%, about 3%, about 4%, or about 5%.

Anti-EGFR IgA antibodies disclosed herein may comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

Methods of substituting or deleting amino acids include, for example, site-directed mutagenesis. Mutagenesis can be performed by synthesizing an oligonucleotide having one or more modifications within the sequence of the constant domain of an antibody to be modified. The antibodies of the present disclosure (e.g., comprising one or more modifications in the IgA heavy chain constant region) can be prepared using any mutagenesis procedure known in the art, such as site-directed mutagenesis, synthetic gene construction, semi-synthetic gene construction, random mutagenesis, shuffling, etc. Site-specific mutagenesis allows the production of mutants through the use of specific oligonucleotide sequences which encode the DNA sequence of the desired mutation, as well as a sufficient number of adjacent oligonucleotides to provide a primer sequence of sufficient size and sequence complexity to form a stable duplex on both sides of the deletion junction being traversed. Typically, a primer of about 17 to about 75 nucleotides or more in length is preferred, with about 10 to about 25 or more residues on both sides of the junction of the sequence being altered. A number of such primers introducing a variety of different mutations at one or more positions may be used to generate a library of mutants.

Synthetic gene construction entails in vitro synthesis of a designed polynucleotide molecule to encode a polypeptide of interest. Gene synthesis can be performed utilizing a number of techniques, such as the multiplex microchip-based technology and similar technologies wherein oligonucleotides are synthesized and assembled upon photo-programmable microfluidic chips. Single or multiple amino acid substitutions, deletions, and/or insertions can be made and tested using methods of mutagenesis, recombination, and/or shuffling, followed by a relevant screening procedure. Other methods that can be used include error-prone PCR, phage display and region-directed mutagenesis.

Mutagenesis/shuffling methods can be combined with high-throughput, automated screening methods to detect activity of cloned, mutagenized polypeptides expressed by host cells. Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Other Covalent Modifications

In some embodiments, an anti-EGFR IgA antibody comprises one or more covalent modifications. They may be made by chemical synthesis or by enzymatic or chemical cleavage of an antibody, if applicable. Other types of covalent modifications of an antibody are introduced into the molecule by reacting targeted amino acid residues of the antibody with an organic derivatizing agent that is capable of reacting with selected side chains or the N- or C-terminal residues.

Cysteinyl residues most commonly are reacted with a haloacetate (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-(5 imidazoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino-terminal residues are reacted with succinic or other carboxylic acid anhydrides. Derivatization with these agents has the effect of reversing the charge of the lysinyl residues. Other suitable reagents for derivatizing alpha amino-containing residues include imidoesters such as methyl picolinimidate, pyridoxal phosphate, pyridoxal, chloro borohydride, trinitrobenzenesulfonic acid, methylisourea, 2,4-pentanedione, and transaminase-catalyzed reaction with glyoxylate.

Arginyl residues are modified by reaction with one or several reagents, among them phenylgly-oxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidazole and tetranitromethane are used to form 0-acetyl tyrosyl species and 3-nitro derivatives, respectively. Tyrosyl residues are iodinated using 125I or 131I to prepare labeled proteins for use in radioimmunoassay. Carboxyl side groups (aspartyl or glutamyl) are selectively modified by reaction with carbodiimides (R—N. dbd.C.dbd.N—R′), where R and R′ are different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues, respectively. These residues are deamidated under neutral or basic conditions. The deamidated form of these residues falls within the scope of this invention. Other modifications include hydroxylation of proline and lysine, phosphorylation of hydroxyl groups of seryl or threonyl residues, methylation of the alpha amino groups of lysine, arginine, and histidine side chains, acetylation of the N-terminal amine, and amidation of any C-terminal carboxyl group.

Another type of covalent modification involves chemically or enzymatically coupling glycosides to an antibody. These procedures are advantageous in that they do not require production of an antibody in a host cell that has glycosylation capabilities for N- or O-linked glycosylation. Depending on a coupling mode used, a sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Removal of any carbohydrate moieties present on an antibody may be accomplished chemically or enzymatically. Chemical deglycosylation requires exposure of an antibody to a compound trifluoromethanesulfonic acid, or an equivalent compound. This treatment results in cleavage of most or all sugars except the linking sugar (N-acetyl-glucosamine or N-acetylgalactosamine), while leaving the antibody intact. Enzymatic cleavage of carbohydrate moieties on antibodies can be achieved by the use of a variety of endo- and exoglycosidases.

Another type of covalent modification of an antibody comprises linking the antibody to one of a variety of nonproteinaceous polymers, e.g., polyethylene glycol, poly-propylene glycol, polyoxyethylated polyols, polyoxyethylated sorbitol, polyoxyethylated glucose, polyoxyethylated glycerol, polyoxyalkylenes, or polysaccharide polymers such as dextran.

### IgA Antibody Target

The anti-EGFR IgA antibodies described herein bind to EGFR expressed by a target cell (e.g., cancer cell). In some embodiments, the target cell is a human cell. In some embodiments, the EGFR is a human EGFR.

EGFR is a transmembrane glycoprotein that promotes cell growth in a variety of normal and transformed tissues. The receptor has several natural ligands including EGF and transforming growth factor-alpha. Binding of a ligand to the receptor stimulates cell proliferation. Blocking this interaction by means of an antibody directed against the receptor inhibits tumor growth in vivo.

In some embodiments, an anti-EGFR IgA antibody or a functional fragment thereof comprises one or more modifications disclosed herein in IgA heavy chain constant region while retaining an ability to specifically bind to an EGFR polypeptide (e.g., human EGFR). A polypeptide and coding nucleic acid sequences of EGFR of human origin and those of a number of animals are publicly available, e.g., from the NCBI website.

In some embodiments, an anti-EGFR IgA antibody binds to EGFR with a binding affinity (Kd) of less than 0.01 nM. In some embodiments, an anti-EGFR IgA antibody binds to EGFR with a binding affinity (Kd) of more than about 1 μM or more. In some embodiments, an anti-EGFR IgA antibody binds to EGFR with a binding affinity (Kd) of in a range of from 0.01 nM to 1 μM. In some embodiments, an anti-EGFR IgA antibody binds to EGFR with a binding affinity (Kd) of about 0.01 nM to about 800 nM, about 0.01 nM to about 500 nM, about 0.01 nM to about 300 nM, about 0.01 nM to about 100 nM, about 0.05 nM to about 800 nM, about 0.05 nM to about 500 nM, about 0.05 nM to about 300 nM, about 0.01 nM to about 100 nM, about 0.1 nM to about 800 nM, about 0.1 nM to about 500 nM, about 0.1 nM to about 300 nM, about 0.1 nM to about 100 nM, about 5 nM to about 800 nM, about 5 nM to about 500 nM, about 5 nM to about 300 nM, about 5 nM to about 100 nM, about 10 nM to about 800 nM, about 10 nM to about 500 nM, about 10 nM to about 300 nM, or about 10 nM to about 100 nM. In some embodiments, an anti-EGFR IgA antibody binds to EGFR with a binding affinity (Kd) of about 0.01 nM, about 0.05 nM, about 0.1 nM, about 1 nM, about 10 nM, about 100 nM, or about 200 nM.

In some embodiments, an anti-EGFR IgA antibody described herein comprises a Fab domain and an Fc domain, wherein the Fab domain binds the human EGFR and the Fc domain binds the FcαRI receptor on immune cell (e.g., neutrophils, macrophages, and eosinophils).

### Immune Effector Functions of Anti-EGFR IgA Antibodies

Provided herein are engineered anti-EGFR IgA antibodies comprising one or more modifications within their heavy chain constant region relative to a corresponding WT IgA antibody comprising a WT heavy chain constant region. In some embodiments, the anti-EGFR IgA antibodies or functional fragments thereof bind an Fc-alpha receptor (FcαR) expressed on an immune effector cell, such as an FcαR for human IgA. FcαRs are present on immune effector cells, for example, monocytes, macrophages, neutrophils, and other myeloid cells. FcαRs can also be found on metamyelocytes, myelocytes, promyelocytes and some myeloblasts from, e.g., bone marrow. Such receptors can also be found on myeloid cell lines, e.g., U937, PLB985, and HL60 cells. It has also been suggested that FcαRs are present on lymphocytes. Expression of FcαRs can be increased by activation of myeloid cells. For example, stimulation of U937 cells and PLB985 cells with Phorbol Myristic Acetate (PMA) increases cell surface level of FcαR several fold. Other agents which can increase surface level of FcαRs include calcitriol, 1-25 dihydroxy vitamin D3, and interferon-γ (IFN-γ).

FcαRs are capable of interacting with IgA1 and IgA2, in the form of monomers, dimers, and polymers. Accordingly, the anti-EGFR IgA antibodies or a functional fragment thereof are capable of triggering at least one Fc-receptor mediated immune effector cell function.

An immune effector cell is a cell which is involved in the effector phase of an immune response, as opposed to the cognitive and activation phases of an immune response. Immune effector cells include lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. An immune effector cell can phagocytose a target antigen, target cell, or microorganism. An immune effector cell can also lyse a target cell or a microorganism.

In some embodiments, an engineered anti-EGFR IgA antibody or a functional fragment thereof binds to a low-affinity Fc receptor FcαRI (CD89) of an immune effector cell. FcαRI interacts with IgA via its Cα1 and Cα2 domains, primarily after antibody-antigen recognition. FcαRI is a 55-75 kDa type I transmembrane receptor composed of two extracellular Ig-like domains, a transmembrane domain, and a cytoplasmic tail. It is expressed on myeloid cells such as neutrophils, eosinophils, activated monocytes, granulocytes, subsets of dendritic cells, Kupffer cells, and macrophages. Binding of an engineered anti-EGFR IgA antibody to FcαRI mediates effector functions such as phagocytosis, trogocytosis, oxidative burst, cytokine release, antigen presentation, and ADCC.

In some embodiments, an anti-EGFR IgA antibody disclosed herein exhibits increased binding affinity to an Fc receptor on an immune effector cell of at least about 2%, at least 5%, at least 10%, at least 12%, at least 15%, at least 20%, at least 25%, at least 50%, at least 65%, at least 70%, at least 75%, at least 85%, at least 90%, at least 95%, or more relative to a corresponding WT IgA or WT IgG.

In some embodiments, an anti-EGFR IgA antibody or a functional fragment thereof exhibits at least one increase in effector function as compared to a corresponding WT IgA antibody or a functional fragment thereof or a corresponding WT IgG antibody. In some embodiments, an anti-EGFR IgA antibody induces complement-dependent cytotoxicity (CDC). In some embodiments, an anti-EGFR IgA antibody induces polymorphonuclear neutrophil (PMN)-mediated tumor cell lysis. In some embodiments, IgA antibodies trigger polymorphonuclear cell (PMN) mediated ADCC more efficiently than IgG antibodies. In some embodiments, an anti-EGFR IgA antibody induces programmed cell death (PCD) via a caspase-independent pathway. In some embodiments, an anti-EGFR IgA antibody induces antibody-dependent cell-mediated cytotoxicity (ADCC).

In some embodiments, an IgA does not bind a B cell, a T cell, a platelet, and/or an erythrocyte. For example, in some embodiments, an anti-EGFR IgA antibody can have low immunogenicity.

### ADCC

ADCC is a cell-mediated reaction in which antigen-nonspecific cytotoxic/immune effector cells that express FcRs (e.g., natural killer (NK) cells, neutrophils, and macrophages) recognize antibody bound to the surface of a target cell and subsequently cause lysis of (i.e., “kill”) the target cell (e.g., cancer cell). Primary mediator cells can be natural killer (NK) cells and neutrophils. ADCC activity can be assessed directly using an in vitro assay, e.g., a 51Cr release assay using peripheral blood mononuclear cells (PBMC) and/or NK effector cells as described in the Examples provided herein. ADCC activity may be expressed as a concentration of antibody at which the lysis of target cells is half-maximal. Accordingly, in some embodiments, the concentration of an antibody or antigen binding functional fragment thereof of the disclosure, at which the lysis level is the same as the half-maximal lysis level by the wild-type control, is at least 2-, 3-, 5-, 10-, 20-, 50-, 100-fold lower than the concentration of the wild-type control itself.

Neutrophils can be found in EGFR-positive tumor microenvironment. Accordingly, in some embodiments, an anti-EGFR IgA antibody described herein exhibits neutrophil-mediated ADCC. In some embodiments, an anti-EGFR IgA antibody has a superior ability to recruit neutrophils for antibody-dependent cell-mediated cytotoxicity (ADCC) compared to a corresponding IgG antibody. In some embodiments, an anti-EGFR IgA antibody requires lower effector:target (E:T) ratios than a corresponding IgG to achieve a comparable level of ADCC. In some embodiments, neutrophil-mediated ADCC involves antibody-mediated trogoptosis. This is a process whereby neutrophils ‘nibble’ on the cancer cell membrane, leading to a loss of membrane integrity and ultimately causing cell death. In some embodiments, neutrophils are capable of inducing direct cytotoxicity through the release of high levels of ROS or by discharging granule content, particularly in the context of antibody-mediated targeting. Release of cytotoxic molecules from primary, secondary, and tertiary granules during degranulation can induce apoptotic elimination of cancer cells. In some embodiments, neutrophils are capable of mediating killing of cancer antigen loss variants, which are a consequence of tumor evolution and often hamper immunotherapies. In addition, neutrophils recruited by an anti-EGFR IgA contribute to recruitment and activation of other immune cells and stimulate adaptive anti-tumor immunity, which further promotes tumor cell elimination.

Additionally, in some embodiments, an anti-EGFR IgA antibody or a functional fragment thereof of the present disclosure can exhibit a higher maximal target cell lysis as compared to a corresponding wild-type IgA antibody or WT IgG antibody. For example, the maximal target cell lysis of an antibody or a functional fragment thereof of the disclosure can be 2%, 3%,4%, 5%, 10%, 15%, 20%, 25% or more higher than that of a corresponding WT IgA antibody or a corresponding WT IgG antibody. In some embodiments, antibodies or functional fragments thereof disclosed herein induce increased ADCC, compared to a corresponding WT IgG antibody comprising an IgG heavy chain constant region. In some embodiments, ADCC is increased by at least 2%, at least 5%, at least 10%, at least 12%, at least 15%, at least 20%, at least 25%, at least 50%, at least 65%, at least 70%, at least 75%, at least 85%, at least 90%, at least 95%, at least 99%, at least 100%, at least 150%, and at least 200%, relative to a corresponding WT IgG antibody.

In some embodiments, tumors comprising a KRAS mutation are sensitive to cell killing via ADCC induced by an anti-EGFR IgA antibody described herein. An effective induction of ADCC is dependent on an interaction between an Fc region of a target cell-bound therapeutic antibody and Fc receptors on the surface of immune effectors cells. In some embodiments, an anti-EGFR IgA antibody inhibits tumor growth and reduces tumor size by opsonization followed by recruitment of neutrophils and ADCC.

In some embodiments, an anti-EGFR IgA antibody can recruit NK cells and macrophages for cell killing via ADCC. In some embodiments, an engineered anti-EGFR IgA antibody-recruited NK cells, macrophages, and or neutrophils kill opsonized cancer cells by a mechanism that involves trogocytosis, which results in a lytic/necrotic type of cell death.

### CDC

A complement activation pathway is initiated by a binding of a first component of the complement system (C1q) to a molecule (e.g., an antibody) complexed with a cognate antigen. To assess complement activation, a CDC assay may be performed.

In some embodiments, an anti-EGFR IgA antibody described herein is capable of inhibiting EGFR, thereby causing cell cycle arrest and apoptosis of cancer cells.

### Biodistribution

In some embodiments, the anti-EGFR IgA antibodies or functional fragments thereof disclosed herein exhibit increased biodistribution relative to a corresponding WT IgA antibody. Various methods can be used to assessed and increased biodistribution of cells or tissues, including, but not limited to: nuclear medicine, whole body autoradiography, micro-autoradiography; phosphor imaging, cryo-imaging, nano-secondary ion mass spectroscopy (nanoSIMS), matrix-assisted laser desorption imaging (MALDI-MS), radiography (X-Ray), magnetic resonance imaging (MRI), computed tomography (CT), micro-ultrasound single photon emission CT (SPECT), positron emission tomography (PET) and the like. An increased biodistribution of an antibody to a target site, includes an at least 5%, 10%, 20%, 30%, 40%, 50%, 60%, 70%, 80%, 90%, 100%, 125%, 150%, 175%, 200%, or greater increase compared to a vehicle or a WT IgA antibody.

### Methods of Making an Antibody

In some embodiments, an anti-EGFR IgA antibody is made in a host cell. In some embodiments, a host cell is CHO. In some embodiments, a host cell is SP20. In some embodiments, a host cell is a HEK 239 host cell. In some embodiments, a HEK 293 host cell is HEK 293 F. In some embodiments, an antibody or a functional antigen binding fragment thereof is isolated from a host cell. In some embodiments, an antibody or a functional antigen binding fragment thereof is prepared in a cell-free system. An isolated nucleic acid molecule encoding an antibody, portion or polypeptide of the present disclosure can be recombined with vector DNA (e.g., expression vector) in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations can be used to construct nucleic acid sequences which encode an antibody molecule or antigen binding region thereof. Accordingly, the disclosure provides for a vector or expression vector comprising the isolated nucleic acids set forth herein. In one embodiment, a nucleic acid coding for a light chain and that coding for a heavy chain are isolated separately by procedures outlined above. In one embodiment, an isolated nucleic acid encoding a light chain and that coding for a heavy chain may be inserted into separate expression plasmids, or together in the same plasmid, so long as each is under suitable promoter and translation control.

Once an isolated nucleic acid molecule is placed into an expression vector, they are then transfected into host cells such as E. coli cells, simian COS cells, human embryonic kidney 293 cells (e.g., 293E cells), Chinese hamster ovary (CHO) cells, or myeloma cells that do not otherwise produce immunoglobulin protein, to synthesize an antibody or a functional antigen-binding fragment thereof in recombinant host cells. Any available vector can be utilized. Vector components generally include, but are not limited to, one or more of the following: a signal sequence, an origin of replication, one or more selective marker genes, an enhancer element, a promoter, and a transcription termination sequence.

The isolated nucleic acid molecules are operably linked to an expression control sequence in vector DNA. Expression control sequence refers to DNA sequences necessary for expression of an operably linked coding sequence in a particular host organism. Control sequences that are suitable for prokaryotes, for example, include a promoter, optionally an operator sequence, and a ribosome binding site. Eukaryotic cells are known to utilize promoters, polyadenylation signals, and enhancers.

Cell, cell line, and cell culture are often used interchangeably and all such designations herein include progeny. Transformants and transformed cells include primary subject cells and cultures derived therefrom without regard for the number of transfers. It is also understood that all progeny may not be precisely identical in DNA content, due to deliberate or inadvertent mutations. Mutant progeny that have the same function or biological activity as screened for in originally transformed cell are included. Where distinct designations are intended, it will be clear from the context. In an alternative embodiment, suitable encoding nucleic acid sequences can be designed according to a universal codon table, based on the known amino acid sequence of an immunoglobulin of interest.

Amino acid sequence variants of a desired antibody may be prepared by introducing appropriate nucleotide changes into an encoding DNA, or by peptide synthesis. Such variants include, for example, deletions from, and/or insertions into and/or substitutions of, residues within the amino acid sequences of the antibodies. Any combination of deletion, insertion, and substitution is made to arrive at the final construct, provided that the final construct possesses the desired characteristics. The amino acid changes also may alter post-translational processes of the monoclonal, human, humanized, or variant antibody, such as changing the number or position of glycosylation sites.

Nucleic acid molecules encoding amino acid sequence variants of an antibody are prepared by various methods. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of an antibody.

The present disclosure also provides isolated nucleic acid molecules encoding for antibodies or functional antigen binding fragments thereof, described herein, optionally operably linked to regulatory control sequences recognized by a host cell, vectors and host cells comprising the nucleic acids, and recombinant techniques for the production of the antibodies, which may comprise culturing the host cells so that the nucleic acid is expressed and, optionally, recovering the antibody from the host cell culture or culture medium.

For recombinant production of an antibody or a functional antigen binding fragment thereof, the nucleic acid molecule encoding it can be isolated and inserted into a replicable vector for further cloning (amplification of the DNA) or for expression. Accordingly provided herein are isolated antibody or functional antigen binding fragment thereof. In some embodiments, the antibodies or the present disclosure or functional antigen binding fragments thereof can be recombinant antibody. In some embodiment, a recombinant antibody has a glycosylation pattern that is different than the glycosylation pattern of an antibody having the same sequence if it were to exist in nature. In one embodiment, a recombinant antibody is expressed in a mammalian host cell which is not a human host cell. Notably, individual mammalian host cells have unique glycosylation patterns.

In some embodiments, the antibodies or functional antigen binding fragments thereof of the present disclosure is synthetic. The polypeptides of the present disclosure can be purified by isolation/purification methods.

In one aspect, provided herein is a host cell comprising the isolated nucleic acid molecules described herein or a vector comprising said isolated nucleic acid molecules described herein. The vector can be a cloning vector or an expression vector. Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41 P), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastors (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NP\7, and such viruses may be used as the virus herein according to the present invention, particularly for transfection of Spodoptera frugiperda cells.

Plant cell cultures of cotton, corn, potato, soybean, petunia, tomato, tobacco, lemna, and other plant cells can also be utilized as hosts. However, interest has been greatest in vertebrate cells, and propagation of vertebrate cells in culture (tissue culture) has become routine procedure. Examples of useful mammalian host cell lines are Chinese hamster ovary cells, including CHOK1 cells (ATCC CCL61), DXB-11, DG-44, and Chinese hamster ovary cells/-DHFR; monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); mouse sertoli cells (TM4); monkey kidney cells (CVI ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; and a human hepatoma line (Hep G2).

Host cells are transformed or transfected with the above-described expression or cloning vectors for antibody production and cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful and preferred for the expression of antibodies, described herein.

For transfection of the expression vectors and production of the chimeric, humanized, or composite human antibodies described herein, a recipient cell line can be a myeloma cell. Myeloma cells can synthesize, assemble and secrete immunoglobulins encoded by transfected immunoglobulin nucleic acid sequences and possess a mechanism for glycosylation of the immunoglobulin. For example, in some embodiments, a recipient cell is a recombinant Ig-producing myeloma cell SP2/0 (ATCC #CRL 8287). SP2/0 cells produce only immunoglobulin encoded by transfected genes. Myeloma cells can be grown in culture or in a peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells. An expression vector carrying a chimeric, humanized, or composite human antibody construct or antibody polypeptide described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment.

Yeast provides certain advantages over bacteria for production of immunoglobulin H and L chains. Yeasts carry out post-translational peptide modifications including glycosylation. A number of recombinant DNA strategies exist that utilize strong promoter sequences and high copy number plasmids which can be used for production of desired proteins in yeast. Yeast recognizes leader sequences of cloned mammalian gene products and secretes peptides bearing leader sequences (i.e., pre-peptides). Yeast gene expression systems can be routinely evaluated for levels of production, secretion and stability of an antibody polypeptide or a functional antigen binding fragment peptide thereof, and assembled chimeric, humanized, or composite human antibodies, functional fragments and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, a promoter and terminator signals of a phosphoglycerate kinase (PGK) gene can be utilized. A number of approaches can be taken for evaluating optimal expression plasmids for expression of cloned immunoglobulin cDNAs in yeast. In some embodiments, a plasmid comprises a non-integrating plasmid. In some embodiments, a plasmid is capable of transiently expressing cDNAs.

Bacterial strains can also be utilized as hosts for the production of the antibody molecules or functional fragments thereof described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. A vector carries a replication site, as well as specific genes which are capable of providing phenotypic selection in transformed cells. A number of approaches can be taken for evaluating the expression plasmids for the production of chimeric, humanized, or composite humanized antibodies and functional fragments thereof encoded by the cloned immunoglobulin cDNAs or CDRs in bacteria.

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein. Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Exemplary eukaryotic cells that can be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, a particular eukaryotic host cell is selected based on its ability to make desired post-translational modifications to the variable heavy chains and/or variable light chains. For example, in some embodiments, CHO cells produce polypeptides that have a higher level of sialylation than the same polypeptide produced in 293 cells.

In some embodiments, polypeptides of the antibodies or functional antigen binding fragments thereof, disclosed herein can be produced in vivo in an animal that has been engineered or transfected with one or more nucleic acid molecules encoding the polypeptides, according to any suitable method.

Many vector systems are available for the expression of H and L chain nucleic acid sequence in mammalian cells. Different approaches can be followed to obtain complete H2L2 antibodies. As discussed above, it is possible to co-express H and L chains in the same cells to achieve intracellular association and linkage of H and L chains into complete tetrameric H2L2 antibodies and/or functional antigen binding fragment peptides. Co-expression can occur by using either the same or different plasmids in the same host. Genes for both H and L chains and/or CDR3 regions peptides can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing functional antigen binding peptide fragments and/or H2L2 molecules via either route could be transfected with plasmids encoding additional copies of peptides, H, L, or H plus L chains in conjunction with additional selectable markers to generate cell lines with enhanced properties, such as higher production of assembled H2L2 antibody molecules or enhanced stability of the transfected cell lines.

In some aspects, provided herein are methods and systems for production of a humanized antibody, which is prepared by a process which comprises maintaining a host transformed with a first expression vector which encodes a light chain of the humanized antibody and with a second expression vector which encodes a heavy chain of the humanized antibody under such conditions that each chain is expressed and isolating the humanized antibody formed by assembly of the thus-expressed chains. The first and second expression vectors can be the same vector. Also provided herein are DNA sequences encoding a light chain or a heavy chain of a humanized antibody; an expression vector which incorporates said DNA sequence; and a host transformed with a said expression vector. Generating a humanized antibody from nucleic acid sequences and information provided herein can be practiced by those of ordinary skill in the art without undue experimentation. In one approach, there are four general steps employed to humanize a monoclonal antibody. These are: (1) determining a nucleotide and predicted amino acid sequence of starting antibody light and heavy variable domains; (2) designing a humanized antibody, i.e., deciding which antibody framework region to use during a humanizing process; (3) an actual humanizing methodologies/techniques; and (4) a transfection and expression of the humanized antibody.

### Purification

Contaminant components of its natural environment are materials that would interfere with diagnostic or therapeutic uses of a polypeptide, and may include enzymes, hormones, and other proteinaceous or non-proteinaceous components. In some embodiments, the antibodies or functional antigen binding fragments thereof of the instant disclosure can be purified by a suitable method. In preferred embodiments, a polypeptide is purified: (1) to greater than 95% by weight of polypeptide as determined by the Lowry method, and most preferably more than 99% by weight; (2) to a degree sufficient to obtain at least 15 residues of N-terminal or internal amino acid sequence by use of a spinning cup sequenator, or (3) to homogeneity as shown by SDS-PAGE under reducing or non-reducing conditions and using Coomassie blue or, preferably, silver staining. Isolated antibody includes the polypeptide in situ within recombinant cells since at least one component of the polypeptide's natural environment will not be present. Ordinarily, however, isolated polypeptide will be prepared by at least one purification step. In one aspect, disclosed herein is a purified antibody or functional antigen-binding fragment as provided herein.

Once expressed, whole antibodies, their dimers, individual light and heavy chains, or other immunoglobulin forms of the present invention can be recovered and purified by techniques such as immunoabsorption or immunoaffinity chromatography, chromatographic methods such as HPLC (high performance liquid chromatography), ammonium sulfate precipitation, gel electrophoresis, or any combination of these. Substantially pure immunoglobulins of at least about 90% to 95% homogeneity are advantageous, as are those with 98% to 99% or more homogeneity, particularly for pharmaceutical uses. When using recombinant techniques, an antibody can be produced intracellularly, in periplasmic space, or directly secreted into a medium, including from microbial cultures. If an antibody is produced intracellularly, as a first step, particulate debris, either host cells or lysed fragments, is removed, for example, by centrifugation or ultrafiltration.

An antibody composition isolated from microbial or mammalian cells can be purified using, for example, hydroxylapatite chromatography cation or avian exchange chromatography, and affinity chromatography, with affinity chromatography being a preferred purification technique. A suitability of protein A as an affinity ligand depends on species and isotype of any immunoglobulin Fc domain that is present in an antibody. Protein A can be used to purify antibodies that are based on human y1, y2, or y4 heavy chains. Protein G is recommended for all mouse isotypes and for human y3. A matrix to which an affinity ligand is attached is most often agarose, but other matrices are available. Mechanically stable matrices such as controlled pore glass or poly(styrenedivinyl)benzene allow for faster flow rates and shorter processing times than can be achieved with agarose. Where an antibody comprises a CH3 domain, a resin that binds specifically to the CH3 domain can be useful for purification. Other techniques for protein purification such as fractionation on an ion-exchange column, ethanol precipitation, Reverse Phase HPLC, chromatography on silica, chromatography on heparin bead chromatography on an anion or cation exchange resin (such as a polyaspartic acid column), chromatofocusing, SDS-PAGE, and ammonium sulfate precipitation are also available depending on an antibody to be recovered. Once purified, partially or to homogeneity as desired, a humanized or composite human antibody can then be used therapeutically or in developing and performing assay procedures, immunofluorescent staining, and the like.

Functional activities of an antibody or a functional antigen-binding fragment disclosed herein. Such functional activities include biological activity and ability to bind to a cancer cell antigen. Additionally, a polypeptide having functional activity means the polypeptide exhibits activity similar, but not necessarily identical to, an activity of an antibody described herein, including mature forms, as measured in a particular assay, such as, for example, a biological assay, with or without dose dependency. In the case where dose dependency does exist, it need not be identical to that of the antibodies of the disclosure, but rather substantially similar to the dose-dependence in a given activity as compared to the antibodies set forth herein (i.e., the candidate polypeptide will exhibit greater activity, or not more than about 25-fold less, about 10-fold less, or about 3-fold less activity relative to the antibodies described herein).

### Nucleic Acid Molecules Encoding Antibodies

Using the information provided herein, for example, nucleic acid and amino acid sequences of the antibodies; a nucleic acid molecule encoding the antibodies or functional antigen-binding fragments thereof can be easily obtained by a skilled artisan. Nucleic acid molecules of the present disclosure may be in the form of RNA, such as mRNA, hnRNA, tRNA or any other form, or in the form of DNA, including but not limited to, cDNA and genomic DNA obtained by cloning or produced synthetically, or any combinations thereof. A DNA may be triplex, duplex or single-stranded, or any combination thereof. Any portion of at least one strand of a DNA or RNA may be a coding strand, also known as a sense strand, or it can be an antisense strand, also known as an antisense strand.

Nucleic acids can be present in whole cells, in a cell lysate, or in a partially purified or substantially pure form. A nucleic acid molecule is isolated or rendered substantially pure when purified away from other cellular components or other contaminants, e.g., other cellular nucleic acids or proteins, by standard techniques, including, but not limited to alkaline/SDS treatment, CsCl banding, column chromatography, agarose gel electrophoresis and others. A nucleic acid according to at least some embodiments of the disclosure can be, for example, DNA or RNA and may or may not contain intronic sequences. In a preferred embodiment, a nucleic acid is a cDNA molecule.

Another aspect of the present disclosure pertains to nucleic acid molecules comprising nucleic acid sequences that encode an antibody polypeptide or a functional fragment thereof described herein or a functional antigen-binding fragment thereof. In some embodiments, an isolated nucleic acid molecule comprises a nucleic acid sequence encoding a modified IgA heavy chain constant region. In some embodiments, an isolated nucleic acid molecule comprises a nucleic acid sequence encoding a light chain variable region polypeptide of an antibody.

Once DNA fragments encoding VH and VL segments are obtained, these DNA fragments can be further manipulated by standard recombinant DNA techniques, for example to convert variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. An isolated DNA encoding a VH region can be converted to a full-length heavy chain gene by operatively linking a VH-encoding DNA to another DNA molecule encoding heavy chain constant regions (CH1, CH2 and CH3). DNA fragments encompassing human heavy chain constant region genes can be obtained by standard PCR amplification. A heavy chain constant region can be an IgA1 or a IgA2 constant region. For a Fab fragment heavy chain gene, a VH-encoding DNA can be operatively linked to another DNA molecule encoding only a heavy chain CH1 constant region.

An isolated DNA encoding a VL region can be converted to a full-length light chain gene (as well as a Fab light chain gene) by operatively linking the VL-encoding DNA to another DNA molecule encoding a light chain constant region, CL. DNA fragments encompassing human light chain constant regions can be obtained by standard PCR amplification. A light chain constant region can be a kappa or lambda constant region, but most preferably is a kappa constant region.

To create a scFv gene, a VH- and VL-encoding DNA fragments are operatively linked to another fragment encoding a flexible linker, e.g., encoding an amino acid sequence (Gly-4-Ser)3 (SEQ ID NO: 220), such that the VH and VL sequences can be expressed as a contiguous single-chain protein, with the VL and VH regions joined by the flexible linker.

Nucleic acid molecules isolated from the present disclosure can include nucleic acid molecules comprising an open reading frame (ORF), optionally with one or more introns, e.g., but not limited to, at least one specified portion of at least a CDR, as CDR1, CDR2 and/or CDR3 of at least one light chain; nucleic acid molecules comprising a coding sequence of a cancer associated antibody disclosed herein or variable region e.g., variable regions of a light chain; and nucleic acid molecules comprising a nucleotide sequence substantially different from those described above but which, due to the degeneracy of the genetic code, still encode at least antibody or functional antigen binding fragment thereof as described herein.

Nucleic acid molecules comprising nucleic acid sequence that encode one or more chains of an antibody are provided herein. In some embodiments, a nucleic acid molecule comprises a nucleic acid sequence that encodes a heavy chain or a light chain of an antibody. In some embodiments, a nucleic acid molecule comprises both a nucleic acid sequence that encodes a heavy chain and a nucleic acid sequence that encodes a light chain, of an antibody. In some embodiments, a first nucleic acid molecule comprises a first nucleic acid sequence that encodes a heavy chain and a second nucleic acid molecule comprises a second nucleic acid sequence that encodes a light chain.

In some embodiments, a heavy chain and a light chain are expressed from one nucleic acid molecule, or from two separate nucleic acid molecules, as two separate polypeptides. In some embodiments, such as when an antibody is an scFv, a single nucleic acid sequence encodes a single polypeptide comprising both a heavy chain and a light chain linked together.

In some embodiments, a nucleic acid sequence encoding a heavy chain or light chain of an antibody disclosed herein comprises a nucleic acid sequence that encodes at least one of the CDRs provided herein. In some embodiments, a nucleic acid sequence encoding a heavy chain or light chain of an antibody disclosed herein comprises a sequence that encodes at least 3 of the CDRs provided herein.

In some embodiments, a nucleic acid sequence encoding a heavy chain or light chain of an antibody comprises a sequence that encodes at least 6 of the CDRs provided herein. In some embodiments, a nucleic acid sequence encoding a heavy chain or light chain of an antibody comprises a nucleotide sequence that encodes a leader sequence, which, when translated, is located at the N terminus of the heavy chain or light chain. A leader sequence may be a native heavy or light chain leader sequence, or may be another heterologous leader sequence. A leader sequence can be cleaved upon export of a polypeptide from a mammalian cell, forming a mature protein. Leader sequences can be natural or synthetic, and they can be heterologous or homologous to a protein to which they are attached.

In some embodiments, a nucleic acid molecule is one that encodes any of the amino acid sequences for the variable light chain and variable heavy chain in TABLE 5 and TABLE 7 herein. In some embodiments, a nucleic acid sequence is one that is at least 80% identical to a nucleic acid encoding any of the amino acid sequences variable light chain and variable heavy chain in TABLE 5 and TABLE 7 herein, for example, at least 60%, at least 70%, at least 75%, at least 80%, at least 85%, at least 90%, at least 95%, or at least 99% identical. In some embodiments, a nucleic acid is one that hybridizes to any one or more of the nucleic acid sequences provided herein. In some embodiments, the hybridization is under moderate conditions. In some embodiments, the hybridization is under highly stringent conditions, such as: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

Nucleic acid molecules can be constructed using recombinant DNA techniques conventional in the art. In some embodiments, a nucleic acid molecule is placed in an expression vector that is suitable for expression in a selected host cell.

Vectors comprising nucleic acid molecules that encode the antibodies or functional antigen binding fragment herein are provided. Vectors comprising nucleic acid molecules that encode a heavy chains and/or a light chains are also provided. Such vectors include, but are not limited to, DNA vectors, phage vectors, viral vectors, retroviral vectors, etc. In one embodiment, a nucleic acid coding for a light chain and that coding for a heavy chain are isolated separately by the procedures outlined above. In one embodiment, an isolated nucleic acid encoding a light chain and that coding for a heavy chain may be inserted into separate expression plasmids, or together in the same plasmid, so long as each is under suitable promoter and translation control. In some embodiments, a heavy chain and light chain are expressed as part of a single polypeptide, such as, for example, when an antibody is an scFv.

In some embodiments, a first vector comprises a nucleic acid molecule that encodes a heavy chain and a second vector comprises a nucleic acid molecule that encodes a light chain. In some embodiments, a first vector and second vector are transfected into host cells in similar amounts (such as similar molar amounts or similar mass amounts). In some embodiments, a mole- or mass-ratio of between 5:1 and 1:5 of a first vector and a second vector is transfected into host cells. In some embodiments, a mass ratio of between 1:1 and 1:5 for a vector encoding a heavy chain and a vector encoding a light chain is used. In some embodiments, a mass ratio of 1:2 for a vector encoding a heavy chain and a vector encoding a light chain is used. In some embodiments, a vector is selected that is optimized for expression of polypeptides in CHO or CHO-derived cells, or in NSO cells.

In one aspect, the present disclosure provides methods for treatment or prevention of cancer comprising administering nucleic acid molecules, wherein the nucleic acid molecules encode a VH, VL, CDR3 region of VH or CDR 3 region of VL or functional antigen binding fragment thereof. In some embodiments, a nucleic acid molecule disclosed herein is used for gene therapy. Gene therapy refers to therapy performed by administration to a subject of an expressed or expressible nucleic acid. In this embodiment, the nucleic acids produce their encoded protein that mediates a prophylactic or therapeutic effect. Any of the methods for gene therapy available in the art can be used according to the embodiments herein.

Delivery of a therapeutic antibody to appropriate cells can be effected via gene therapy ex vivo, in situ, or in vivo by use of any suitable approach, including by use of physical DNA transfer methods (e.g., liposomes or chemical treatments) or by use of viral vectors (e.g., adenovirus, adeno-associated virus, or a retrovirus). For example, for in vivo therapy, a nucleic acid encoding a desired antibody, either alone or in conjunction with a vector, liposome, or precipitate may be injected directly into a subject, and in some embodiments, may be injected at a site where expression of the antibody compound is desired. For ex vivo treatment, a subject's cells are removed, a nucleic acid is introduced into these cells, and modified cells are returned to the subject either directly or, for example, encapsulated within porous membranes which are implanted into the patient (subject). There are a variety of techniques available for introducing nucleic acids into viable cells. The techniques vary depending upon whether a nucleic acid is transferred into cultured cells in vitro, or in vivo in cells of an intended host. Techniques suitable for a transfer of nucleic acid into mammalian cells in vitro include use of liposomes, electroporation, microinjection, cell fusion, DEAE-dextran, and calcium phosphate precipitation. A commonly used vector for ex vivo delivery of a nucleic acid is a retrovirus.

Other in vivo nucleic acid transfer techniques include transfection with viral vectors (such as adenovirus, Herpes simplex I virus, or adeno-associated virus) and lipid-based systems. The nucleic acid and transfection agent are optionally associated with a microparticle. Exemplary transfection agents include calcium phosphate or calcium chloride co-precipitation, DEAE-dextran-mediated transfection, quaternary ammonium amphiphile DOTMA ((dioleoyloxypropyl) trimethylammonium bromide, commercialized as Lipofectin by GIBCO-BRL)); lipophilic glutamate diesters with pendent trimethylammonium heads; a metabolizable parent lipids such as a cationic lipid dioctadecylamido glycylspermine (DOGS) and dipalmitoylphosphatidyl ethanolamylspermine (DPPES); metabolizable quaternary ammonium salts (DOTB, N-(1-[2,3-dioleoyloxy]propyl)-N,N,N-trimethylammonium methylsulfate (DOTAP), polyethyleneimine (PEI), dioleoyl esters, ChoTB, ChoSC, DOSC); 3beta[N—(N′,N′-dimethylaminoethane)-carbamoyl]cholesterol (DC-Chol), dioleoylphosphatidyl
ethanolamine (DOPE)/3beta[N—(N′,N′-dimethylaminoethane)-carbamoyl]cholesterol-DC-Chol in one to one mixtures, spermine, spermidine, lipopolyamines, lipophilic polylysines (LPLL), [[(1, 1,3,3 tetramethylbutyl)cresoxy]ethoxy]ethyl]dimethylbenzylammonium hydroxide (DEBDA hydroxide) with excess phosphatidylcholine/cholesterol, cetyltrimethylammonium bromide (CTAB)/DOPE mixtures, lipophilic diester of glutamic acid (TMAG) with DOPE, CTAB, DEBDA, didodecylammonium bromide (DDAB), and stearylamine in admixture with phosphatidylethanolamine, and oligogalactose bearing lipids. Exemplary transfection enhancer agents that increase efficiency of transfer include, for example, DEAE-dextran, polybrene, lysosome-disruptive peptide, chondroitan-based proteoglycans, sulfated proteoglycans, polyethylenimine, polylysine, integrin-binding peptide CYGGRGDTP (SEQ ID NO: 33), linear dextran nonasaccharide, glycerol, cholesteryl groups tethered at the Y-terminal internucleoside link of an oligonucleotide, lysophosphatide, lysophosphatidylcholine,
lysophosphatidylethanolamine, and 1-oleoyl lysophosphatidylcholine.

In some situations, it may be desirable to deliver a nucleic acid with an agent that directs the nucleic acid containing vector to target cells. Such agents include antibodies specific for a cell-surface membrane protein on a target cell, or a ligand for a receptor on the target cell. Where liposomes are employed, proteins which bind to a cell-surface membrane protein associated with endocytosis may be used for targeting and/or to facilitate uptake. Examples of such proteins include capsid proteins and fragments thereof tropic for a particular cell type, antibodies for proteins which undergo internalization in cycling, and proteins that target intracellular localization and enhance intracellular half-life. In other embodiments, receptor-mediated endocytosis can be used.

### Immunoconjugates

The antibodies or functional antigen binding fragments thereof, disclosed herein, may be administered in their “naked” or unconjugated form, or may have therapeutic agents conjugated to them. In one embodiment, antibodies or functional antigen binding fragments thereof are used as a radiosensitizer. In such embodiments, the antibodies or functional antigen binding fragments are conjugated to a radiosensitizing agent. In some embodiments, the radiosensitizer is a molecule, preferably a low molecular weight molecule, administered to animals in therapeutically effective amounts to increase sensitivity of cells to be radiosensitized to electromagnetic radiation and/or to promote treatment of diseases that are treatable with electromagnetic radiation. Diseases that are treatable with electromagnetic radiation include neoplastic diseases, benign and malignant tumors, and cancerous cells. In some embodiments, electromagnetic radiation and radiation include, but are not limited to, radiation having a wavelength of 10-20 to 100 meters. Preferred embodiments of the present disclosure can employ for example, an electro-magnetic radiation of: gamma-radiation c10-20 to 10-13 m), X-ray radiation (10-12 to 10-9 m), ultraviolet light (10 nm to 400 nm), visible light (400 nm to 700 nm), infrared radiation (700 nm to 1.0 mm), and microwave radiation (1 mm to 30 cm).

Radiosensitizers are known to increase sensitivity of cancerous cells to toxic effects of electromagnetic radiation. Many cancer treatment protocols currently employ radiosensitizers activated by an electromagnetic radiation of X-rays. Examples of X-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromode-oxyuridine (BUdR), 5-iododeoxyuridine (IUdR), bromode-oxycytidine, fluorodeoxyuridine (FUdR), hydroxyurea, cisplatin, and therapeutically effective analogs and derivatives of the same.

Photodynamic therapy (PDT) of cancers employs visible light as a radiation activator of a sensitizing agent. Examples of photodynamic radiosensitizers include the following, but are not limited to: hematoporphyrin derivatives, benzoporphyrin derivatives, NPe6, tin etioporphyrin (SnET2), pheoborbide-a, bacteriochlorophyll-a, naphthalocyanines, phthalocyanines, zinc phthalocyanine, and therapeutically effective analogs and derivatives of the same.

In another embodiment, an antibody may be conjugated to a receptor (such streptavidin) for utilization in tumor pretargeting, wherein an antibody-receptor conjugate is administered to a patient, followed by removal of unbound conjugate from circulation using a clearing agent and then administration of a ligand (e.g., avidin) which is conjugated to a cytotoxic agent (e.g., a radionuclide).

The present disclosure further provides the above-described antibodies or antigen binding thereof in detectably labeled form. Antibodies can be detectably labeled through use of radioisotopes, affinity labels (such as biotin, avidin, etc.), enzymatic labels (such as horseradish peroxidase, alkaline phosphatase, etc.) fluorescent or luminescent or bioluminescent labels (such as FITC or rhodamine, etc.), paramagnetic atoms, and the like.

Exemplary therapeutic immunoconjugates comprise an antibody described herein conjugated to a cytotoxic agent such as a chemotherapeutic agent, toxin (e.g., an enzymatically active toxin of bacterial, fungal, plant or animal origin, or fragments thereof), or a radioactive isotope (i.e., a radioconjugate). Fusion proteins are described in further detail below.

In some embodiments, antibodies and functional antigen binding fragments thereof disclosed herein can be conjugated to a therapeutic agent such as a chemotherapeutic cytotoxin, such as a cytostatic or cytocidal agent (e.g., paclitaxol, cytochalasin B or diphtheria toxin, taxol, cytochalasin B, gramicidin D, ethidium bromide, emetine, mitomycin, etoposide, tenoposide, vincristine, vinblastine, colchicin, doxorubicin, daunorubicin, dihydroxy anthracindione, mitoxantrone, mithramycin, actinomycin D, 1-dehydrotestosterone, glucocorticoids, procaine, tetracaine, lidocaine, propranolol, and puromycin and analogs or homologs thereof.), antimetabolites (e.g., methotrexate, 6-mercaptopurine, 6-thioguanine, cytarabine, fludarabin, 5-fluorouracil decarbazine), alkylating agents (e.g., mechlorethamine, thioepa chlorambucil, melphalan, carmustine (BSNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC), and anti-mitotic agents, a thrombotic or anti-angiogenic agent or a radioactive label. In another embodiment, antibodies and functional antigen binding fragments thereof disclosed herein are conjugated to a detectable substrate such as, e.g., an enzyme, fluorescent marker, chemiluminescent marker, bioluminescent material, or radioactive material. In some embodiments, antibodies and functional antibody fragments thereof disclosed herein are conjugated to a toxin (e.g., an enzymatically active toxin of bacterial, fungal, plant or animal origin, or fragments thereof), a small molecule, an siRNA, a nanoparticle, a targeting agent (e.g., a microbubble), or a radioactive isotope (i.e., a radioconjugate). Such conjugates are referred to herein as “immunoconjugates”. Such immunoconjugates can be used, for example, in diagnostic, theranostic, or targeting methods.

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin and the tricothecenes. A variety of radioisotopes are available for production of radioconjugate antibodies. Examples include, but are not limited to, 212 Bi, 131 I, 131 In, 90Y and 186Re.

Conjugates of the antibodies or functional antigen binding fragments thereof described herein and a cytotoxic agent can be made using any of a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared. Carbon-14-labeled 1-isothiocyanatobenzyl-3-methyldiethylene triaminepentaacetic acid (MX-DTPA) is an exemplary chelating agent for conjugation of radionucleotide to the antibody.

In other embodiments, an antibody or a portion thereof can be conjugated to a receptor (e.g., streptavidin) for utilization in tumor pre-targeting, wherein the antibody-receptor conjugate is administered to the subject, followed by removal of unbound conjugate from the circulation using a clearing agent and then administration of a ligand (e.g., avidin) which is conjugated to a cytotoxic agent (e.g. a radionucleotide). In some embodiments, an antibody or a functional fragment thereof can be conjugated to biotin, and the biotin conjugated antibody or functional antibody fragment thereof can be further conjugated or linked to a streptavidin-bound or -coated agent, such as a streptavidin-coated microbubble, for use in, for example, molecular imaging of angiogenesis.

Immunoconjugates can be prepared by indirectly conjugating a therapeutic agent to an antibody component. A general method involves reacting an antibody component having an oxidized carbohydrate portion with a carrier polymer that has at least one free amine function and that is loaded with a plurality of drug, toxin, chelator, boron addends, or other therapeutic agent. This reaction results in an initial Schiff base (imine) linkage, which can be stabilized by reduction to a secondary amine to form a final conjugate.

A carrier polymer is preferably an aminodextran or polypeptide of at least 50 amino acid residues, although other substantially equivalent polymer carriers can also be used. Preferably, a final immunoconjugate is soluble in an aqueous solution, such as mammalian serum, for ease of administration and effective targeting for use in therapy. Thus, solubilizing functions on a carrier polymer will enhance serum solubility of a final immunoconjugate. In particular, an aminodextran will be preferred.

A process for preparing an immunoconjugate with an aminodextran carrier typically begins with a dextran polymer, advantageously a dextran of average molecular weight of about 10,000-100,000. A dextran is reacted with an oxidizing agent to affect a controlled oxidation of a portion of its carbohydrate rings to generate aldehyde groups. An oxidation is conveniently affected with glycolytic chemical reagents such as NaI04, according to conventional procedures.

An oxidized dextran is then reacted with a polyamine, preferably a diamine, and more preferably, a mono- or polyhydroxy diamine. Suitable amines include ethylene diamine, propylene diamine, or other like polymethylene diamines, diethylene triamine or like polyamines, 1,3-diamino-2-hydroxypropane, or other like hydroxylated diamines or polyamines, and the like. An excess of amine relative to aldehyde groups of the dextran is used to ensure substantially complete conversion of aldehyde functions to Schiff base groups.

A reducing agent, such as NaBH4, NaBH3CN or the like, is used to effect reductive stabilization of a resultant Schiff base intermediate. A resultant adduct can be purified by passage through a conventional sizing column to remove cross-linked dextrans. Other conventional methods of derivatizing a dextran to introduce amine functions can also be used, e.g., reaction with cyanogen bromide, followed by reaction with a diamine. The anminodextran is then reacted with a derivative of the particular drug, toxin, chelator, immunomodulator, boron addend, or other therapeutic agent to be loaded, in an activated form, preferably, a carboxyl-activated derivative, prepared by conventional means, e.g., using dicyclohexylcarbodiimide (DCC) or a water-soluble variant thereof, to form an intermediate adduct.

Alternatively, polypeptide toxins such as poke-weed antiviral protein or ricin A-chain, and the like, can be coupled to aminodextran by glutaraldehyde condensation or by reaction of activated carboxyl groups on a protein with amines on the aminodextran. Chelators for radiometals or magnetic resonance enhancers, such as derivatives of ethylenediaminetetraacetic acid (EDTA) and diethylen-etriaminepentaacetic acid (DTPA), typically have groups on a side chain by which a chelator can be attached to a carrier. Such groups include, e.g., benzylisothiocyanate, by which the DTPA or EDTA can be coupled to the amine group of a carrier. Alternatively, carboxyl groups or amine groups on a chelator can be coupled to a carrier by activation or prior derivatization and then coupling.

Boron addends, such as carboranes, can be attached to antibody components by conventional methods. For example, carboranes can be prepared with carboxyl functions on pendant side chains. Attachment of such carboranes to a carrier, e.g., aminodextran, can be achieved by activation of the carboxyl groups of the carboranes and condensation with amines on the carrier to produce an intermediate conjugate. Such intermediate conjugates are then attached to antibody components to produce therapeutically useful immunoconjugates, as described below.

A polypeptide carrier can be used instead of aminodextran, but the polypeptide carrier should have at least 50 amino acid residues in a chain, preferably 100-5000 amino acid residues. At least some of the amino acids should be lysine residues or glutamate or aspartate residues. The pendant amines of lysine residues and pendant carboxylates of glutamine and aspartate are convenient for attaching a drug, toxin, immunomodulator, chelator, boron addend or other therapeutic agent. Examples of suitable polypeptide carriers include polylysine, polyglutamic acid, polyaspartic acid, co-polymers thereof, and mixed polymers of these amino acids and others, e.g., serines, to confer desirable solubility properties on the resultant loaded carrier and immunoconjugate.

Conjugation of the intermediate conjugate with an antibody component is effected by oxidizing carbohydrate portion of the antibody component and reacting the resulting aldehyde (and ketone) carbonyls with amine groups remaining on the carrier after loading with a drug, toxin, chelator, immunomodulator, boron addend, or other therapeutic agent. Alternatively, an intermediate conjugate can be attached to an oxidized antibody component via amine groups that have been introduced in the intermediate conjugate after loading with the therapeutic agent. Oxidation is conveniently effected either chemically, e.g., with NaI04 or other glycolytic reagent, or enzymatically, e.g., with neuraminidase and galactose oxidase. In the case of an aminodextran carrier, not all of the amines of the aminodextran are typically used for loading a therapeutic agent. The remaining amines of aminodextran condense with the oxidized antibody component to form Schiff base adducts, which are then reductively stabilized, normally with a borohydride reducing agent.

Analogous procedures are used to produce other immunoconjugates according to the invention. Loaded polypeptide carriers preferably have free lysine residues remaining for condensation with the oxidized carbohydrate portion of an antibody component. Carboxyls on the polypeptide carrier can, if necessary, be converted to amines by, e.g., activation with DCC and reaction with an excess of a diamme.

A final immunoconjugate is purified using conventional techniques, such as sizing chromatography on Sephacryl S-300 or affinity chromatography using one or more CD84Hy epitopes. Alternatively, immunoconjugates can be prepared by directly conjugating an antibody component with a therapeutic agent. The general procedure is analogous to the indirect method of conjugation except that a therapeutic agent is directly attached to an oxidized antibody component. It will be appreciated that other therapeutic agents can be substituted for the chelators described herein. Those of skill in the art will be able to devise conjugation schemes without undue experimentation.

As a further illustration, a therapeutic agent can be attached at a hinge region of a reduced antibody component via disulfide bond formation. For example, tetanus toxoid peptides can be constructed with a single cysteine residue that is used to attach a peptide to an antibody component. As an alternative, such peptides can be attached to an antibody component using a heterobifunctional cross-linker, such as N-succinyl 3-(2-pyridyldithio)proprionate (SPDP).

Conjugates of an antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared. Carbon-14-labeled 1-isothiocyanatobenzyl-3-methyldiethylene triaminepentaacetic acid (MX-DTPA) is an exemplary chelating agent for conjugation of radionuclide to an antibody.

As described above, carbohydrate moieties in an Fc region of an antibody can be used to conjugate a therapeutic agent. However, an Fc region may be absent if a functional antibody fragment is used as an antibody component of an immunoconjugate. Nevertheless, it is possible to introduce a carbohydrate moiety into a light chain variable region of an antibody or functional antibody fragment. An engineered carbohydrate moiety is then used to attach a therapeutic agent. In addition, those of skill in the art will recognize numerous possible variations of the conjugation methods. For example, a carbohydrate moiety can be used to attach polyethyleneglycol in order to extend a half-life of an intact antibody, or a functional antigen-binding fragment thereof, in blood, lymph, or other extracellular fluids. Moreover, it is possible to construct a divalent immunoconjugate by attaching therapeutic agents to a carbohydrate moiety and to a free sulfhydryl group. Such a free sulfhydryl group may be located in a hinge region of an antibody component.

### Methods of Treatment

Disclosed herein, in some embodiments, are methods of treating a subject in need thereof, comprising administering to a subject, a therapeutic dose of a therapeutic anti-EGFR IgA antibody described herein or a pharmaceutical composition comprising said therapeutic anti-EGFR IgA antibody described herein. In some embodiments, a subject has a cancer, an infectious disease, or an autoimmune disease.

Most of the research to investigate the potential of IgA antibodies for immunotherapeutic approaches has been based on in vitro experiments. In vivo pre-clinical research investigating the therapeutic potential of FcαRI-IgA binding has been conducted using transgenic (Tg) mouse models. This is due to the lack of expression of FcαRI in rodents.

In some embodiments, IgA exerts potent proinflammatory effector functions, such as induction of oxidative burst, phagocytosis and ADCC, after binding to FcαRI. In some embodiments, IgA forms more efficient pairing with a FcRg-chain in a transmembrane domain of FcαRI relative to FcγRI. In some embodiments, IgA induced effector function is stronger when triggering FcαRI on polymorphonuclear leukocytes (PMNs) relative to triggering FcγRI. Accordingly, in some embodiments, tumor cell killing by bispecific antibodies (bsAbs) engaging both, tumor antigen (e.g., EGFR) and FcRs, is more efficient when FcαRI is targeted over FcγRI.

EGFR is expressed in many types of cancer cells, such as HNSCC, CRC, NSCLC and Renal and other cancers. Accordingly, in some embodiments, an engineered anti-EGFR IgA antibody targeting EGFR is useful in cancer therapy of any cancer expressing EGFR (e.g., a cancer associated with the expression of EGFR and/or expression of EGFR variants having one or more mutations). In some embodiments, EGFR variant comprises EGFRvIII, exon 19 deletions, L858R substitutions in exon 21, a C797S substitution, or a T790M substitution. In some embodiments, anti-EGFR therapies include antibodies or small molecule inhibitors that bind to EGFR and prevent it's signaling, leading to inhibition of cancer cells growth and survival. In some embodiments, anti-EGFR therapy is used for the treatment of several types of cancer, including metastatic CRC, NSCLC and HNSCC. In some embodiments, anti-EGFR therapies comprise use of a combination therapy comprising at least one anti-EGFR antibody and at least one small molecule inhibitor. In some embodiments, the at least one small molecule inhibitor includes erlotinib and gefitinib, which reversibly inhibit the EGFR tyrosine kinase domain by competitively binding with ATP. In some embodiments, at least one antibody includes an engineered antibody described herein. In some embodiments, at least one antibody further includes cetuximab (a chimeric mouse-human IgG1 antibodies), necitumumab (a fully humanized IgG1 antibodies), matuzumab (a fully humanized IgG1 antibodies) and panitumumab (a fully humanized IgG2 antibodies). All antibodies block ligand binding to an extracellular domain of EGFR, promote receptor internalization and mediate antibody- and complement-mediated cytotoxicity.

In cancer treatment, drug resistance is often encountered, leading to disease progression and poor outcomes. Multiple primary and secondary resistance mechanisms exist for EGFR-TKIs. Point mutations in exon 18, deletions or insertions in exon 19, insertions, duplications, and point mutations in exon 20, as well as a point mutation in exon 21 of the EGFR gene (e.g., L858R substitutions), are among the primary resistance mechanisms. T790M gene mutation is prevalent in 50%-60% of NSCLC patients with the EGFR mutation and is associated with acquired resistance. EGFR amplification and KRAS mutation-driven disease resistance also limit effectiveness of current treatment modalities. This drug resistance, coupled with low response rates of panitumumab and cetuximab, reveals a large unmet clinical need for new therapies. Further, a C797S substitution at an ATP binding site can result in drug resistance (for example, resistant to treatment with osimertinib). As such, an engineered antibody described herein can be used to treat a cancer associated with expression of EGFR having any of these mutations.

In some embodiments, a subject has a cancer. In some embodiments, a subject has an inflammatory disorder. In some embodiments, a cancer is associated with expression of a tumor associated antigen described herein. In some embodiments, a cancer is associated with expression of CD47, CD20, GD2, CD38, CD19, EGFR, HER2, PD-L1, CD25, CD33, BCMA, CD44, α-Folate receptor, CAIX, CD30, ROR1, CEA, EGP-2, EGP-40, HER3, Folate-binding Protein, GD3, IL-13R-a2, KDR, EDB-F, mesothelin, CD22, EGFR, MUC-1, MAGE-A1, MUC16, h5T4, PSMA, TAG-72, EGFRvIII, CD123, VEGF-R2, or a combination thereof. In some embodiments, a cancer is associated with expression of EGFR, MET, cMet, CD28, HER2, HER3, IGF-IR, CD3, PD1, PD-L1, VEGFR2, FcGR3, 4-1BB, or a combination thereof. In some embodiments, an antibody or a functional fragment thereof binds to EGFR, and one or more of MET, cMet, CD28, HER2, HER3, IGF-IR, CD3, PD1, PD-L1, VEGFR2, FcGR3, and 4-1BB.

In some embodiments, a cancer is a metastatic cancer expressing EGFR. In other embodiments, a cancer is a relapsed or refractory cancer. In some embodiments, a cancer is a solid tumor or a hematologic malignancy. In some embodiments, a cancer is a solid tumor. In other embodiments, a cancer is a hematologic malignancy. In some embodiments, a cancer is a metastatic cancer. In some embodiments, a cancer is a relapsed or refractory cancer.

In some embodiments, a cancer is a solid tumor expressing EGFR. Exemplary solid tumors include, but are not limited to, anal cancer, appendix cancer; bile duct cancer (i.e., cholangiocarcinoma); bladder cancer, brain tumor; breast cancer; cervical cancer, colon cancer, cancer of Unknown Primary (CUP); esophageal cancer, eye cancer; fallopian tube cancer, gastroenterological cancer, glioblastoma (e.g., glioma); head and neck cancer; kidney cancer; liver cancer; lung cancer, medulloblastoma; melanoma; mesothelioma; oral cancer, ovarian cancer; pancreatic cancer, parathyroid disease; penile cancer; pituitary tumor, prostate cancer; rectal cancer; skin cancer, stomach cancer; testicular cancer; throat cancer, thyroid cancer; uterine cancer; vaginal cancer, vulvar cancer, or glioblastoma.

In some embodiments, a cancer is selected from the group consisting of: lung cancer, head and neck cancer, colon cancer, rectal cancer, pancreatic cancer, breast cancer, ovarian cancer, bladder cancer, kidney cancer, mesothelioma and a glioblastoma. In some embodiments, a cancer is an adenocarcinoma, a squamous cell carcinoma, or a large-cell carcinoma. In some embodiments, a cancer is a colorectal cancer. In some embodiments, a cancer is a head and neck squamous cell carcinoma. In some embodiments, a cancer is non-small cell lung cancer.

In some embodiments, a cancer expressing EGFR is a hematologic malignancy. In some embodiments, a hematologic malignancy comprises a lymphoma, a leukemia, a myeloma, or a B-cell malignancy. In some embodiments, a hematologic malignancy comprises a lymphoma, a leukemia or a myeloma. In some embodiments, exemplary hematologic malignancies include chronic lymphocytic leukemia (CLL), small lymphocytic lymphoma (SLL), high risk CLL, non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some embodiments, the hematologic malignancy comprises a myeloid leukemia. In some embodiments, a hematologic malignancy comprises acute myeloid leukemia (AML) or chronic myeloid leukemia (CML).

In some embodiments, an anti-EGFR IgA antibody is administered with one or more additional therapeutic agents. In some embodiments, an anti-EGFR IgA antibody and one or more additional therapeutic agents are co-administered. In some embodiments, an anti-EGFR IgA antibody and one or more additional therapeutic agents are sequentially administered. In some embodiments, an additional therapeutic agent comprises an anti-cancer agent, a chemotherapeutic agent, radiation therapy, a cytotoxic agent, a corticosteroid, an immunotherapy agent, a dietary supplement, an antioxidant, or a combination thereof.

In some embodiments, a combination therapy can include one or more antibodies of the disclosure co-formulated with, and/or co-administered with, one or more additional therapeutic agents, e.g., chemotherapeutic or anti-neoplastic agents, such as cytokine and growth factor inhibitors, immunosuppressants, anti-inflammatory agents, metabolic inhibitors, enzyme inhibitors, and/or cytotoxic or cytostatic agents. Exemplary chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, saproin, tamoxifen, taxol, topotecan hydrochloride, vinblastine, vincristine and vinorelbine tartrate. In some embodiments, additional therapeutic agents comprise Janus kinase (JAK) inhibitors. In some embodiments, JAK inhibitors comprise ruxolitinib, pacritinib, fedratinib, tofacitinib, oclacitinib, peficitinib, upadacitinib, deucravacitinib, delgocitinib, or a combination thereof. In some embodiments, additional therapeutic agents comprise KRAS inhibitors. In some embodiments, KRAS inhibitors comprise sotorasib, adagrasib, or a combination thereof. In some embodiments, an additional therapeutic agent binds to MET, cMet, CD28, HER2, HER3, IGF-IR, CD3, PD1, PD-L1, VEGFR2, FcGR3, 4-1BB, or a combination thereof.

In some embodiments, the IgA antibodies described herein can be combined with an effective dose of other antibodies that have been used in treatment of cancer including, without limitation the following FDA approved monoclonal antibodies: rituximab (CD20: chimeric IgG1), trastuzumab (HER2: chimeric IgG1), alemtuzumab (CD52: humanized IgG1), ibritumomab tiuxetan (CD20: murine IgG1 radiolabeled, tositumomab-I-131 (CD20, murine, IgG2a, radiolabeled (Iodine 131)), cetuximab (EGFR: cjimeric, IgG1), bevacizumab (VEGF: humanized, IgG4), panitumumab (EGFR: human IgG2), ofatumumab (CD20: human IgG1), ipilimumab (CTLA-4: human IgG1), brentiuximab vedotin (CD30: chimeric, IgG1, drug-conjugate), pertuzumab (HER2: humanized IgG1, drug conjugate), adotrastuzumab ematansine (HER2: humanized, IgG1, drug-conjugate), obinutuzumab (CD20: humanized and glycol-engineered), nivolumab and pembrolizumab (anti-PD-1s), etc.

In some embodiments, an anti-EGFR IgA antibody described herein is effective in treating cancer in subjects with EGFR-positive cancers. In some embodiments, the subjects include those with either intrinsic or acquired resistance to EGFR-targeting antibodies and tyrosine kinase inhibitors (TKIs). In some embodiments, a subject has a cancer that is a previously-treated, advanced or metastatic solid tumor cancer that express EGFR. Accordingly, in some embodiments, the subjects represent subject populations with high unmet medical need due to the associated poor prognosis and limited number of effective treatment options available. In some embodiments, a treatment with an anti-EGFR IgA antibody described herein overcomes the limitations of EGFR-targeting standard of care (SOC) therapies in the current treatment armamentarium. In some embodiments, an anti-EGFR IgA antibody described herein is designed to unleash full cytotoxic potential of neutrophils and, thereby, offer a new therapeutic option for treating the subjects.

### Dosages

Provided herein are compositions comprising IgA antibodies or functional antigen binding fragments thereof for treatment (including prevention) of a disease (e.g., cancer). In some embodiments, the compositions are pharmaceutical compositions comprising a pharmaceutically acceptable carrier. The compositions are administered in an amount effective for treatment (including prophylaxis) of cancer. In some embodiments, the compositions (e.g., the antibodies or the functional antigen binding fragments thereof or the nucleic acid molecules encoding said antibody or functional antigen binding fragments thereof) are administered in an amount effective for enhancing an immune response and/or increasing T cell activation in a subject. The compositions are to be used for in vivo administration to a subject by any available means, such as parenteral administration. For administration to a subject, a composition or medicament comprising the antibodies or functional antigen binding fragments thereof described herein can be sterile, which can readily accomplished by filtration through sterile filtration membranes or other filtration methods. In one embodiment, a composition or medicament has been treated to be free of pyrogens or endotoxins. Testing pharmaceutical compositions or medicaments for pyrogens or endotoxins and preparing pharmaceutical compositions or medicaments free of pyrogens or endotoxins or preparing pharmaceutical compositions or medicaments that have endotoxins at a clinically-acceptable level, are well understood to one of ordinary skill in the art. Commercial kits are available to test pharmaceutical compositions or medicaments for pyrogens or endotoxins.

The compositions to be used for in vivo administration, such as parenteral administration, in the methods described herein can be sterile, which is readily accomplished by filtration through sterile filtration membranes or other filtration methods.

The IgA antibodies or functional antigen binding fragments thereof, describe herein, are formulated, dosed, and administered in a fashion consistent with good medical practice. Factors for consideration in this context include a particular disorder being treated, a particular subject being treated, a clinical condition of the individual subject, a cause of the disorder, a site of delivery of the agent, a method of administration, a scheduling of administration, and other factors. The IgA antibodies or functional antigen binding fragments thereof can be provided in a therapeutically effective amount as disclosed herein. A therapeutically effective amount of a substance/molecule, agonist or antagonist may vary according to factors such as a disease state, age, sex, and weight of an individual, and an ability of the substance/molecule, agonist or antagonist to elicit a desired response in the individual. A therapeutically effective amount is also one in which any toxic or detrimental effects of a substance/molecule, agonist or antagonist are outweighed by therapeutically beneficial effects. A therapeutically effective amount may be delivered in one or more administrations. A therapeutically effective amount to be administered will be governed by such considerations, and refers to a minimum amount necessary to ameliorate, treat, or stabilize, cancer; to increase the time until progression (duration of progression free survival) or to treat or prevent the occurrence or recurrence of a tumor, a dormant tumor, or a micrometastases. The antibodies or functional antigen binding fragments thereof, disclosed herein, is optionally formulated with one or more additional therapeutic agents currently used to prevent or treat cancer or a risk of developing a cancer. An effective amount of such other agents depends on an amount of antibody or functional antigen binding fragment thereof present in a formulation, a type of disorder or treatment, and other factors discussed above. These are generally used in the same dosages and with administration routes as used herein before or about from 1 to 99% of the heretofore employed dosage.

A dose of an antibody may vary depending upon age and size of a subject to be administered, target disease, conditions, route of administration, and the like. A preferred dose is typically calculated according to body weight or body surface area. When an antibody or a functional antigen binding fragment thereof disclosed herein is used for treating a condition or disease in an adult patient, it may be advantageous to intravenously administer an antibody of the present invention normally at a single dose of about 0.01 to about 20 mg/kg body weight, more preferably about 0.02 to about 7, about 0.03 to about 5, or about 0.05 to about 3 mg/kg, about 5 mg/kg, about 7.5 mg/kg, about 10 mg/kg, or about 15 mg/kg body weight. Depending on severity of a condition, frequency and duration of a treatment can be adjusted. Effective dosages and schedules for administering may be determined empirically; for example, patient progress can be monitored by periodic assessment, and dose adjusted accordingly. Moreover, interspecies scaling of dosages can be performed.

In some embodiments, the compositions herein can comprise a prophylactically effective amount, e.g., when administering to a subject at a risk of cancer or in earlier stages of a disease. Typically, since a prophylactic dose is used in subjects prior to or at an earlier stage of disease, a prophylactic dose is lower than a therapeutic dose.

Advantageously, the pharmaceutical compositions for oral or parenteral use described above are prepared into dosage forms in a unit dose suited to fit a dose of an active ingredients. Such dosage forms in a unit dose include, for example, tablets, pills, capsules, injections (ampoules), suppositories, etc.

An administration can be, for example, by one or more separate administrations, or by continuous infusion. For repeated administrations over several days or longer, depending on the condition, the treatment is sustained until, for example, a cancer is treated. However, other dosage regimens can be useful. In one non-limiting example, an antibody or a functional antigen binding fragment thereof, disclosed herein is administered once every week, every two weeks, or every three weeks, at a dose range from about 5 mg/kg to about 15 mg/kg, including but not limited to 5 mg/kg, 7.5 mg/kg, 10 mg/kg or 15 mg/kg. The progress of using the methods described herein can be easily monitored by conventional techniques and assays. The duration of a therapy using the methods described herein will continue for as long as medically indicated or until a desired therapeutic effect (e.g., those described herein) is achieved. In some embodiments, an administration of one or more antibodies or functional antigen binding fragments thereof, or compositions, described herein, is continued for 1 month, 2 months, 4 months, 6 months, 8 months, 10 months, 1 year, 2 years, 3 years, 4 years, 5 years, 10 years, 20 years, or for a period of years up to a lifetime of a subject.

In some embodiments, an anti-EGFR IgA antibody described herein is administered at a dose ranging from 1 mg/kg to 25 mg/kg. In some embodiments, an anti-EGFR IgA antibody described herein is administered at a single dose of 25 mg/kg. In some embodiments, an anti-EGFR IgA antibody described herein is administered at a dose of 11 mg/kg twice daily.

### Efficacy of Treatment

An efficacy of the treatment methods for example, for cancer, comprising administering the IgA antibodies or functional antigen binding fragments thereof, or pharmaceutical compositions of the present disclosure can be measured by various endpoints commonly used in evaluating cancer treatments, including but not limited to, tumor regression, tumor weight or size shrinkage, time to progression, duration of survival, progression free survival, overall response rate, duration of response, and quality of life. The antibodies or functional antigen binding fragments thereof disclosed herein can require unique measures and definitions of clinical responses to drugs. In the case of cancers, the therapeutically effective amount of the antibodies, functional antigen binding fragments thereof disclosed herein or compositions comprising the same can reduce the number of cancer cells; reduce tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antibodies or functional antigen binding fragments thereof, disclosed herein, act to prevent growth and/or kill existing cancer cells; it can be cytostatic and/or cytotoxic. For cancer therapy, efficacy in vivo can, for example, be measured by assessing duration of survival, duration of progression free survival (PFS), response rates (RR), duration of response, and/or quality of life. In some embodiments, the IgA antibodies or a functional fragment thereof disclosed herein inhibits tumor growth by at least about 2%, 3%, 5%, 6%, 10%, 15%, 20%, 25%, 30%, 35%, 40%, 45%, 50%, 60%, 70%, 80%, 90% or more relative to an untreated subject. In some embodiments, the IgA antibodies or a functional fragment thereof disclosed herein inhibits tumor engraftment by at least about 2%, 3%, 5%, 6%, 10%, 15%, 20%, 25%, 30%, 35%, 40%, 45%, 50%, 60%, 70%, 80%, 90% or more relative to an untreated subject. In some embodiments, the IgA antibodies or a functional fragment thereof disclosed herein induce cytolysis of tumor cell. In some embodiments, the IgA antibodies or a functional fragment thereof disclosed herein induce increased cytolysis of tumor cell by at least about 2%, 3%, 5%, 6%, 10%, 15%, 20%, 25%, 30%, 35%, 40%, 45%, 50%, 60%, 70%, 80%, 90% or more relative to a corresponding WT IgA.

In other embodiments, described herein are methods for increasing progression free survival of a human subject susceptible to or diagnosed with a cancer, for example, skin cancer, such as cutaneous melanoma. Time to disease progression is defined as a time from administration of a drug until disease progression or death. In a preferred embodiment, a combination treatment of the invention using an antibody or a functional antigen binding fragment thereof, disclosed herein, and one or more chemotherapeutic agents may significantly increase progression free survival by at least about 1 month, 1.2 months, 2 months, 2.4 months, 2.9 months, 3.5 months, such as by about 1 to about 5 months, when compared to a treatment with chemotherapy alone. In another embodiment, the methods described herein may significantly increase response rates in a group of human subjects susceptible to or diagnosed with a cancer that are treated with various therapeutics. Response rate is defined as a percentage of treated subjects who responded to a treatment. In one embodiment, a combination treatment described herein using an antibody or a functional antigen binding fragment thereof, disclosed herein, such as a recombinant antibody or a functional antigen binding fragment thereof, and one or more chemotherapeutic agents significantly increases response rate in a treated subject group compared to a group treated with chemotherapy alone.

In some embodiments, the methods described herein comprise administering an effective amount of the antibodies or functional antigen binding fragments thereof, described herein, to a subject in order to alleviate a symptom of a disease, for example, cancer.

Effective amounts, toxicity, and therapeutic efficacy can be determined by standard pharmaceutical procedures in cell cultures or experimental animals, e.g., for determining LD50 (a dose lethal to 50% of the population) and an ED50 (a dose therapeutically effective in 50% of the population). A dosage can vary depending upon a dosage form employed and a route of administration utilized. A dose ratio between toxic and therapeutic effects is a therapeutic index and can be expressed as a ratio LD50/ED50—Compositions and methods that exhibit large therapeutic indices are preferred. A therapeutically effective dose can be estimated initially from cell culture assays. Also, a dose can be formulated in animal models to achieve a circulating plasma concentration range that includes an IC50 (i.e., a concentration of an antibody or a functional antigen binding fragment thereof), which achieves a half-maximal inhibition of symptoms as determined in cell culture, or in an appropriate animal model.

Levels in plasma can be measured, for example, by high performance liquid chromatography. The effects of any particular dosage can be monitored by a suitable bioassay. A dosage can be determined by a physician and adjusted, as necessary, to suit observed effects of a treatment.

A treatment and/or prevention of cancer includes, but is not limited to, alleviating symptoms associated with cancer, inhibition of progression of cancer, promotion of the regression of cancer, promotion of immune response, inhibition of tumor growth, inhibition of tumor size, inhibition of metastasis, inhibition of cancer cell growth, inhibition of cancer cell proliferation, or cause cancer cell death.

### Modes of Administration

The IgA antibodies or functional antigen binding fragments thereof, described herein, can be administered to a subject in need thereof by any appropriate route which results in an effective treatment in a subject. In some embodiments, the antibodies or functional antigen binding fragments thereof, described herein, or compositions comprising the same is administered to a subject having a cancer, to be inhibited by any mode of administration that delivers an agent systemically or to a desired surface or target, and can include, but is not limited to, injection, infusion, instillation, and inhalation administration. Oral administration forms are also contemplated herein. The antibodies or functional antigen binding fragments thereof, described herein, or compositions comprising the same can be administered by injection, which includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intracranial, intraspinal, intracerebro spinal, and intrasternal injection and infusion.

In some embodiments, the antibodies, or functional antigen binding fragments thereof, described herein, or compositions comprising the same can be administered via intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Local administration, for example, to a tumor or cancer site where angiogenesis is occurring, is particularly desired if extensive side effects or toxicity is associated with the use of the antibodies, or functional antigen binding fragments thereof, described herein, or compositions comprising the same. An ex vivo strategy can also be used for therapeutic applications in some embodiments. Ex vivo strategies involve transfecting or transducing cells obtained from a subject with a nucleic acid sequence, disclosed herein. Transfected or transduced cells are then returned to a subject. Cells can be any of a wide range of types including, without limitation, hematopoietic cells (e.g., bone marrow cells, macrophages, monocytes, dendritic cells, T cells, or B cells), fibroblasts, epithelial cells, endothelial cells, keratinocytes, or muscle cells.

In some embodiments, an antibody or a functional antigen binding fragment thereof, disclosed herein, or a composition comprising the same is administered by any suitable means, including parenteral, subcutaneous, intraperitoneal, intrapulmonary, and intranasal, and, if desired for local immunosuppressive treatment, intralesional administration.

Parenteral infusions include intramuscular, intravenous, intraarterial, intraperitoneal, or subcutaneous administration. In some embodiments, an antibody or a functional antigen binding fragment thereof or compositions of the disclosure are suitably administered by pulse infusion, particularly with declining doses of the antibody. Preferably a dosing is given by injections, most preferably intravenous or subcutaneous injections, depending in part on whether an administration is brief or chronic. In some embodiments, an antibody or a functional antigen binding fragment thereof or compositions of the disclosure are administered locally, e.g., by direct injections, when a disorder or location of a tumor permits, and injections can be repeated periodically. In some embodiments, an antibody or a functional antigen binding fragment thereof or compositions of the disclosure can also be delivered systemically to a subject or directly to tumor cells, e.g., to a tumor or a tumor bed following surgical excision of a tumor, in order to prevent or reduce local recurrence or metastasis, for example of a dormant tumor or micrometastases.

Antibody-targeted sonoporation methods are contemplated for use in some embodiments of the methods for inhibiting tumors described herein, in order to enhance efficacy and potency of therapeutic compositions comprising antibodies and functional antigen binding fragments thereof provided herein. As used herein, “sonoporation” refers to the use of sound, preferably at ultrasonic frequencies, or interaction of ultrasound with contrast agents (e.g., stabilized microbubbles) for temporarily modifying permeability of cell plasma membranes, thus allowing uptake of large molecules, such as therapeutic agents. Membrane permeability caused by sonoporation is transient, leaving agents trapped inside cells after ultrasound exposure. Sonoporation employs acoustic cavitation of microbubbles to enhance delivery of large molecules.

Accordingly, in some embodiments of the methods, an antibody or a functional antigen binding fragment thereof described herein, mixed with ultrasound contrast agents, such as microbubbles, can be injected locally or systemically into a subject in need of treatment for cancer, and ultrasound can be coupled and even focused into a defined area, e.g., tumor site, to achieve targeted delivery. In some embodiments, the methods use focused ultrasound methods to achieve targeted delivery. As used herein, HIFU or “High Intensity Focused Ultrasound” refers to a non-invasive therapeutic method using high-intensity ultrasound to heat and destroy malignant or pathogenic tissue without causing damage to overlying or surrounding health tissue. HIFU can also be used as a means of delivery of therapeutic agents, such as antibodies or functional antibody fragments thereof.

Methods using contrast-enhanced ultrasound (CEUS) are also contemplated for use with an antibody or a functional antigen binding fragment thereof, described herein. Contrast-enhanced ultrasound (CEUS) refers to an application of ultrasound contrast medium and ultrasound contrast agents to traditional medical sonography. Ultrasound contrast agents refer to agents that rely on different ways in which sound waves are reflected from interfaces between substances. A variety of microbubble contrast agents are available for use with the compositions and methods described herein. Microbubbles can differ in their shell makeup, gas core makeup, and whether or not they are targeted. Targeting ligands that bind to receptors characteristic of angiogenic disorders, can be conjugated to microbubbles, enabling the microbubble complex to accumulate selectively in areas of interest, such as diseased or abnormal tissues. This form of molecular imaging, known as targeted contrast-enhanced ultrasound, will only generate a strong ultrasound signal if targeted microbubbles bind in the area of interest. Targeted contrast-enhanced ultrasound has many applications in both medical diagnostics and medical therapeutics. In some embodiments, an antibody or a functional antigen binding fragment thereof, described herein, is administered to a subject in need of treatment for a cancer or a tumor, using a targeted ultrasound delivery.

### Pharmaceutical Compositions and Dosage Forms

Disclosed herein, in some embodiments, are pharmaceutical compositions comprising an anti-EGFR IgA antibody or a functional fragment thereof disclosed herein for administration in a subject.

In some embodiments, pharmaceutical compositions comprising an anti-EGFR IgA antibody described herein are formulated in a conventional manner using one or more physiologically acceptable carriers including excipients and auxiliaries which facilitate processing of the active compounds into preparations which can be used pharmaceutically. Proper formulation is dependent upon route of administration chosen.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In some embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the compositions in an acceptable range.

In other embodiments, compositions may also include one or more salts in an amount required to bring osmolality of the compositions into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some embodiments, a pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, the pharmaceutical compositions are formulated into capsules. In some embodiments, the pharmaceutical compositions are formulated into solutions (for example, for IV administration). In some embodiments, a pharmaceutical composition is formulated as an infusion. In some embodiments, a pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In some embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, an antibody, a functional fragment thereof, or composition comprising the same, can be administered to a subject in need thereof (e.g. a subject having cancer). In some embodiments, a cancer is a solid tumor or a hematologic malignancy. In some embodiments, a cancer is a solid tumor. In other embodiments, a cancer is a hematologic malignancy. In some embodiments, a cancer is a metastatic cancer. In some embodiments, a cancer is a relapsed or refractory cancer. In some embodiments, a cancer is a solid tumor. Exemplary solid tumors include, but are not limited to, anal cancer; appendix cancer, bile duct cancer (i.e., cholangiocarcinoma); bladder cancer, brain tumor, breast cancer; cervical cancer; colon cancer; cancer of Unknown Primary (CUP); esophageal cancer; eye cancer, fallopian tube cancer, gastroenterological cancer, glioblastoma (e.g., glioma); head and neck cancer; kidney cancer; liver cancer; lung cancer, medulloblastoma; melanoma; mesothelioma; oral cancer; ovarian cancer; pancreatic cancer, parathyroid disease; penile cancer; pituitary tumor; prostate cancer; rectal cancer, skin cancer; stomach cancer, testicular cancer; throat cancer; thyroid cancer; uterine cancer; vaginal cancer; vulvar cancer; or glioblastoma. In some embodiments leukemia can be, for instance, acute lymphoblastic leukemia (ALL), acute myeloid leukemia (AML), chronic lymphocytic leukemia (CLL) and chronic myeloid leukemia (CML).

An antibody, a functional fragment thereof, or composition thereof can be administered by injection, such as an intravenous (i.v.) injection, sub-cutaneous (s.c.) injection, intradermal (i.d.) injection, intraperitoneal (i.p.) injection, or intramuscular (i.m.) injection. One or more such routes may be employed. Parenteral administration can be, for example, by bolus injection or by gradual perfusion over time. Alternatively, or concurrently, administration may be by the oral route. Additionally, administration may also be by surgical deposition of a bolus or pellet of cells, or positioning of a medical device. In an embodiment, a composition of the present disclosure may comprise engineered cells or host cells expressing nucleic acid sequences described herein, or a vector comprising at least one nucleic acid sequence described herein, in an amount that is effective to treat or prevent proliferative disorders. A pharmaceutical composition may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, an antifoaming agent can be added to reduce foaming during processing which can result in coagulation of aqueous dispersions, bubbles in a finished film, or generally impair processing. Exemplary anti-foaming agents include silicon emulsions or sorbitan sesquioleate.

In some embodiments, an antioxidants such as butylated hydroxytoluene (BHT), sodium ascorbate, ascorbic acid, sodium metabisulfite and tocopherol can be added. In some embodiments, antioxidants enhance chemical stability where required.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

In some embodiments, a binders can be added to impart cohesive qualities. Exemplary binders include, e.g., alginic acid and salts thereof; cellulose derivatives such as carboxymethylcellulose, methylcellulose, hydroxypropylmethylcellulose, hydroxyethylcellulose, hydroxypropylcellulose, ethylcellulose, and microcrystalline cellulose; microcrystalline dextrose; amylose; magnesium aluminum silicate; polysaccharide acids; bentonites; gelatin; polyvinylpyrrolidone/vinyl acetate copolymer, crospovidone; povidone; starch; pregelatinized starch; tragacanth, dextrin, a sugar, such as sucrose, glucose, dextrose, molasses, mannitol, sorbitol, xylitol, and lactose; a natural or synthetic gum such as acacia, tragacanth, ghatti gum, mucilage of isapol husks, polyvinylpyrrolidone, larch arabogalactan, polyethylene glycol, waxes, sodium alginate, and the like.

In some embodiments, a carrier or carrier material can be added. A carrier or carrier material includes any commonly used excipients in pharmaceutics and should be selected on the basis of compatibility with compounds disclosed herein, such as, compounds of ibrutinib and an anticancer agent, and release profile properties of a desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials may include, but are not limited to, acacia, gelatin, colloidal silicon dioxide, calcium glycerophosphate, calcium lactate, maltodextrin, glycerine, magnesium silicate, polyvinylpyrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like.

In some embodiments, a dispersing agent can be added to control diffusion and homogeneity of a drug through liquid media or a granulation method or blend method. These agents also facilitate effectiveness of a coating or eroding matrix. Exemplary dispersing agents include, e.g., hydrophilic polymers, electrolytes, polyoxyethylene sorbitan monostearate, polyoxyethylene sorbitan monooleate, PEG, polyvinylpyrrolidone (PVP), and carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., block copolymers of ethylene oxide and propylene oxide); and poloxamines (e.g., tetrafunctional block copolymers derived from sequential addition of propylene oxide and ethylene oxide to ethylenediamine), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., a polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

In some instances, a diluent can be added to dilute a compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In some embodiments, diluents increase bulk of a composition to facilitate compression or create sufficient bulk for homogenous blend for capsule filling. Such compounds include e.g., lactose, starch, mannitol, sorbitol, dextrose, microcrystalline cellulose, dibasic calcium phosphate, dicalcium phosphate dihydrate; tricalcium phosphate, calcium phosphate; anhydrous lactose, spray-dried lactose; pregelatinized starch, compressible sugar, mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In some instances, a filling agent can be added, which includes compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In some instances, a lubricant or glidant can be added to prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil, a surfactant, and the like.

In some instances, a plasticizer can be added to soften microencapsulation materials or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

In some instances, a solubilizer can be added, such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

In some instances, a stabilizers can be added, such as antioxidation agents, buffers, acids, preservatives and the like.

In some instances, a suspending agents can be added, which includes compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., a polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

In some instances, a surfactants can be added, which includes compounds such as sodium lauryl sulfate, sodium docusate, Polyethylene glycol sorbitan monostearate or polyoxyethylene sorbitan monooleate, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

In some instances, a viscosity enhancing agent can be added, which includes e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

In some instances, a wetting agents can be added, which includes compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, polyoxyethylene sorbitan monooleate, vitamin E TPGS, ammonium salts and the like.

Optionally, the formulations comprising the compositions described herein contain a pharmaceutically acceptable salt, typically, e.g., sodium chloride, and preferably at about physiological concentrations. Optionally, the formulations of the invention can contain a pharmaceutically acceptable preservative. In some embodiments, a preservative concentration ranges from 0.1 to 2.0%, typically v/v.

Suitable preservatives include, for example, benzyl alcohol, phenol, m-cresol, methylparaben, and propylparaben are examples of preservatives. Optionally, the formulations of the invention can include a pharmaceutically acceptable surfactant at a concentration of 0.005 to 0.02%.

The compositions described herein can be specially formulated for administration of an antibody or a functional antigen binding fragment thereof to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, an antibody or a functional antigen binding fragment thereof, or compositions of the present disclosure can be implanted into a patient or injected using a drug delivery system.

The compositions disclosed herein, comprising an antibody or a functional antigen binding fragment, described herein, can also contain more than one active compound as necessary for the particular indication being treated, preferably those with complementary activities that do not adversely affect each other. For example, a composition can further comprise a cytotoxic agent, cytokine, growth inhibitory agent and/or an angiogenesis inhibitor such as a VEGFR antagonist. Such molecules are suitably present in combination in amounts that are effective for the purpose intended. The active ingredients of the compositions comprising an antibody or a functional antigen binding fragment thereof described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microparticle, microemulsions, nano-particles and nanocapsules) or in macroemulsions. A pharmaceutical composition can be also delivered in a vesicle, in particular a liposome. Liposomes include emulsions, foams, micelles, insoluble monolayers, phospholipid dispersions, lamellar layers and the like, and can serve as vehicles to target the M-CSF antibodies to a particular tissue as well as to increase a half-life of the composition. A variety of methods are available for preparing liposomes.

Particularly useful liposomes can be generated by a reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody of the present invention can be conjugated to liposomes via a disulfide interchange reaction. A chemotherapeutic agent (such as Doxorubicin) is optionally contained within a liposome.

In some embodiments, sustained-release preparations can be used. Suitable examples of sustained-release preparations include semipermeable matrices of solid hydrophobic polymers containing an antibody or a functional antigen binding fragment of the present disclosure, in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and y ethyl-L-glutamate, non-degradable ethylene-vinyl acetate, degradable lactic acid-glycolic acid copolymers such as injectable microspheres composed of lactic acid-glycolic acid copolymer and leuprolide acetate, and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on a mechanism involved. For example, if aggregation mechanism is discovered to be intermolecular S—S bond formation through thiodisulfide interchange, stabilization can be achieved by modifying sulfhydryl residues, lyophilizing from acidic solutions, controlling moisture content, using appropriate additives, and developing specific polymer matrix compositions. In some embodiments, a pharmaceutical composition can be delivered in a controlled release system. In one embodiment, a pump may be used. In another embodiment, polymeric materials can be used. In yet another embodiment, a controlled release system can be placed in proximity of a composition's target, thus requiring only a fraction of a systemic dose.

A pharmaceutical composition of the present disclosure can be delivered, e.g., subcutaneously, intravenously or intraperitoneally with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical compositions within a cartridge has been administered and the cartridge is empty, the empty cartridge can readily be discarded and replaced with a new cartridge that contains the pharmaceutical composition. A pen delivery device can then be reused. In a disposable pen delivery device, there is no replaceable cartridge. Rather, a disposable pen delivery device comes prefilled with a pharmaceutical composition held in a reservoir within the device. Once a reservoir is emptied of a pharmaceutical composition, an entire device is discarded. Numerous reusable pen and autoinjector delivery devices have applications in the subcutaneous delivery of a pharmaceutical composition of the present invention.

The injectable preparations may include dosage forms for intravenous, subcutaneous, intracutaneous and intramuscular injections, drip infusions, etc. The injectable preparations may be prepared, e.g., by dissolving, suspending or emulsifying an antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. An injection thus prepared is preferably filled in an appropriate ampoule.

Compositions of the present disclosure can be in the form of, for example, granules, powders, tablets, capsules, syrup, suppositories, injections, emulsions, elixirs, suspensions or solutions. The amount of an aforesaid antibody contained can be about 5 to about 500 mg per dosage form in a unit dose; especially in a form of injection, it is preferred that the aforesaid antibody is contained in about 5 to about 100 mg and in about 10 to about 250 mg for the other dosage forms.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials.

Liquid dosage forms for oral administration may be in the form of pharmaceutically acceptable emulsions, syrups, elixirs, suspensions, and solutions, which may contain an inactive diluent, such as water. In some embodiments, pharmaceutical formulations and medicaments may be prepared as liquid suspensions or aqueous solutions, for example, using a sterile liquid, such as, but not limited to, an oil, water, an alcohol, and combinations of these. In some embodiments, pharmaceutical compositions can be prepared in a lyophilized form. The lyophilized preparations can comprise a cryoprotectant, which includes agents that provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

As noted above, suspensions may include oils. Such oils include, but are not limited to, peanut oil, sesame oil, cottonseed oil, corn oil and olive oil. Suspension preparation may also contain esters of fatty acids such as ethyl oleate, isopropyl myristate, fatty acid glycerides and acetylated fatty acid glycerides. Suspension formulations may include alcohols, such as, but not limited to, ethanol, iso-propyl alcohol, hexadecyl alcohol, glycerol and propylene glycol. Ethers, such as but not limited to, poly(ethyleneglycol), petroleum hydrocarbons such as mineral oil and petrolatum; and water may also be used in suspension formulations.

For nasal administration, the pharmaceutical formulations and medicaments may be a spray or aerosol containing an appropriate solvent(s) and optionally other compounds such as, but not limited to, stabilizers, antimicrobial agents, antioxidants, pH modifiers, surfactants, bio-availability modifiers and combinations of these. A propellant for an aerosol formulation may include compressed air, nitrogen, carbon dioxide, or a hydrocarbon based low boiling solvent.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringers solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, a pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

For rectal administration, the pharmaceutical formulations and medicaments may be in the form of a suppository, an ointment, an enema, a tablet or a cream for release of compound in the intestines, sigmoid flexure and/or rectum. Rectal suppositories are prepared by mixing one or more compounds of the instant invention, or pharmaceutically acceptable salts or tautomers of the compound, with acceptable vehicles, for example, cocoa butter or polyethylene glycol, which is present in a solid phase at normal storing temperatures, and present in a liquid phase at those temperatures suitable to release a drug inside the body, such as in the rectum. Oils may also be employed in the preparation of formulations of the soft gelatin type and suppositories. Water, saline, aqueous dextrose and related sugar solutions, and glycerols may be employed in the preparation of suspension formulations which may also contain suspending agents such as pectins, carbomers, methyl cellulose, hydroxypropyl cellulose or carboxymethyl cellulose, as well as buffers and preservatives.

A concentration of an antibody or a functional antigen binding fragment thereof in these compositions can vary widely, i.e., from less than about 10%, usually at least about 25% to as much as 75% or 90% by weight and will be selected primarily by fluid volumes, viscosities, etc., in accordance with a particular mode of administration selected.

In another embodiment of the invention, an article of manufacture containing materials useful for a treatment of diseases, disorders or conditions described above is provided, including for treatment of cancer. An article of manufacture comprises a container and a label. Suitable containers include, for example, bottles, vials, syringes, and test tubes. The containers may be formed from a variety of materials such as glass or plastic. A container holds a composition which is effective for treating the condition and may have a sterile access port (for example the container may be an intravenous solution bag or a vial having a stopper pierceable by a hypodermic injection needle). An active agent in a composition is an antibody of the invention. A label on or associated with a container indicates that a composition is used for treating the condition of choice. An article of manufacture may further comprise a second container comprising a pharmaceutically-acceptable buffer, such as phosphate-buffered saline, Ringer's solution and dextrose solution. It may further include other materials desirable from a commercial and user stand-point, including other buffers, diluents, filters, needles, syringes, and package inserts with instructions for use. Pharmaceutical compositions and medicaments described herein are useful in treating a cancerous disease.

### Diagnostic and Other Uses

Provided herein are methods of using the antibodies for detection, diagnosis and monitoring of a disease, disorder or condition associated with antigen expression (either increased or decreased relative to a normal sample, and/or inappropriate expression, such as presence of expression in tissues(s) and/or cell(s) that normally lack the epitope expression). Provided herein are methods of determining whether a patient will respond to antibody therapy.

In some embodiments, a method comprises detecting whether a patient has cells that express target antigen using an antibody disclosed herein. In some embodiments, a method of detection comprises contacting a sample with an antibody or a functional antigen binding fragment thereof of the disclosure, and determining whether level of binding differs from that of a reference or comparison sample (such as a control). In some embodiments, a method may be useful to determine whether the antibodies or polypeptides described herein are an appropriate treatment for a subject.

In some embodiments, the cells or cell/tissue lysate are contacted with an antibody and a binding between the antibody and the cell is determined. When test cells show binding activity as compared to a reference cell of the same tissue type, it may indicate that a subject would benefit from treatment with an antibody. In some embodiments, test cells are from human tissues. In some embodiments, test cells are from human blood.

Various methods for detecting specific antibody-antigen binding can be used. Exemplary immunoassays which can be conducted include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to subject antibodies and is selected so as to meet the needs of various uses of the method which are often dictated by the availability of assay equipment and compatible immunoassay procedures.

Appropriate labels include, without limitation, radionuclides (for example 125I, 131I, 35S, 3H, or 32P), enzymes (for example, alkaline phosphatase, horseradish peroxidase, luciferase, or β-galactosidase), fluorescent moieties or proteins (for example, fluorescein, rhodamine, phycoerythrin, GFP, or BFP), or luminescent moieties.

For purposes of diagnosis, the antibodies or functional antigen binding fragment thereof can be labeled with a detectable moiety including but not limited to radioisotopes, fluorescent labels, and various enzyme-substrate labels know in the art.

In some embodiments, the antibodies need not be labeled, and presence thereof can be detected using a second labeled antibody which binds to a first antibody. The antibodies or functional antigen binding fragment thereof of the present invention may be used as affinity purification agents for a cancer associated antigen or in diagnostic assays for a cancer associated antigen protein, e.g., detecting its expression in specific cells, tissues, or serum. The antibodies or functional antigen binding fragment thereof, disclosed herein, may also be used for in vivo diagnostic assays. Generally, for these purposes, an antibody is labeled with a radionuclide (such as u1In, 99Tc, 14C, 131I, 12sI, 3H, 32p or 3sS) so that a tumor can be localized using immunoscintiography.

The antibodies of the present invention may be employed in an assay, such as competitive binding assays, direct and indirect sandwich assays, such as ELISAs, and immunoprecipitation assays. The antibodies may also be used for immunohistochemistry to label tumor samples. As a matter of convenience, an antibody of the present invention can be provided in a kit, i.e., a packaged combination of reagents in predetermined amounts with instructions for performing a diagnostic assay. Where an antibody is labeled with an enzyme, a kit will include substrates and cofactors required by an enzyme (e.g., a substrate precursor which provides a detectable chromophore or fluorophore). In addition, other additives may be included such as stabilizers, buffers (e.g., a block buffer or lysis buffer) and the like. The relative amounts of various reagents may be varied widely to provide for concentrations in solution of reagents which substantially optimize the sensitivity of the assay. Particularly, the reagents may be provided as dry powders, usually lyophilized, including excipients which on dissolution will provide a reagent solution having the appropriate concentration.

### Kits

Provided herein are also kits, medicines, compositions, and unit dosage forms for use in any of the methods described herein. Provided herein is a kit comprising a therapeutically effective amount of at least one anti-EGFR IgA antibody or functional antigen binding fragment thereof disclosed herein. In some embodiments, a kit further comprises a second therapeutic agent (e.g., a chemotherapeutic agent). In some embodiments, an antibody or functional antigen binding fragment thereof is an aqueous form or a lyophilized form. A kit further comprises a diluent or a reconstitution solution.

Kits can include one or more containers comprising an antibody (or unit dosage forms and/or articles of manufacture). In some embodiments, a unit dosage is provided wherein a unit dosage contains a predetermined amount of a composition comprising an antibody (e.g., a therapeutically effective amount), with or without one or more additional agents. In some embodiments, such a unit dosage is supplied in single-use prefilled syringe for injection. In some embodiments, the compositions comprising an antibody or a functional antigen binding fragment thereof can comprise saline, sucrose, or the like; a buffer, such as phosphate, or the like; and/or be formulated within a stable and effective pH range. In some embodiments, an antibody or a functional antigen binding fragment thereof can be provided as a lyophilized powder that may be reconstituted upon addition of an appropriate liquid, for example, sterile water. In some embodiments, an antibody or a functional antigen binding fragment thereof further comprises one or more substances that inhibit protein aggregation, including, but not limited to, sucrose and arginine. In some embodiments, an antibody or a functional antigen binding fragment thereof further comprises heparin and/or a proteoglycan.

In some embodiments, kits further comprise instructions for use in the treatment of cancer in accordance with any of the methods described herein. A kit may further comprise a description of selection an individual suitable or treatment. Instructions supplied in the kits are typically written instructions on a label or package insert (for example, a paper sheet included in a kit), but machine-readable instructions (for example, instructions carried on a magnetic or optical storage disk) are also acceptable. In some embodiments, a kit further comprises another therapeutic agent (e.g., an anti-cancer antibody or a chemotherapeutic agent)

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### EXAMPLES

### Example 1—Engineered Anti-EGFR IgA Design and Production Methods

### Design of Engineered Anti-EGFR IgA

Engineered Anti-EGFR IgA Antibody Variants were Designed, Each Having an EGFR-Binding Variable Region and a Modified IgA Constant Region.

The genetic sequence of the IgA2(m1) heavy chain is the backbone for the engineered anti-EGFR IgA variants where the N-linked glycosylation motifs are silenced and stabilizing mutations are introduced (Table 10; FIGS. 4A-4D). This results in a series of molecules: anti-EGFR IgA3.0−(min), anti-EGFR IgA3.0+(plus) and anti-EGFR IgA4.0.

The anti-EGFR IgA3.0+ molecule contains a CH1-P124R mutation that allows for covalent binding between the heavy- and light-chain. Two Cysteine residues are modified or removed (CH2-C92S; CH3_CHS-C147del_Y148del) to prevent the formation of cysteine bridges with serum proteins, preventing dimeric aggregates and/or complex formation. Furthermore, three N-linked glycosylation motifs are silenced, by substitution of critical amino acids in these motifs (CH1-N45.2G; CH2-N120T; CH3_CHS-N135Q).

The anti-EGFR IgA3.0min molecule contains the same mutations in the CH1 and CH2 as anti-EGFR IgA3.0+, but in contrast to anti-EGFR IgA3.0+, almost the entire tailpiece is deleted in anti-EGFR IgA3.0min (CH3_CHS-P131-Y148del).

The anti-EGFR IgA4.0 molecules are created by silencing the only remaining N-linked glycosylation motif (CH2-N20) by 4 separate amino acid substitutions, thereby creating 4 fully aglycosylated IgA2-based molecules.

TABLE 10
List of mutations in engineered anti-EGFR IgA: IgA3.0+,
IgA3.0 min, and IgA4.0 relative to the wild type (WT) IgA2(m1)

| | | | IgA2 | | IgA3.0 | |
|---|---|---|---|---|---|---|
| Domain | Modification | Description | (m1) | IgA3.0+ | min | IgA4.0 |
| CH1 | N45.2G | Remove N-linked | X | ✓ | ✓ | ✓ |
| | | glycosylation motif | | | | |
| | P124R | Covalent heavy-/light-chain | X | ✓ | ✓ | ✓ |
| | | interaction | | | | |
| CH2 | N20G | Remove N-linked | X | X | X | ✓ |
| | | glycosylation motif | | | | |
| | N20Q | Remove N-linked | X | X | X | ✓ |
| | | glycosylation motif | | | | |
| | N20T | Remove N-linked | X | X | X | ✓ |
| | | glycosylation motif | | | | |
| | L21I | Remove N-linked | | | | |
| | | glycosylation motif | | | | |
| | T22S | Remove N-linked | | | | |
| | | glycosylation motif | | | | |
| | C92S | Prevent cysteine bridging | X | ✓ | ✓ | ✓ |
| | N120T | Remove N-linked | X | ✓ | ✓ | ✓ |
| | | glycosylation motif | | | | |
| | I121L | Remove N-linked | | | | |
| | | glycosylation motif | | | | |
| | T122S | Remove N-linked | | | | |
| | | glycosylation motif | | | | |
| CH3- | N135Q | Remove N-linked | X | ✓ | X | X |
| CHS | | glycosylation motif | | | | |
| | C147del | Deletion preventing cysteine | X | ✓ | X | X |
| | | bridging | | | | |
| | Y148del | Deletion | X | ✓ | X | X |
| | P131-Y148del | Deletion tailpiece | X | X | ✓ | ✓ |

### Cloning Anti-EGFR IgA3.0/IgA4.0

To clone the anti-EGFR IgA3.0+ and anti-EGFR IgA3.0min molecules, synthetic DNA covering the entire IgA constant region (CH1-CH2-CH3-CHS) was ordered, and the cassette encoding each was cloned in the pEE14.4 vector, replacing the IgA2(m1) sequence. Anti-EGFR IgA4.0 molecules were cloned by replacing the CH1-CH2-CH3-CHS region of anti-EGFR IgA3.0min in the pcDNA3.4 vector with gBlocks (IDT) containing the corresponding mutations for IgA4.0.

### Production

HEK293F cells were used For the production of the anti-EGFR IgA3.0+ and anti-EGFR IgA3.0min, while ExpiCHO-S cells were used for the production of anti-EGFR IgA4.0. For DNA complexing, separate vectors for anti-EGFR IgA3.0+, anti-EGFR IgA3.0min heavy chain, or anti-EGFR IgA 4.0 heavy chain (HC), along with vectors for kappa light chain (LC) and pAdvantage (pAdv; Promega) were mixed in optimal HC:LC:pAdv ratios and complexed with either 293fectin or Expifectamine prior to transfection.

### Purification

The procedure for the purification of anti-EGFR IgA is identical for all anti-EGFR IgA variants, and was performed by capturing kappa light chains from clarified and filtered cell culture supernatant using FPCL with a HiTrap KappaSelect column (GE Healthcare), followed by a separation by a Size Exclusion Chromatography procedure using a HiPrep 26/60 Sephacryl S-300 HR column.

### Example 2—Characterization of Anti-EGFR IgA Variants

### Binding Assays

To determine binding of the engineered antibodies, both the binding of the variable portion and the Fc portion are assessed.

Clarified supernatants of anti-EGFR IgA3.0min or anti-EGFR IgA3.0+ from HEK293F productions are tested on EGFR-expressing eukaryotic cells in a FACS binding experiment. Clarified supernatants of anti-EGFR IgA4.0 from ExpiCHO-S productions are used undiluted in a FACS binding experiment.

In short, supernatants are incubated with eukaryotic cells for 1 hour on ice. After washing, cells are incubated with PE-labeled anti-IgA antibody (Southern Biotech) for 45 minutes. After washing, cells are fixed with PFA and measured on a Canto II (BD). EGFR recognizing control antibodies are added at concentration of 5-10 μg/mL.

To determine whether an antibody Fc-region is able to bind to FcαR, anti-EGFR IgA2(m1) and anti-EGFR IgA3.0min (25 μg/mL) are coated overnight in an ELISA plate in carbonate buffer pH 9.0. Plates are blocked with 1% BSA after which CD89-expressing Calcein-labeled healthy donor polymorphonuclear neutrophils (PMN) are allowed to bind the plate for 45 minutes at 37° C. Subsequently, after every two washing steps, binding is determined by comparing the remaining signal to the input signal (no wash). An additional control to determine if coating concentrations are equal, is performed by staining an ELISA plate overnight with serially diluted anti-EGFR IgA2(m1) and anti-EGFR IgA3.0min antibody after which their presence is detected with anti-hIgA-HRP (Southern Biotech).

### ADCC

Target cells are loaded with 51Cr (Perkin-Elmer), washed twice and incubated with serially diluted anti-EGFR IgA antibodies and healthy donor PMN's for 4 hours. Chromium release is measured in the supernatant and specific lysis is calculated using following the formula: ((experimental cpm−basal cpm)/(maximal cpm−basal cpm))×100, with maximal lysis determined by incubating labelled cells with 1.25% TRITON™ (2-[4-(2,4,4-trimethylpentan-2-yl)phenoxy]ethanol) and minimal lysis in the absence of antibodies and effector cells. In the case of anti-EGFR IgA4.0, undiluted supernatants of ExpiCHO-S productions are assessed.

### Thermostability

Thermal stability is analyzed in thermal shift assay using Sypro Orange (Life Technologies). A total of 12.5 μg of anti-EGFR IgA antibody diluted in 25 μL PBS and 3×SYPRO Orange (final concentration) is transferred to a white 96-well thin-wall PCR plate (Roche) and sealed with Optical-Quality Sealing Tape (Roche). Plates are heated in a ViiA7 (Roche) from 37° C. to 99° C. with a heat-rate of 1.6° C./second and 1 minute incubation at each degree. Fluorescence is recorded simultaneously using 490 and 575 nm as excitation and emission wavelengths, respectively.

To assess functionality of destabilized anti-EGFR IgA antibodies in PBS, each anti-EGFR IgA antibody is incubated at different temperatures (4° C., 23° C.-95° C. in incremental steps of 12° C.) for 5 minutes in a thermal cycler. After incubation, complete medium is added and the anti-EGFR IgA antibodies are used directly in an ADCC at a final concentration of 10 μg/mL.

### Glycosylation Analysis

PNGase F treatments are performed according to manufacturer's instructions (NEB). In short, anti-EGFR IgA antibodies are first denatured for 10 minutes at 100° C., and subsequently NP-40, Glycobuffer and PNGase F enzyme are added and incubated for 1 hour at 37° C. Samples are taken in Laemmli buffer with 20 mM DTT and run on a 10% Mini Protean TGX SDS-PAGE (Bio-Rad). Gels are stained with InstantBlue (Expedeon) for 10 minutes, and rinsed with water.

Glycan identification and quantification is determined by mass spectrometry. For the anti-EGFR IgA2(m1) antibody, a reflectron positive mode MALDI-TOF-MS analysis of the released N-glycosylation of IgA2 antibodies after linkage-specific sialic acid derivatization is performed. For the anti-EGFR IgA3.0+, anti-EGFR IgA3.0min, and anti-EGFR IgA4.0 variants an LC/MS2 approach has been taken.

Antibodies are denatured, reduced, alkylated and proteolytically digested with GluC (Roche (Indianapolis, IN)) and trypsin (Sigma-Aldrich (Steinheim, Germany)). For this, 10 μg of antibody was brought to 100 mM Tris-HCl (pH 8.5) (Tris(hydroxymethyl)aminomethane hydrochloride), 5 mM Tris(2-carboxyethyl)-phosphine (TCEP, Sigma-Aldrich (Steinheim, Germany)), 30 mM chloroacetamide (CAA, Sigma-Aldrich (Steinheim, Germany)) and 1% sodium deoxychelate (SDC, Sigma-Aldrich (Steinheim, Germany)), water (MQ) (generated from a Q-POD or Q-Gard 1 system (Millipore), operated at ≥18.2 MΩ). This mixture is incubated for 4 h at 37° C. with GluC with an enzyme:protein ratio of 1:75 w/w, followed by an overnight incubation at 37° C. with trypsin (1:100 w/w). Hereafter, the SDC is precipitated by bringing the samples to 0.5% trifluoric acid (TFA, Sigma-Aldrich (Steinheim, Germany)) and centrifugation at maximum speed for 10 min. The supernatant is collected for solid-phase extraction (SPE).

For SPE, the use of an Oasis μElution HLB 96-well plate (Waters, Wexford, Ireland) positioned on a vacuum manifold is made. The plate is conditioned with acetonitrile (ACN, BioSolve Valkenswaard, The Netherlands) equilibrated with 0.5% TFA, loaded with the supernatant, washed with 0.5% TFA, and peptides are eluted with 50% ACN 0.5% TFA. The recovered eluate is dried by means of rotary evaporation, and reconstituted in 2% formic acid for subsequent LC-MS2 analysis.

For each of the digested and desalted samples, 100 ng is analyzed by use of an Agilent 1290 Infinity HPLC system (Agilent Technologies, Waldbronn, Germany), equipped with flow-splitter to achieve nanoflow, hyphenated to an Orbitrap Fusion Tribrid mass spectrometer (Thermo Fisher Scientific, Bremen, Germany). The samples are separated on a 2 cm trap column (100 μm inner diameter, packed with 3 μm ReproSil-Pur C18-AQ; Dr. Maisch GmbH, Ammerbuch-Entringen, German) coupled to a 50 cm analytical column (50 μm inner diameter, packed with 2.7 μm Poroshell 120 EC-C18; Agilent Technologies, Amstelveen, The Netherlands). Buffer A consists of 0.1% formic acid, buffer B of 0.1% formic acid in 80% ACN. The LC gradient is as follows: 0-5 min: 100% A (the rest being B), 5-53 min: 87% A to 60% A, 53-58 min 0% A, 58-65 min 100% A.

Mass spectrometry is performed in positive ion mode, with electrospray ionization from a coated fused silica emitter at 2 kV spray voltage. Each sample is measured in triplicate, using the same MS1 acquisition methods, but different MS2 methods. For the MS1 scans, the mass range is set from m/z 350 to 2000 with a resolution of 60,000, an AGC target of 400,000 with maximum injection time of 50 ms. Each of the three MS2 methods initiates HCD fragmentation (30% normalized collision energy; NCE) on the highest charge state, lowest m/z signals within a 3 s cycle time, using an exclusion time of 30 s. MS2 by HCD is recorded with a resolution of 30,000 from m/z 120 to 4000, with an AGC target of 50,000 and a maximum injection time of 50 ms. For MS2 method 1, only HCD fragmentation is performed. For MS2 method 2, the detection of at least 3 oxonium ions (Hex: 127.0390, 145.0495, 163.0601; HexNAc: 138.0550, 168.0655, 186.0761, 204.0867; PhosphoHex: 243.0264; NeuAc: 274.0921, 292.1027; Complex: 366.1395, 405.0793, 407.1660, 512.1974, 657.2349) within the HCD spectrum triggers stepping-HCD on the same precursor signal, combining the HCD fragments with NCEs of 10%, 25% and 40%. Stepping-HCD is recorded with a resolution of 30,000 from m/z 120 to 4000, with an AGC target of 200,000 and a maximum injection time of 250 ms. For MS2 method 3, detection of the oxonium ions triggers EThcD (30% supplemental activation), which is recorded with a resolution of 30,000 from m/z 120 to 4000, with an AGC target of 200,000 and a maximum injection time of 250 ms.

Bottom-up data is interpreted with Byonic v3.3.11 (Protein Metrics Inc.). Raw data is searched with C-terminal cleavage sites at Arg and Lys (trypsin), and Glu and Asp (GluC). 3 missed cleavages is allowed, using a precursor mass tolerance of 10 ppm and fragment mass tolerance of 20 ppm. Cys carbamidomethylation is included as fixed modification and Met oxidation as variable modification. For N-glycosylation 279 compositions is included following the pathways for N-glycan biosynthesis.

Skyline (v3.7.0.11317) is used to perform relative quantification. For this, each of the peptides found glycosylated by Byonic are integrated, which included all major miscleavages and oxidation variants. For each of these, the aforementioned 279 glycan compositions are integrated from each LC-MS2 run. Integrations obtained as such are subsequently curated to adhere to the following criteria: 1): ≤5 ppm error to the theoretical mass, 2) having an idotp of ≥0.85 with the theoretical isotopic pattern, 3) eluting within ±2 min of the mean retention time for that peptide, 4) having no apparent overlapping isotopic patterns. The resulting list of glycopeptides is in agreement with the annotation by Byonic, and further is used for relative quantification. For each of the curated peptide glycoforms, the MS1 areas are integrated and peptides informing on the same N-glycosylation site are combined. As alternative way of quantification, the number of peptide spectrum matches (PSMs) is counted for each glycopeptide combination that informs on a given glycosylation site.

For visualization of glycan species, the recommendations of the Consortium for Functional Glycomics are followed. Glycan cartoons are constructed using GlycoWorkbench (v2.1 build 146). For native MS analysis antibodies are buffer exchanged to 150 mM ammonium acetate pH 7.5, using a Vivaspin 500 30 kDa molecular weight cut-off filter (Sartorius Stedim Biotech, Germany) by 10×15 min centrifugation at 15.000×g. Following buffer exchange, the antibodies are adjusted to an approximate concentration of 3 μM with 150 mM ammonium acetate pH 7.5.

Native MS is performed on a modified Exactive Plus Orbitrap instrument with extended mass range (EMR) (Thermo Fisher Scientific, Bremen), calibrated using a 25 mg/mL CsI solution. Voltage settings of the transport multipoles and ion lenses are manually optimized to provide good transmission at the required m/z range. Electrospray ionization is achieved from a gold-coated glass capillary, employing a capillary voltage of 1.2 kV, while the MS is operated with a source fragmentation of 80 V, a source temperature of 250° C., a collision energy of 80 V, and a resolution of 35000 at m/z 200. Desolvation of molecules is further achieved by addition of nitrogen to the HCD cell to reach a gas pressure of approximately 3.7×10-10 bar. Masses are calculated from the charge state distributions by fitting the charges to the lowest standard deviation on the mass (typically resulting in mass errors lower than 1 Da).

### Pharmacokinetic/Pharmacodynamic Studies

For pharmacokinetics/pharmacodynamics of the anti-EGFR IgA antibodies, BALB/cByJ mice (Jackson Laboratory) are injected i.v. with 100 μg (1 mg/mL) of anti-EGFR IgA antibody. Blood is collected at the indicated time points, and centrifuged to separate and collect serum. ELISA is performed on diluted serum.

### Biodistribution

Each anti-EGFR IgA antibody is conjugated to a chelator prior to radio-labeling. The anti-EGFR IgA Antibody is spun through a 30 kDa centrifugal filter at 12,000 g for 8 min. Subsequently, 500 μL of 0.1 M sodium bicarbonate (pH 8.2) is added to the centrifugal filter and spun again at 12,000 g for 8 min. The centrifugal filter is inverted into a new tube and centrifuged at 1,000 g for 2 min, yielding about 40 μL to which 60 μL of sodium bicarbonate buffer is added, resulting in 100 μL. A 20-fold molar excess of p-SCN-Bn-DTPA (2 mg/mL in dry DMSO (used 1 mg in 500 μl)) is added and vortexed for 30 sec, and incubated 1 hour at 37° C. Next, the BnDTPA-antibody conjugate is run through a G50 column, eluting with 0.5 M MES buffer (pH 5.4) in 100 μL fractions for 12 fractions. The 5 most concentrated fractions are pooled and spun through a 30 kDa centrifugal filter at 12,000 g for 8 mins. A volume of 500 μL 0.5 M MES buffer (pH 5.4) is added to the centrifugal filter and centrifuged for further 8 min at 12,300 g. The centrifugal filter is inverted into a new microcentrifuge tube and centrifuged at 1,000 g for 2 min. Protein concentration is measured by Nanodrop.

To radiolabel an antibody 150 μL Indium solution (55.5 MBq) is added to 150 μg antibody in MES buffer in a microcentrifuge tube and incubated for 45 mins at room temperature. The reaction mixture is run through a G50 column, and eluted with PBS (pH 7.4). Sample is collected in a microcentrifuge tube in 100 μL fractions (3drops) for 16 fractions, activity is checked and highest fractions are combined.

To check purity of the radiolabeled antibody by an iTLC strip, a total of 2 μL of the reaction mixture is pipetted onto the iTLC and dried for 2 minutes. A volume of 0.1 M citrate buffer is added to measurement cylinder covering the bottom. The iTLC strip is placed into measurement cylinder and allowed to draw the citrate buffer up until it is about 1 cm from the top. The strip is removed and scanned using radio-TLC.

### Example 3—Manufacturing of Anti-EGFR IgA3.0min Drug Substance (DS)

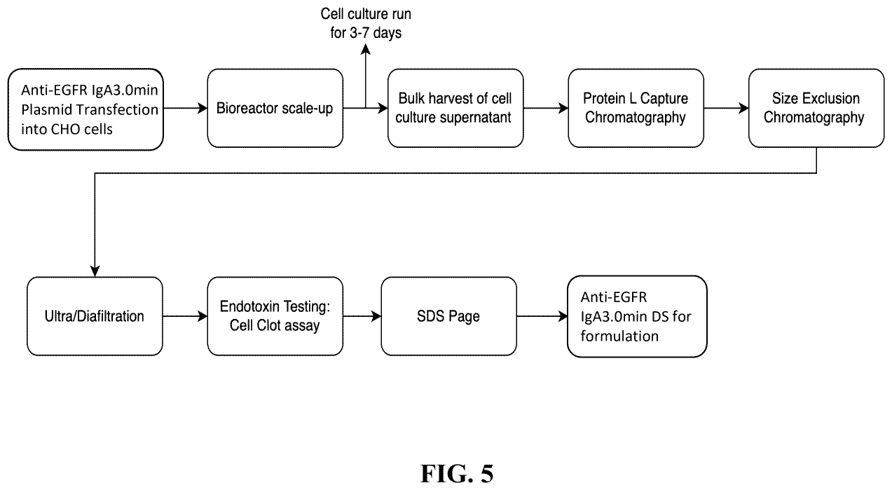

Plasmids encoding anti-EGFR IgA3.0min was produced recombinantly in Chinese hamster ovary (CHO) cells, purified to produce anti-EGFR IgA3.0min drug substance 1 (DS1), and formulated in buffer to form the anti-EGFR IgA3.0min drug product (DP), wherein the anti-EGFR IgA3.0min comprised a VH sequence of SEQ ID NO: 173 and a VL sequence of SEQ ID NO: 211. Briefly, the DNA plasmid encoding anti-EGFR IgA3.0min was transfected into CHO-K1 cells and the anti-EGFR IgA3.0min antibody was expressed, purified, characterized, and buffer exchanged into the formulation buffer. An overview of manufacturing of anti-EGFR IgA3.0min is illustrated in FIG. 5.

### Design of Anti-EGFR IgA3.00min Amino Acid Sequence

The anti-EGFR IgA3.0min has a modified IgA2 Fc backbone as described in Example 1, and an anti-EGFR Fv region. The anti-EGFR Fv region comprises a heavy chain variable region (VH) sequence (SEQ ID NO: 173) and a light chain variable region (VL) sequence (SEQ ID NO: 211). Anti-EGFR IgA3.0min antibody further included a 19 amino acid signal peptide (MGWSCIILFLVATATGVHS (SEQ ID NO: 221)) and a 20 amino acid signal peptide (METDTLLLWVLLLWVPGSTG (SEQ ID NO: 167)) that are present at the N-terminus of the constant regions of the heavy chain and light chain, respectively, to promote efficient transport, folding, assembly, and post-translational modifications. The final anti-EGFR IgA3.0min antibody was expected to yield 141.4 kDa molecular weight protein.

Cloning of Expression Vector and Transfection into CHO Cells

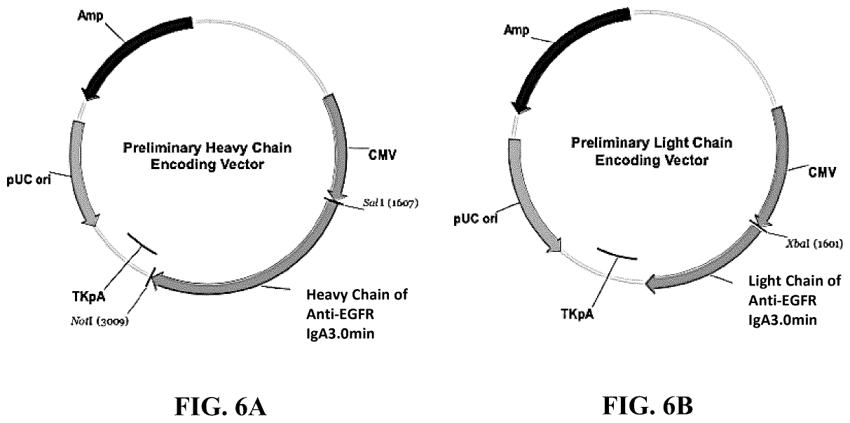

The corresponding DNA sequences that encode both the heavy and light chain sequences of anti-EGFR IgA3.0min were codon optimized for mammalian expression and synthesized by overlap extension PCR. The heavy and light chain DNA sequences were cloned into individual expression plasmids. The vector components are illustrated in FIGS. 6A-6B and their roles described in Table 11. Large-scale plasmid preparation was performed in order to obtain sufficient DNA for transient transfection into CHO-K1 cells. Vector plasmids were transfected into the CHO-K1 cells via the Lipofectamine method under standard conditions.

TABLE 11
Vector components in Preliminary Anti-EGFR IgA3.0min Expression Plasmids

| Name | Type | Description | Notes |
|---|---|---|---|
| Amp | ORF | Ampicillin resistance gene | Allows E. coli to be resistant to |
| | | | ampicillin. |
| pUC ori | Rep origin | pUC origin of replication | Facilitates plasmid replication |
| | | | in E. coli; regulates high-copy |
| | | | plasmid number |
| CMV | Promoter | Cytomegalovirus promoter | The plasmid vector contains the |
| | | | mammalian CMV promoter to |
| | | | drive strong gene expression |
| TKpA | PolyA signal | HSV TK polyadenylation | Allows transcription termination |
| | | signal | and polyadenylation of mRNA |

### Recombinant Expression of Preliminary Anti-EGFR IgA3.00min in CHO Cells

Anti-EGFR IgA3.0min was recombinantly produced in CHO cells under serum-free conditions, using strain CHO-K1. After transfection of the anti-EGFR IgA3.0min expression vector into the CHO-K1 cells, the CHO-K1 culture was scaled up to the required manufacturing volume in a fed-batch bioreactor system. Transient expression and secretion of anti-EGFR IgA3.0min antibody occurred into the cell culture medium and subsequently, the cell culture supernatant was harvested after 3-7 days of expression.

### Purification of Anti-EGFR IgA3.0min

Anti-EGFR IgA3.0min was purified from the harvested cell culture supernatant via a multi-step procedure. Firstly, a protein L-binding step was performed as an efficient purification method to isolate IgA antibody from the supernatant. The supernatant was run through a protein L resin, which combines a rigid, high-flow agarose matrix with the immunoglobulin-binding recombinant protein L ligand, which has a strong affinity to the variable region of antibody kappa light chains. Captured antibodies were eluted and subsequently, size exclusion chromatography (SEC) was performed to isolate properly sized fragments consisting of monomeric forms of anti-EGFR IgA3.0min. The properly sized anti-EGFR IgA3.0min was collected and formulated to produce the drug substance.

### Anti-EGFR IgA3.0min Formulation

Anti-EGFR IgA3.0min DS1 was formulated with 20 mM His and 150 mM NaCl (pH 5.5) during the formulation of the final product. Histidine is a widely used buffer in antibody formulations due to its role in stabilizing the antibody during storage and reducing aggregation, in addition to its buffering function.

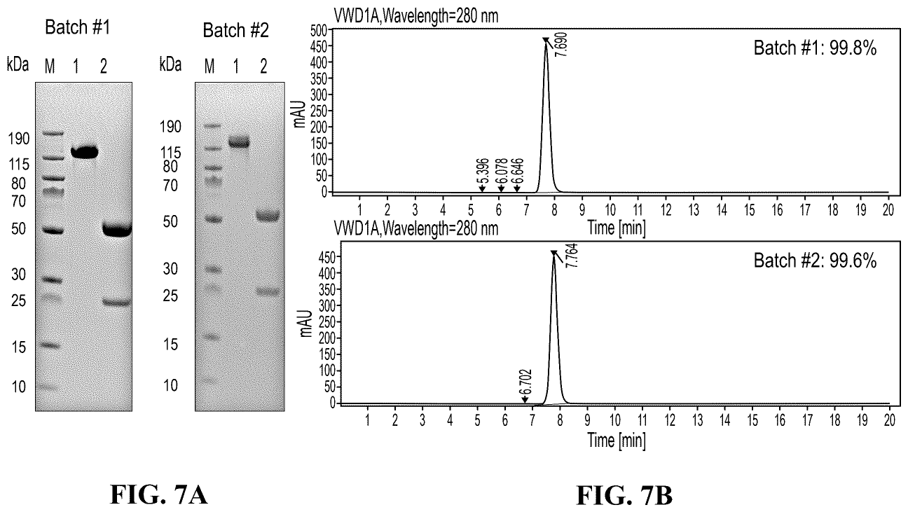

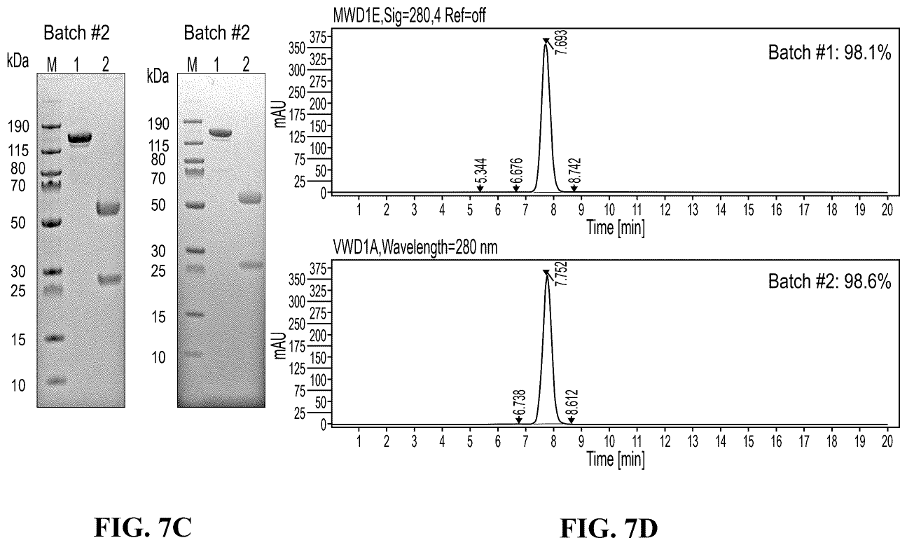

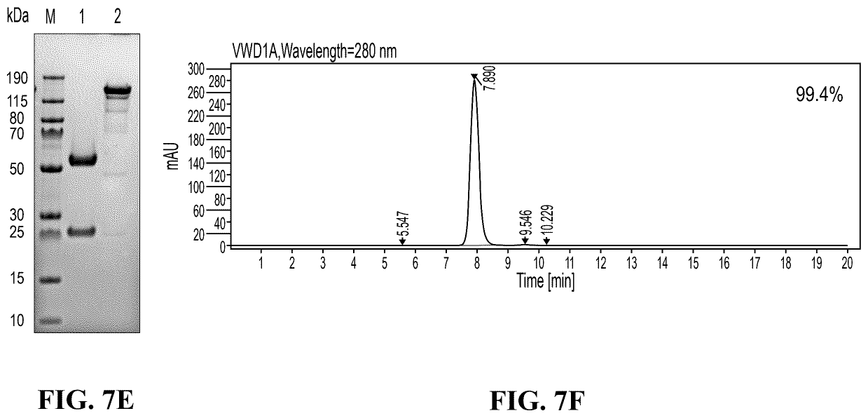

Each step in the purification procedure was quality controlled via a non-reducing SDS-PAGE to visualize the intact molecule and a reducing SDS-PAGE to visualize the separate heavy and light chains. Both conditions showed bands of the expected size; approximately 150 kDa for non-reducing, and 75 kDa for heavy chain and 25 kDa for light chain under reducing conditions. Lastly, after ultrafiltration/diafiltration the sample was run on analytical SEC-HPLC to assess the purity of anti-EGFR IgA3.0min DS1 and gel-clot assays were performed to assess the endotoxin levels. The anti-EGFR IgA3.0min DS manufacturing process achieved a high purity (>98%) of purified anti-EGFR IgA3.0min DS1 (FIGS. 7A-7B).

Using the same method, anti-EGFR IgA3.0min DS2 and anti-EGFR IgA3.0min DS3 were also manufactured. Anti-EGFR IgA3.0min DS2 comprised a VH sequence of SEQ ID NO: 172 and a VL sequence of SEQ ID NO: 198. Anti-EGFR IgA3.0min DS3 comprised a VH sequence of SEQ ID NO: 182 and a VL sequence of SEQ ID NO: 207. The manufacturing process, as described above, also achieved a high purity (>98%) for anti-EGFR IgA3.0min DS2 and anti-EGFR IgA3.0min DS3 (FIGS. 7C-7F).

### Example 4—Manufacturing Anti-EGFR IgA3.0min Drug Product (DP)

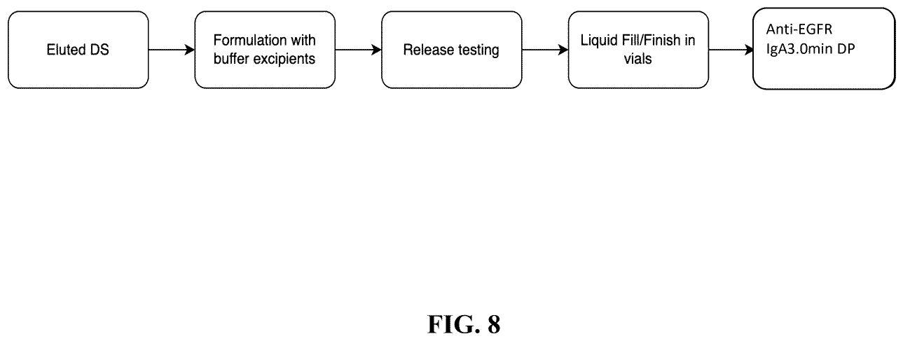

An exemplary, non-limiting drug product formulation, anti-EGFR IgA3.0min DP, was produced and purified as described for the anti-EGFR IgA3.0min DS. The anti-EGFR IgA3.0min DP is the same as the anti-EGFR IgA3.0min DS, but was formulated for release testing (FIG. 8).

### Example 5—Testing of Anti-EGFR IgA3.0min DS/DP

Release testing is performed according to the DS and DP testing strategy provided in Table 12.

TABLE 12
Testing Strategy for IgA-EGFR DS/DP

| | | | Characterization |
|---|---|---|---|
| | | | (C)/ Release (R)/ |
| Attribute | Test | DS / DP | Stability (S) |
| Potency | Binding to EGFR: FACS/SPR | DS/ DP | R/ S |
| | Binding to CD89 | DS | C |
| | Cell viability assay: in vitro | DS | C |
| | ADCC assay: in vitro | DS | C |
| Structural | Glycosylation | DS | C |
| Characteristics | CD | DS | C |
| | Extinction Coefficient Determination | DS | C |
| | Mass spectrometry: Whole mass, | DS | C |
| | Deglycosylated mass, Deglycosylated | | |
| | reduced mass | | |
| | Peptide Mapping | DS | C |
| | Melting Point Analysis: Differential | DS | C |
| | Scanning Calorimetry, Differential | | |
| | Scanning Fluorimetry | | |
| | Disulfide Mapping | DS | C |
| Purity | SEC-HPLC | DS/ DP | R/ S |
| | CE-SDS (Reduced/ Non-reduced) | DS/ DP | R/ S |
| | Capillary isoelectric focusing | DS/ DP | R/ S |
| | Host Cell Protein | DS | R |
| | Host Cell DNA | DS | R |
| | Residuals (Media/ Resins) | DS | R |
| | Protein Concentration | DS/ DP | R/ S |
| Identity | Peptide Map | DS/ DP | R |
| | ELISA | DS/ DP | R |
| Safety | Bioburden | DS | R |
| | Sterility | DP | R |
| | CCIT | DP | S |
| | Endotoxin | DS/ DP | R |
| Other | pH | DS/ DP | R / S |
| Characteristics | Osmolality | DS/ DP | R |
| | Extractable volume | DP | R |
| | Appearance | DS/ DP | R / S |
| | Sub visible particles | DP | R / S |

### Stability Testing

Anti-EGFR IgA3.0min DP is manufactured, formulated, and aseptically filled into clear vials. The DP is then subjected to a stability testing program over 24 months to evaluate critical parameters under the proposed storage conditions. The stability testing program is for determining the anti-EGFR IgA3.0min DP shelf life and support storage, shipping, and handling in the final dosing formulation. The stability testing program is conducted according to International Council for Harmonisation of Technical Requirements for Pharmaceuticals for Human Use (ICH) guidelines for at least 24 months. DS is also tested for stability over at least 24 months per ICH guidelines.

### Example 6—Anti-EGFR IgA3.00min DP Binding Assay, Proliferation Assay and ADCC Assay

Anti-EGFR IgA3.0min DP consists of an engineered anti-EGFR IgA3.0 antibody. The anti-EGFR IgA3.0min antibody acts as a competitive antagonist at the ligand binding site of EGFR to inhibit binding and signaling mediated by EGF and transforming growth factor α, the natural ligands for this receptor. In addition, binding to EGFR results in the selective recruitment of FcαRI (CD89) expressing immune cells such as neutrophils. Neutrophils are the most abundant immune cell type in human circulation, comprising up to 70% of circulating leukocytes. Neutrophils can kill opsonized cancer cells by a mechanism that involves trogocytosis, which can eventually result in a lytic/necrotic type of cell death. In addition, neutrophils can potentially contribute to the recruitment and activation of other immune cells and stimulate adaptive anti-tumor immunity, to further promote tumor cell elimination.

Anti-EGFR IgA3.0min DP selectively recruits FcαR1 (CD89) expressing immune cells, such as neutrophils, to the EGFR tumor antigen, resulting in targeted ADCC. The ability of anti-EGFR IgA3.0min DP in combination with neutrophils to inhibit growth of EGFR-expressing tumor cells was tested using in vitro methods and in vivo in tumor-xenografted mice. Anti-EGFR IgA3.0min DP additionally inhibits and reduces tumor cell growth independently of immune effector cells via inhibition of EGFR signaling. To activate EGFR, the ligand EGF (a monomer) binds simultaneously and cross-links two adjacent receptor chains. The cross-linking enables intracellular kinase domains of the receptor chains to phosphorylate each other on multiple tyrosines. The tyrosine kinase activity of the receptor chains is thus increased and in turn activate several signaling pathways such as the Ras-induced Map kinase pathway, the PI3-kinase pathway and the JAK/STAT pathway. Specific abrogation of EGFR results in cell cycle arrest and apoptosis. Four anti-EGFR IgA3.0min DP, anti-EGFR IgA3.0min DP1, anti-EGFR IgA3.0min DP2, anti-EGFR IgA3.0min DP3 and anti-EGFR IgA3.0min DP4, were designed for administering intravenously.

### In Vitro Pharmacology Studies of Anti-EGFR IgA3.00min DP

In vitro characterization of each of anti-EGFR IgA3.0min DP activity, including EGFR binding, EGF displacement, direct cytotoxicity, and ADCC were conducted as described below.

### Anti-EGFR IgA3.0min DP Binding Affinity to EGFR

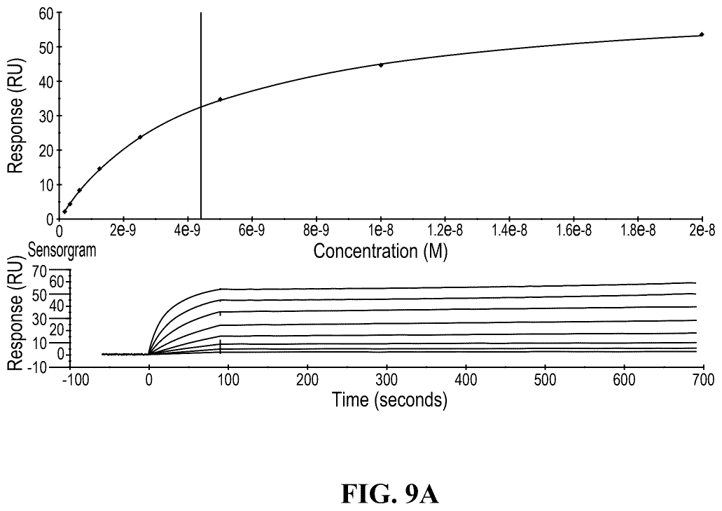

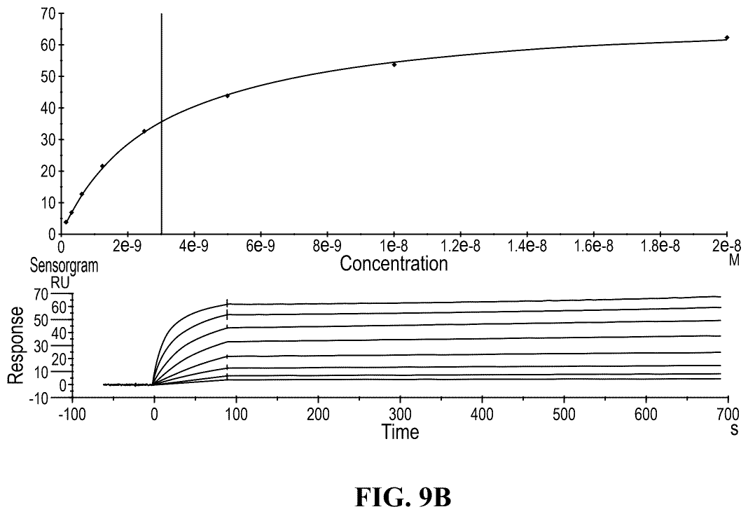

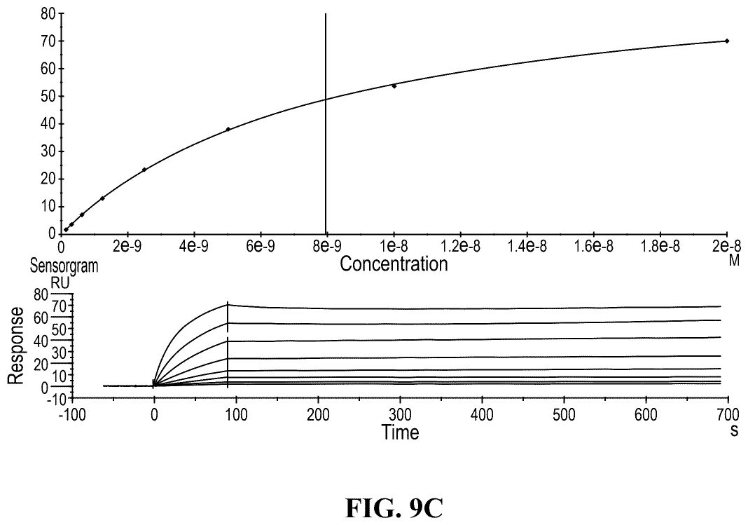

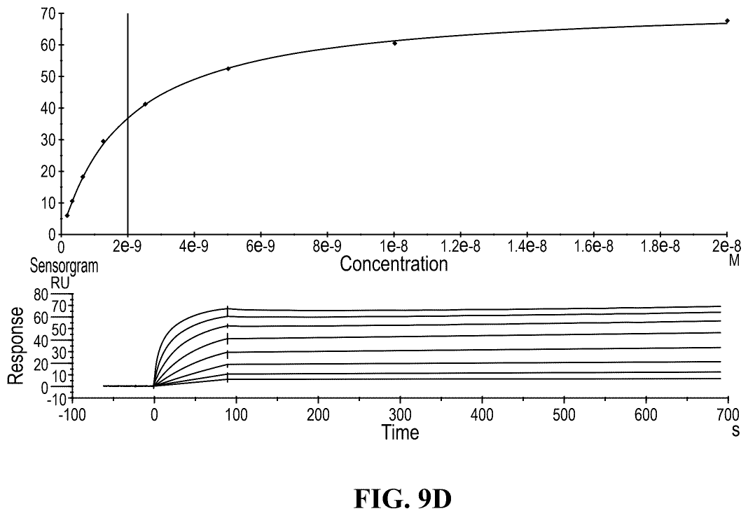

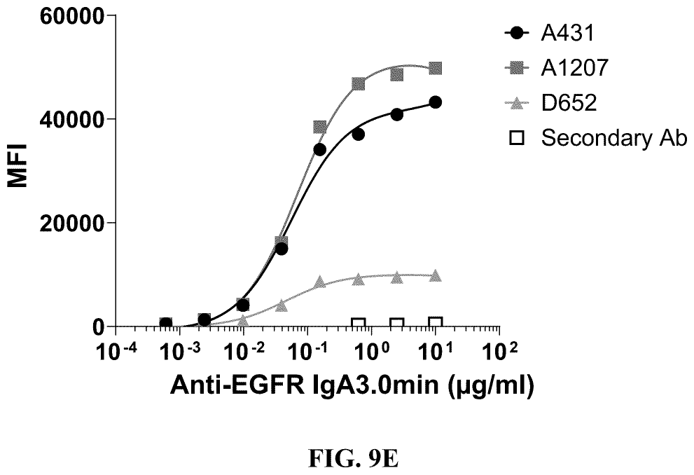

The affinity of each anti-EGFR IgA3.0min DP to the EGFR was established by Surface Plasmon resonance (SPR) (FIGS. 9A-9D) and Fluorescence-activated cell sorting (FACS) (FIG. 9E). For the SPR assay, the binding of each anti-EGFR IgA3.0min DP to immobilized His-tagged EGFR (Recombinant Human EGFR His-tag Avi-tag Protein, R&D Systems) was measured, which was captured on a Ni²⁺-nitrilotriacetic acid sensor chip (Series S Sensor Chip, Cytiva). All measurements were carried out on System, BiaCore T200. For the FACS assay, the binding affinity of anti-EGFR IgA3.0min DP1 was measured to A431, A1207, and D562 cell lines which expressed EGFR at varying levels. Different concentrations of anti-EGFR IgA3.0min DP1 ranging from 0.0007 μg/ml to 10 μg/ml were incubated with each cell line for 45 minutes at 40° C. followed by addition of fluorescently labeled anti-IgA secondary antibody for detection by flow cytometry. Dissociation constants (K_{D}) values were determined to be 4.39 nM by SPR and 0.3 nM by FACS. An analysis of FIG. 9E indicates that anti-EGFR IgA3.0min DP1 binding to EGFR is dose dependent up to a concentration of approximately 1 μg/ml, and is also dependent on the expression level of EGFR on cells as shown by the variation in maximal mean fluorescent intensity (MFI) across the A431, A1207, and D562 cell lines.

### Anti-EGFR IgA3.0min DP Dependent EGF Binding Inhibition

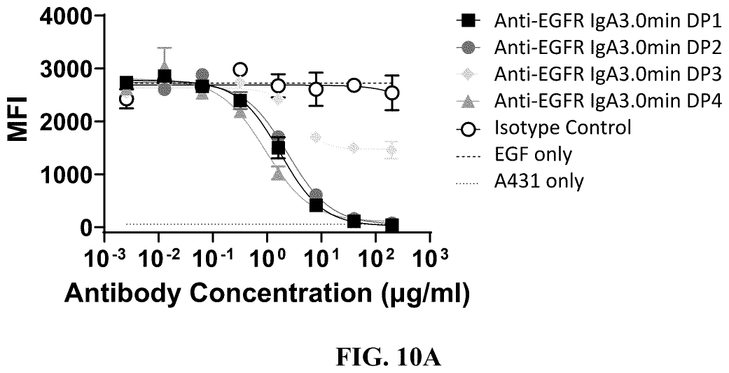

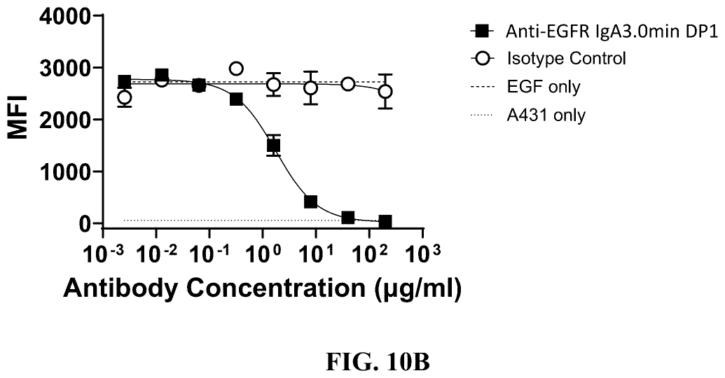

Anti-EGFR IgA3.0min DP acts as a competitive antagonist at the ligand binding site of EGFR to inhibit binding and signaling mediated by EGF. To evaluate the ability of each anti-EGFR IgA3.0min DP to effectively bind to the ligand binding site of EGFR and subsequently inhibit EGF binding, the EGFR-expressing A431 cell line was treated with varying concentrations of each anti-EGFR IgA3.0min DP up to 2 μg/ml. This was followed by cell treatment with fluorescently labeled EGF (EGF-AL488, ThermoFisher Scientific, US) and MFI was measured via a competitive ELISA assay. Each anti-EGFR IgA3.0min DP effectively blocks EGF binding to EGFR in a dose-dependent manner as observed by the reduction in MFI with increasing concentrations of IgA antibody (FIGS. 10A-10B). An analysis of FIGS. 1A-10B further indicated that each anti-EGFR IgA3.0min DP variants had no effect on MFI reduction in the isotype control sample, demonstrating specific binding to EGFR.

### Anti-EGFR IgA3.0min DP Dependent CDC

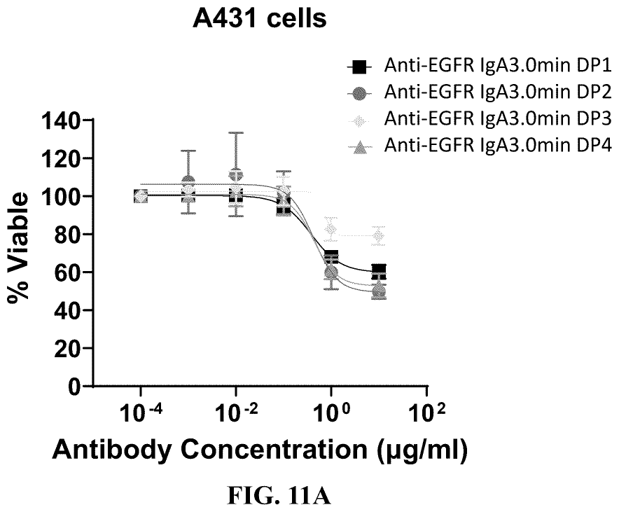

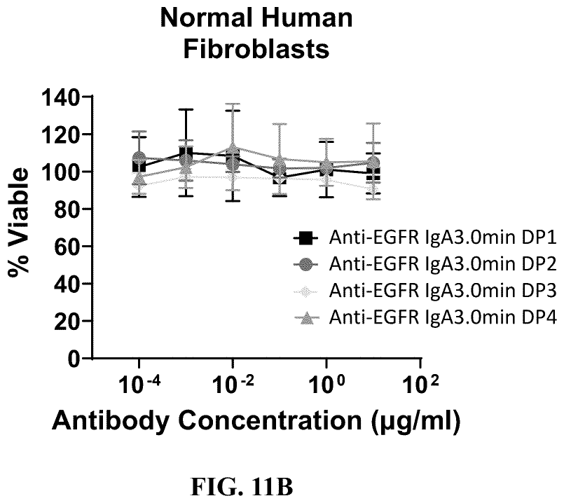

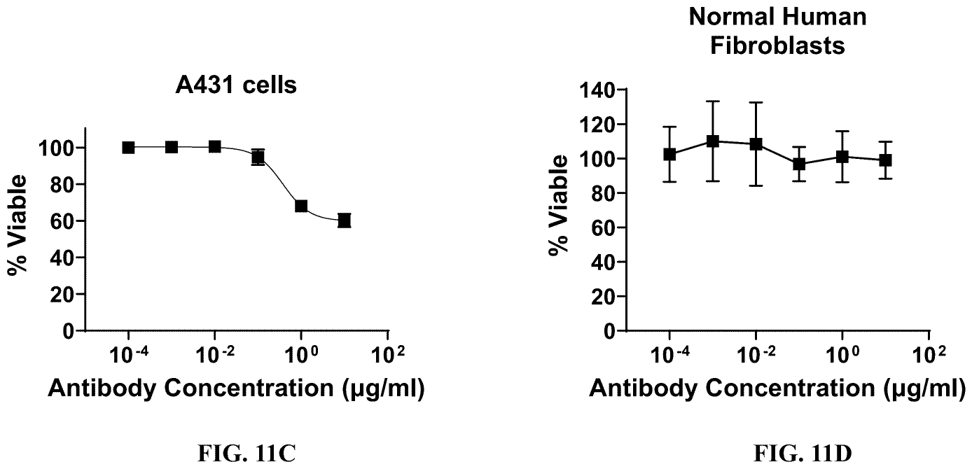

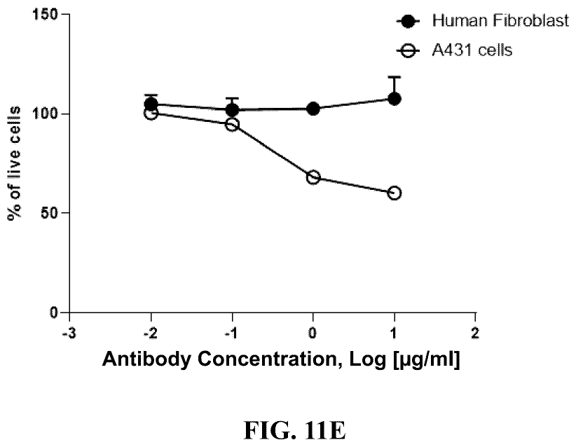

The effect of each anti-EGFR IgA3.0min DP treatment on cell survival was evaluated to determine the ability to induce cell cycle arrest and/or apoptosis, independently of FcαRI (CD89) expressing immune cells such as neutrophils. Cell viability was measured via the Sulforhodamine B Assay Kit (Abcam). Each anti-EGFR IgA3.0min DP reduces cell viability in A431 cells (high expression of EGFR) in a dose dependent manner (FIGS. 11A-11E). No impact on cell survival was observed in normal human fibroblasts which express EGFR at low levels, demonstrating the selectivity of each anti-EGFR IgA3.0min DP to EGFR-high cancer cells.

### Anti-EGFR IgA3.0min DP Dependent ADCC

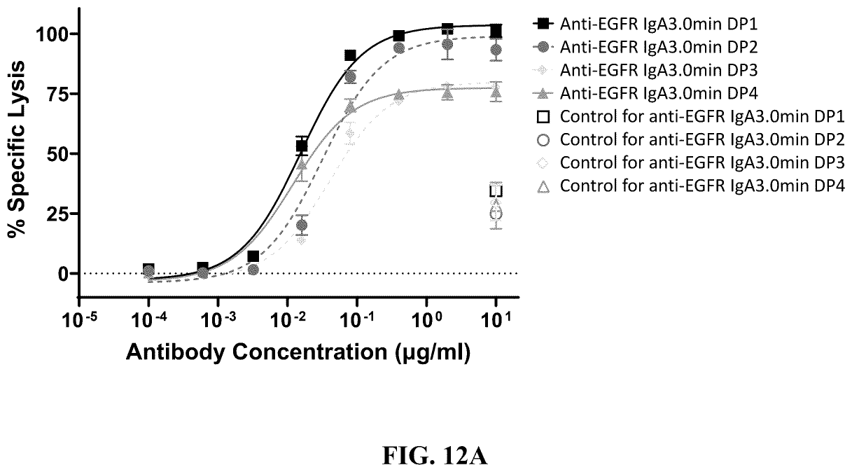

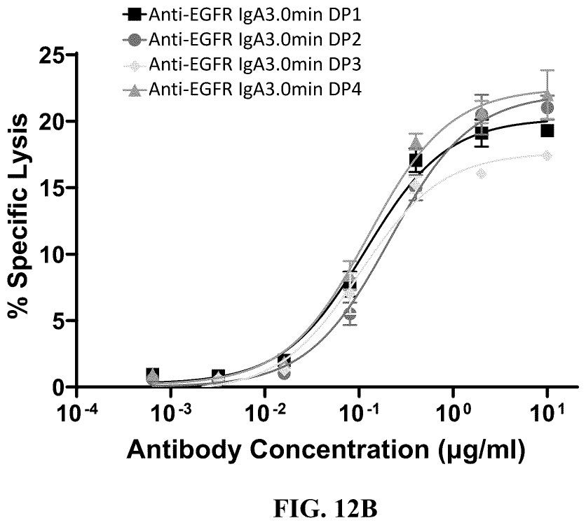

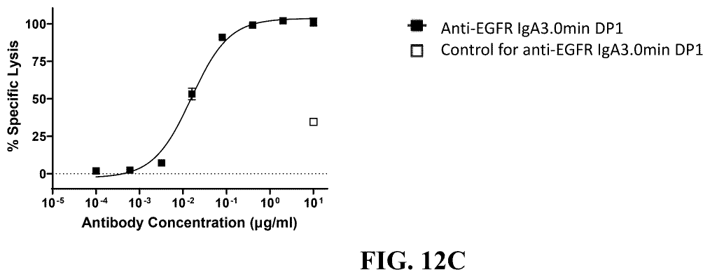

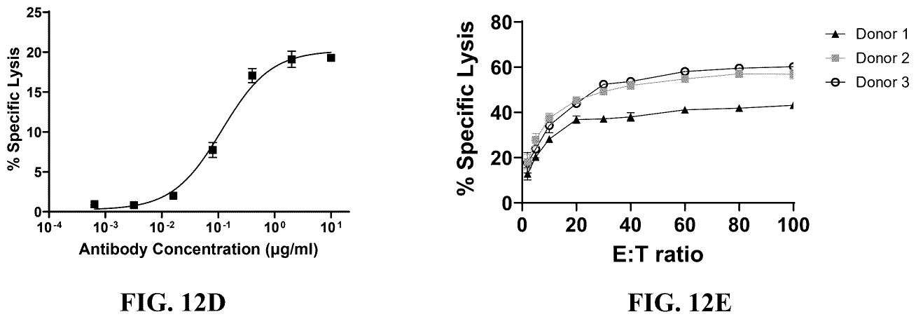

The ability of each anti-EGFR IgA3.0min DP in combination with effector cells (purified neutrophils or whole blood leukocytes) to inhibit growth of EGFR-expressing tumor cells was tested in vitro (FIGS. 12A-12E). ADCC assays using 51Cr-labelled, EGFR-positive A431 target cells were performed as described previously (Dechant et al., 2007). The ADCC assay was performed with increasing concentrations of each anti-EGFR IgA3.0min DP in the presence of either purified neutrophils or whole blood pretreated with erythrocyte lysis buffer. The percentage of specific tumor cell lysis was calculated using the following formula: ((experimental cpm−basal cpm)/(maximal cpm−basal cpm))×100, with maximal lysis determined in the presence of 5% triton and basal lysis in the absence of antibody and effectors. In the presence of purified neutrophils, specific cell lysis increased in a dose-dependent manner for each anti-EGFR IgA3.0min DP. Maximal (100%) specific lysis was achieved above concentration of 0.1 μg/ml for each anti-EGFR IgA3.0min DP (FIGS. 12A and 12C). The Effector:Target (E:T) ratio was observed as 40:1 for purified neutrophils versus cancer cells (FIG. 12E). Antibody-dependent cytotoxicity for each anti-EGFR IgA3.0min DP was also observed in whole-blood leukocyte assays (FIGS. 12B and 12D).

### Evaluation of Tumor Inhibition in Xenograft Tumor Bearing Mice

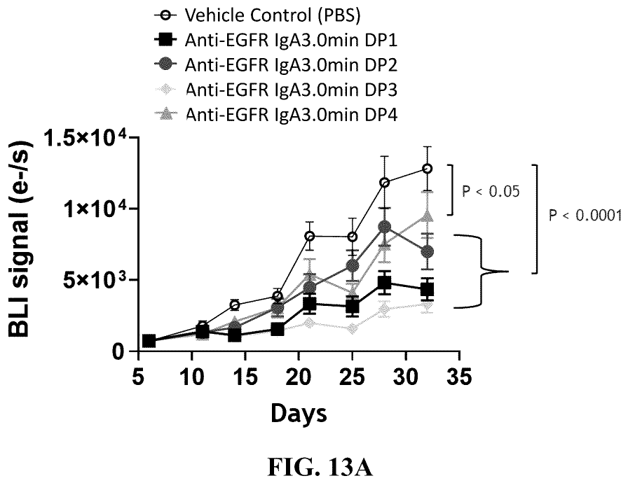

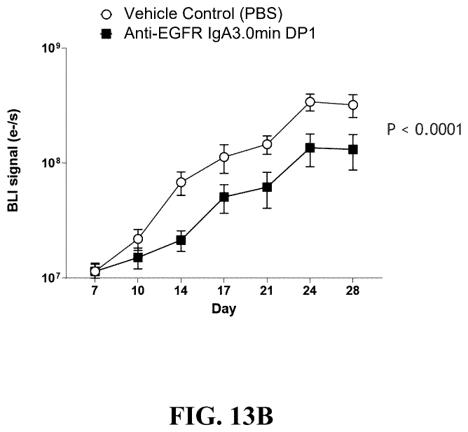

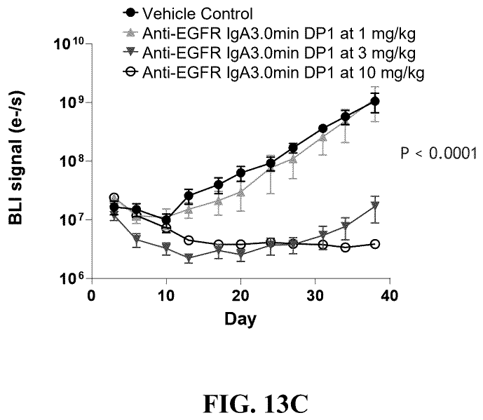

The in vivo activity of each anti-EGFR IgA3.0min DP was tested in a long-term xenograft tumor model using 2 different cell lines, expressing high (A431 cells) or low (A549 cells) levels of EGFR (FIGS. 13A-13C). Both cell lines were transduced with a luciferase gene (luc2) allowing longitudinal monitoring of tumor growth by bioluminescent imaging (BLI). The human epidermoid carcinoma cell line A431 endogenously expresses high levels of EGFR and is partially dependent on EGFR signaling for proliferation. Since there is no expression of FcαRI in mice, human FcαRI Tg NXG mice expressing human FcαRI were used in a physiologically relevant pattern. 5×10⁵ A431-luc2 cells in 100 μL PBS were intravenously injected into the lateral teil vein of FcαRI Tg NOD Xenograft Gamma (NXG) female adult mice. After 4 days groups were randomized and treated with one of 3 doses of anti-EGFR IgA3.0min DP1 (vehicle, 1 mg/kg, 3 mg/kg or 10 mg/kg, N=8 per dosage group) twice weekly, intravenously. Tumor growth was monitored by BMI every 3 days for up to 5 weeks, clinical condition and tumor burden allowing. In the first half of the study, detectable tumor outgrowth was restricted to the lungs, with metastatic growth into various tissues occurring after the third week primarily in the vehicle and 1 mg/kg dose groups (FIG. 13C). Animals treated with 3 mg/kg or 10 mg/kg dosing reported no tumor growth during the study period. The follow up study was performed in A549-luc2 cell xenograft model to investigate the activity of anti-EGFR IgA3.0min DP1 (5 mg/kg) against lowly expressing EGFR tumors with known KRAS mutation (FIGS. 13A and 13B). Although anti-EGFR IgA3.0min DP showed reduced activity against A549-derived tumors, there was still significant tumor growth reduction versus PBS control. These data demonstrating both dose and target dependent activity illustrate the EGFR selectivity for each anti-EGFR IgA3.0min DP.

### Example 7—In Vivo Pharmacology Study for Anti-EGFR IgA30 min DP

A dose response, pharmacodynamics study in mice is conducted for anti-EGFR IgA3.0min DP as per design of Table 13. The study is conducted to determine pharmacodynamics in both A431-xenograft tumor bearing CD89 transgenic mice.

TABLE 13
GLP Toxicology Study Design

| | |
|---|---|
| Compliance | Non-GLP |
| Species/Strain | FcαRI Tg NOD Xenograft Gamma (NXG) |
| Dosing Regimen | Twice Weekly, 35-40 days |
| Route of | Intravenous (IV) Teil Vein Injection |
| Administration | |
| (RoA) | |
| Test System | 12-15 female adult mice are used. |
| | A431 xenograft tumor mice receives 1, 3 or 10 mg/kg of IgA |
| | antibody, delivered IV, twice weekly. |
| Mortality | All animals, once daily (AM) |
| Observations | |
| Clinical Observations | All animals, at least once during pre-dose and weekly thereafter |
| Body Weight | All animals, twice weekly |
| Food Consumption | All animals, daily |
| Terminal Procedures, | Full necropsy and post necropsy analysis of all animals following completion |
| Necropsy after | of dosing~Day 35-40. |
| completion of the | |
| study | |

### Example 8—Pharmacokinetic Study for Anti-EGFR IgA3.0min DP1

### Anti-EGFR IgA3.0min DP1 Pharmacokinetics in Immunocompromised Mice

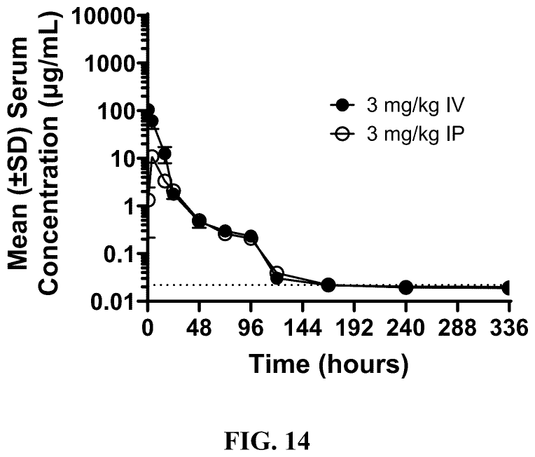

NSG mice received a single dose of 3 mg/kg of anti-EGFR IgA3.0min DP1 delivered either intravenous (IV) or intraperitoneal (IP) (N=4 per delivery group), wherein the anti-EGFR IgA3.0min DP1 comprised a VH sequence of SEQ ID NO: 173 and a VL sequence of SEQ ID NO: 211. Blood samples were taken during a 14-day period and plasma concentrations of anti-EGFR IgA3.0min DP1 was measured at each time point. The following plasma parameters were evaluated to determine the PK prof ile: maximum concentration (Cₘₐₓ), area under the concentration-time curve (AUC), and terminal elimination half-life (t_{1/2}). The PK parameters of anti-EGFR IgA3.0min DP1 following single IV and IP dose are presented in Table 14. The mean plasma concentration curves showed a consistent bi-exponential decline in plasma concentrations of anti-EGFR IgA3.0min DP1, with a t_{1/2} of 18.2 (IV) and 21.2 (IP) (FIG. 14). Anti-EGFR IgA3.0min DP1 plasma concentration was below the Limit of Quantitation (BLQ) at 240 hours post dosage.

TABLE 14
Summary of Pharmacokinetic Parameters of Anti-EGFR IgA3.0
min DP1 Following Single 3 mg/kg Intravenous Dosage in Mice

| | | Cₘₐₓ | | Cₗₐₛₜ | | | AUC_{0-120 h} | AUCₗₐₛₜ | AUC_{inf} |
|---|---|---|---|---|---|---|---|---|---|
| | | (μg/ | Tₘₐₓ | (μg/ | tₗₐₛₜ | t_{1/2} | (hr*μg/ | (hr*μg/ | (hr*μg/ |
| Route | Statistic | mL) | (hr) | mL) | (hr) | (hr) | mL) | mL) | m) |
| IV | N | 4 | 4 | 4 | 4 | 4 | 4 | 4 | 4 |
| | Mean | 105 | 0.5 | 0.03 | 120 | 18.2 | 861 | 861 | 862 |
| | SD | 24.9 | 0 | 0.00238 | 0 | 0.662 | 171 | 171 | 171 |
| IP | N | 4 | 4 | 4 | 4 | 4 | 4 | 4 | 4 |
| | Mean | 10.9 | 4 | 0.04 | 132 | 21.9 | 177 | 177 | 178 |
| | SD | 2.09 | 0 | 0.01 | 24 | 3.76 | 13.2 | 12.8 | 12.6 |

| | | | | AUC_{0-120 h}/ | AUCₗₐₛₜ/ | | |
|---|---|---|---|---|---|---|---|
| | | | | D | D | | |
| | | | Cₘₐₓ/D | (hr*μg/ | (hr*μg/ | Cl* | |
| | | AUC % | (μg/mL/ | mL/(mg/ | mL/(mg/ | (mL/ | Vz* |
| Route | Statistic | Extrap | (mg/kg)) | kg)) | kg) | h) | (mL) |
| IV | N | 4 | 4 | 4 | 4 | 4 | 4 |
| | Mean | 0.096 | 34.9 | 287 | 287 | 0.0718 | 1.89 |
| | SD | 0.0219 | 8.34 | 57.2 | 57.2 | 0.0149 | 0.395 |
| IP | N | 4 | 4 | 4 | 4 | 4 | 4 |
| | Mean | 0.618 | 3.62 | 59.0 | 59.2 | 0.337 | 10.7 |
| | SD | 0.152 | 0.702 | 4.36 | 4.20 | 0.0225 | 2.36 |

Cmax: Maximum concentration, AUC: area under the concentration-time curve, t1/2: terminal elimination half-life, last: last measured timepoint, Inf: Extrapolated to infinity, Cl: Clearance, Vz: volume of distribution.

### Anti-EGFR IgA3.0min DP1 Pharmacokinetics in Non-Human Primates

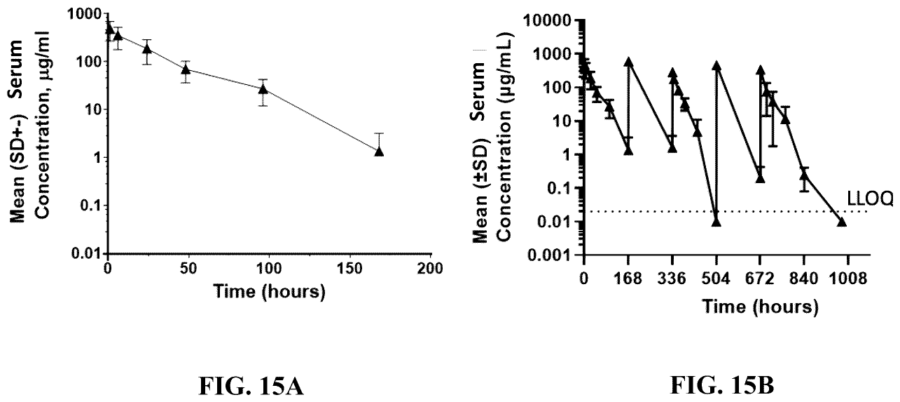

Cynomolgus monkeys were used to establish the pharmacokinetics profile in non-human primates (NHP) following single or multiple doses of anti-EGFR IgA3.0min DP1. The following plasma parameters were evaluated to determine the pharmacokinetics profile: Cₘₐₓ, AUC₀₋₄₈ₕ, t_{1/2}, and drug clearance (CL). The pharmacokinetics parameters of anti-EGFR IgA3.0min DP1 following single intravenous dose administration in NHP are presented in Table 15. In the single-administration pharmacokinetics study, animals received a single intravenous dose of 25 mg/kg of anti-EGFR IgA3.0min DP1 (N=5). Blood samples were taken during a 7-day period and plasma concentrations of anti-EGFR IgA3.0min DP1 was measured at each time point. The mean plasma concentration curve shows a consistent bi-exponential decline in plasma concentration of anti-EGFR IgA3.0min DP1, with a t_{1/2} of 16.9 hours (FIG. 15A). In the multiple-dose study, animals received 4 doses of 25 mg/kg and 1 dose of 12.5 mg/kg of anti-EGFR IgA3.0min DP1 intravenously, spaced 7 days apart. The reduction in dosage at one timepoint was due to material limitations of anti-EGFR IgA3.0min DP1, which needed to be conserved until the next test article batch was available. PK parameters were evaluated over the 42-day study period. No evidence of anti-EGFR IgA3.0min DP1 accumulation was observed with multiple doses, with anti-EGFR IgA3.0min DP1 plasma concentration falling below the lower limit of quantification (LLOQ) after the fifth dosing (FIG. 15B).

TABLE 15
Summary of Pharmacokinetic Parameters of anti-EGFR IgA3.0min
DP1 Following Single and Multiple Intravenous Dosage in NHP

| Dose | Dose | Cₘₐₓ | Half-life | AUC_{0-48 h} | CL |
|---|---|---|---|---|---|
| (mg/kg) | Number | (μg/mL) | (hr) | (hr*μg/mL) | (mL/hr) |
| 25 | Single | 483 | 16.9 | 13900 | 4.11 |
| | Multiple (5) | 343 | 17.1 | 8130 | 6.9 |

### Non-GLP Non-NHP Toxicology Study

A pivotal GLP toxicity study was conducted to assess safety of anti-EGFR IgA3.0min DP1 administered to NHP. NHP were chosen as rodents were not a pharmacologically relevant model because anti-EGFR IgA3.0min DP1 does not bind mouse EGFR. The toxicology of anti-EGFR IgA3.0min DP1 was evaluated in a non-GLP, 5-week multi-dose NHP study. N=2 cynomolgus monkeys were dosed weekly across 5 weeks with 25 mg/kg anti-EGFR IgA3.0min DP1 intravenously, except for the third dose, which was delivered at 12.5 mg/kg. As a control, N=2 NHPs received the approved anti-EGFR IgG antibody, panitumumab, with the same dosage regime. Anti-EGFR IgA3.0min DP1 was well-tolerated with only the occurrence of known EGFR targeting effects during the 5-week multi-dose NHP study (Table 16). These adverse effects appeared milder with anti-EGFR IgA3.0min DP1 compared to anti-EGFR IgG2 (panitumumab) treatment.

TABLE 16
Occurrence of Adverse Effects in NHP Toxicology Study

| | Anti-EGFR IgA3.0min DP1 | | Panitumumab (IgG) | |
|---|---|---|---|---|
| | Animal 1 | Animal 2 | Animal 1 | Animal 2 |
| Day | (854139888) | (8482877379) | (EC728) | (MB809) |
| 1 | Coarse hair | N | N | N |
| 8 | Coarse hair | Loose stool | Loose stool | Loose stool |
| | | Erythema | | |
| 15 | Coarse hair | Erythema | Erythema | Loose stool |
| 22 | Coarse hair | Erythema | Coarse hair | N |
| | | | Xeroderma | |
| | | | Erythema | |
| 29 | Coarse hair | Coarse hair | Coarse hair | Coarse hair |
| | | Xeroderma | Xeroderma | Xeroderma |
| | | Erythema | Erythema | Erythema |
| | | Blepharitis | | |
| 36 | Coarse hair | Erythema | Coarse hair | Coarse hair |
| | | | Xeroderma | Xeroderma |
| | | | | Erythema |
| | | | | Alopecia |
| 43 | Coarse hair | Erythema | Coarse hair | N |
| | Xeroderma | | Xeroderma | |
| | | | Erythema | |
| | | | Anorexia | |
| 50 | Coarse hair | Coarse hair | Coarse hair | Loose stool |
| | | Erythema | Xeroderma | Coarse hair |
| | | | Erythema | Xeroderma |
| | | | | Erythema |
| 56 | Coarse hair | Coarse hair | Coarse hair | Xeroderma |
| | | | Xeroderma | Alopecia |
| | | | Alopecia | |

### Example 9—NHP GLP Toxicology Study

A four-week toxicity study in cynomolgus monkey, male and female, is conducted to assess safety of the anti-EGFR IgA3.0min DP administered to NHP by intravenous injection. A summary of the study design is shown in Table 17. The safety study includes a dose escalation across three concentrations of anti-EGFR IgA3.0min DP.

TABLE 17
Design of Pivotal Safety Study for Anti-EGFR IgA3.0 min DP

| Compliance | GLP, 4 -week study | | | | | |
|---|---|---|---|---|---|---|
| Species/Strain | Cynomolgus monkey | | | | | |
| Dosing Regimen | Twice a week (on Days 1, 4, 8, 12 15, 19, 22, 25 and 29) | | | | | |
| Route of | Intravenous (IV) (slow bolus) Injection (5-10 minutes). | | | | | |
| Administration | Animals are restrained and dosed via indwelling catheter. | | | | | |
| (RoA) | | | | | | |
| Test System | Group | Dose | Main Study | | Recovery Study | |
| | | (mg/kg/day) | Males | Females | Males | Females |
| | 1 (Control) | 0 | 3 | 3 | 2 | 2 |
| | 2 | TBD | 3 | 3 | — | — |
| | 3 | TBD | 3 | 3 | — | — |
| | 4 | TBD | 3 | 3 | 2 | 2 |

| | |
|---|---|
| Pretreatment Period | Two weeks |
| Mortality | All animals, once daily (AM) |
| Observations | |
| Clinical Observations | All animals, at least once during pre-dose and weekly thereafter |
| Body Weight | All animals, twice pre study and weekly thereafter |
| Food Consumption | All animals, daily (qualitative) |
| Ophthalmology | All animals, Pretest, week 4 and last week of recovery. |
| | Funduscopic and slit-lamp examinations are performed. Exams are performed |
| | by a board-certified veterinary ophthalmologist. |
| Electrocardiology | All animals, Pretest, Week 4 and last week of recovery. Qualitative |
| (ECG) | evaluation of the data is performed by a board-certified veterinary |
| | cardiologist. |
| | Parameters: Heart Rate, RR, PR, QRS, QT and QTc intervals by eight lead |
| | ECG on temporarily restrained animals. |
| Blood Pressure | All animals, Pretest, Week 2, and last week of recovery. |
| | Parameters: Diastolic, Systolic, and Mean Arterial pressure |
| Clinical Pathology | Hematology, Serum chemistry, and Coagulation (PT, APTT and |
| Parameters | Fibrinogen): All animals, Pretest, Week 4, and last week of recovery. |
| | Urinalysis: All animals, Pretest, Week 4, and last week of recovery. |
| Terminal Procedures, | Full necropsy and post necropsy analysis of 24 animals Week 5 (following |
| Necropsy | completion of dosing ~Day 36) and 8 recovery animals ~Day 73. |
| Assessment of | Assessment of toxicokinetic parameters such as Cmax, tmax, AUC and |
| toxicokinetic | impact on the toxicokinetic profiles due to the presence of anti-drug |
| parameters | antibodies are assessed and included in the toxicokinetic report |

### Example 10—Phase 1/2a Oncology Basket Trial for Anti-EGFR IgA3.0min DP

An open-label, non-randomized, multi-center, dose-escalation study is conducted in adults with advanced/metastatic cancers that are known to express EGFR, who have exhausted or are intolerant to or are considered inappropriate for standard of care therapy, at multiple centers. The phase 1 stage is conducted to determine a recommended phase 2 dose before expansion to phase 2a stage, allowing for the evaluation of the safety, tolerability, pharmacokinetics, and pharmacodynamics of single and multiple intravenous doses of anti-EGFR IgA3.0min antibody. Table 18 provides study design of the Phase 1/2 oncology basket trial for anti-EGFR IgA3.0min antibody.

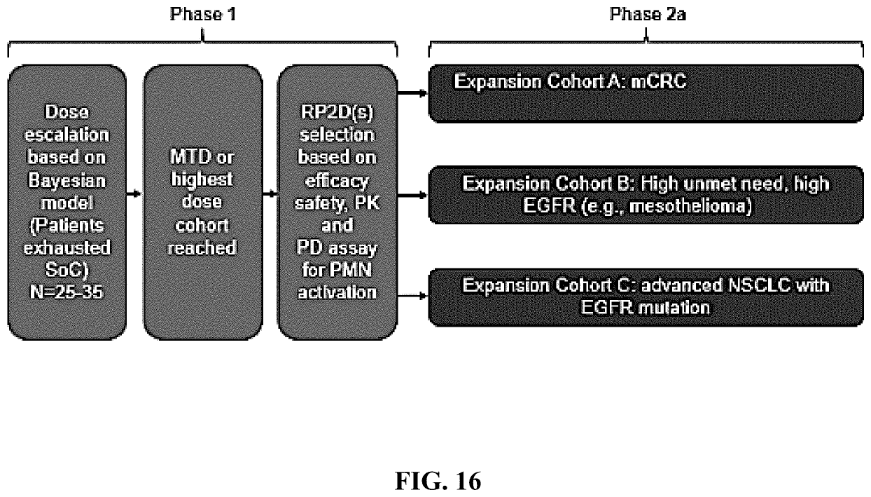

TABLE 18
Study Design

| Investigational | IgA antibody | |
|---|---|---|
| Product | Objectives | Endpoints |
| Study | Phase 1 (Dose Escalation) | |
| Objectives | Primary | |
| and | To determine the maximum | Adverse events (AEs) are assessed by |
| Endpoints | tolerated dose (MTD) and/or | the incidence and severity of dose- |
| | recommended phase 2 dose | limiting toxicity (DLT)s within the |
| | (RP2D) of IGA ANTIBODY | DLT observation period (Cycle 1) |
| | Secondary | |
| | To assess the safety and | Incidence of patients with treatment- |
| | tolerability of IgA antibody | emergent adverse events (TEAEs) |
| | To evaluate the preliminary | and serious adverse events (SAEs) |
| | antitumor activity of IgA | Objective response rate (ORR) using |
| | antibody | Response Evaluation Criteria in |
| | To characterize the | Solid Tumors (RECIST) v1.1 is |
| | pharmacokinetic (PK) profile of | determined by Investigator |
| | IgA antibody administered | assessment |
| | intravenously (i.v.) | PK parameters of IgA antibody: |
| | To assess the immunogenicity of | Cₘₐₓ, Tₘₐₓ, Cₘᵢₙ, and AUCₜₐᵤ |
| | IgA antibody | Frequency of patients developing |
| | | anti-drug antibodies (ADAs) against |
| | | IgA antibody |
| | Exploratory | |
| | To assess the preliminary | ORR using iRECIST |
| | antitumor activity of IgA | Frequency of patients developing |
| | antibody using the Response | neutralizing ADAs against IgA |
| | Evaluation Criteria in Solid | antibody |
| | Tumors for immunotherapy | |
| | (iRECIST) | |
| | To assess the pharmacodynamic | |
| | (PD) effects of IgA antibody | |
| | To assess the predictive potential | |
| | of biomarkers measured in blood | |
| | in response to treatment | |
| | To assess the biological effects of | |
| | IgA antibody demonstrated by | |
| | changes in immune cells, | |
| | immune cell markers, serum | |
| | cytokines, and gene expression | |
| | patterns | |
| | Phase 2a (Expansion) | |
| | Primary | |
| | To evaluate the antitumor activity | Objective response rate (ORR) using |
| | of IgA antibody | Response Evaluation Criteria in |
| | | Solid Tumors (RECIST) v1.1 is |
| | | determined by Investigator |
| | | assessment |
| | Secondary | |
| | To assess preliminary efficacy of | Progression-free survival (PFS) |
| | IgA antibody | according to RECIST v1.1 is |
| | To assess the safety and | determined by investigator |
| | tolerability of IgA antibody | assessment |
| | To characterize the PK profile of | Duration of response (DOR) |
| | IgA antibody | according to RECIST v1.1 is |
| | To assess the immunogenicity of | determined by investigator |
| | IgA antibody | assessment |
| | | Clinical benefit rate (CBR) according |
| | | to RECIST v1.1 by investigator |
| | | assessment is determined |
| | | Incidence of patients with TEAEs and |
| | | SAEs |
| | | PK parameters of IgA antibody: Cₘₐₓ, |
| | | Tₘₐₓ, Cₘᵢₙ, and AUCₜₐᵤ |
| | | Frequency of patients developing |
| | | anti-drug antibodies (ADAs) against |
| | | IgA antibody |
| | Exploratory | |
| | To assess preliminary efficacy of | Overall survival (OS) via RECIST |
| | IgA antibody | v1.1ORR using iRECIST |
| | To assess the preliminary | Frequency of patients developing |
| | antitumor activity of IgA | neutralizing ADAs against IgA |
| | antibody using the Response | antibody |
| | Evaluation Criteria in Solid | |
| | Tumors for immunotherapy | |
| | (iRECIST) | |
| | To assess the pharmacodynamic | |
| | (PD) effects of IgA antibody | |
| | To assess the predictive potential | |
| | of biomarkers measured in blood | |
| | in response to treatment | |
| | To assess the biological effects of | |
| | IgA antibody demonstrated by | |
| | changes in immune cells, | |
| | immune cell markers, serum | |
| | cytokines, and gene expression | |
| | patterns | |

| | |
|---|---|
| | Abbreviations: AUCₜₐᵤ: area under the concentration-time curve over the dose |
| | interval; Cₘₐₓ: maximum plasma concentration; Cₘᵢₙ: minimum plasma |
| | concentration; Tₘₐₓ: time to Cₘₐₓ |
| Methodology | IgA antibody is a first in human (FIH), Phase 1/2a, open-label, non-randomized, |
| | multicenter, dose escalation/expansion study evaluating IgA antibody as |
| | monotherapy in subjects with advanced solid malignancies whose disease has |
| | progressed after treatment with previous anticancer therapies. |
| | Two parts to this study includes: a dose escalation phase (Phase 1) and an |
| | expansion phase (Phase 2a). The aim of the dose escalation phase is to |
| | determine the MTD and to establish 1 or more RP2Ds. An adaptive 2-parameter |
| | Bayesian logistic regression model (BLRM) guided by the dose escalation with |
| | overdose control (EWOC) is used in the escalation phase to guide determination |
| | of the MTD and/or RP2D in subjects with advanced solid malignancies. |
| | The dose escalation phase (Phase 1) is followed by the expansion phase (Phase |
| | 2a) once the MTD and/or 1 or more RP2Ds of IgA antibody monotherapy has |
| | been determined. The expansion phase (Phase 2a) of the study is intended to |
| | collect preliminary evidence of antitumor efficacy and to further confirm the |
| | safety of IgA antibody as monotherapy in distinct patient populations. The |
| | expansion phase has 3 cohorts based on tumor type. An overview of study |
| | design is provided in FIG. 16. |
| | The expansion cohorts is optionally opened in parallel or subsequently. Based |
| | on the findings in the Phase 1 part of the study, additional doses are optionally |
| | explored in the Phase 2a expansion cohorts. |
| | For each subject, the study starts with the provision of written informed |
| | consent, followed by a screening phase lasting up to 21 days. Eligible subjects |
| | are treated with IgA antibody administered as an i.v. infusion as long as they |
| | continue to show clinical benefit as judged by the Investigator, until disease |
| | progression, intolerable toxicity, Investigator discretion, or subject withdrawal |
| | of consent. |
| | After the end of treatment, subjects are followed for safety, duration of response |
| | (DOR), progression-free-survival (PFS) and overall survival (OS) until the end |
| | of the study, which is defined as the date the last subject who has received |
| | treatment has completed the first long-term follow-up visit. |
| | To minimize the number of subjects treated at potentially subtherapeutic dose |
| | levels in the dose escalation phase (Phase 1), Cohorts 1 and 2 enroll a minimum |
| | of 2 subjects, whereas Cohorts 3 and above enroll a minimum of 3 subjects. In |
| | Cohorts 3 and above, a fourth subject are enrolled if treatment of the fourth |
| | subject is anticipated to begin within 14 days of treatment initiation of the third |
| | subject in the cohort. Treatment in each cohort begins in a staggered approach |
| | with at least 7 days between the first dose of the first subject and the first dose |
| | of the subsequent subjects. After the first subject in each cohort has begun |
| | treatment and been observed for at least 7 days, additional subjects within the |
| | cohort, optionally, begin treatment concurrently. |
| | In Cohorts 1 and 2, a minimum of 2 subjects in each cohort are treated and |
| | complete the 28-day dose-limiting toxicity (DLT) observation period before the |
| | Safety Review Committee (SRC) convene and decide on the dose level for the |
| | next cohort of subjects. In Cohorts 3 and above, a minimum of 3 subjects in |
| | each cohort are treated and be evaluable for DLT assessment to allow for a dose |
| | escalation decision by the SRC. All subjects who start treatment need to |
| | complete the 28-day DLT observation period or experience a DLT before the |
| | SRC meeting and dose escalation decision (minimum of 2 patients in Cohorts |
| | 1/2 and 3 patients in Cohorts 3/4). The dose cohorts is optionally escalated, de- |
| | escalated or expanded to up to 6 subjects based on the SRC decision. |
| | Dose escalation decisions are made by the SRC, consisting of the Principal |
| | Investigators at each study center and the Sponsor's Medical representative(s), |
| | and additional clinical experts, as needed. The BLRM is assessed for all |
| | subjects in the dose-determining set (DDS) consisting of all subjects who |
| | received at least 1 dose of IgA antibody, and either experienced a DLT at any |
| | time during Cycle 1 or meet the minimum treatment and safety evaluation |
| | requirements without experiencing a DLT within Cycle 1. After the completion |
| | of a given dose cohort, or at any time the BLRM is updated, the decision for |
| | dose escalation and the actual dose chosen is based on the recommendation of |
| | the BLRM regarding the highest admissible dose according to the EWOC |
| | principle and the medical review of all available clinical, laboratory, and PK |
| | data. The outcome of these analyses and the respective datasets are reviewed by |
| | the SRC. The SRC is then decides the dose level selection of the next dose |
| | cohort prior to enrollment. In the event of a Common Terminology Criteria for |
| | Adverse Events (CTCAE) Grade 5 adverse event (AE) or a second CTCAE |
| | Grade 4 AE considered by the Investigator to be at least possibly related to IgA |
| | antibody at any time during Phase 1, the Sponsor will suspend further accrual |
| | and the Sponsor will perform a safety analysis that will be reviewed by an ad- |
| | hoc SRC meeting to decide on further progression of the study. |
| | Once at least 1 RP2D dose has been determined, enrollment into 1 or more |
| | expansion cohorts for selected cancer indication in Phase 2a begins (FIG. 16). |
| | Other expansion cohorts, optionally, follow with the same or a different RP2D |
| | dose level. An optimum Simon's two-stage design is applied for the preliminary |
| | efficacy analyses for the following expansion cohorts. Each expansion cohort |
| | enrolls up to a total of 20 subjects; an interim analysis (IA) is conducted after |
| | 10 subjects. |
| | Expansion Cohort A enrolls subjects with microsatellite stable |
| | colorectal cancer (CRC) |
| | Expansion Cohort B enrolls subjects with head and neck squamous |
| | cell carcinoma (HNSCC) |
| | Expansion Cohort C enrolls subjects with advanced non-small cell |
| | lung cancer (NSCLC) with an EGFR mutation |
| Number of | Approximately 25 to 35 subjects are enrolled in the dose escalation phase |
| Subjects | (Phase 1), dependent on the tested dose cohorts and the safety profile of IgA |
| (planned) | antibody. |
| | Additionally, 3 expansion cohorts, each with up to a total of 20 subjects in |
| | Cohorts A, B, and C, respectively, are enrolled in the expansion phase (Phase |
| | 2a) for a maximum of 95 subjects. Depending on the preliminary efficacy |
| | observed in the dose escalation phase (Phase 1) of the study, additional |
| | expansion cohorts are added by a protocol amendment. |
| Diagnosis and | Subjects are considered eligible to be enrolled in the study in part based on |
| Main Criteria | meeting ALL of the selected inclusion criteria and NONE of the selected |
| | exclusion criteria are met as defined below. |
| | SELECTED INCLUSION CRITERIA |
| | The study enrols subjects with advanced solid malignancies whose disease has |
| | progressed after treatment with previous anticancer therapies. |
| | Subjects are eligible only if all the following criteria are met: |
| | 1) For dose escalation phase (Phase 1): subjects with advanced or |
| | metastatic solid malignancies that are known to express EGFR. |
| | Subjects must have evaluable disease and must have (i.e., |
| | mandatory) at least 1 tumor site that is accessible to biopsy and |
| | that is considered by the Investigator to be low risk and of |
| | sufficient size to undergo a core biopsy procedure on at least 2 |
| | separate occasions (i.e., pre-treatment and on treatment, and if |
| | possible, on progression). |
| | Expansion phase (Phase 2a): subjects have measurable disease |
| | by RECIST v1.1 and measurable disease per RECIST v1.1 is |
| | defined as at least 1 measurable lesion ≥10 mm by computed |
| | tomography [CT] scan or magnetic resonance imaging (MRI) |
| | or ≥20 mm by chest X-ray; malignant lymph nodes are |
| | considered measurable if the short axis is ≥15 mm as assessed |
| | by CT scan. The last imaging has been performed within 28 |
| | days before Cycle 1 Day 1 (C1D1). |
| | For both Phase 1 and Phase 2a: provide archival tumor if |
| | available from the primary tumor (a paraffin embedded tumor |
| | tissue block sufficient to obtain at least 10 sections of 4 to 5 |
| | micrometer thickness). |
| | 2) For both phases (Phase 1 and Phase 2a): Subjects who have |
| | exhausted or intolerant to or are considered inappropriate for |
| | standard of care (SOC) therapy by the Investigator |
| | 3) For both Phase 1 and Phase 2a: subjects have documented |
| | radiological progression during or after their latest therapy |
| | 4) Male or female aged ≥18 years on the day of signing informed |
| | consent. |
| | 5) Eastern Cooperative Oncology Group (ECOG) Performance Score (PS) |
| | 0 or 1 |
| | 6) Adequate organ function |
| | 7) Serum potassium, calcium, magnesium, and phosphate within normal |
| | limits or not worse than CTCAE v5.0 Grade 1 and asymptomatic. |
| | 8) Subjects in expansion phase (Phase 2a) have histologically confirmed |
| | advanced or metastatic EGFR-positive malignancy such as those listed |
| | below for each expansion cohort: |
| | Cohort A: Subjects with KRAS-wildtype, microsatellite stable CRC |
| | whose disease has progressed after having received ≥2 prior lines |
| | of therapy, which has included oxaliplatin, irinotecan, |
| | fluoropyrimidine, bevacizumab, and an anti-EGFR therapy |
| | (cetuximab or panitumumab). |
| | Cohort B: Subjects with advanced or metastatic HNSCC |
| | Cohort C: Subjects with advanced or metastatic NSCLC harboring |
| | a targetable EGFR kinase domain mutation and whose disease has |
| | progressed on or after having received ≥1 prior lines of therapy for |
| | advanced disease including ≥1 prior TKI approved for EGFR |
| | mutated NSCLC, such as gefitinib, erlotinib, afatinib, dacomitinib |
| | or osimertinib. Subjects who were treated with a 1^{st} or 2^{nd} |
| | generation TKI in 1^{st} line and developed a documented T790M |
| | mutation have received osimertinib to be eligible. Subjects have |
| | documentation of EGFR mutated NSCLC as assessed by an |
| | approved test using genomic sequencing of tumor or circulating |
| | free tumor DNA. |
| | SELECTED EXCLUSION CRITERIA |
| | 1) Subjects with known IgA deficiency |
| | 2) Subjects with active or history of IgA nephropathy (i.e., Berger's |
| | disease) |
| | 3) Currently participating in a study and receiving study therapy, or |
| | participated in a study of an investigational agent and received study |
| | therapy or used an investigational device within 4 weeks of the first |
| | dose of study drug. |
| | 4) Treatment with systemic anticancer therapy within 4 weeks (6 weeks if |
| | therapy was mitomycin C and/or nitrosoureas), or within 5 half-lives of |
| | the agent if half-life is known and it is shorter, before first dose of study |
| | drug. Anticancer therapies include cytotoxic chemotherapy, targeted |
| | inhibitors, and immunotherapies, but do not include hormonal therapy |
| | or radiotherapy. |
| | 5) Radiation therapy within 2 weeks before first dose of study drug or |
| | unresolved (National Cancer Institute [NCI] CTCAE v5.0 >Grade 1) |
| | toxicity from previous radiotherapy (e.g., radiation dermatitis). |
| Investigational | During the dose escalation phase (Phase 1) IgA antibody is administered as bi- |
| product, | weekly infusion. Based on results from the dose escalation phase (Phase 1), |
| dosage and | additional doses are, optionally, explored in the expansion phase (Phase 2a). |
| mode of | For dose escalation phase (Phase 1) and expansion phase (Phase 2a) |
| administration | Bi-weekly (twice per week) dosing regimen: i.e., on Day 1, Day 4, Day 8, |
| | Day 11, Day 15, Day 18, Day 22 and Day 25 of a 28 day cycle. |
| | The baseline infusion time does not take less than approximately 1 hour with a |
| | mandatory post-infusion observation period of 2 hours (Note: infusion time is |
| | approximately 1 hour, any infusion time ≥50 minutes is not a deviation) for at |
| | least the first 2 IgA antibody infusions. It is also noted that as the dose |
| | escalation continues into the higher dosing cohorts, the baseline infusion time, |
| | optionally, increase to >1 hour accompanied by a longer post-infusion |
| | observation period based on a given subject's tolerability. For the higher dosing |
| | cohorts that are administered over >1 hour, the infusion time is, optionally, |
| | decreased to a minimum of 1 hour after at least 2 consecutive IgA antibody |
| | infusions with no infusion-related reaction (IRR)/cytokine release syndrome |
| | (CRS) >Grade 1 at the discretion of the Investigator along with a potential |
| | reduction of post infusion observation period to a minimum of 2 hours. |
| | Investigators are allowed to make 1 modification per infusion. |
| | Subjects, optionally, receive IgA antibody as long as they continue to show |
| | clinical benefit as judged by the Investigator until disease progression, |
| | intolerable toxicity, Investigator discretion or subject withdrawal of consent. |
| | For the dose escalation phase (Phase 1), the starting first-in-human (FIH) dose |
| | for IgA antibody is (TBD) mg. Four additional provisional dose levels are |
| | defined for the dose escalation (increasing incrementally at 200% per dose. If |
| | the first dose level is not well tolerated, a 50% lower dose, optionally, is |
| | evaluated. It is possible for additional, intermediate dose levels to be tested |
| | during the dose escalation (Phase 1) or as part of Phase 2a to fine tune the |
| | RP2D. |
| Criteria for | Safety is assessed by periodic vital signs, physical examinations, ECOG PS, |
| evaluation | 12-lead ECGs, clinical laboratory assessments, and monitoring of AEs. Adverse |
| | events are graded using the NCI CTCAE, v5.0. |
| | Disease response is assessed by the Investigator, using local RECIST v1.1 and |
| | iRECIST. In Phase 1 and 2a, imaging results are also sent for independent |
| | central review. Tumor assessment with CT and/or MRI occur at Screening as |
| | well as at timepoints specified in the respective SoA tables for weekly and |
| | every-2week dose regimens (see APPEND-IX A [Section 14.1] and |
| | APPENDIX B [Section 14.2], respectively). Partial or complete response needs |
| | to be confirmed with repeated assessment at least 4 weeks after the initial |
| | assessment. |
| | The PK profile is assessed by determining serum levels of IgA antibody at |
| | intervals throughout the study. |
| | The pharmacodynamics (PD) is assessed by measuring cytokine levels and |
| | various lymphocyte subsets (e.g., neutrophils/lymphocytes ratio) in peripheral |
| | blood. |
| Statistical | The safety set consists of all subjects who received at least 1 dose of IgA |
| methods | antibody. The safety set is the primary population for all safety related |
| | endpoints except determination of the dose-DLT relationship, and for all |
| | efficacy related endpoints. |
| | The DDS consists of all subjects in the safety set who have either (a) |
| | experienced DLT at any time during Cycle 1, or (b) met the minimum treatment |
| | and safety evaluation requirements without experiencing DLT within Cycle 1. |
| | During the study, for the first 2 dose levels of IgA antibody treatment, at least 2 |
| | subjects are evaluated for dose escalation to occur. For the remaining cohorts, a |
| | minimum of 3 subjects evaluable for the DDS are treated per dose cohort until |
| | determination of the MTD and/or RP2D. Subjects receiving ≥80% of their |
| | assigned IgA antibody dose in Cycle 1 and complete the 28-day DLT |
| | observation period or have had a DLT within the first cycle of treatment is |
| | considered evaluable for DLT. |
| | The DDS is used in the BLRM to estimate the dose-DLT relationship. |
| | The PK set consists of all subjects who have received at least 1 adequately |
| | documented dose of study drug and have at least 1 adequately documented post |
| | dose PK measurement. |
| | Data is summarized with respect to demographic and baseline characteristics, |
| | medical history, prior cancer history and anticancer therapies, prior medication, |
| | safety measurements, efficacy measurements and all relevant PK and PD |
| | measurements using descriptive statistics (quantitative data) and contingency |
| | (frequency and percentages) tables (qualitative data) by dose cohort. |
| | Primary Analysis |
| | Phase 1 (Dose Escalation) |
| | The safety analysis consists of listings and summaries of AEs (frequency tables |
| | based on CTCAE grades), summaries of laboratory abnormalities (CTCAE |
| | grade shift tables, low/normal/high shift tables based on laboratory normal |
| | ranges, and summary statistics tables) and flagging of notable values in listings, |
| | with results of other tests (e.g., ECG, vital signs) being listed and summarized. |
| | A 2-parameter BLRM guided by the EWOC principle is used for dose |
| | escalation of the IgA antibody therapy. Standardized doses are used such that 1 |
| | of the doses (d*) equals 1, e.g., doses are rescaled as d/d*. Consequently, α is |
| | equal to the odds of the probability of toxicity at d*. All information currently |
| | available about the dose-DLT relationship of IgA antibody is summarized in a |
| | prior distribution. For this study, this includes preclinical data about the starting |
| | dose and predicted MTD of IgA antibody within different animal species. This |
| | prior distribution is then updated after each cohort of subjects with all the DLT |
| | data available in the DDS from the current study. Once updated, the distribution |
| | summarizes the probability that the true rate of DLT for each dose lies in the |
| | following categories: |
| | 0% to <16%: under-dosing; |
| | ≥16% to <33%: targeted toxicity; and |
| | ≥33% to 100%: excessive toxicity. |
| | The frequency of DLTs is tabulated by dose for subjects in the dose escalation |
| | phase (Phase 1) (especially for Cycle 1 and subjects in the DDS as the primary |
| | endpoint tabulation) and information about all DLTs is listed by dose. |
| | Phase 2a (Expansion) |
| | Once the MTD and/or 1 or more RP2Ds is determined, enrollment of subjects |
| | into 1 of several expansion cohorts for selected tumor indications begin. |
| | The primary objective of the expansion phase is to determine preliminary |
| | efficacy as objective response (OR) using a Simon's two-stage design. The |
| | primary endpoint is based on the objective response assessed by local |
| | investigators according to RECIST v1.1. The primary analysis takes place once |
| | all subjects per cohort had at least 1 confirmed response assessment (i.e., ≥12 |
| | weeks post-baseline) or have been withdrawn from the study. |
| | Secondary Analyses |
| | Phase 1 (Dose Escalation) & Phase 2a (Expansion) |
| | Overall response as defined by achieving confirmed complete response (CR) |
| | and/or partial response (PR) is assessed by RECIST v1.1. DOR, PFS, and OS is |
| | summarized using descriptive statistics and Kaplan-Meier estimates. |
| | The PK analysis plan is described separately. Where feasible, non- |
| | compartmental analysis is conducted using concentration-time data of IgA |
| | antibody. Summary statistics of PK parameters such as area under the |
| | concentration-time curve over the dose interval, maximum plasma |
| | concentration, time to maximum plasma concentration, and minimum plasma |
| | concentration, are reported by dose group. Additional parameters or model- |
| | based analysis is, optionally, calculated depending on the available data. |
| | Due to sparse data sampling, an exploratory population PK analysis is, |
| | optionally, conducted to provide more comprehensive PK parameters for these |
| | subjects in the expansion cohorts. If a population PK analysis is conducted, all |
| | available clinical PK data from IgA antibody is used. |
| | Immunogenicity parameters are summarized by descriptive statistics. |
| | Exploratory analyses is summarized by descriptive or summary statistics. |
| | Potential relationships between dose, safety, PK, and exploratory PD endpoints |
| | are examined. |
| | Interim Analyses |
| | Phase 1 (Dose Escalation) |
| | An interim analysis (IA) is conducted at each dose escalation step. The BLRM |
| | is updated with the respective number of subjects treated and the number of |
| | DLTs observed in the last cohort. The updated model is then given a statistical |
| | recommendation for the next escalation step. In addition, a risk-benefit |
| | assessment that includes a comprehensive analysis of safety and available |
| | clinical information are done to decide on the next escalation steps. |
| | In the event of a CTCAE Grade 5 AE or a second CTCAE Grade 4 AE at least |
| | possibly related to IgA antibody at any time during Phase 1, the Sponsor will |
| | suspend further enrollment and an interim safety analysis will be done. The |
| | interim safety analysis will be reviewed by an ad-hoc SRC meeting to decide on |
| | further progression of the study. |
| | At least 1 IA (end of Phase I) is performed after all subjects treated in Phase 1 |
| | have completed their first post-baseline disease assessment and its confirmation |
| | (2^{nd} postbaseline assessment, i.e., ≥12 weeks post-baseline) or have withdrawn |
| | from the study. In case several RP2Ds or MTD are determined, additional IAs |
| | are, optionally, performed after the respective dose level. No formal interim |
| | report is written. All subjects still ongoing at this timepoint continue until |
| | disease progression, intolerable toxicity, investigator discretion, or subject's |
| | withdrawal of consent and all assessments is included in final analysis at the |
| | end of study. |
| | i)Phase 2a (Expansion) |
| | An IA, without a formal IA report, is performed for each cohort independently, |
| | once the defined number of subjects (i.e., 10 subjects) in the respective cohort |
| | have completed a confirmed disease assessment or have withdrawn from the |
| | study. Recruitment is not stopped in the respective cohort until the IA is |
| | completed. In case of a negative result, the respective cohort is, optionally, |
| | stopped for futility. The results of the IA are non-binding. Safety criteria is also |
| | evaluated to guide the decision to continue the study cohorts. |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

What is claimed is:

1. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein:
(i) the VH region comprises:
(I) a VH complementary determining region 1 (CDR-H1) comprising any one of amino acid sequences of SEQ ID NO: 34-54, or a variant thereof comprising 1 to 3 substitutions, deletions or insertions,
(II) a VH complementary determining region 2 (CDR-H2) comprising any one of amino acid sequences of SEQ ID NO: 57-78, or a variant thereof comprising 1 to 3 substitutions, deletions or insertions, and
(III) a VH complementary determining region 3 (CDR-H3) comprising any one of amino acid sequences of SEQ ID NO: 81-102, or a variant thereof comprising 1 to 3 substitutions, deletions or insertions,
(ii) the VL region comprises:
(I) a VL complementary determining region 1 (CDR-L1) comprising any one of amino acid sequences of SEQ ID NO: 105-126, or a variant thereof comprising 1 to 3 substitutions, deletions or insertions,
(II) a VL complementary determining region 2 (CDR-L2) comprising any one of amino acid sequences of SEQ ID NO: 129-143, or a variant thereof comprising 1 to 3 substitutions, deletions or insertions, and
(III) a VL complementary determining region 3 (CDR-L3) comprising any one of amino acid sequences of SEQ ID NO: 146-166, or a variant thereof comprising 1 to 3 substitutions, deletions or insertions, and
(b) an immunoglobulin A (IgA) heavy chain constant region that comprises at least one mutation, relative to a wild-type IgA heavy chain constant region having an amino acid sequence of SEQ ID NO: 1, wherein the mutation results in one or more of: reduced glycosylation, reduced dimerization, reduced aggregation, improved thermal stability, or increased circulatory half-life, each relative to a corresponding antibody that comprises the wild-type IgA heavy chain constant region.

2. The engineered antibody of claim 1, wherein:
(a) the CDR-H1 comprises the amino acid sequence of any one of SEQ ID NO: 34-54;
(b) the CDR-H2 comprises the amino acid sequence of any one of SEQ ID NO: 57-78;
(c) the CDR-H3 comprises the amino acid sequence of any one of SEQ ID NO: 81-102;
(d) the CDR-L1 comprises the amino acid sequence of any one of SEQ ID NO: 105-126;
(e) the CDR-L2 comprises the amino acid sequence of any one of SEQ ID NO: 129-143; and
(f) the CDR-L3 comprises the amino acid sequence of any one of SEQ ID NO: 146-166.

3. The engineered antibody of claim 1, wherein the VH region comprises an amino acid sequence that is at least 80% identical to any one of the VH region amino acid sequences recited in TABLE 5.

4. The engineered antibody of claim 1, wherein the VL region comprises an amino acid sequence that is at least 80% identical to any one of the VL region amino acid sequences recited in TABLE 7.

5. The engineered antibody of claim 1, wherein the VH region amino acid sequence and the VL region amino acid sequence are according to any one of combinations provided in TABLE 8.

6. The engineered antibody of claim 1, wherein the engineered antibody comprises an IgA light chain constant region that comprises an amino acid sequence that is at least 80% identical to SEQ ID NO: 23.

7. The engineered antibody of claim 1, wherein the IgA heavy chain constant region comprises an IgA CH1 domain, an IgA CH2 domain, and an IgA CH3 domain.

8. The engineered antibody of claim 7, wherein the at least one mutation is present in the IgA CH1 region and is an N45.2 substitution, a P124 substitution, or a combination thereof, numbering according to IMGT scheme, each relative to a corresponding antibody that comprises the wild-type IgA heavy chain constant region of SEQ ID NO: 1.

9. The engineered antibody of claim 8, wherein the at least one mutation is:
(a) an N45.2 substitution selected from the group consisting of: N45.2G and N45.2A;
(b) a P124R substitution; or
(c) any combination thereof,
numbering according to IMGT scheme, each relative to a corresponding antibody that comprises the wild-type IgA heavy chain constant region of SEQ ID NO: 1.

10. The engineered antibody of claim 7, wherein the at least one mutation is present in the IgA CH2 region and is:
(a) an N20 substitution;
(b) an L21 substitution;
(c) a T22 substitution;
(d) a C92 substitution;
(e) an N120 substitution;
(f) an I121 substitution; or
(g) a T122 substitution,
numbering according to IMGT scheme, each relative to a corresponding antibody that comprises the wild-type IgA heavy chain constant region of SEQ ID NO: 1.

11. The engineered antibody of claim 10, wherein the at least one mutation is:
(a) an N20 substitution selected from the group consisting of: N20G, N20Q, and N20T;
(b) an L21I substitution;
(c) a T22S substitution;
(d) a C92S substitution;
(e) an N120T substitution;
(f) an I121L substitution;
(g) a T122S substitution; or
(h) any combination thereof,
numbering according to IMGT scheme, each relative to a corresponding antibody that comprises the wild-type IgA heavy chain constant region of SEQ ID NO: 1.

12. The engineered antibody of claim 7, wherein the at least one mutation is present in the IgA CH3 region and is:
(a) an H5 substitution;
(b) an L7 substitution;
(c) a P10 substitution;
(d) a T22 substitution;
(e) an L79 substitution;
(f) a W81 substitution;
(g) an A85.1 substitution;
(h) a T86 substitution;
(i) a 188 substitution;
(j) an N135 substitution;
(k) a C147 deletion;
(l) a Y148 deletion;
(m) a deletion of P131-Y148; or
(n) a combination thereof,
numbering according to IMGT scheme, relative to the corresponding residue in the wild-type IgA heavy chain constant region of SEQ ID NO: 1.

13. The engineered antibody of claim 12, wherein the at least one mutation is:
(a) an H5 substitution selected from the group consisting of: HSC, H5Y, HSF, H5M and H5W;
(b) an L7 substitution selected from the group consisting of: L7F, L7Y, L7M, L7W, L7H and L71;
(c) a P10C substitution;
(d) a T22 substitution selected from the group consisting of: T22V, T22I, T22L and T22A;
(e) an L79 substitution selected from the group consisting of: L79V, L79T, L79A and L791;
(f) a W81 substitution selected from the group consisting of: W81T, W81L, W81A, W81V and W81I;
(g) an A85.1 substitution selected from the group consisting of: A85.1F, A85.1Y, A85.1M, A85.1W and A85.1H;
(h) a T86 substitution selected from the group consisting of: T86Y, T86F, T86M, T86W and T86H;
(i) a 188 substitution selected from the group consisting of: I88L, 188A, 188V and 188T; or
(j) any combination thereof,
numbering according to IMGT scheme, relative to the corresponding residue in the wild-type IgA heavy chain constant region of SEQ ID NO: 1.

14. The engineered antibody of claim 12, wherein the at least one mutation is:
(a) an N135Q substitution;
(b) a C147 deletion;
(c) a Y148 deletion; or
(d) any combination thereof,
numbering according to IMGT scheme, relative to the corresponding residue in the wild-type IgA heavy chain constant region of SEQ ID NO: 1.

15. The engineered antibody of claim 12, wherein the at least one mutation is a deletion of P131-Y148, numbering according to IMGT scheme, relative to the corresponding residue in the wild-type IgA heavy chain constant region of SEQ ID NO: 1.

16. The engineered antibody of claim 1, wherein the IgA heavy chain constant region comprises an amino acid sequence that is at least 80% identical to the IgA heavy chain constant region of SEQ ID NO: 5.

17. The engineered antibody of claim 1, wherein the engineered antibody is monomeric.

18. The engineered antibody of claim 1, wherein the EGFR binding domain binds to an EGFR polypeptide variant, wherein the EGFR variant comprises EGFRvIII, exon 19 deletions, L858R substitutions in exon 21, a C797S substitution, or a T790M substitution.

19. The engineered antibody of claim 1, wherein the VH region comprises an amino acid sequence of SEQ ID NO: 173, and the VL region comprises an amino acid sequence of SEQ ID NO: 211.

20. The engineered antibody of claim 1, wherein the VH region comprises an amino acid sequence of SEQ ID NO: 172, and the VL region comprises an amino acid sequence of SEQ ID NO: 198.

21. The engineered antibody of claim 1, wherein the VH region comprises an amino acid sequence of SEQ ID NO: 182, and the VL region comprises an amino acid sequence of SEQ ID NO: 207.

22. The engineered antibody of claim 1, wherein the VH region comprises an amino acid sequence of SEQ ID NO: 184, and the VL region comprises an amino acid sequence of SEQ ID NO: 209.

23. The engineered antibody of claim 1, wherein the engineered antibody is capable of inducing antibody dependent cell cytotoxicity (ADCC) via an immune effector cell.

24. The engineered antibody of claim 23, wherein the immune effector cell is a neutrophil, a T cell, an eosinophil, or a macrophage.

25. The engineered antibody of claim 1, wherein the engineered antibody is a chimeric antibody, a single chain antibody, a humanized antibody, a human antibody, a monoclonal antibody, a deimmunized antibody, a bispecific antibody, a multispecific antibody, a multivalent antibody, or a combination thereof.

26. The engineered antibody of claim 25, wherein the engineered antibody is a bispecific antibody.

27. The engineered antibody of claim 26, wherein the engineered antibody further comprises a binding domain that binds to a polypeptide antigen selected from the group consisting of: MET, cMet, CD28, HER2, HER3, IGF-IR, CD3, PD1, PD-L1, VEGFR2, CD47, FcGR3, and 4-1BB.

28. An engineered epidermal growth factor receptor (EGFR) binding antibody or a functional EGFR binding fragment thereof that comprises:
(a) an EGFR binding domain that binds to domain III of an EGFR polypeptide or a variant thereof; and
(b) an immunoglobulin A (IgA) constant region that comprises an IgA heavy chain constant region having at least one mutation, relative to a wild-type IgA heavy chain constant region having an amino acid sequence of SEQ ID NO: 1, wherein the mutation results in one or more of: reduced glycosylation, reduced aggregation, improved thermal stability, or increased circulatory half-life, each relative to a corresponding antibody that comprises the wild-type IgA heavy chain constant region.

29. The engineered antibody of claim 28, wherein the IgA constant region comprises:
(a) an IgA heavy chain constant region having an amino acid sequence of SEQ ID NO: 5; and
(b) an IgA light chain constant domain having an amino acid sequence of SEQ ID NO: 23.

30. The engineered antibody of claim 28, wherein the IgA constant domain comprises an IgA heavy chain constant region that comprises an IgA CH1, CH2, and CH3 domain, wherein the IgA heavy chain constant region comprises the following mutations:
(a) an N45.2G substitution in the CH1 domain;
(b) a P124R substitution in the CH1 domain;
(c) a C92S substitution in the CH2 domain;
(d) an N120T substitution in the CH2 domain;
(e) an I121L substitution in the CH2 domain; and
(f) a T122S substitution in the CH2 domain,
(g) numbering according to IMGT scheme, each relative to a corresponding antibody that comprises the wild-type IgA heavy chain constant region of SEQ ID NO: 1.

31. The engineered antibody of claim 28, wherein the EGFR binding domain binds to epitope of the EGFR polypeptide or a variant thereof that comprises any one of the following EGFR amino acid residues: P349, F352, D355, P362, D355, Q384, P387, Q408, H409, F412, I438, K443, K465, I467, or S468.

32. An engineered epidermal growth factor receptor (EGFR) binding antibody or a functional EGFR binding fragment thereof that comprises:
(a) an EGFR binding domain that binds to domain II of an EGFR polypeptide or a variant thereof; and
(b) an immunoglobulin A (IgA) constant domain that comprises an IgA heavy chain constant region having at least one mutation, relative to a wild-type IgA heavy chain constant region having an amino acid sequence of SEQ ID NO: 1, wherein the mutation results in one or more of: reduced glycosylation, reduced aggregation, improved thermal stability, or increased circulatory half-life, each relative to a corresponding antibody that comprises the wild-type IgA heavy chain constant region.

33. The engineered antibody of claim 32, wherein the IgA constant region comprises:
(a) an IgA heavy chain constant region having an amino acid sequence of SEQ ID NO: 5; and
(b) an IgA light chain constant domain having an amino acid sequence of SEQ ID NO: 23.

34. The engineered antibody of claim 32, wherein the IgA constant region comprises an IgA heavy chain constant region that comprises an IgA CH1, CH2, and CH3 domain, wherein the IgA heavy chain constant region comprises the following mutations:
(a) an N45.2G substitution in the CH1 domain;
(b) a P124R substitution in the CH1 domain;
(c) a C92S substitution in the CH2 domain;
(d) an N120T substitution in the CH2 domain;
(e) an I121L substitution in the CH2 domain; and
(f) a T122S substitution in the CH2 domain,
numbering according to IMGT scheme, each relative to a corresponding antibody that comprises the wild-type IgA heavy chain constant region of SEQ ID NO: 1.

35. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein:
(i) the VH region comprises:
(I) a VH complementary determining region 1 (CDR-H1) comprising any one of amino acid sequences of SEQ ID NO: 34-54, or a variant thereof comprising 1 to 3 substitutions, deletions or insertions,
(II) a VH complementary determining region 2 (CDR-H2) comprising any one of amino acid sequences of SEQ ID NO: 57-78, or a variant thereof comprising 1 to 3 substitutions, deletions or insertions, and
(III) a VH complementary determining region 3 (CDR-H3) comprising any one of amino acid sequences of SEQ ID NO: 81-102, or a variant thereof comprising 1 to 3 substitutions, deletions or insertions,
(ii) the VL region comprises:
(I) a VL complementary determining region 1 (CDR-L1) comprising any one of amino acid sequences of SEQ ID NO: 105-126, or a variant thereof comprising 1 to 3 substitutions, deletions or insertions,
(II) a VL complementary determining region 2 (CDR-L2) comprising any one of amino acid sequences of SEQ ID NO: 129-143, or a variant thereof comprising 1 to 3 substitutions, deletions or insertions, and
(III) a VL complementary determining region 3 (CDR-L3) comprising any one of amino acid sequences of SEQ ID NO: 146-166, or a variant thereof comprising 1 to 3 substitutions, deletions or insertions; and
(b) an immunoglobulin A (IgA) constant domain that comprises:
(i) a heavy chain constant region having an amino acid sequence of SEQ ID NO: 5; and
(ii) a light chain constant domain having an amino acid sequence of SEQ ID NO: 23.

36. The engineered antibody of claim 35, wherein the VH region comprises an amino acid sequence of SEQ ID NO: 173, and the VL region comprises an amino acid sequence of SEQ ID NO: 211.

37. The engineered antibody of claim 35, wherein the VH region comprises an amino acid sequence of SEQ ID NO: 172, and the VL region comprises an amino acid sequence of SEQ ID NO: 198.

38. The engineered antibody of claim 35, wherein the VH region comprises an amino acid sequence of SEQ ID NO: 182, and the VL region comprises an amino acid sequence of SEQ ID NO: 207.

39. The engineered antibody of claim 35, wherein the VH region comprises an amino acid sequence of SEQ ID NO: 184, and the VL region comprises an amino acid sequence of SEQ ID NO: 209.

40. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein:
(i) the VH region comprises:
(I) a VH complementary determining region 1 (CDR-H1) comprising any one of amino acid sequences of SEQ ID NO: 34-54, or a variant thereof comprising 1 to 3 substitutions, deletions or insertions,
(II) a VH complementary determining region 2 (CDR-H2) comprising any one of amino acid sequences of SEQ ID NO: 57-78, or a variant thereof comprising 1 to 3 substitutions, deletions or insertions, and
(III) a VH complementary determining region 3 (CDR-H3) comprising any one of amino acid sequences of SEQ ID NO: 81-102, or a variant thereof comprising 1 to 3 substitutions, deletions or insertions,
(ii) the VL region comprises:
(I) a VL complementary determining region 1 (CDR-L1) comprising any one of amino acid sequences of SEQ ID NO: 105-126, or a variant thereof comprising 1 to 3 substitutions, deletions or insertions,
(II) a VL complementary determining region 2 (CDR-L2) comprising any one of amino acid sequences of SEQ ID NO: 129-143, or a variant thereof comprising 1 to 3 substitutions, deletions or insertions, and
(III) a VL complementary determining region 3 (CDR-L3) comprising any one of amino acid sequences of SEQ ID NO: 146-166, or a variant thereof comprising 1 to 3 substitutions, deletions or insertions, and
(b) an immunoglobulin A (IgA) heavy chain constant region that comprises an IgA CH1, CH2, and CH3 domain, wherein the IgA heavy chain constant region comprises the following mutations:
(i) an N45.2G substitution in the CH1 domain,
(ii) a P124R substitution in the CH1 domain,
(iii) a C92S substitution in the CH2 domain,
(iv) an N120T substitution in the CH2 domain,
(v) an I121L substitution in the CH2 domain, and
(vi) a T122S substitution in the CH2 domain,
numbering according to IMGT scheme, each relative to a corresponding antibody that comprises the wild-type IgA heavy chain constant region of SEQ ID NO: 1.

41. The engineered antibody of claim 40, wherein the IgA heavy chain constant region further comprises the following mutations in the CH3 domain:
(a) an N135Q substitution;
(b) a C147 deletion; and
(c) a Y148 deletion,
numbering according to IMGT scheme, each relative to a corresponding antibody that comprises the wild-type IgA heavy chain constant region of SEQ ID NO: 1.

42. The engineered antibody of claim 40, wherein the IgA heavy chain constant region further comprises a deletion of P131-Y148 in the CH3 domain, numbering according to IMGT scheme, relative to the corresponding residue in the wild-type IgA heavy chain constant region of SEQ ID NO: 1.

43. The engineered antibody of claim 42, wherein the IgA heavy chain constant region further comprises the following mutations in the CH2 domain:
(a) an N20 substitution selected from the group consisting of: N20G, N20Q, and N20T;
(b) an L211 substitution; and
(c) a T22S substitution,
numbering according to IMGT scheme, each relative to a corresponding antibody that comprises the wild-type IgA heavy chain constant region of SEQ ID NO: 1.

44. The engineered antibody of any one of claims 40-43, wherein the VH region comprises an amino acid sequence of SEQ ID NO: 173, and the VL region comprises an amino acid sequence of SEQ ID NO: 211.

45. The engineered antibody of any one of claims 40-43, wherein the VH region comprises an amino acid sequence of SEQ ID NO: 172, and the VL region comprises an amino acid sequence of SEQ ID NO: 198.

46. The engineered antibody of any one of claims 40-43, wherein the VH region comprises an amino acid sequence of SEQ ID NO: 182, and the VL region comprises an amino acid sequence of SEQ ID NO: 207.

47. The engineered antibody of any one of claims 40-43, wherein the VH region comprises an amino acid sequence of SEQ ID NO: 184, and the VL region comprises an amino acid sequence of SEQ ID NO: 209.

48. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 173 and the VL region comprises an amino acid sequence of SEQ ID NO: 211; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

49. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 172 and the VL region comprises an amino acid sequence of SEQ ID NO: 198; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

50. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 182 and the VL region comprises an amino acid sequence of SEQ ID NO: 207; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

51. An engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 184 and the VL region comprises an amino acid sequence of SEQ ID NO: 209; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

52. A pharmaceutical composition comprising the engineered antibody of any one of the claims 1-51, and a pharmaceutically acceptable carrier.

53. A method of treating a cancer in a subject in need thereof, the method comprising administering to the subject an effective amount of an engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein:
(i) the VH region comprises:
(I) a VH complementary determining region 1 (CDR-H1) comprising any one of amino acid sequences of SEQ ID NO: 34-54, or a variant thereof comprising 1 to 3 substitutions, deletions or insertions,
(II) a VH complementary determining region 2 (CDR-H2) comprising any one of amino acid sequences of SEQ ID NO: 57-78, or a variant thereof comprising 1 to 3 substitutions, deletions or insertions, and
(III) a VH complementary determining region 3 (CDR-H3) comprising any one of amino acid sequences of SEQ ID NO: 81-102, or a variant thereof comprising 1 to 3 substitutions, deletions or insertions,
(ii) the VL region comprises:
(I) a VL complementary determining region 1 (CDR-L1) comprising any one of amino acid sequences of SEQ ID NO: 105-126, or a variant thereof comprising 1 to 3 substitutions, deletions or insertions,
(II) a VL complementary determining region 2 (CDR-L2) comprising any one of amino acid sequences of SEQ ID NO: 129-143, or a variant thereof comprising 1 to 3 substitutions, deletions or insertions, and
(III) a VL complementary determining region 3 (CDR-L3) comprising any one of amino acid sequences of SEQ ID NO: 146-166, or a variant thereof comprising 1 to 3 substitutions, deletions or insertions; and
(b) an immunoglobulin A (IgA) heavy chain constant region that comprises at least one mutation, relative to a wild-type IgA heavy chain constant region having an amino acid sequence of SEQ ID NO: 1, wherein the mutation results in one or more of: reduced glycosylation, reduced aggregation, improved thermal stability, or increased circulatory half-life, each relative to a corresponding antibody that comprises the wild-type IgA heavy chain constant region.

54. The method of claim 53, wherein the IgA heavy chain constant region comprises an IgA CH1, CH2, and CH3 domain, wherein the IgA heavy chain constant region comprises the following mutations:
(a) an N45.2G substitution in the CH1 domain;
(b) a P124R substitution in the CH1 domain;
(c) a C92S substitution in the CH2 domain;
(d) an N120T substitution in the CH2 domain;
(e) an I121L substitution in the CH2 domain; and
(f) a T122S substitution in the CH2 domain,
numbering according to IMGT scheme, each relative to a corresponding antibody that comprises the corresponding residue in the wild-type IgA heavy chain constant region of SEQ ID NO: 1.

55. The method of claim 53, wherein the IgA heavy chain constant region further comprises the following mutations in the CH3 domain:
(a) an N135Q substitution;
(b) a C147 deletion; and
(c) a Y148 deletion,
numbering according to IMGT scheme, each relative to a corresponding antibody that comprises the wild-type IgA heavy chain constant region of SEQ ID NO: 1.

56. The method of claim 54, wherein the IgA heavy chain constant region further comprises a deletion of P131-Y148 in the CH3 domain, numbering according to IMGT scheme, relative to the corresponding residue in the wild-type IgA heavy chain constant region of SEQ ID NO: 1.

57. The method of claim 56, wherein the IgA heavy chain constant region further comprises the following mutations in the CH2 domain:
(a) an N20 substitution selected from the group consisting of: N20G, N20Q, and N20T;
(b) an L211 substitution; and
(c) a T22S substitution,
numbering according to IMGT scheme, each relative to a corresponding antibody that comprises the wild-type IgA heavy chain constant region of SEQ ID NO: 1.

58. The method of claim 53, wherein the cancer is a solid tumor cancer.

59. The method of claim 53, wherein the cancer is selected from the group consisting of: lung cancer, head and neck cancer, colon cancer, rectal cancer, pancreatic cancer, breast cancer, ovarian cancer, bladder cancer, kidney cancer, mesothelioma and a glioblastoma.

60. The method of claim 53, wherein the cancer is an adenocarcinoma, a squamous cell carcinoma, or a large-cell carcinoma.

61. The method of claim 53, wherein the cancer is colorectal cancer.

62. The method of claim 53, wherein the cancer is head and neck squamous cell carcinoma.

63. The method of claim 53, wherein the cancer is non-small cell lung cancer.

64. The method of claim 53, wherein the engineered antibody inhibits tumor growth associated with the cancer.

65. The method of claim 53, wherein the administering is subcutaneous, intravenous, intradermal, intraperitoneal, oral, intramuscular, or intracranial.

66. The method of claim 53, wherein the engineered antibody is administered to the subject in combination with a second therapeutic agent.

67. The method of claim 66, wherein the second therapeutic agent comprises an anti-cancer agent, a chemotherapeutic agent, radiation therapy, a cytotoxic agent, a corticosteroid, an immunotherapy agent, a dietary supplement, or an antioxidant.

68. The method of claim 53, wherein the second therapeutic agent is administered prior to, concurrently, or after administering the engineered antibody.

69. The method of claim 53, wherein the subject is a rodent, a non-human primate or a human.

70. The method of claim 69, wherein the subject is a rodent, and wherein the effective amount is administered at a dosage of from 1 mg/kg to 25 mg/kg administered subcutaneously, intravenously or intraperitoneally twice weekly for 35-40 days.

71. The method of claim 69, wherein the effective amount is administered intravenously every seven days for 5 weeks at a dosage of 25 mg/kg except for the third dose, which is delivered at 12.5 mg/kg.

72. The method of claim 69, wherein the effective amount is administered intravenously at a dosage of from 1 mg/kg to 25 mg/kg twice weekly.

73. A method of treating a cancer in a subject in need thereof, the method comprising administering to the subject an effective amount of an engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 173 and the VL region comprises an amino acid sequence of SEQ ID NO: 211; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

74. A method of treating a cancer in a subject in need thereof, the method comprising administering to the subject an effective amount of an engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 172 and the VL region comprises an amino acid sequence of SEQ ID NO: 198; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

75. A method of treating a cancer in a subject in need thereof, the method comprising administering to the subject an effective amount of an engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 182 and the VL region comprises an amino acid sequence of SEQ ID NO: 207; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

76. A method of treating a cancer in a subject in need thereof, the method comprising administering to the subject an effective amount of an engineered epidermal growth factor receptor (EGFR) binding antibody or a functional EGFR binding fragment thereof that comprises:
(a) an EGFR binding domain that comprises a heavy chain variable (VH) region and a light chain variable (VL) region, wherein the VH region comprises an amino acid sequence of SEQ ID NO: 184 and the VL region comprises an amino acid sequence of SEQ ID NO: 209; and
(b) an immunoglobulin A (IgA) constant domain that comprises a heavy chain constant region having an amino acid sequence of SEQ ID NO: 5 and a light chain constant domain having an amino acid sequence of SEQ ID NO: 23.

77. The method of any one of claims 73-76, wherein the cancer is colorectal cancer, head and neck squamous cell carcinoma, non-small cell lung cancer, or a combination thereof.

78. An isolated nucleic acid encoding the engineered antibody of any one of the claims 1-51.

79. A host cell expressing the engineered antibody of any one of the claims 1-51.

80. A composition comprising a first therapeutic agent and a second therapeutic agent, wherein the first therapeutic agent comprises the engineered antibody of any one of the claims 1-51 and the second therapeutic agent binds to MET, cMet, CD28, HER2, HER3, IGF-IR, CD3, PD1, PD-L1, VEGFR2, FcGR3, 4-1BB, or a combination thereof.
